(19) United States
(12) Patent Application Publication
(10) Pub. No.: US 2026/0354015 A1
(43) Pub. Date: Nov. 5, 2026

(54) COMPOUND FOR ORGANIC ELECTROLUMINESCENT DEVICE AND ORGANIC ELECTROLUMINESCENT DEVICE COMPRISING SAME

(71) Applicant: SHIJIAZHUANG CHENGZHI YONGHUA DISPLAY MATERIAL CO., LTD., Shijiazhuang (CN)

(72) Inventors: Hualong DING, Shijiazhuang (CN); Hao ZHANG, Shijiazhuang (CN); Jianchao WANG, Shijiazhuang (CN); Guanghui ZHANG, Shijiazhuang (CN)

(73) Assignee: SHIJIAZHUANG CHENGZHI YONGHUA DISPLAY MATERIAL CO., LTD., Shijiazhuang (CN)

(21) Appl. No.: 19/346,340

(22) Filed: Sep. 30, 2025

(30) Foreign Application Priority Data
Apr. 18, 2025 (CN) 202510497069.8

(51) Int. Cl. H10K 85/60; C07D 498/04; H10K 50/15; H10K 50/17

(52) U.S. Cl. CPC H10K 85/657; C07D 498/04; H10K 85/654; H10K 85/6574; H10K 50/15; H10K 50/17

(57) ABSTRACT

The present invention provides a compound for an organic electroluminescent device and an organic electroluminescent device comprising the compound. The general structural formula of the compound is as represented by formula I, wherein at least one of Ar¹ and Ar² has a structure represented by formula II. The structure of formula II has a unique double-X five-membered ring structure. This unique five-membered ring structure has good thermal stability and electron-withdrawing ability and can not only enhance the sublimation yield of a material, but can also improve the hole injection ability in a hole injection layer during the preparation of an organic electroluminescent device, thereby reducing the voltage of the device and increasing the efficiency and lifetime of the device.

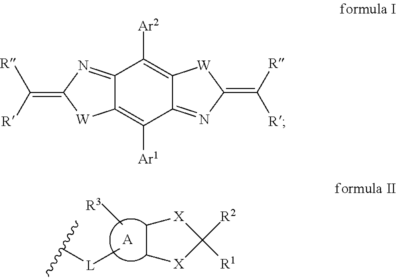

### TECHNICAL FIELD

The present invention belongs to the technical field of OLEDs and in particular comprises a compound for an organic electroluminescent device and an organic electroluminescent device comprising the compound.

### BACKGROUND ART

Organic electronic devices include, but are not limited to, the following categories: organic light-emitting diodes (OLEDs), organic field effect transistors (O-FETs), organic light-emitting transistors (OLETs), organic photovoltaic devices (OPVs), dye-sensitized solar cells (DSSCs), organic optical detectors, organic photoreceptors, organic field effect devices (OFQDs), light-emitting electrochemical cells (LECs), organic laser diodes, and organic electroluminescent devices.

In 1987, Tang and Van Slyke of Eastman Kodak reported a two-layer organic electroluminescent device, comprising an arylamine hole transport layer and a tris(8-hydroxyquinoline)aluminum layer as an electron transport layer and a light-emitting layer (Applied Physics Letters, 1987, 51(12):913-915). Once a bias voltage is applied to the device, green light is emitted from the device. This invention laid the foundation for the development of modern organic light-emitting diodes (OLEDs). The most advanced OLEDs may comprise a plurality of layers, e.g., charge injection and transport layers, charge and exciton barrier layers, and one or more light-emitting layers between a cathode and an anode. Since OLEDs are self-luminous solid-state devices, they provide great potential for display and lighting applications. In addition, the inherent characteristics of organic materials, such as their flexibility, can make them very suitable for special applications, such as the manufacture of flexible substrates.

OLEDs can be divided into three different types according to their luminous mechanisms. The OLED invented by Tang and Van Slyke is a fluorescent OLED, which only uses singlet state light emission, and the triplet state generated in the device is wasted via a non-radiation attenuation channel. Therefore, the internal quantum efficiency (IQE) of the fluorescent OLED is only 25%, which hinders the commercialization of OLEDs. In 1997, Forrest and Thompson reported a phosphorescent OLED, which utilizes triplet-state emission from a heavy metal-containing complex as a light emitter. Therefore, singlet and triplet states can be harvested and 100% IQE is achieved. Due to its high efficiency, the discovery and development of the phosphorescent OLED directly contribute to the commercialization of active matrix OLEDs (AMOLEDs). Recently, Adachi achieved high efficiency by the thermally activated delayed fluorescence (TADF) of organic compounds. These light emitters have small singlet-triplet gaps, which makes it possible for excitons to return from the triplet state to the singlet state. In TADF devices, triplet-state excitons can generate singlet-state excitons through reverse intersystem crossing, resulting in high IQE.

OLEDs can also be classified into small molecules and polymer OLEDs according to the form of the material used. The small molecule refers to any non-polymeric organic or organometallic material. The molecular weight of the small molecule can be very large, as long as it has an accurate structure. Dendritic polymers with well-defined structures are considered as small molecules. Polymer OLEDs include conjugated polymers and non-conjugated polymers with pendant luminescent groups. If post-polymerization occurs during the manufacturing process, the small molecule OLEDs can become polymer OLEDs.

There have been various OLED manufacturing methods. Small molecule OLEDs are usually manufactured by vacuum thermal evaporation. Polymer OLEDs are manufactured by solution methods, such as spin coating, inkjet printing, and nozzle printing. If the material can be dissolved or dispersed in the solvent, small molecule OLEDs can also be manufactured by a solution method.

The luminescent colors of OLEDs can be realized by the structural design of luminescent materials. An OLED can comprise one or more light-emitting layers to achieve the desired spectrum. Green, yellow, and red OLEDs and phosphorescent materials have been successfully commercialized. Blue phosphorescent devices still have problems such as blue unsaturation, short device lifetime and high working voltage. Commercial full-color OLED displays typically employ a hybrid strategy, which utilizes blue fluorescence and phosphorescent yellow, or red and green. Currently, a rapid decrease in the efficiency of phosphorescent OLEDs at high brightness is still challenging. In addition, it is desirable to have a more saturated luminescence spectrum, higher efficiency and longer device lifetime.

p-Type doping materials (PD materials) are a class of materials that play a critically important role in OLED devices. These materials are mainly used in combination with hole transport materials to form hole injection layers or p-type charge generation layers. When using a PD material to prepare an OLED display panel, a typical process involves co-evaporation and thermal sublimation of the PD material and a hole transport material, followed by deposition onto a surface of a substrate to form a thin film, onto which other functional layers and light-emitting layers are then gradually evaporated. Therefore, excellent PD materials are required to have good thermal stability and withstand a long-term high-temperature environment during the evaporation process while ensuring the performance of the evaporated device. Good device performance also necessitates a superior hole injection layer. The PD material is required to have a relatively low LUMO energy level to achieve good matching with the HOMO energy level of the hole transport material, thereby enhancing the hole injection performance, so that the PD material is further required to contain various electron-withdrawing groups. Therefore, studying PD materials with various substituents to find PD materials exhibiting both superior thermal stability and superior doping capability is the key to the development of such materials.

### SUMMARY OF THE INVENTION

The present invention aims to provide a range of organic compounds having dehydrogenated fused-ring structures with specific substituents so as to solve at least some of the above-mentioned problems. These organic compounds can be used as charge transport materials, charge injection materials, or similar materials in organic electroluminescent devices. Due to the unique structural design, these novel organic compounds exhibit superior hole injection ability and relatively high thermal stability. These novel organic compounds can endow organic electroluminescent devices with excellent performance, such as reduced device voltage and improved device efficiency and lifetime.

To this end, a first aspect of the present invention provides a compound for an organic electroluminescent device. The general structural formula of the compound is as represented by formula I:

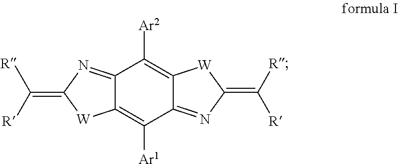

in formula I,
W is, at each occurrence, the same or different and selected from O, S, or Se;
Ar¹ and Ar² can be the same or different and each independently selected from the group consisting of any one of or any combination of some of hydrogen, deuterium, halogen, carbonyl, cyano, sulfinyl, sulfonyl, phosphoroso, a substituted or unsubstituted alkyl group with 1-20 carbon atoms, a substituted or unsubstituted cycloalkyl group with 3-20 ring carbon atoms, a substituted or unsubstituted alkoxy group with 1-20 ring carbon atoms, a substituted or unsubstituted aryloxy group with 6-30 carbon atoms, a substituted or unsubstituted aryl group with 6-30 carbon atoms, and a substituted or unsubstituted heteroaryl group with 3-30 carbon atoms; and at least one of Ar¹ and Ar² has a structure as represented by the following formula II,

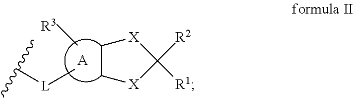

in formula II,
X is, at each occurrence, the same or different and selected from any one of or any combination of some of O, S, Se,

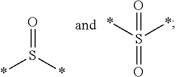

L is, at each occurrence, the same or different and selected from a single bond, O, a substituted or unsubstituted arylene group with 6-30 carbon atoms, a substituted or unsubstituted heteroarylene group with 3-30 carbon atoms, and a combination thereof,
R³ represents a substituent on ring A and, at each occurrence, represents monosubstitution, polysubstitution, or no substitution;
ring A is selected from any one of a substituted or unsubstituted aromatic ring with 6-30 carbon atoms and a substituted or unsubstituted heteroaromatic ring with 3-30 carbon atoms;
R¹, R², and R³ are, at each occurrence, each independently selected from the group consisting of any one of or any combination of some of hydrogen, deuterium, hydroxy, mercapto, halogen, nitroso, nitro, acyl, carbonyl, carboxy, an ester group, cyano, isocyano, SCN, OCN, SF₅, boranyl, sulfinyl, sulfonyl, phosphoroso, a substituted or unsubstituted alkyl group with 1-20 carbon atoms, a substituted or unsubstituted cycloalkyl group with 3-20 ring carbon atoms, a substituted or unsubstituted heteroalkyl group with 1-20 carbon atoms, a substituted or unsubstituted heterocyclic group with 3-20 ring atoms, a substituted or unsubstituted aralkyl group with 7-30 carbon atoms, a substituted or unsubstituted alkoxy group with 1-20 ring carbon atoms, a substituted or unsubstituted aryloxy group with 6-30 carbon atoms, a substituted or unsubstituted alkenyl group with 2-20 carbon atoms, a substituted or unsubstituted alkynyl group with 2-20 carbon atoms, a substituted or unsubstituted aryl group with 6-30 carbon atoms, a substituted or unsubstituted heteroaryl group with 3-30 carbon atoms, a substituted or unsubstituted alkylsilyl group with 3-20 carbon atoms, a substituted or unsubstituted arylsilyl group with 6-carbon atoms, a substituted or unsubstituted alkylgermyl group with 3-20 carbon atoms, a substituted or unsubstituted arylgermyl group with 6-20 carbon atoms, and a substituted or unsubstituted amino group with 0-20 carbon atoms;
R′ and R″ are each independently selected from the group consisting of any one of or any combination of some of hydroxy, mercapto, halogen, nitroso, nitro, acyl, carbonyl, carboxy, an ester group, cyano, isocyano, SCN, OCN, SF₅, sulfinyl, sulfonyl, phosphoroso, a substituted or unsubstituted alkyl group with 1-20 carbon atoms, a substituted or unsubstituted cycloalkyl group with 3-20 ring carbon atoms, a substituted or unsubstituted heteroalkyl group with 1-20 carbon atoms, a substituted or unsubstituted heterocyclic group with 3-20 ring atoms, a substituted or unsubstituted aralkyl group with 7-30 carbon atoms, a substituted or unsubstituted alkoxy group with 1-20 ring carbon atoms, a substituted or unsubstituted aryloxy group with 6-30 carbon atoms, a substituted or unsubstituted alkenyl group with 2-20 carbon atoms, a substituted or unsubstituted alkynyl group with 2-20 carbon atoms, a substituted or unsubstituted aryl group with 6-30 carbon atoms, and a substituted or unsubstituted heteroaryl group with 3-30 carbon atoms;
at least one of Ar¹, Ar², R′, and R″ is a group containing at least one electron-withdrawing group; and in Ar¹, Ar², R′, R″, R¹, R², R³, and L, any adjacent substituents can be bonded to each other to form a ring; and
[symbol] represents the position of connection of L to formula I, and * represents the position of connection of X to another group.

Furthermore, the compound has a structure as represented by formula I-1:

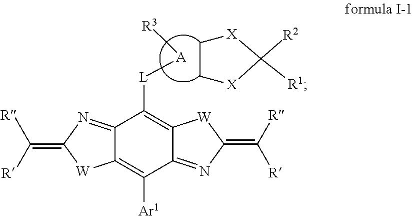

wherein
in formula I-1,
W is, at each occurrence, the same or different and is selected from O, S, or Se;
X is, at each occurrence, the same or different and is selected from any one of or any combination of some of O, S, Se, and

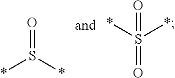

L is, at each occurrence, the same or different and is selected from a single bond, O, a substituted or unsubstituted arylene group with 6-30 carbon atoms, a substituted or unsubstituted heteroarylene group with 3-30 carbon atoms, and a combination thereof,
R³ represents a substituent on ring A and, at each occurrence, represents monosubstitution, polysubstitution, or no substitution;
ring A is selected from any one of a substituted or unsubstituted aromatic ring with 6-30 carbon atoms and a substituted or unsubstituted heteroaromatic ring with 3-30 carbon atoms;
Ar¹ is selected from the group consisting of any one of or any combination of some of hydrogen, deuterium, halogen, carbonyl, cyano, sulfinyl, sulfonyl, phosphoroso, a substituted or unsubstituted alkyl group with 1-20 carbon atoms, a substituted or unsubstituted cycloalkyl group with 3-20 ring carbon atoms, a substituted or unsubstituted alkoxy group with 1-20 ring carbon atoms, a substituted or unsubstituted aryloxy group with 6-30 carbon atoms, a substituted or unsubstituted aryl group with 6-30 carbon atoms, and a substituted or unsubstituted heteroaryl group with 3-30 carbon atoms;
R¹, R², and R³ are, at each occurrence, each independently selected from the group consisting of any one of or any combination of some of hydrogen, deuterium, hydroxy, mercapto, halogen, nitroso, nitro, acyl, carbonyl, carboxy, an ester group, cyano, isocyano, SCN, OCN, SF₅, boranyl, sulfinyl, sulfonyl, phosphoroso, a substituted or unsubstituted alkyl group with 1-20 carbon atoms, a substituted or unsubstituted cycloalkyl group with 3-20 ring carbon atoms, a substituted or unsubstituted heteroalkyl group with 1-20 carbon atoms, a substituted or unsubstituted heterocyclic group with 3-20 ring atoms, a substituted or unsubstituted aralkyl group with 7-30 carbon atoms, a substituted or unsubstituted alkoxy group with 1-20 ring carbon atoms, a substituted or unsubstituted aryloxy group with 6-30 carbon atoms, a substituted or unsubstituted alkenyl group with 2-20 carbon atoms, a substituted or unsubstituted alkynyl group with 2-20 carbon atoms, a substituted or unsubstituted aryl group with 6-30 carbon atoms, a substituted or unsubstituted heteroaryl group with 3-30 carbon atoms, a substituted or unsubstituted alkylsilyl group with 3-20 carbon atoms, a substituted or unsubstituted arylsilyl group with 6-20 carbon atoms, a substituted or unsubstituted alkylgermyl group with 3-20 carbon atoms, a substituted or unsubstituted arylgermyl group with 6-20 carbon atoms, and a substituted or unsubstituted amino group with 0-20 carbon atoms;
R′ and R″ are each independently selected from the group consisting of any one of or any combination of some of hydroxy, mercapto, halogen, nitroso, nitro, acyl, carbonyl, carboxy, an ester group, cyano, isocyano, SCN, OCN, SF₅, sulfinyl, sulfonyl, phosphoroso, a substituted or unsubstituted alkyl group with 1-20 carbon atoms, a substituted or unsubstituted cycloalkyl group with 3-20 ring carbon atoms, a substituted or unsubstituted heteroalkyl group with 1-20 carbon atoms, a substituted or unsubstituted heterocyclic group with 3-20 ring atoms, a substituted or unsubstituted aralkyl group with 7-30 carbon atoms, a substituted or unsubstituted alkoxy group with 1-20 ring carbon atoms, a substituted or unsubstituted aryloxy group with 6-30 carbon atoms, a substituted or unsubstituted alkenyl group with 2-20 carbon atoms, a substituted or unsubstituted alkynyl group with 2-20 carbon atoms, a substituted or unsubstituted aryl group with 6-30 carbon atoms, and a substituted or unsubstituted heteroaryl group with 3-30 carbon atoms;
wherein in R′, R″, R¹, R², R³, and L, any adjacent substituents can be bonded to each other to form a ring.

Furthermore, Ar¹ and Ar² are, at each occurrence, the same or different and selected from the group consisting of hydrogen, deuterium, halogen, cyano, a substituted or unsubstituted alkyl group with 1-20 carbon atoms, a substituted or unsubstituted alkoxy group with 1-20 ring carbon atoms, a substituted or unsubstituted aryl group with 6-30 carbon atoms, a substituted or unsubstituted heteroaryl group with 3-30 carbon atoms, and a combination thereof.

Furthermore, when Ar¹ and Ar² have substituents, the substituents in Ar¹ and Ar² comprise at least one electron-withdrawing group.

Furthermore, the compound has a structure as represented by formula I-2:

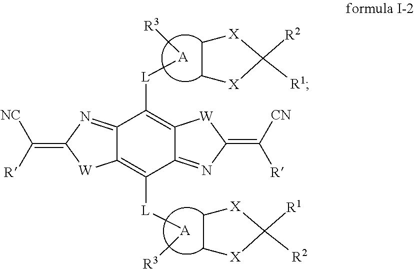

wherein
in formula I-2,
W is, at each occurrence, the same or different and selected from O, S, or Se;
X is, at each occurrence, the same or different and selected from any one of or any combination of some of O, S, Se,

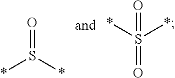

L is, at each occurrence, the same or different and selected from a single bond, O, a substituted or unsubstituted arylene group with 6-30 carbon atoms, a substituted or unsubstituted heteroarylene group with 3-30 carbon atoms, and a combination thereof,
R³ represents a substituent on ring A and, at each occurrence, represents monosubstitution, polysubstitution, or no substitution;
ring A is selected from any one of a substituted or unsubstituted aromatic ring with 6-30 carbon atoms and a substituted or unsubstituted heteroaromatic ring with 3-30 carbon atoms;
R¹, R², and R³ are, at each occurrence, each independently selected from the group consisting of any one of or any combination of some of hydrogen, deuterium, hydroxy, mercapto, halogen, nitroso, nitro, acyl, carbonyl, carboxy, an ester group, cyano, isocyano, SCN, OCN, SF₅, boranyl, sulfinyl, sulfonyl, phosphoroso, a substituted or unsubstituted alkyl group with 1-20 carbon atoms, a substituted or unsubstituted cycloalkyl group with 3-20 ring carbon atoms, a substituted or unsubstituted heteroalkyl group with 1-20 carbon atoms, a substituted or unsubstituted heterocyclic group with 3-20 ring atoms, a substituted or unsubstituted aralkyl group with 7-30 carbon atoms, a substituted or unsubstituted alkoxy group with 1-20 ring carbon atoms, a substituted or unsubstituted aryloxy group with 6-30 carbon atoms, a substituted or unsubstituted alkenyl group with 2-20 carbon atoms, a substituted or unsubstituted alkynyl group with 2-20 carbon atoms, a substituted or unsubstituted aryl group with 6-30 carbon atoms, a substituted or unsubstituted heteroaryl group with 3-30 carbon atoms, a substituted or unsubstituted alkylsilyl group with 3-20 carbon atoms, a substituted or unsubstituted arylsilyl group with 6-20 carbon atoms, a substituted or unsubstituted alkylgermyl group with 3-20 carbon atoms, a substituted or unsubstituted arylgermyl group with 6-20 carbon atoms, and a substituted or unsubstituted amino group with 0-20 carbon atoms;
R′ and R″ are each independently selected from the group consisting of any one of or any combination of some of hydroxy, mercapto, halogen, nitroso, nitro, acyl, carbonyl, carboxy, an ester group, cyano, isocyano, SCN, OCN, SF₅, sulfinyl, sulfonyl, phosphoroso, a substituted or unsubstituted alkyl group with 1-20 carbon atoms, a substituted or unsubstituted cycloalkyl group with 3-20 ring carbon atoms, a substituted or unsubstituted heteroalkyl group with 1-20 carbon atoms, a substituted or unsubstituted heterocyclic group with 3-20 ring atoms, a substituted or unsubstituted aralkyl group with 7-30 carbon atoms, a substituted or unsubstituted alkoxy group with 1-20 ring carbon atoms, a substituted or unsubstituted aryloxy group with 6-30 carbon atoms, a substituted or unsubstituted alkenyl group with 2-20 carbon atoms, a substituted or unsubstituted alkynyl group with 2-20 carbon atoms, a substituted or unsubstituted aryl group with 6-30 carbon atoms, and a substituted or unsubstituted heteroaryl group with 3-30 carbon atoms;
in R¹, R², R³, and L, any adjacent substituents can be bonded to each other to form a ring.

Furthermore, the structure represented by formula II is selected from one of structures represented by formulas II-1 to II-6:

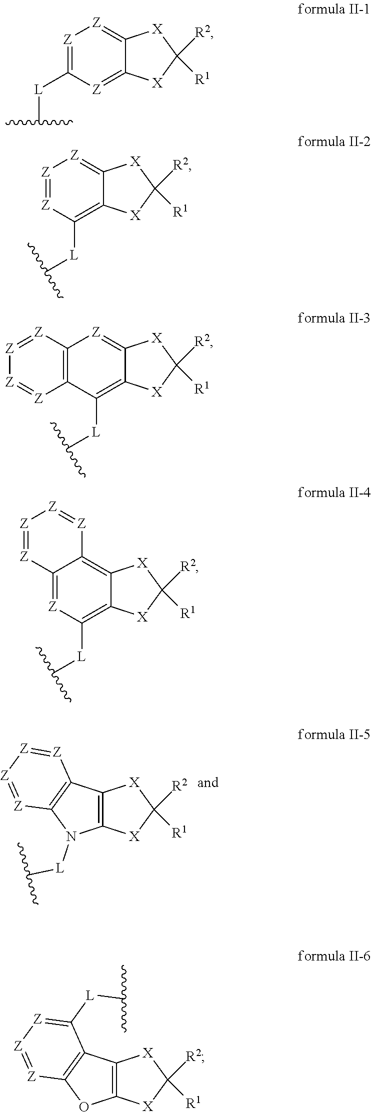

wherein
Z is, at each occurrence, the same or different and is selected from CR³ or N;
X is, at each occurrence, the same or different and is selected from any one of or any combination of some of O, S, Se,

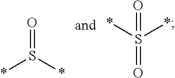

L is, at each occurrence, the same or different and is selected from a single bond, O, a substituted or unsubstituted arylene group with 6-30 carbon atoms, a substituted or unsubstituted heteroarylene group with 3-30 carbon atoms, and a combination thereof,
R¹, R², and R³ are, at each occurrence, the same or different and are selected from the group consisting of hydrogen, deuterium, hydroxy, mercapto, halogen, nitroso, nitro, acyl, carbonyl, carboxy, an ester group, cyano, isocyano, SCN, OCN, SF₅, boranyl, sulfinyl, sulfonyl, phosphoroso, a substituted or unsubstituted alkyl group with 1-20 carbon atoms, a substituted or unsubstituted cycloalkyl group with 3-20 ring carbon atoms, a substituted or unsubstituted heteroalkyl group with 1-20 carbon atoms, a substituted or unsubstituted heterocyclic group with 3-20 ring atoms, a substituted or unsubstituted aralkyl group with 7-30 carbon atoms, a substituted or unsubstituted alkoxy group with 1-20 ring carbon atoms, a substituted or unsubstituted aryloxy group with 6-30 carbon atoms, a substituted or unsubstituted alkenyl group with 2-20 carbon atoms, a substituted or unsubstituted alkynyl group with 2-20 carbon atoms, a substituted or unsubstituted aryl group with 6-30 carbon atoms, a substituted or unsubstituted heteroaryl group with 3-30 carbon atoms, a substituted or unsubstituted alkylsilyl group with 3-20 carbon atoms, a substituted or unsubstituted arylsilyl group with 6-20 carbon atoms, a substituted or unsubstituted alkylgermyl group with 3-20 carbon atoms, a substituted or unsubstituted arylgermyl group with 6-20 carbon atoms, a substituted or unsubstituted amino group with 0-20 carbon atoms, and a combination thereof,
in R¹, R², and R³, any adjacent substituents can be bonded to each other to form a ring; and
[symbol] represents the position of connection of L to formula I, and * represents the position of connection of X to another group.

Furthermore, the structure represented by formula II is selected from structures represented by formula II-1 or II-2.

Furthermore, R¹ and R² are, at each occurrence, the same or different and are selected from the group consisting of any one of or any combination of some of hydrogen, deuterium, halogen, nitroso, nitro, carbonyl, cyano, SF₅, sulfinyl, sulfonyl, phosphoroso, a substituted or unsubstituted alkyl group with 1-20 carbon atoms, a substituted or unsubstituted cycloalkyl group with 3-20 ring carbon atoms, a substituted or unsubstituted heteroalkyl group with 1-20 carbon atoms, a substituted or unsubstituted heterocyclic group with 3-20 ring atoms, a substituted or unsubstituted aralkyl group with 7-30 carbon atoms, a substituted or unsubstituted alkoxy group with 1-20 ring carbon atoms, a substituted or unsubstituted aryloxy group with 6-30 carbon atoms, a substituted or unsubstituted alkenyl group with 2-20 carbon atoms, a substituted or unsubstituted alkynyl group with 2-20 carbon atoms, a substituted or unsubstituted aryl group with 6-30 carbon atoms, and a substituted or unsubstituted heteroaryl group with 3-30 carbon atoms; and in R¹ and R², any adjacent substituents can be bonded to each other to form a ring.

Furthermore, W is selected from 0; and/or
R′ and R″ are, at each occurrence, the same or different and are selected from the group consisting of any one of or any combination of some of fluoro, cyano, SCN, sulfonyl, a substituted or unsubstituted alkyl group with 1-20 carbon atoms, a substituted or unsubstituted cycloalkyl group with 3-20 ring carbon atoms, a substituted or unsubstituted heteroalkyl group with 1-20 carbon atoms, a substituted or unsubstituted aralkyl group with 7-30 carbon atoms, a substituted or unsubstituted alkoxy group with 1-20 ring carbon atoms, a substituted or unsubstituted aryloxy group with 6-30 carbon atoms, a substituted or unsubstituted aryl group with 6-30 carbon atoms, and a substituted or unsubstituted heteroaryl group with 3-30 carbon atoms; and/or
R¹ and R² are, at each occurrence, the same or different and are selected from the group consisting of hydrogen, deuterium, fluoro, carbonyl, cyano, SF₅, a substituted or unsubstituted alkyl group with 1-20 carbon atoms, a substituted or unsubstituted aralkyl group with 7-30 carbon atoms, a substituted or unsubstituted alkoxy group with 1-20 ring carbon atoms, a substituted or unsubstituted aryloxy group with 6-30 carbon atoms, a substituted or unsubstituted aryl group with 6-30 carbon atoms, a substituted or unsubstituted heteroaryl group with 3-30 carbon atoms, and a combination thereof.

Furthermore, when R¹ and R² are, at each occurrence, the same or different and are selected from a substituted alkyl group with 1-20 carbon atoms, a substituted aralkyl group with 7-30 carbon atoms, a substituted alkoxy group with 1-20 ring carbon atoms, a substituted aryloxy group with 6-30 carbon atoms, a substituted aryl group with 6-30 carbon atoms, and a substituted heteroaryl group with 3-30 carbon atoms, and the substituents in R¹ and R² comprise at least one electron-withdrawing group.

Furthermore, the electron-withdrawing group is selected from the group consisting of halogen, nitroso, nitro, acyl, carbonyl, carboxy, an ester group, cyano, isocyano, SCN, OCN, SF₅, boranyl, sulfinyl, sulfonyl, phosphoroso, heteroaryl ring group, and any one of the following groups substituted with one or more of halogen, nitroso, nitro, acyl, carbonyl, carboxy, an ester group, cyano, isocyano, SCN, OCN, SF₅, boranyl, sulfinyl, sulfonyl, phosphoroso, and an aza-aromatic ring group: an alkyl group with 1-20 carbon atoms, a cycloalkyl group with 3-20 ring carbon atoms, a heteroalkyl group with 1-20 carbon atoms, an aralkyl group with 7-30 carbon atoms, an alkoxy group with 1-20 carbon atoms, an aryloxy group with 6-30 carbon atoms, an alkenyl group with 2-20 carbon atoms, an alkynyl group with 2-20 carbon atoms, an aryl group with 6-30 carbon atoms, a heteroaryl group with 3-30 carbon atoms, an alkylsilyl group with 3-20 carbon atoms, an arylsilyl group with 6-20 carbon atoms, and a combination thereof.

Furthermore, the electron-withdrawing group is selected from the group consisting of fluoro, cyano, isocyano, SCN, OCN, SF₅, and any one of the following groups substituted with one or more of fluoro, cyano, isocyano, SCN, OCN, and SF₅: an alkyl group with 1-20 carbon atoms, a cycloalkyl group with 3-20 ring carbon atoms, a heteroalkyl group with 1-20 carbon atoms, an aralkyl group with 7-30 carbon atoms, an alkoxy group with 1-20 carbon atoms, an aryloxy group with 6-30 carbon atoms, an aryl group with 6-30 carbon atoms, a heteroaryl group with 3-30 carbon atoms, an alkylsilyl group with 3-20 carbon atoms, an arylsilyl group with 6-20 carbon atoms, and a combination thereof.

Furthermore, the electron-withdrawing group is selected from the group consisting of F, CF₃, OCF₃, SF₅, SO₂CF₃, cyano, isocyano, SCN, OCN, pyrimidinyl, triazinyl, and a combination thereof.

Furthermore, X is, at each occurrence, the same or different and is selected from O, S, or

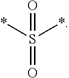

Furthermore, X is selected from 0; and/or
at least one of R′ and R″ is selected from cyano.

Furthermore, L is, at each occurrence, the same or different and is selected from a single bond, O, a substituted or unsubstituted arylene group with 6-30 carbon atoms, a substituted or unsubstituted heteroarylene group with 3-30 carbon atoms, and a combination thereof;
ring A is a substituted or unsubstituted aromatic ring with 6-30 carbon atoms or a substituted or unsubstituted heteroaromatic ring with 3-30 carbon atoms.

Furthermore, when ring A has a substituent, the ring A can be, at each occurrence, substituted with a single one or more R³, and L can be, at each occurrence, substituted with a single one or more R⁴; and

R³ and R⁴ are, at each occurrence, the same or different and are selected from the group consisting of any one of or any combination of some of hydrogen, deuterium, hydroxy, mercapto, halogen, carbonyl, cyano, SF₅, sulfonyl, phosphoroso, a substituted or unsubstituted alkyl group with 1-20 carbon atoms, a substituted or unsubstituted cycloalkyl group with 3-20 ring carbon atoms, a substituted or unsubstituted heteroalkyl group with 1-20 carbon atoms, a substituted or unsubstituted aralkyl group with 7-30 carbon atoms, a substituted or unsubstituted alkoxy group with 1-20 ring carbon atoms, a substituted or unsubstituted aryloxy group with 6-30 carbon atoms, a substituted or unsubstituted aryl group with 6-30 carbon atoms, and a substituted or unsubstituted heteroaryl group with 3-30 carbon atoms; and adjacent R³ and R⁴ can be optionally connected to form a ring.

Furthermore, L is, at each occurrence, the same or different and is selected from a single bond, O, a substituted or unsubstituted phenylene group, a substituted or unsubstituted pyridinylene group, a substituted or unsubstituted biphenylene group, a substituted or unsubstituted terphenylene group, a substituted or unsubstituted naphthylene group, a substituted or unsubstituted naphthyridinylene group, a substituted or unsubstituted phenanthrenylene group, a substituted or unsubstituted phenanthrolinylene group, a substituted or unsubstituted anthracenylene group, a substituted or unsubstituted fluorenylidene group, a substituted or unsubstituted dibenzofuranylene group, a substituted or unsubstituted dibenzothienylene group, a substituted or unsubstituted carbazolylene group, and a combination thereof.

Furthermore, L can be, at each occurrence, substituted with a single one or more R⁴, and R⁴ is, at each occurrence, the same or different and is selected from the group consisting of any one of or any combination of some of hydrogen, deuterium, hydroxy, mercapto, halogen, carbonyl, cyano, SF₅, sulfonyl, phosphoroso, a substituted or unsubstituted alkyl group with 1-20 carbon atoms, a substituted or unsubstituted cycloalkyl group with 3-20 ring carbon atoms, a substituted or unsubstituted heteroalkyl group with 1-20 carbon atoms, a substituted or unsubstituted aralkyl group with 7-30 carbon atoms, a substituted or unsubstituted alkoxy group with 1-20 ring carbon atoms, a substituted or unsubstituted aryloxy group with 6-30 carbon atoms, a substituted or unsubstituted aryl group with 6-30 carbon atoms, and a substituted or unsubstituted heteroaryl group with 3-30 carbon atoms; and adjacent R⁴ can be optionally connected to form a ring.

Furthermore, L is, at each occurrence, the same or different and is selected from a single bond, O, and the following structures, and a combination thereof:

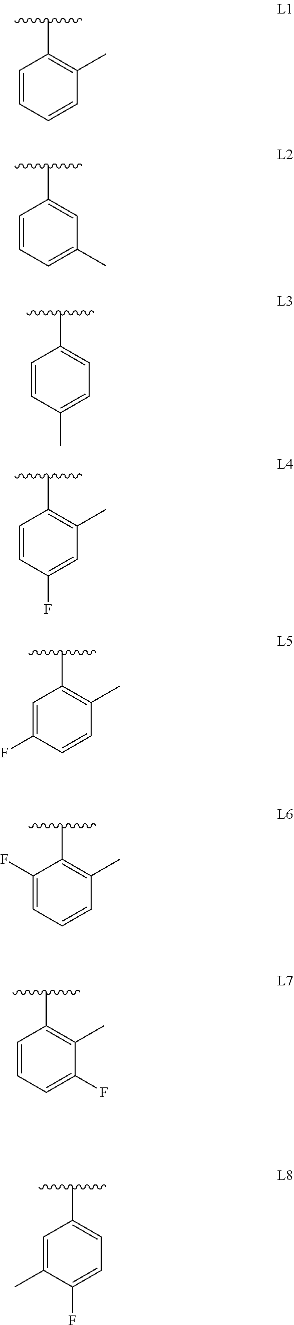

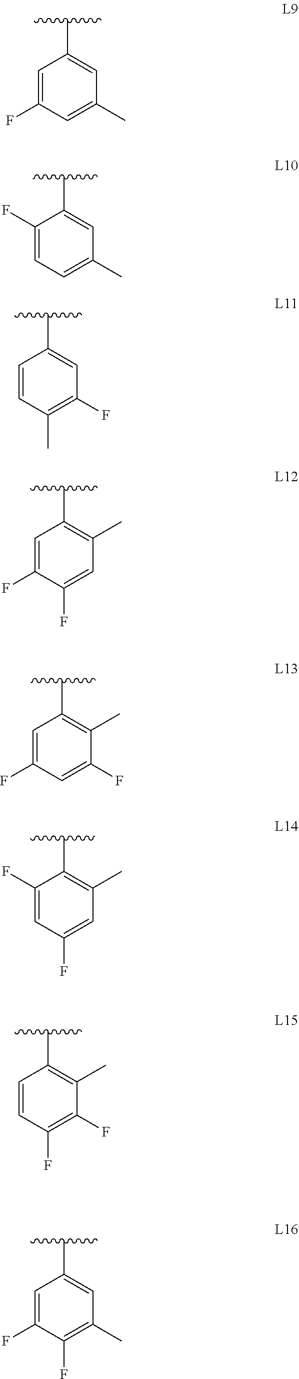

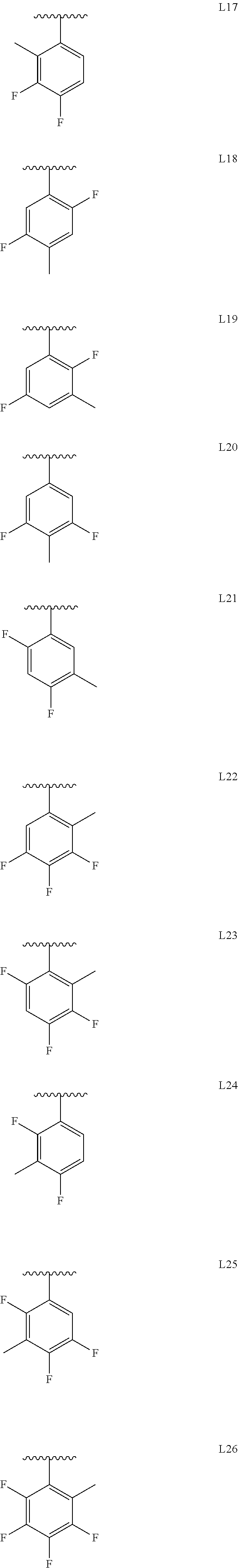

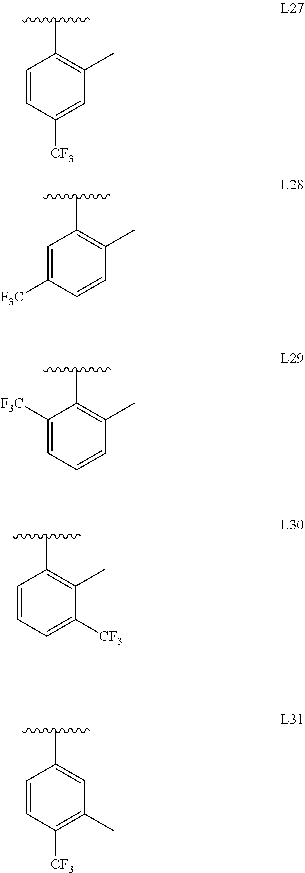 [SMILES: *c1ccc(C(F)(F)F)cc1C ; *c1cc(C(F)(F)F)ccc1C ; *c1c(C)cccc1C(F)(F)F ; *c1cccc(C(F)(F)F)c1C ; *c1ccc(C(F)(F)F)c(C)c1]

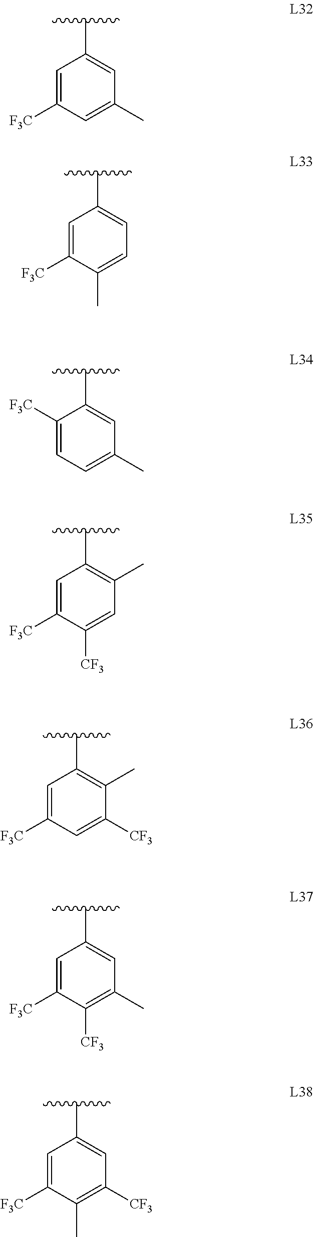

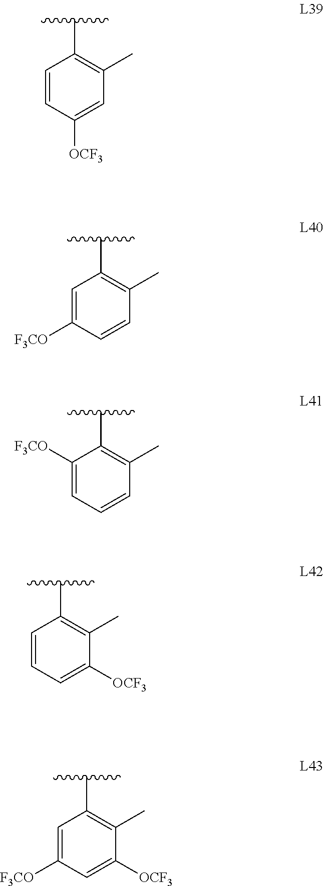 [SMILES: *c1ccc(OC(F)(F)F)cc1C ; *c1cc(OC(F)(F)F)ccc1C ; *c1c(C)cccc1OC(F)(F)F ; *c1cccc(OC(F)(F)F)c1C ; *c1cc(OC(F)(F)F)cc(OC(F)(F)F)c1C]

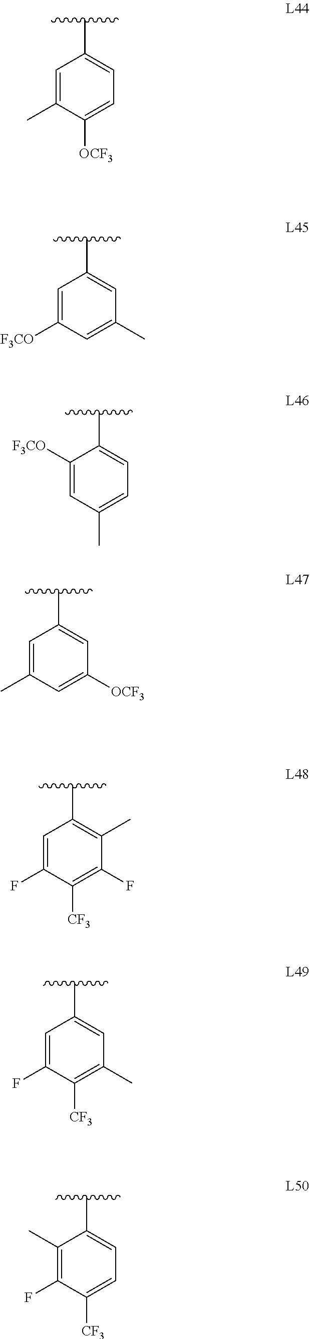

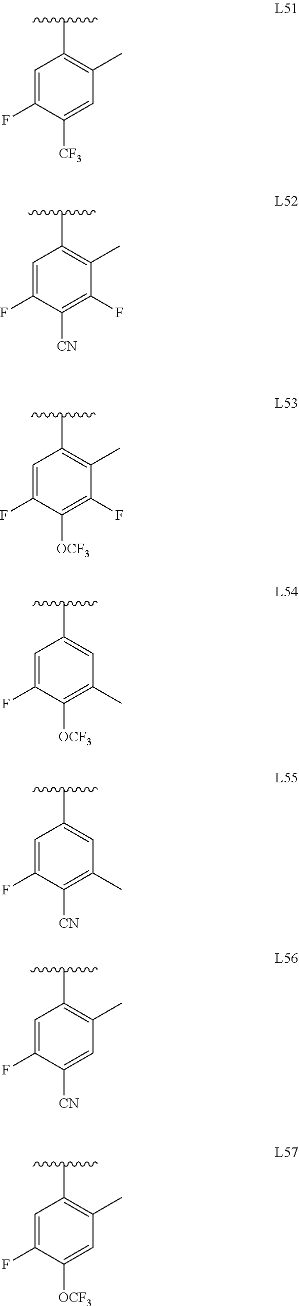

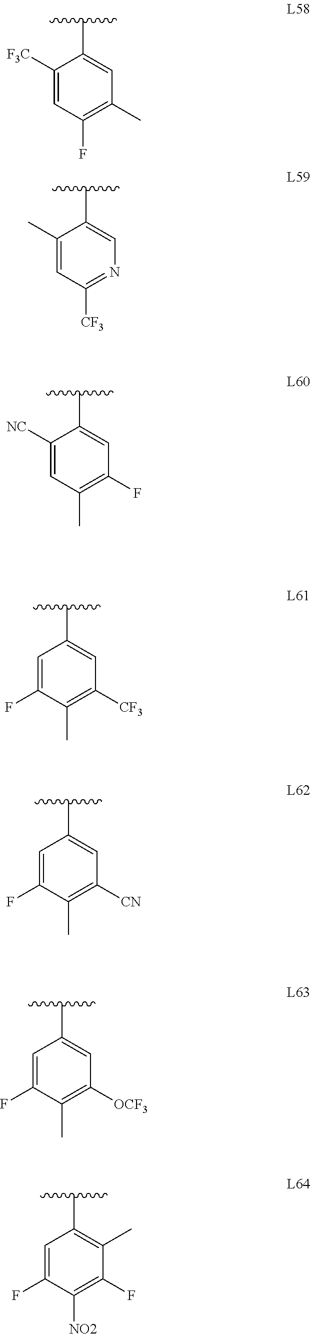

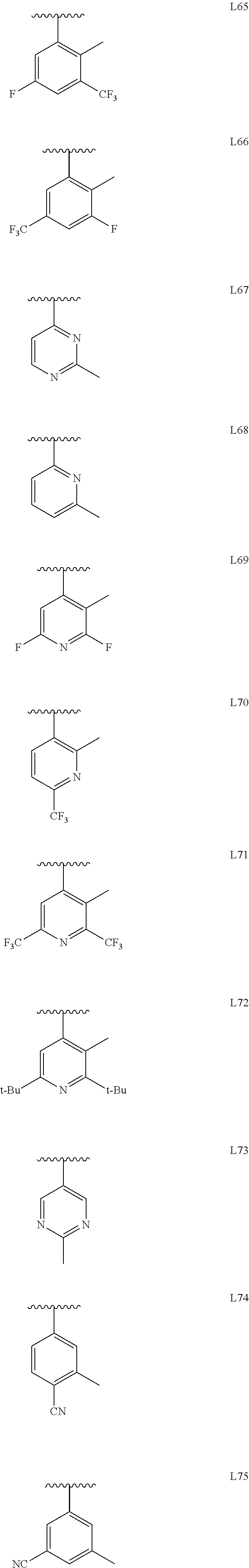

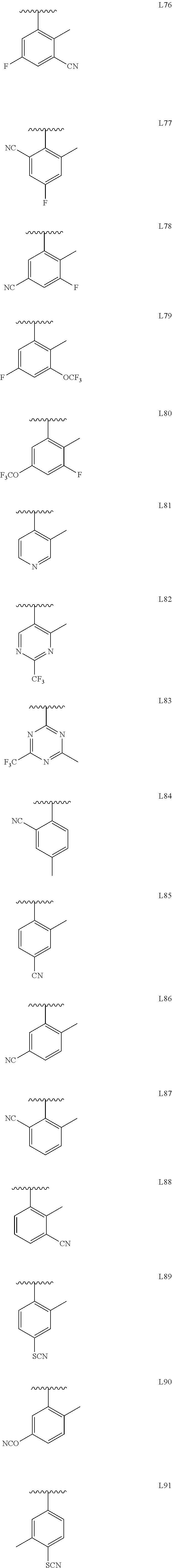

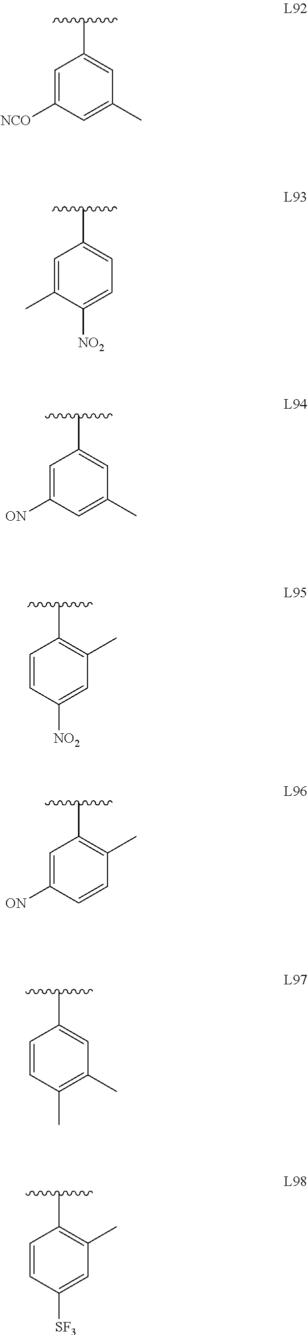

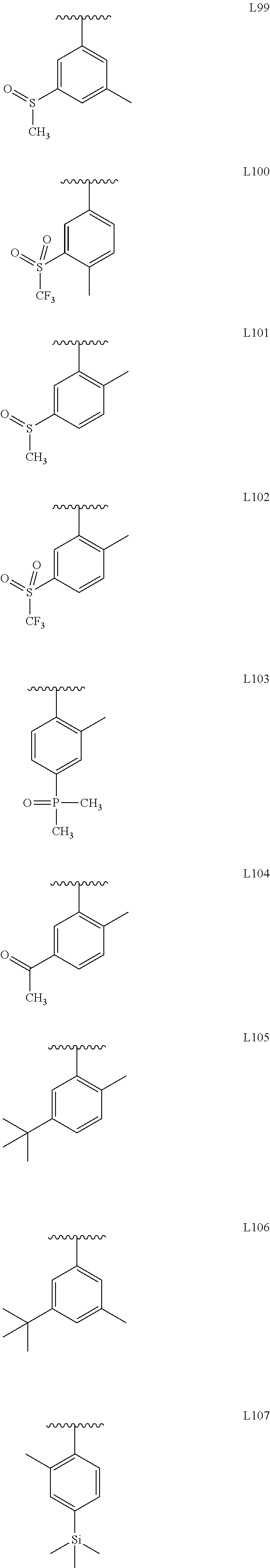

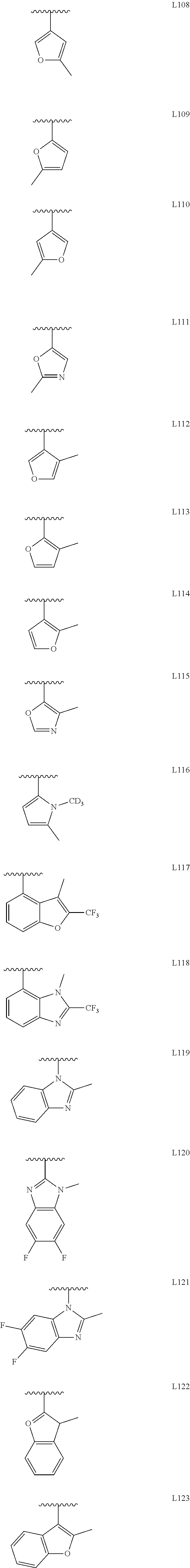

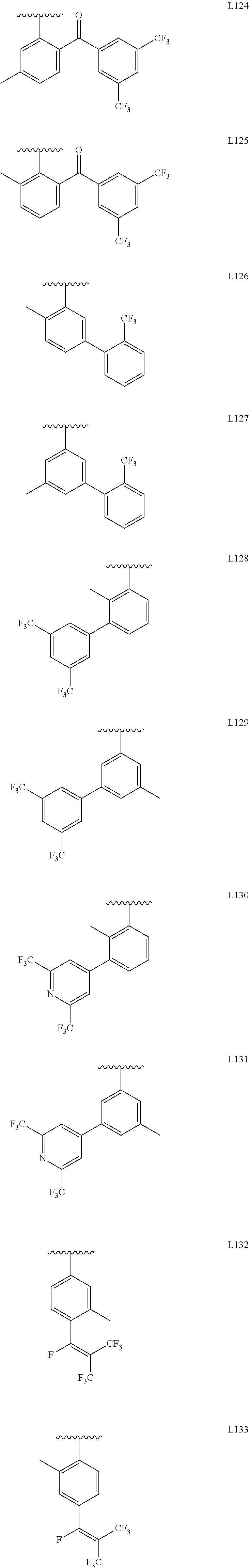

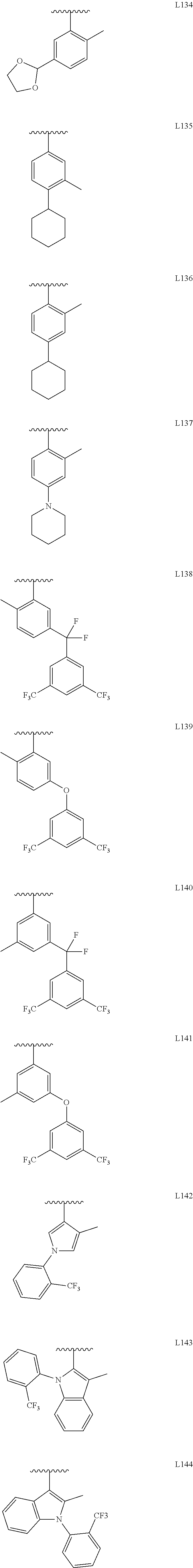

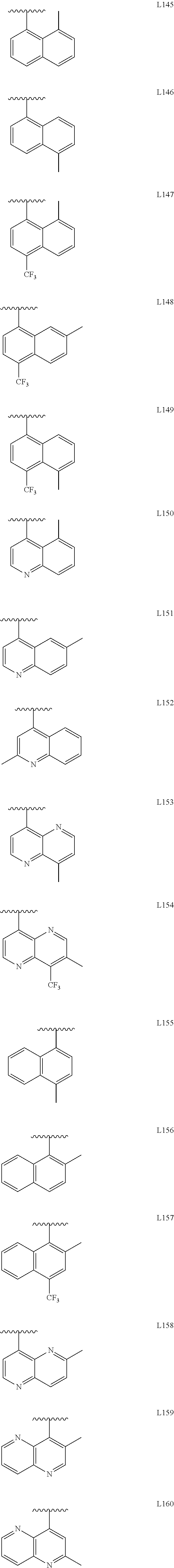

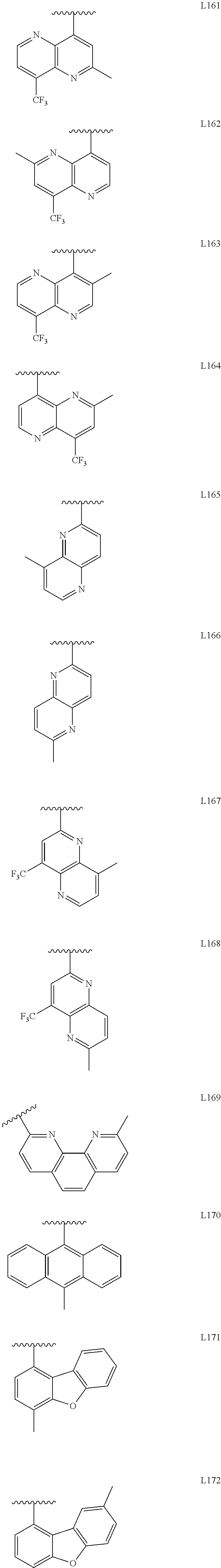

hydrogens in the compounds with the above structures can be partially or completely replaced by deuterium; and
“ [symbol] ” represents the position of connection of L to formula I or another L, and “ [symbol] ” represents the position of connection of L to ring A or another L.

Furthermore, Ar¹, Ar², R′, R″, R¹, R², R³, and R⁴ are, at each occurrence, the same or different and are selected from the group consisting of the following structures:

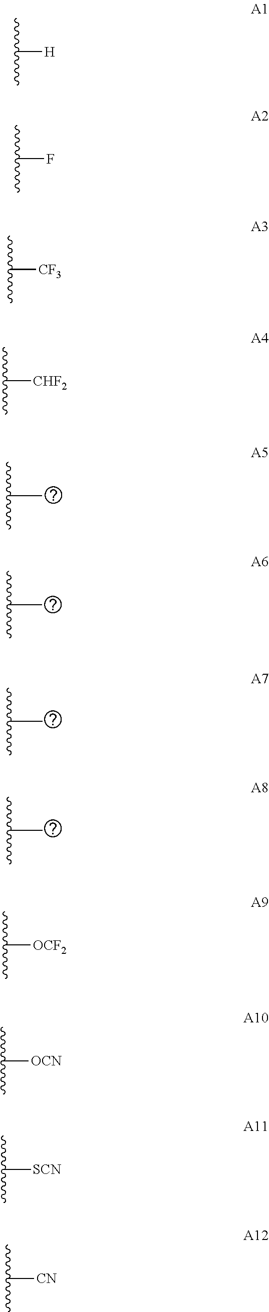

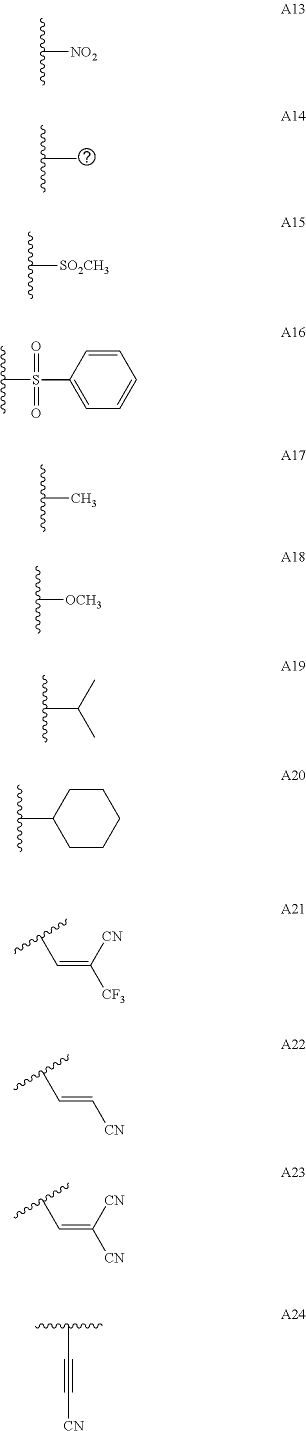

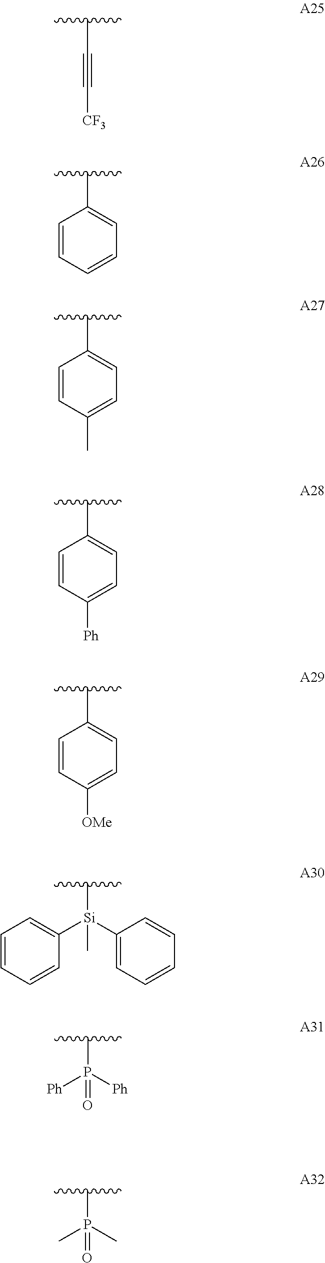

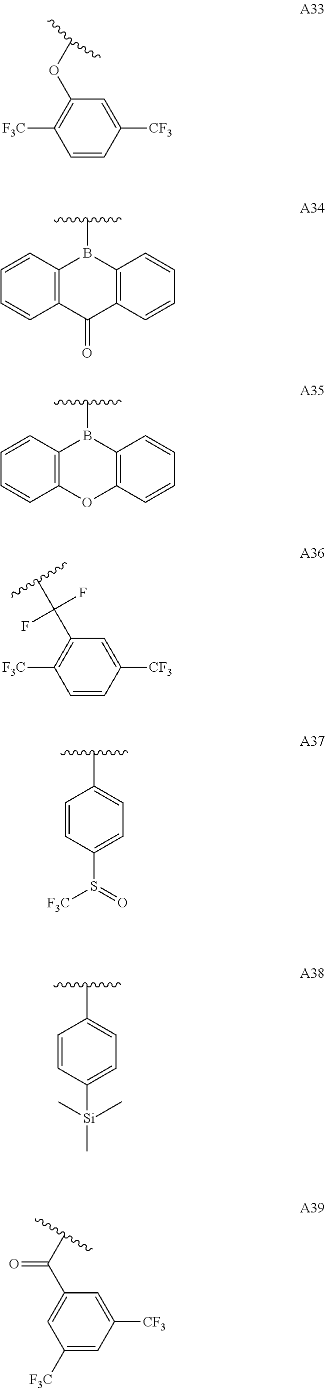

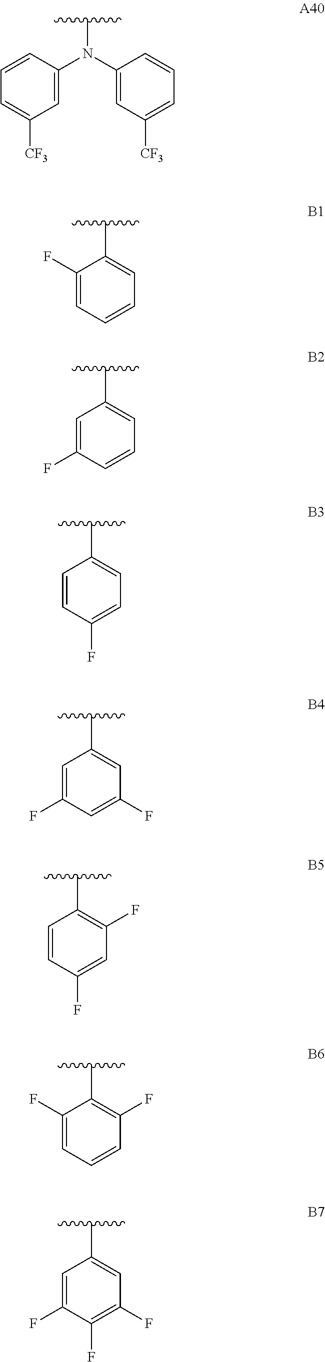

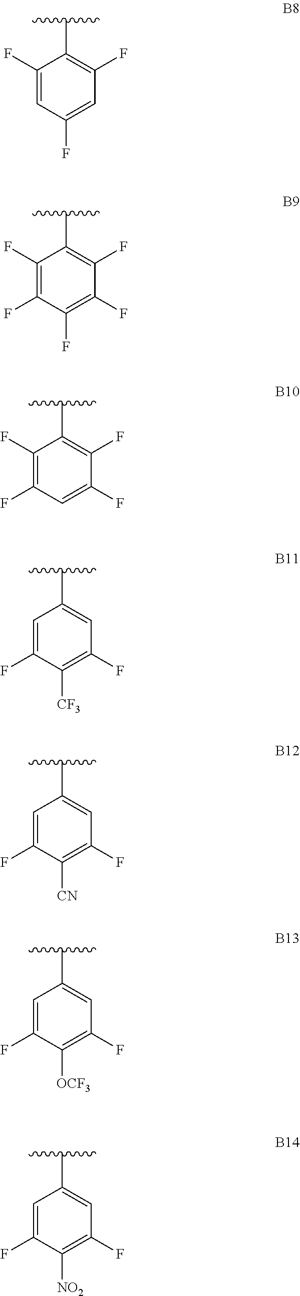

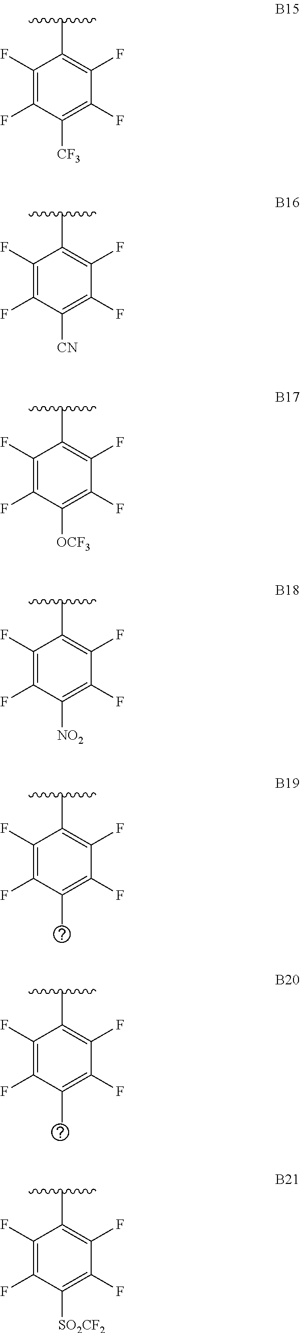

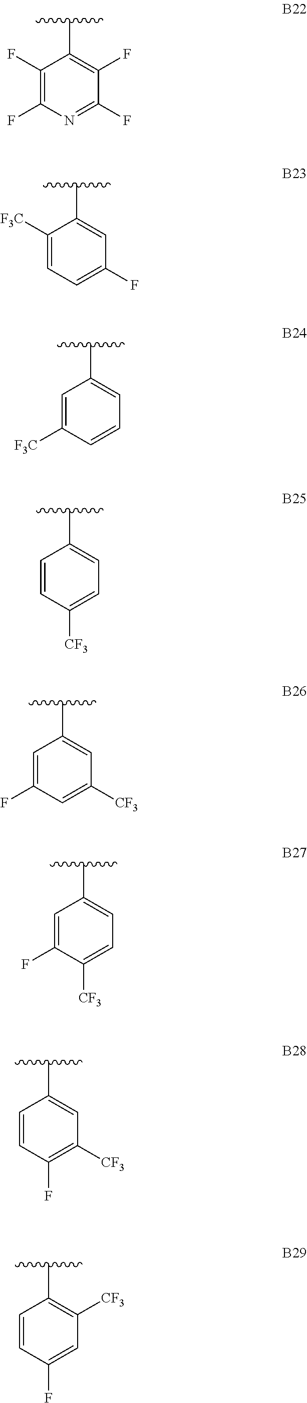

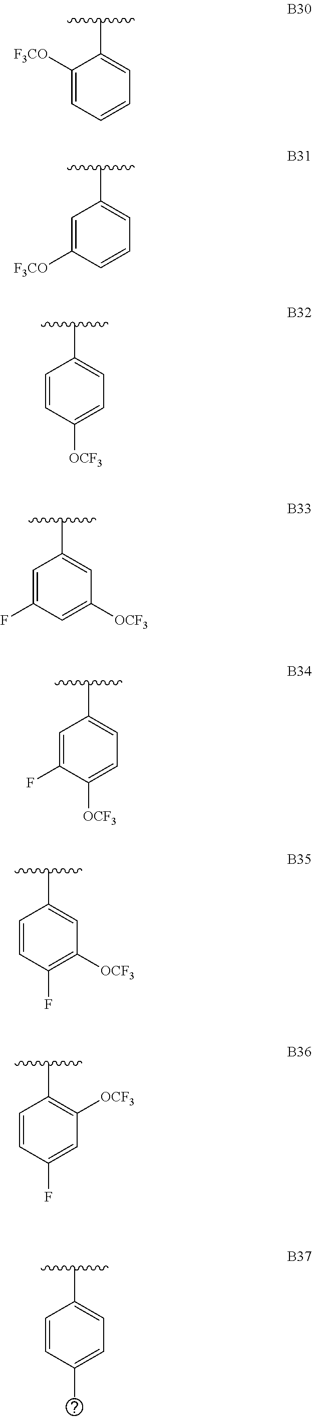

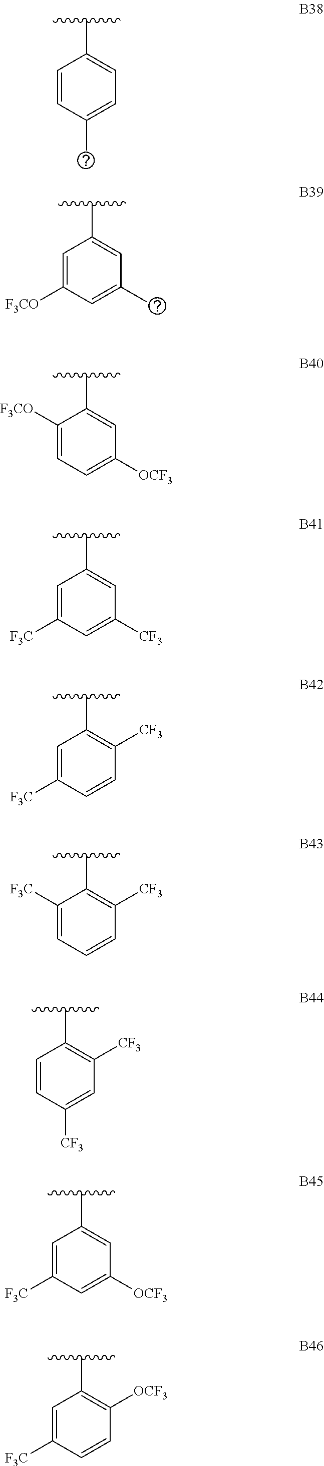

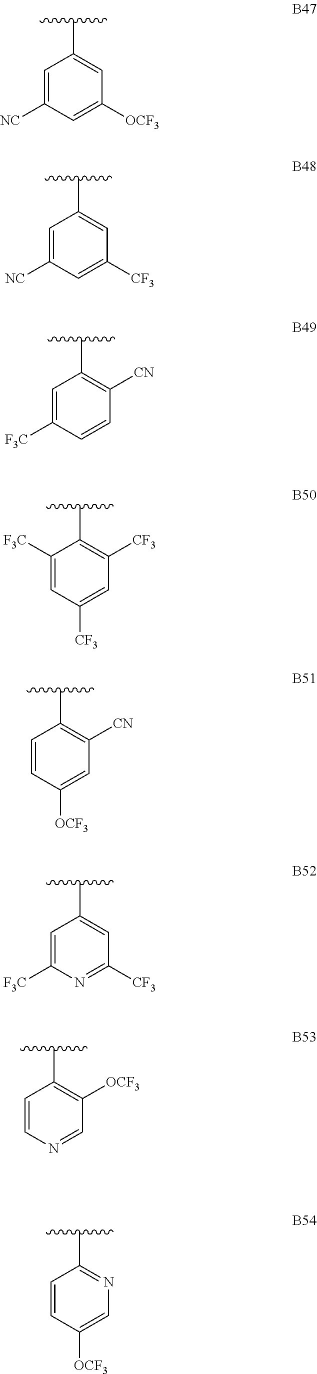

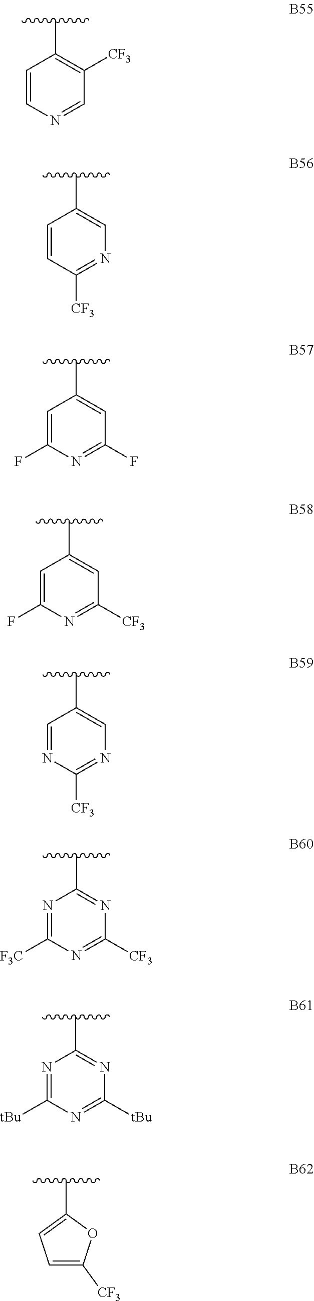

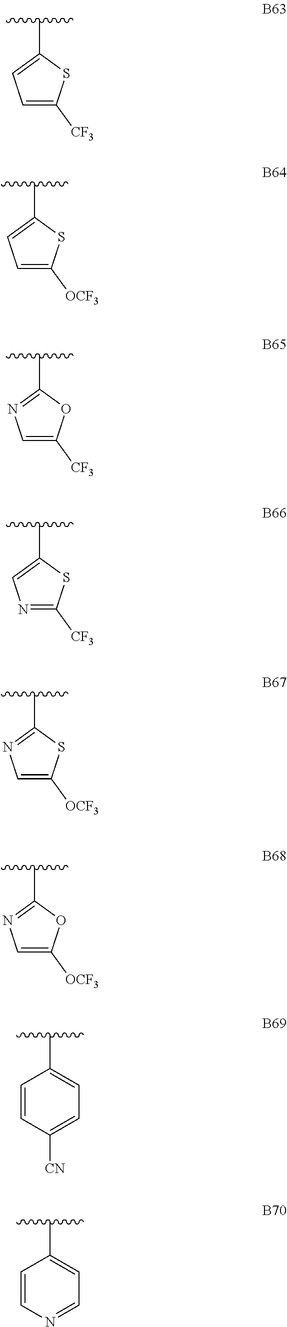

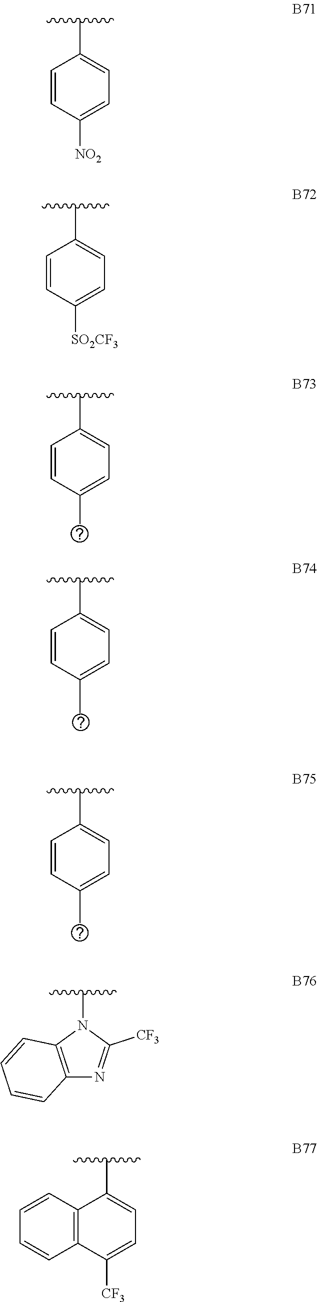

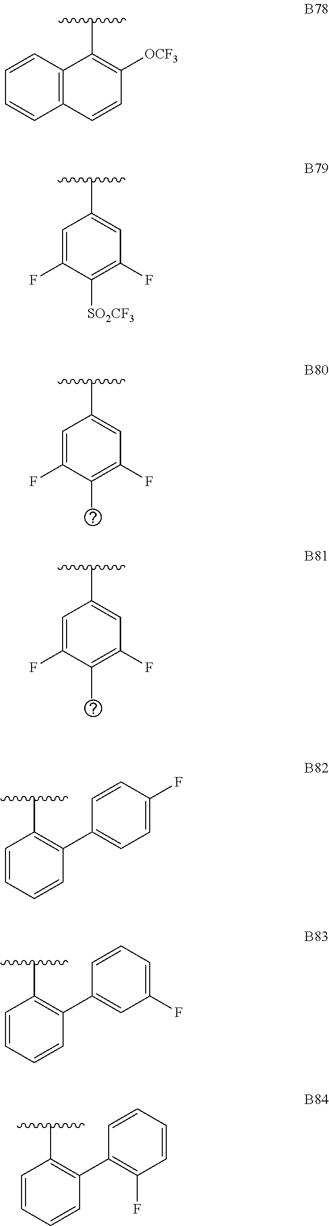

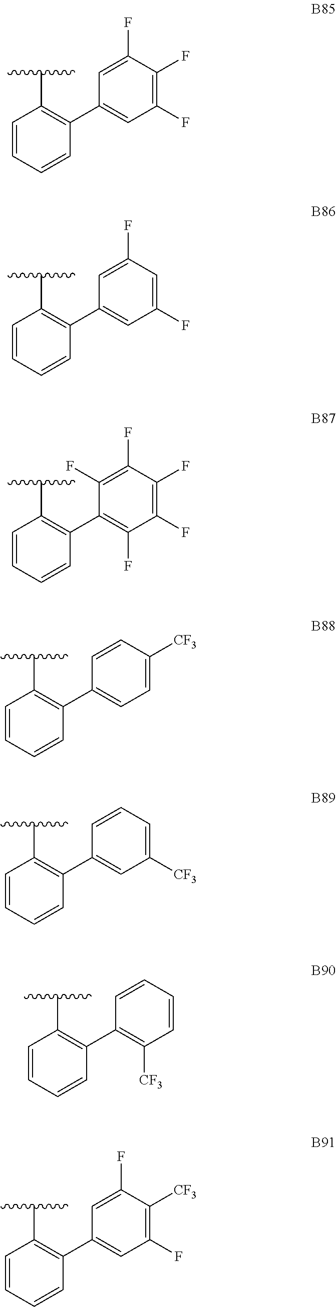

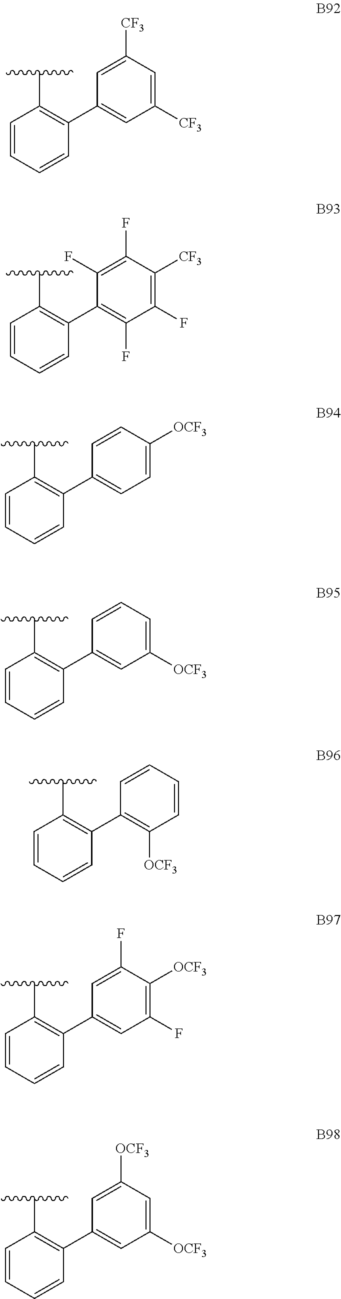

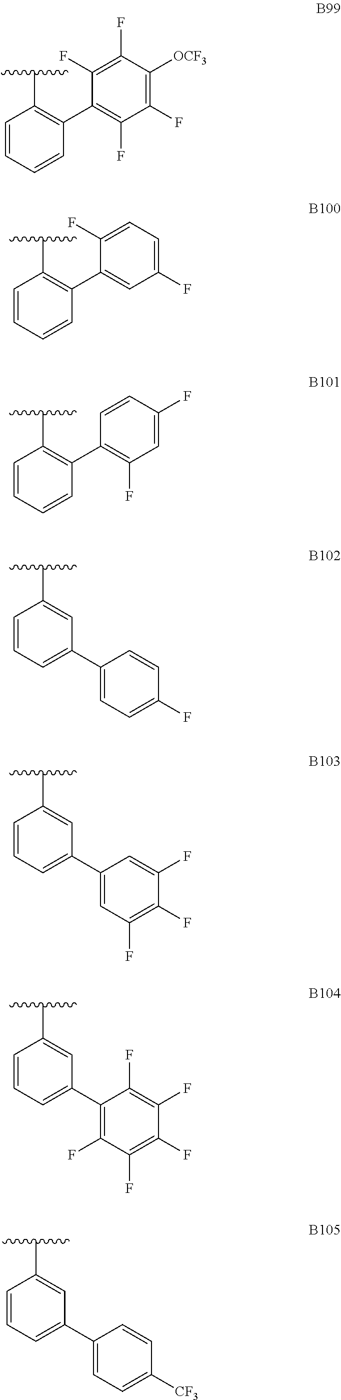

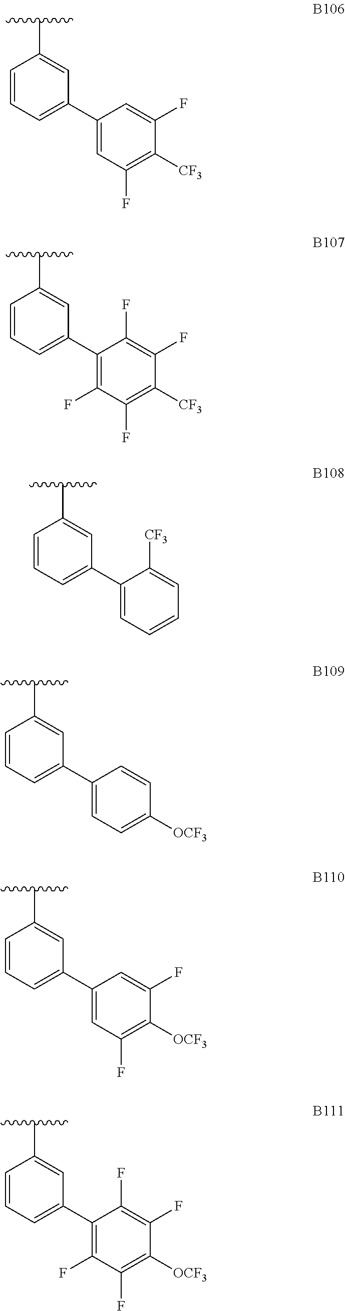 [SMILES: *c1cccc(-c2cc(F)c(C(F)(F)F)c(F)c2)c1 ; *c1cccc(-c2c(F)c(F)c(C(F)(F)F)c(F)c2F)c1 ; *c1cccc(-c2ccccc2C(F)(F)F)c1 ; *c1cccc(-c2ccc(OC(F)(F)F)cc2)c1 ; *c1cccc(-c2cc(F)c(OC(F)(F)F)c(F)c2)c1 ; *c1cccc(-c2c(F)c(F)c(OC(F)(F)F)c(F)c2F)c1]

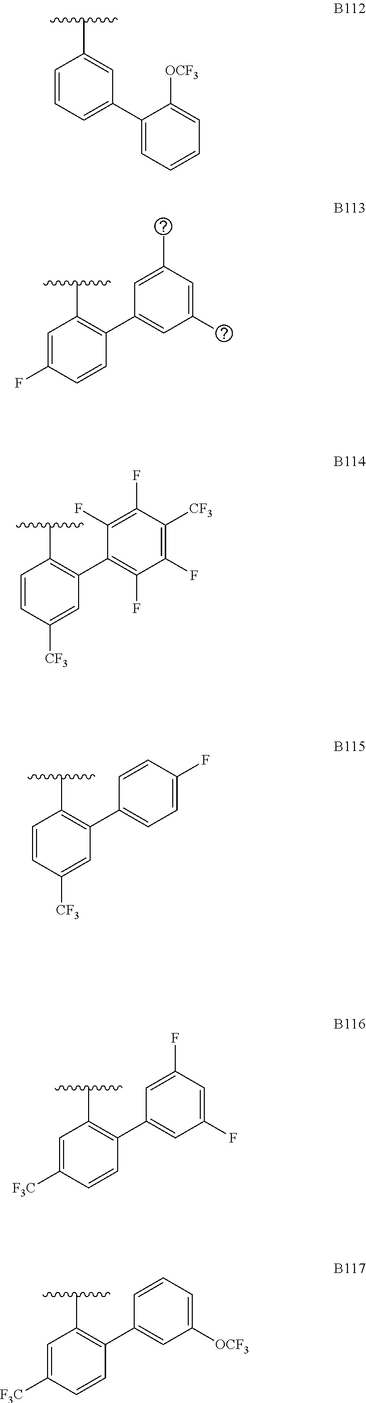 [SMILES: *c1cccc(-c2ccccc2OC(F)(F)F)c1 ; *c1cc(F)ccc1-c1cc(C)cc(C)c1 ; *c1ccc(C(F)(F)F)cc1-c1c(F)c(F)c(C(F)(F)F)c(F)c1F ; *c1ccc(C(F)(F)F)cc1-c1ccc(F)cc1 ; *c1cc(C(F)(F)F)ccc1-c1cc(F)cc(F)c1 ; *c1cc(C(F)(F)F)ccc1-c1cccc(OC(F)(F)F)c1]

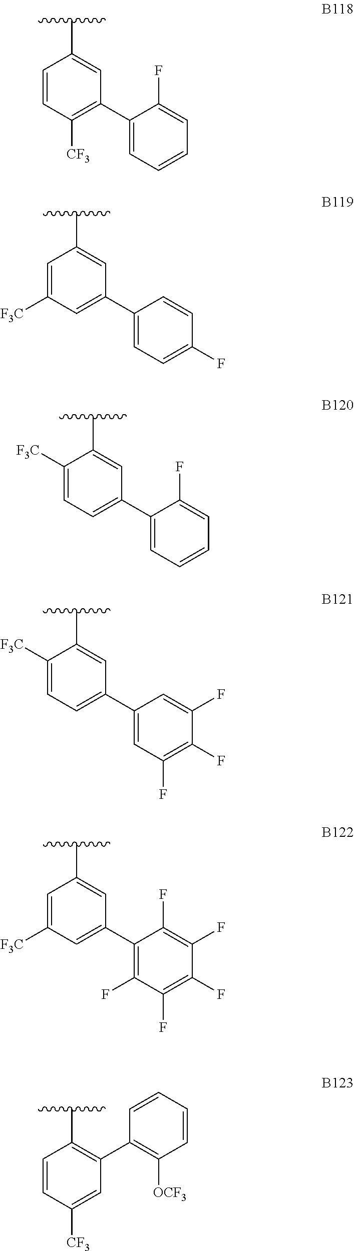 [SMILES: *c1ccc(C(F)(F)F)c(-c2ccccc2F)c1 ; *c1cc(-c2ccc(F)cc2)cc(C(F)(F)F)c1 ; *c1cc(-c2ccccc2F)ccc1C(F)(F)F ; *c1cc(-c2cc(F)c(F)c(F)c2)ccc1C(F)(F)F ; *c1cc(-c2c(F)c(F)c(F)c(F)c2F)cc(C(F)(F)F)c1 ; *c1ccc(C(F)(F)F)cc1-c1ccccc1OC(F)(F)F]

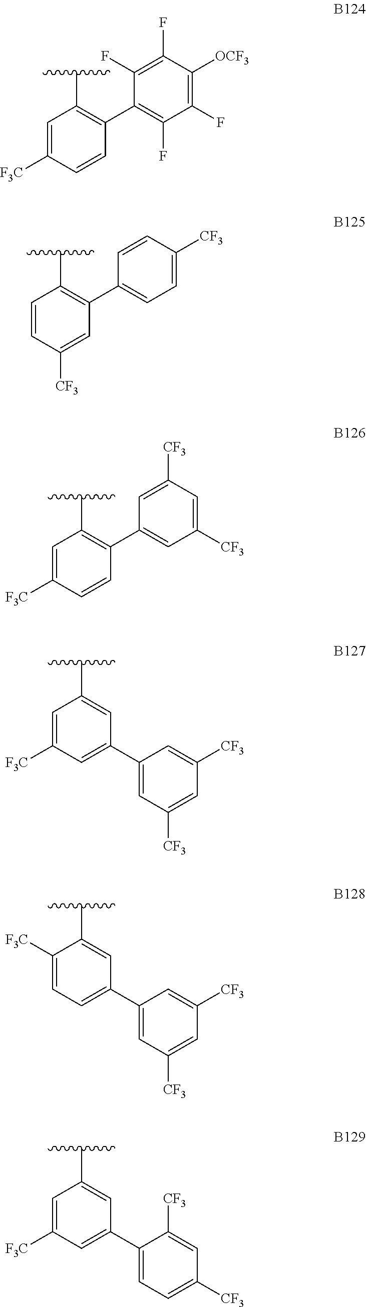 [SMILES: *c1cc(C(F)(F)F)ccc1-c1c(F)c(F)c(OC(F)(F)F)c(F)c1F ; *c1ccc(C(F)(F)F)cc1-c1ccc(C(F)(F)F)cc1 ; *c1cc(C(F)(F)F)ccc1-c1cc(C(F)(F)F)cc(C(F)(F)F)c1 ; *c1cc(-c2cc(C(F)(F)F)cc(C(F)(F)F)c2)cc(C(F)(F)F)c1 ; *c1cc(-c2cc(C(F)(F)F)cc(C(F)(F)F)c2)ccc1C(F)(F)F ; *c1cc(-c2ccc(C(F)(F)F)cc2C(F)(F)F)cc(C(F)(F)F)c1]

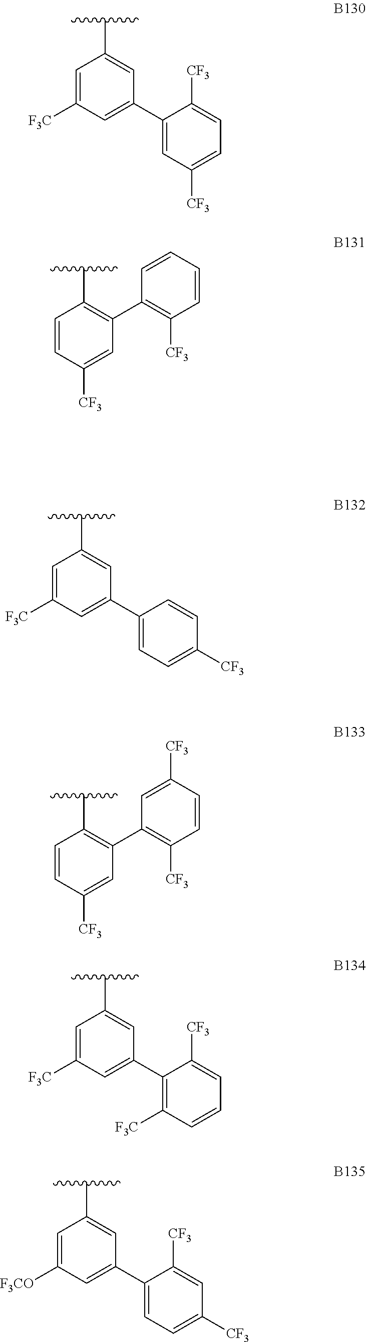 [SMILES: *c1cc(-c2cc(C(F)(F)F)ccc2C(F)(F)F)cc(C(F)(F)F)c1 ; *c1ccc(C(F)(F)F)cc1-c1ccccc1C(F)(F)F ; *c1cc(-c2ccc(C(F)(F)F)cc2)cc(C(F)(F)F)c1 ; *c1ccc(C(F)(F)F)cc1-c1cc(C(F)(F)F)ccc1C(F)(F)F ; *c1cc(-c2c(C(F)(F)F)cccc2C(F)(F)F)cc(C(F)(F)F)c1 ; *c1cc(OC(F)(F)F)cc(-c2ccc(C(F)(F)F)cc2C(F)(F)F)c1]

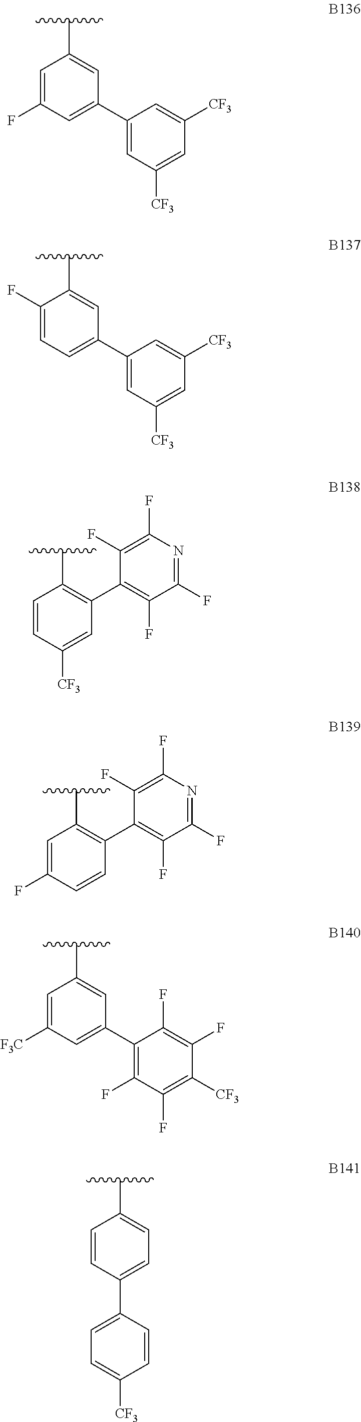 [SMILES: *c1cc(F)cc(-c2cc(C(F)(F)F)cc(C(F)(F)F)c2)c1 ; *c1cc(-c2cc(C(F)(F)F)cc(C(F)(F)F)c2)ccc1F ; *c1ccc(C(F)(F)F)cc1-c1c(F)c(F)nc(F)c1F ; *c1cc(F)ccc1-c1c(F)c(F)nc(F)c1F ; *c1cc(-c2c(F)c(F)c(C(F)(F)F)c(F)c2F)cc(C(F)(F)F)c1 ; *c1ccc(-c2ccc(C(F)(F)F)cc2)cc1]

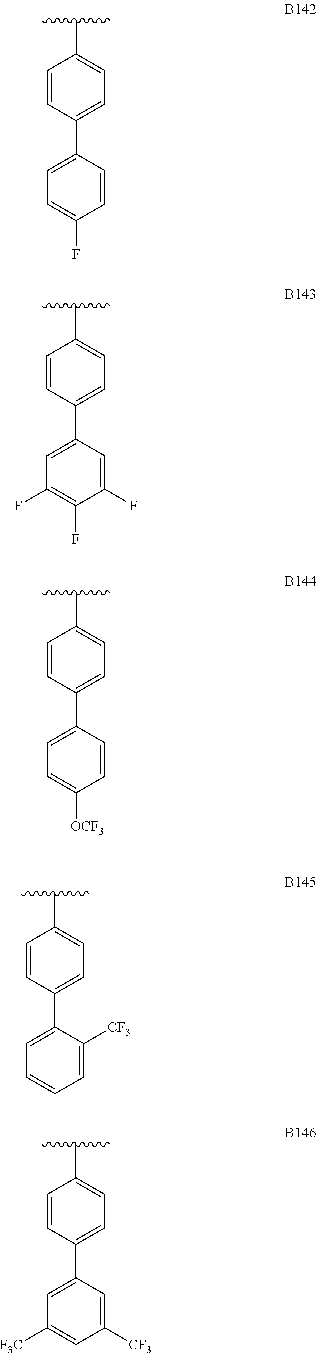 [SMILES: *c1ccc(-c2ccc(F)cc2)cc1 ; *c1ccc(-c2cc(F)c(F)c(F)c2)cc1 ; *c1ccc(-c2ccc(OC(F)(F)F)cc2)cc1 ; *c1ccc(-c2ccccc2C(F)(F)F)cc1 ; *c1ccc(-c2cc(C(F)(F)F)cc(C(F)(F)F)c2)cc1]

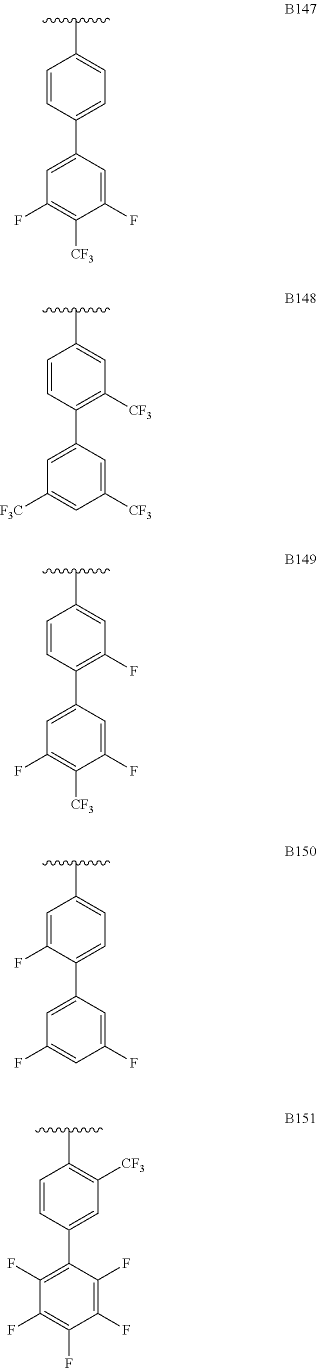 [SMILES: *c1ccc(-c2cc(F)c(C(F)(F)F)c(F)c2)cc1 ; *c1ccc(-c2cc(C(F)(F)F)cc(C(F)(F)F)c2)c(C(F)(F)F)c1 ; *c1ccc(-c2cc(F)c(C(F)(F)F)c(F)c2)c(F)c1 ; *c1ccc(-c2cc(F)cc(F)c2)c(F)c1 ; *c1ccc(-c2c(F)c(F)c(F)c(F)c2F)cc1C(F)(F)F]

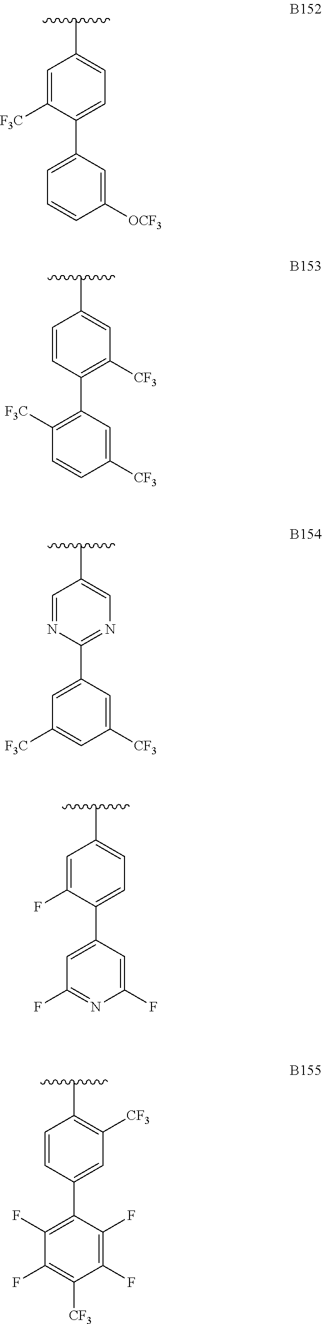 [SMILES: *c1ccc(-c2cccc(OC(F)(F)F)c2)c(C(F)(F)F)c1 ; *c1ccc(-c2cc(C(F)(F)F)ccc2C(F)(F)F)c(C(F)(F)F)c1 ; *c1cnc(-c2cc(C(F)(F)F)cc(C(F)(F)F)c2)nc1 ; *c1ccc(-c2cc(F)nc(F)c2)c(F)c1 ; *c1ccc(-c2c(F)c(F)c(C(F)(F)F)c(F)c2F)cc1C(F)(F)F]

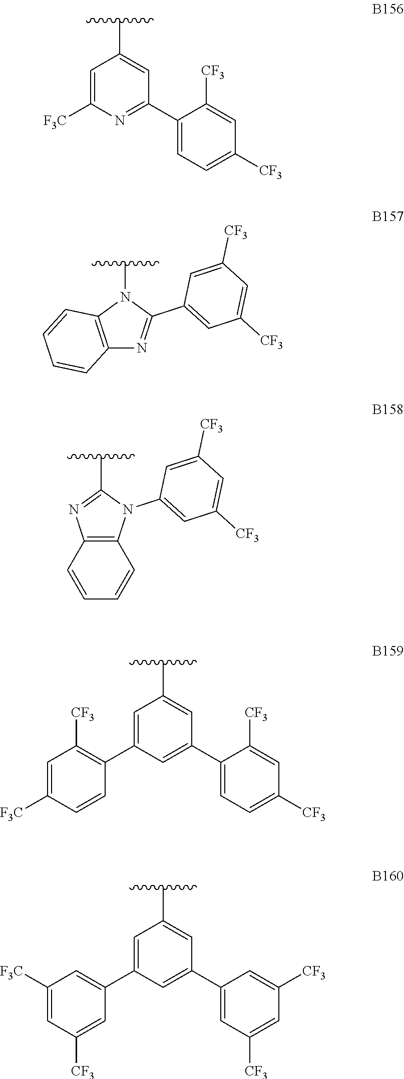 [SMILES: *c1cc(-c2ccc(C(F)(F)F)cc2C(F)(F)F)nc(C(F)(F)F)c1 ; *n1c(-c2cc(C(F)(F)F)cc(C(F)(F)F)c2)nc2ccccc21 ; *c1nc2ccccc2n1-c1cc(C(F)(F)F)cc(C(F)(F)F)c1 ; *c1cc(-c2ccc(C(F)(F)F)cc2C(F)(F)F)cc(-c2ccc(C(F)(F)F)cc2C(F)(F)F)c1 ; *c1cc(-c2cc(C(F)(F)F)cc(C(F)(F)F)c2)cc(-c2cc(C(F)(F)F)cc(C(F)(F)F)c2)c1]

hydrogens in the compounds with the above structures can be partially or completely replaced by deuterium; and
“ [symbol] ” represents the position of connection of the above structures to other structures.

Furthermore, the compound is selected from the group consisting of compounds P1-1 to P1-1028, compounds P2-1 to P2-1028, compounds P3-1 to P3-1028, and compounds P4-1 to P4-1028; and
the compounds P1-1 to P1-1028 have structures represented by formula I-1-1; wherein R¹ and W² are the same, and W, Ar¹, R¹, R², X, and L respectively correspond to atoms or groups selected from the following table 1:

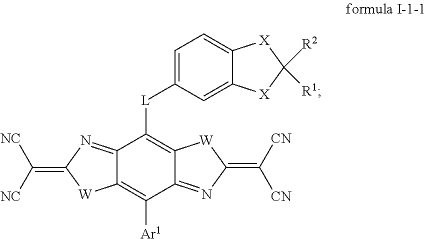

TABLE 1

| Compound No. | W | Ar¹ | R¹ and R² | X | L |
|---|---|---|---|---|---|
| P1-1 | O | A2 | A1 | O | Single bond |
| P1-2 | O | A2 | A1 | O | O |
| P1-3 | O | A2 | A1 | O | L1 |
| P1-4 | O | A2 | A1 | O | L5 |
| P1-5 | O | A2 | A1 | O | L9 |
| P1-6 | O | A2 | A1 | O | L18 |
| P1-7 | O | A2 | A1 | O | L22 |
| P1-8 | O | A2 | A1 | O | L27 |
| P1-9 | O | A2 | A1 | O | L29 |
| P1-10 | O | A2 | A1 | O | L36 |
| P1-11 | O | A2 | A1 | O | L40 |
| P1-12 | O | A2 | A1 | O | L53 |
| P1-13 | O | A2 | A1 | O | L59 |
| P1-14 | O | A2 | A1 | O | L65 |
| P1-15 | O | A2 | A1 | O | L68 |
| P1-16 | O | A2 | A1 | O | L119 |
| P1-17 | O | A2 | A1 | O | L128 |
| P1-18 | O | A2 | A1 | O | L148 |
| P1-19 | O | B4 | A1 | O | Single bond |
| P1-20 | O | B4 | A1 | O | O |
| P1-21 | O | B4 | A1 | O | L1 |
| P1-22 | O | B4 | A1 | O | L5 |
| P1-23 | O | B4 | A1 | O | L9 |
| P1-24 | O | B4 | A1 | O | L18 |
| P1-25 | O | B4 | A1 | O | L22 |
| P1-26 | O | B4 | A1 | O | L27 |
| P1-27 | O | B4 | A1 | O | L29 |
| P1-28 | O | B4 | A1 | O | L36 |
| P1-29 | O | B4 | A1 | O | L40 |
| P1-30 | O | B4 | A1 | O | L53 |
| P1-31 | O | B4 | A1 | O | L59 |
| P1-32 | O | B4 | A1 | O | L65 |
| P1-33 | O | B4 | A1 | O | L68 |
| P1-34 | O | B4 | A1 | O | L119 |
| P1-35 | O | B4 | A1 | O | L128 |
| P1-36 | O | B4 | A1 | O | L148 |
| P1-37 | O | B23 | A1 | O | Single bond |
| P1-38 | O | B23 | A1 | O | O |
| P1-39 | O | B23 | A1 | O | L1 |
| P1-40 | O | B23 | A1 | O | L5 |
| P1-41 | O | B23 | A1 | O | L9 |
| P1-42 | O | B23 | A1 | O | L18 |
| P1-43 | O | B23 | A1 | O | L22 |
| P1-44 | O | B23 | A1 | O | L27 |
| P1-45 | O | B23 | A1 | O | L29 |
| P1-46 | O | B23 | A1 | O | L36 |
| P1-47 | O | B23 | A1 | O | L40 |
| P1-48 | O | B23 | A1 | O | L53 |
| P1-49 | O | B23 | A1 | O | L59 |
| P1-50 | O | B23 | A1 | O | L65 |
| P1-51 | O | B23 | A1 | O | L68 |
| P1-52 | O | B23 | A1 | O | L119 |
| P1-53 | O | B23 | A1 | O | L128 |
| P1-54 | O | B23 | A1 | O | L148 |
| P1-55 | O | B36 | A1 | O | Single bond |
| P1-56 | O | B36 | A1 | O | O |
| P1-57 | O | B36 | A1 | O | L1 |
| P1-58 | O | B36 | A1 | O | L5 |
| P1-59 | O | B36 | A1 | O | L9 |
| P1-60 | O | B36 | A1 | O | L18 |
| P1-61 | O | B36 | A1 | O | L22 |
| P1-62 | O | B36 | A1 | O | L27 |
| P1-63 | O | B36 | A1 | O | L29 |
| P1-64 | O | B36 | A1 | O | L36 |
| P1-65 | O | B36 | A1 | O | L40 |
| P1-66 | O | B36 | A1 | O | L53 |
| P1-67 | O | B36 | A1 | O | L59 |
| P1-68 | O | B36 | A1 | O | L65 |
| P1-69 | O | B36 | A1 | O | L68 |
| P1-70 | O | B36 | A1 | O | L119 |
| P1-71 | O | B36 | A1 | O | L128 |
| P1-72 | O | B36 | A1 | O | L148 |
| P1-73 | O | B44 | A1 | O | Single bond |
| P1-74 | O | B44 | A1 | O | O |
| P1-75 | O | B44 | A1 | O | L1 |
| P1-76 | O | B44 | A1 | O | L5 |
| P1-77 | O | B44 | A1 | O | L9 |
| P1-78 | O | B44 | A1 | O | L18 |
| P1-79 | O | B44 | A1 | O | L22 |
| P1-80 | O | B44 | A1 | O | L27 |
| P1-81 | O | B44 | A1 | O | L29 |
| P1-82 | O | B44 | A1 | O | L36 |
| P1-83 | O | B44 | A1 | O | L40 |
| P1-84 | O | B44 | A1 | O | L53 |
| P1-85 | O | B44 | A1 | O | L59 |
| P1-86 | O | B44 | A1 | O | L65 |
| P1-87 | O | B44 | A1 | O | L68 |
| P1-88 | O | B44 | A1 | O | L119 |
| P1-89 | O | B44 | A1 | O | L128 |
| P1-90 | O | B44 | A1 | O | L148 |
| P1-91 | O | B92 | A1 | O | Single bond |
| P1-92 | O | B92 | A1 | O | O |
| P1-93 | O | B92 | A1 | O | L1 |
| P1-94 | O | B92 | A1 | O | L5 |
| P1-95 | O | B92 | A1 | O | L9 |
| P1-96 | O | B92 | A1 | O | L18 |
| P1-97 | O | B92 | A1 | O | L22 |
| P1-98 | O | B92 | A1 | O | L27 |
| P1-99 | O | B92 | A1 | O | L29 |
| P1-100 | O | B92 | A1 | O | L36 |
| P1-101 | O | B92 | A1 | O | L40 |
| P1-102 | O | B92 | A1 | O | L53 |
| P1-103 | O | B92 | A1 | O | L59 |
| P1-104 | O | B92 | A1 | O | L65 |
| P1-105 | O | B92 | A1 | O | L68 |
| P1-106 | O | B92 | A1 | O | L119 |
| P1-107 | O | B92 | A1 | O | L128 |
| P1-108 | O | B92 | A1 | O | L148 |
| P1-109 | O | A2 | A2 | O | Single bond |
| P1-110 | O | A2 | A2 | O | O |
| P1-111 | O | A2 | A2 | O | L1 |
| P1-112 | O | A2 | A2 | O | L2 |
| P1-113 | O | A2 | A2 | O | L3 |
| P1-114 | O | A2 | A2 | O | L4 |
| P1-115 | O | A2 | A2 | O | L5 |
| P1-116 | O | A2 | A2 | O | L6 |
| P1-117 | O | A2 | A2 | O | L7 |
| P1-118 | O | A2 | A2 | O | L9 |
| P1-119 | O | A2 | A2 | O | L11 |
| P1-120 | O | A2 | A2 | O | L14 |
| P1-121 | O | A2 | A2 | O | L18 |
| P1-122 | O | A2 | A2 | O | L22 |
| P1-123 | O | A2 | A2 | O | L27 |
| P1-124 | O | A2 | A2 | O | L28 |
| P1-125 | O | A2 | A2 | O | L29 |
| P1-126 | O | A2 | A2 | O | L30 |
| P1-127 | O | A2 | A2 | O | L32 |
| P1-128 | O | A2 | A2 | O | L36 |
| P1-129 | O | A2 | A2 | O | L40 |
| P1-130 | O | A2 | A2 | O | L48 |
| P1-131 | O | A2 | A2 | O | L53 |
| P1-132 | O | A2 | A2 | O | L59 |
| P1-133 | O | A2 | A2 | O | L65 |
| P1-134 | O | A2 | A2 | O | L68 |
| P1-135 | O | A2 | A2 | O | L79 |
| P1-136 | O | A2 | A2 | O | L89 |
| P1-137 | O | A2 | A2 | O | L102 |
| P1-138 | O | A2 | A2 | O | L111 |
| P1-139 | O | A2 | A2 | O | L119 |
| P1-140 | O | A2 | A2 | O | L128 |
| P1-141 | O | A2 | A2 | O | L148 |
| P1-142 | O | A2 | A2 | O | L156 |
| P1-143 | O | A2 | A2 | O | L167 |
| P1-144 | O | A2 | A2 | O | L169 |
| P1-145 | O | A2 | A2 | O | L172 |
| P1-146 | O | B4 | A2 | O | Single bond |
| P1-147 | O | B4 | A2 | O | O |
| P1-148 | O | B4 | A2 | O | L1 |
| P1-149 | O | B4 | A2 | O | L2 |
| P1-150 | O | B4 | A2 | O | L3 |
| P1-151 | O | B4 | A2 | O | L4 |
| P1-152 | O | B4 | A2 | O | L5 |
| P1-153 | O | B4 | A2 | O | L6 |
| P1-154 | O | B4 | A2 | O | L7 |
| P1-155 | O | B4 | A2 | O | L9 |
| P1-156 | O | B4 | A2 | O | L11 |
| P1-157 | O | B4 | A2 | O | L14 |
| P1-158 | O | B4 | A2 | O | L18 |
| P1-159 | O | B4 | A2 | O | L22 |
| P1-160 | O | B4 | A2 | O | L27 |
| P1-161 | O | B4 | A2 | O | L28 |
| P1-162 | O | B4 | A2 | O | L29 |
| P1-163 | O | B4 | A2 | O | L30 |
| P1-164 | O | B4 | A2 | O | L32 |
| P1-165 | O | B4 | A2 | O | L36 |
| P1-166 | O | B4 | A2 | O | L40 |
| P1-167 | O | B4 | A2 | O | L48 |
| P1-168 | O | B4 | A2 | O | L53 |
| P1-169 | O | B4 | A2 | O | L59 |
| P1-170 | O | B4 | A2 | O | L65 |
| P1-171 | O | B4 | A2 | O | L68 |
| P1-172 | O | B4 | A2 | O | L79 |
| P1-173 | O | B4 | A2 | O | L89 |
| P1-174 | O | B4 | A2 | O | L102 |
| P1-175 | O | B4 | A2 | O | L111 |
| P1-176 | O | B4 | A2 | O | L119 |
| P1-177 | O | B4 | A2 | O | L128 |
| P1-178 | O | B4 | A2 | O | L148 |
| P1-179 | O | B4 | A2 | O | L156 |
| P1-180 | O | B4 | A2 | O | L167 |
| P1-181 | O | B4 | A2 | O | L169 |
| P1-182 | O | B4 | A2 | O | L172 |
| P1-183 | O | B23 | A2 | O | Single bond |
| P1-184 | O | B23 | A2 | O | O |
| P1-185 | O | B23 | A2 | O | L1 |
| P1-186 | O | B23 | A2 | O | L2 |
| P1-187 | O | B23 | A2 | O | L3 |
| P1-188 | O | B23 | A2 | O | L4 |
| P1-189 | O | B23 | A2 | O | L5 |
| P1-190 | O | B23 | A2 | O | L6 |
| P1-191 | O | B23 | A2 | O | L7 |
| P1-192 | O | B23 | A2 | O | L9 |
| P1-193 | O | B23 | A2 | O | L11 |
| P1-194 | O | B23 | A2 | O | L14 |
| P1-195 | O | B23 | A2 | O | L18 |
| P1-196 | O | B23 | A2 | O | L22 |
| P1-197 | O | B23 | A2 | O | L27 |
| P1-198 | O | B23 | A2 | O | L28 |
| P1-199 | O | B23 | A2 | O | L29 |
| P1-200 | O | B23 | A2 | O | L30 |
| P1-201 | O | B23 | A2 | O | L32 |
| P1-202 | O | B23 | A2 | O | L36 |
| P1-203 | O | B23 | A2 | O | L40 |
| P1-204 | O | B23 | A2 | O | L48 |
| P1-205 | O | B23 | A2 | O | L53 |
| P1-206 | O | B23 | A2 | O | L59 |
| P1-207 | O | B23 | A2 | O | L65 |
| P1-208 | O | B23 | A2 | O | L68 |
| P1-209 | O | B23 | A2 | O | L79 |
| P1-210 | O | B23 | A2 | O | L89 |
| P1-211 | O | B23 | A2 | O | L102 |
| P1-212 | O | B23 | A2 | O | L111 |
| P1-213 | O | B23 | A2 | O | L119 |
| P1-214 | O | B23 | A2 | O | L128 |
| P1-215 | O | B23 | A2 | O | L148 |
| P1-216 | O | B23 | A2 | O | L156 |
| P1-217 | O | B23 | A2 | O | L167 |
| P1-218 | O | B23 | A2 | O | L169 |
| P1-219 | O | B23 | A2 | O | L172 |
| P1-220 | O | B36 | A2 | O | Single bond |
| P1-221 | O | B36 | A2 | O | O |
| P1-222 | O | B36 | A2 | O | L1 |
| P1-223 | O | B36 | A2 | O | L2 |
| P1-224 | O | B36 | A2 | O | L3 |
| P1-225 | O | B36 | A2 | O | L4 |
| P1-226 | O | B36 | A2 | O | L5 |
| P1-227 | O | B36 | A2 | O | L6 |
| P1-228 | O | B36 | A2 | O | L7 |
| P1-229 | O | B36 | A2 | O | L9 |
| P1-230 | O | B36 | A2 | O | L11 |
| P1-231 | O | B36 | A2 | O | L14 |
| P1-232 | O | B36 | A2 | O | L18 |
| P1-233 | O | B36 | A2 | O | L22 |
| P1-234 | O | B36 | A2 | O | L27 |
| P1-235 | O | B36 | A2 | O | L28 |
| P1-236 | O | B36 | A2 | O | L29 |
| P1-237 | O | B36 | A2 | O | L30 |
| P1-238 | O | B36 | A2 | O | L32 |
| P1-239 | O | B36 | A2 | O | L36 |
| P1-240 | O | B36 | A2 | O | L40 |
| P1-241 | O | B36 | A2 | O | L48 |
| P1-242 | O | B36 | A2 | O | L53 |
| P1-243 | O | B36 | A2 | O | L59 |
| P1-244 | O | B36 | A2 | O | L65 |
| P1-245 | O | B36 | A2 | O | L68 |
| P1-246 | O | B36 | A2 | O | L79 |
| P1-247 | O | B36 | A2 | O | L89 |
| P1-248 | O | B36 | A2 | O | L102 |
| P1-249 | O | B36 | A2 | O | L111 |
| P1-250 | O | B36 | A2 | O | L119 |
| P1-251 | O | B36 | A2 | O | L128 |
| P1-252 | O | B36 | A2 | O | L148 |
| P1-253 | O | B36 | A2 | O | L156 |
| P1-254 | O | B36 | A2 | O | L167 |
| P1-255 | O | B36 | A2 | O | L169 |
| P1-256 | O | B36 | A2 | O | L172 |
| P1-257 | O | B44 | A2 | O | Single bond |
| P1-258 | O | B44 | A2 | O | O |
| P1-259 | O | B44 | A2 | O | L1 |
| P1-260 | O | B44 | A2 | O | L2 |
| P1-261 | O | B44 | A2 | O | L3 |
| P1-262 | O | B44 | A2 | O | L4 |
| P1-263 | O | B44 | A2 | O | L5 |
| P1-264 | O | B44 | A2 | O | L6 |
| P1-265 | O | B44 | A2 | O | L7 |
| P1-266 | O | B44 | A2 | O | L9 |
| P1-267 | O | B44 | A2 | O | L11 |
| P1-268 | O | B44 | A2 | O | L14 |
| P1-269 | O | B44 | A2 | O | L18 |
| P1-270 | O | B44 | A2 | O | L22 |
| P1-271 | O | B44 | A2 | O | L27 |
| P1-272 | O | B44 | A2 | O | L28 |
| P1-273 | O | B44 | A2 | O | L29 |
| P1-274 | O | B44 | A2 | O | L30 |
| P1-275 | O | B44 | A2 | O | L32 |
| P1-276 | O | B44 | A2 | O | L36 |
| P1-277 | O | B44 | A2 | O | L40 |
| P1-278 | O | B44 | A2 | O | L48 |
| P1-279 | O | B44 | A2 | O | L53 |
| P1-280 | O | B44 | A2 | O | L59 |
| P1-281 | O | B44 | A2 | O | L65 |
| P1-282 | O | B44 | A2 | O | L68 |
| P1-283 | O | B44 | A2 | O | L79 |
| P1-284 | O | B44 | A2 | O | L89 |
| P1-285 | O | B44 | A2 | O | L102 |
| P1-286 | O | B44 | A2 | O | L111 |
| P1-287 | O | B44 | A2 | O | L119 |
| P1-288 | O | B44 | A2 | O | L128 |
| P1-289 | O | B44 | A2 | O | L148 |
| P1-290 | O | B44 | A2 | O | L156 |
| P1-291 | O | B44 | A2 | O | L167 |
| P1-292 | O | B44 | A2 | O | L169 |
| P1-293 | O | B44 | A2 | O | L172 |
| P1-294 | O | B92 | A2 | O | Single bond |
| P1-295 | O | B92 | A2 | O | O |
| P1-296 | O | B92 | A2 | O | L1 |
| P1-297 | O | B92 | A2 | O | L2 |
| P1-298 | O | B92 | A2 | O | L3 |
| P1-299 | O | B92 | A2 | O | L4 |
| P1-300 | O | B92 | A2 | O | L5 |
| P1-301 | O | B92 | A2 | O | L6 |
| P1-302 | O | B92 | A2 | O | L7 |
| P1-303 | O | B92 | A2 | O | L9 |
| P1-304 | O | B92 | A2 | O | L11 |
| P1-305 | O | B92 | A2 | O | L14 |
| P1-306 | O | B92 | A2 | O | L18 |
| P1-307 | O | B92 | A2 | O | L22 |
| P1-308 | O | B92 | A2 | O | L27 |
| P1-309 | O | B92 | A2 | O | L28 |
| P1-310 | O | B92 | A2 | O | L29 |
| P1-311 | O | B92 | A2 | O | L30 |
| P1-312 | O | B92 | A2 | O | L32 |
| P1-313 | O | B92 | A2 | O | L36 |
| P1-314 | O | B92 | A2 | O | L40 |
| P1-315 | O | B92 | A2 | O | L48 |
| P1-316 | O | B92 | A2 | O | L53 |
| P1-317 | O | B92 | A2 | O | L59 |
| P1-318 | O | B92 | A2 | O | L65 |
| P1-319 | O | B92 | A2 | O | L68 |
| P1-320 | O | B92 | A2 | O | L79 |
| P1-321 | O | B92 | A2 | O | L89 |
| P1-322 | O | B92 | A2 | O | L102 |
| P1-323 | O | B92 | A2 | O | L111 |
| P1-324 | O | B92 | A2 | O | L119 |
| P1-325 | O | B92 | A2 | O | L128 |
| P1-326 | O | B92 | A2 | O | L148 |
| P1-327 | O | B92 | A2 | O | L156 |
| P1-328 | O | B92 | A2 | O | L167 |
| P1-329 | O | B92 | A2 | O | L169 |
| P1-330 | O | B92 | A2 | O | L172 |
| P1-331 | O | A2 | A3 | O | Single bond |
| P1-332 | O | A2 | A3 | O | O |
| P1-333 | O | A2 | A3 | O | L1 |
| P1-334 | O | A2 | A3 | O | L2 |
| P1-335 | O | A2 | A3 | O | L3 |
| P1-336 | O | A2 | A3 | O | L4 |
| P1-337 | O | A2 | A3 | O | L5 |
| P1-338 | O | A2 | A3 | O | L6 |
| P1-339 | O | A2 | A3 | O | L7 |
| P1-340 | O | A2 | A3 | O | L9 |
| P1-341 | O | A2 | A3 | O | L11 |
| P1-342 | O | A2 | A3 | O | L14 |
| P1-343 | O | A2 | A3 | O | L18 |
| P1-344 | O | A2 | A3 | O | L22 |
| P1-345 | O | A2 | A3 | O | L27 |
| P1-346 | O | A2 | A3 | O | L28 |
| P1-347 | O | A2 | A3 | O | L29 |
| P1-348 | O | A2 | A3 | O | L30 |
| P1-349 | O | A2 | A3 | O | L32 |
| P1-350 | O | A2 | A3 | O | L36 |
| P1-351 | O | A2 | A3 | O | L40 |
| P1-352 | O | A2 | A3 | O | L48 |
| P1-353 | O | A2 | A3 | O | L53 |
| P1-354 | O | A2 | A3 | O | L59 |
| P1-355 | O | A2 | A3 | O | L65 |
| P1-356 | O | A2 | A3 | O | L68 |
| P1-357 | O | A2 | A3 | O | L79 |
| P1-358 | O | A2 | A3 | O | L89 |
| P1-359 | O | A2 | A3 | O | L102 |
| P1-360 | O | A2 | A3 | O | L111 |
| P1-361 | O | A2 | A3 | O | L119 |
| P1-362 | O | A2 | A3 | O | L128 |
| P1-363 | O | A2 | A3 | O | L148 |
| P1-364 | O | A2 | A3 | O | L156 |
| P1-365 | O | A2 | A3 | O | L167 |
| P1-366 | O | A2 | A3 | O | L169 |
| P1-367 | O | A2 | A3 | O | L172 |
| P1-368 | O | B4 | A3 | O | Single bond |
| P1-369 | O | B4 | A3 | O | O |
| P1-370 | O | B4 | A3 | O | L1 |
| P1-371 | O | B4 | A3 | O | L2 |
| P1-372 | O | B4 | A3 | O | L3 |
| P1-373 | O | B4 | A3 | O | L4 |
| P1-374 | O | B4 | A3 | O | L5 |
| P1-375 | O | B4 | A3 | O | L6 |
| P1-376 | O | B4 | A3 | O | L7 |
| P1-377 | O | B4 | A3 | O | L9 |
| P1-378 | O | B4 | A3 | O | L11 |
| P1-379 | O | B4 | A3 | O | L14 |
| P1-380 | O | B4 | A3 | O | L18 |
| P1-381 | O | B4 | A3 | O | L22 |
| P1-382 | O | B4 | A3 | O | L27 |
| P1-383 | O | B4 | A3 | O | L28 |
| P1-384 | O | B4 | A3 | O | L29 |
| P1-385 | O | B4 | A3 | O | L30 |
| P1-386 | O | B4 | A3 | O | L32 |
| P1-387 | O | B4 | A3 | O | L36 |
| P1-388 | O | B4 | A3 | O | L40 |
| P1-389 | O | B4 | A3 | O | L48 |
| P1-390 | O | B4 | A3 | O | L53 |
| P1-391 | O | B4 | A3 | O | L59 |
| P1-392 | O | B4 | A3 | O | L65 |
| P1-393 | O | B4 | A3 | O | L68 |
| P1-394 | O | B4 | A3 | O | L79 |
| P1-395 | O | B4 | A3 | O | L89 |
| P1-396 | O | B4 | A3 | O | L102 |
| P1-397 | O | B4 | A3 | O | L111 |
| P1-398 | O | B4 | A3 | O | L119 |
| P1-399 | O | B4 | A3 | O | L128 |
| P1-400 | O | B4 | A3 | O | L148 |
| P1-401 | O | B4 | A3 | O | L156 |
| P1-402 | O | B4 | A3 | O | L167 |
| P1-403 | O | B4 | A3 | O | L169 |
| P1-404 | O | B4 | A3 | O | L172 |
| P1-405 | O | B23 | A3 | O | Single bond |
| P1-406 | O | B23 | A3 | O | O |
| P1-407 | O | B23 | A3 | O | L1 |
| P1-408 | O | B23 | A3 | O | L2 |
| P1-409 | O | B23 | A3 | O | L3 |
| P1-410 | O | B23 | A3 | O | L4 |
| P1-411 | O | B23 | A3 | O | L5 |
| P1-412 | O | B23 | A3 | O | L6 |
| P1-413 | O | B23 | A3 | O | L7 |
| P1-414 | O | B23 | A3 | O | L9 |
| P1-415 | O | B23 | A3 | O | L11 |
| P1-416 | O | B23 | A3 | O | L14 |
| P1-417 | O | B23 | A3 | O | L18 |
| P1-418 | O | B23 | A3 | O | L22 |
| P1-419 | O | B23 | A3 | O | L27 |
| P1-420 | O | B23 | A3 | O | L28 |
| P1-421 | O | B23 | A3 | O | L29 |
| P1-422 | O | B23 | A3 | O | L30 |
| P1-423 | O | B23 | A3 | O | L32 |
| P1-424 | O | B23 | A3 | O | L36 |
| P1-425 | O | B23 | A3 | O | L40 |
| P1-426 | O | B23 | A3 | O | L48 |
| P1-427 | O | B23 | A3 | O | L53 |
| P1-428 | O | B23 | A3 | O | L59 |
| P1-429 | O | B23 | A3 | O | L65 |
| P1-430 | O | B23 | A3 | O | L68 |
| P1-431 | O | B23 | A3 | O | L79 |
| P1-432 | O | B23 | A3 | O | L89 |
| P1-433 | O | B23 | A3 | O | L102 |
| P1-434 | O | B23 | A3 | O | L111 |
| P1-435 | O | B23 | A3 | O | L119 |
| P1-436 | O | B23 | A3 | O | L128 |
| P1-437 | O | B23 | A3 | O | L148 |
| P1-438 | O | B23 | A3 | O | L156 |
| P1-439 | O | B23 | A3 | O | L167 |
| P1-440 | O | B23 | A3 | O | L169 |
| P1-441 | O | B23 | A3 | O | L172 |
| P1-442 | O | B36 | A3 | O | Single bond |
| P1-443 | O | B36 | A3 | O | O |
| P1-444 | O | B36 | A3 | O | L1 |
| P1-445 | O | B36 | A3 | O | L2 |
| P1-446 | O | B36 | A3 | O | L3 |
| P1-447 | O | B36 | A3 | O | L4 |
| P1-448 | O | B36 | A3 | O | L5 |
| P1-449 | O | B36 | A3 | O | L6 |
| P1-450 | O | B36 | A3 | O | L7 |
| P1-451 | O | B36 | A3 | O | L9 |
| P1-452 | O | B36 | A3 | O | L11 |
| P1-453 | O | B36 | A3 | O | L14 |
| P1-454 | O | B36 | A3 | O | L18 |
| P1-455 | O | B36 | A3 | O | L22 |
| P1-456 | O | B36 | A3 | O | L27 |
| P1-457 | O | B36 | A3 | O | L28 |
| P1-458 | O | B36 | A3 | O | L29 |
| P1-459 | O | B36 | A3 | O | L30 |
| P1-460 | O | B36 | A3 | O | L32 |
| P1-461 | O | B36 | A3 | O | L36 |
| P1-462 | O | B36 | A3 | O | L40 |
| P1-463 | O | B36 | A3 | O | L48 |
| P1-464 | O | B36 | A3 | O | L53 |
| P1-465 | O | B36 | A3 | O | L59 |
| P1-466 | O | B36 | A3 | O | L65 |
| P1-467 | O | B36 | A3 | O | L68 |
| P1-468 | O | B36 | A3 | O | L79 |
| P1-469 | O | B36 | A3 | O | L89 |
| P1-470 | O | B36 | A3 | O | L102 |
| P1-471 | O | B36 | A3 | O | L111 |
| P1-472 | O | B36 | A3 | O | L119 |
| P1-473 | O | B36 | A3 | O | L128 |
| P1-474 | O | B36 | A3 | O | L148 |
| P1-475 | O | B36 | A3 | O | L156 |
| P1-476 | O | B36 | A3 | O | L167 |
| P1-477 | O | B36 | A3 | O | L169 |
| P1-478 | O | B36 | A3 | O | L172 |
| P1-479 | O | B44 | A3 | O | Single bond |
| P1-480 | O | B44 | A3 | O | O |
| P1-481 | O | B44 | A3 | O | L1 |
| P1-482 | O | B44 | A3 | O | L2 |
| P1-483 | O | B44 | A3 | O | L3 |
| P1-484 | O | B44 | A3 | O | L4 |
| P1-485 | O | B44 | A3 | O | L5 |
| P1-486 | O | B44 | A3 | O | L6 |
| P1-487 | O | B44 | A3 | O | L7 |
| P1-488 | O | B44 | A3 | O | L9 |
| P1-489 | O | B44 | A3 | O | L11 |
| P1-490 | O | B44 | A3 | O | L14 |
| P1-491 | O | B44 | A3 | O | L18 |
| P1-492 | O | B44 | A3 | O | L22 |
| P1-493 | O | B44 | A3 | O | L27 |
| P1-494 | O | B44 | A3 | O | L28 |
| P1-495 | O | B44 | A3 | O | L29 |
| P1-496 | O | B44 | A3 | O | L30 |
| P1-497 | O | B44 | A3 | O | L32 |
| P1-498 | O | B44 | A3 | O | L36 |
| P1-499 | O | B44 | A3 | O | L40 |
| P1-500 | O | B44 | A3 | O | L48 |
| P1-501 | O | B44 | A3 | O | L53 |
| P1-502 | O | B44 | A3 | O | L59 |
| P1-503 | O | B44 | A3 | O | L65 |
| P1-504 | O | B44 | A3 | O | L68 |
| P1-505 | O | B44 | A3 | O | L79 |
| P1-506 | O | B44 | A3 | O | L89 |
| P1-507 | O | B44 | A3 | O | L102 |
| P1-508 | O | B44 | A3 | O | L111 |
| P1-509 | O | B44 | A3 | O | L119 |
| P1-510 | O | B44 | A3 | O | L128 |
| P1-511 | O | B44 | A3 | O | L148 |
| P1-512 | O | B44 | A3 | O | L156 |
| P1-513 | O | B44 | A3 | O | L167 |
| P1-514 | O | B44 | A3 | O | L169 |
| P1-515 | O | B44 | A3 | O | L172 |
| P1-516 | O | B92 | A3 | O | Single bond |
| P1-517 | O | B92 | A3 | O | O |
| P1-518 | O | B92 | A3 | O | L1 |
| P1-519 | O | B92 | A3 | O | L2 |
| P1-520 | O | B92 | A3 | O | L3 |
| P1-521 | O | B92 | A3 | O | L4 |
| P1-522 | O | B92 | A3 | O | L5 |
| P1-523 | O | B92 | A3 | O | L6 |
| P1-524 | O | B92 | A3 | O | L7 |
| P1-525 | O | B92 | A3 | O | L9 |
| P1-526 | O | B92 | A3 | O | L11 |
| P1-527 | O | B92 | A3 | O | L14 |
| P1-528 | O | B92 | A3 | O | L18 |
| P1-529 | O | B92 | A3 | O | L22 |
| P1-530 | O | B92 | A3 | O | L27 |
| P1-531 | O | B92 | A3 | O | L28 |
| P1-532 | O | B92 | A3 | O | L29 |
| P1-533 | O | B92 | A3 | O | L30 |
| P1-534 | O | B92 | A3 | O | L32 |
| P1-535 | O | B92 | A3 | O | L36 |
| P1-536 | O | B92 | A3 | O | L40 |
| P1-537 | O | B92 | A3 | O | L48 |
| P1-538 | O | B92 | A3 | O | L53 |
| P1-539 | O | B92 | A3 | O | L59 |
| P1-540 | O | B92 | A3 | O | L65 |
| P1-541 | O | B92 | A3 | O | L68 |
| P1-542 | O | B92 | A3 | O | L79 |
| P1-543 | O | B92 | A3 | O | L89 |
| P1-544 | O | B92 | A3 | O | L102 |
| P1-545 | O | B92 | A3 | O | L111 |
| P1-546 | O | B92 | A3 | O | L119 |
| P1-547 | O | B92 | A3 | O | L128 |
| P1-548 | O | B92 | A3 | O | L148 |
| P1-549 | O | B92 | A3 | O | L156 |
| P1-550 | O | B92 | A3 | O | L167 |
| P1-551 | O | B92 | A3 | O | L169 |
| P1-552 | O | B92 | A3 | O | L172 |
| P1-553 | O | A2 | A12 | O | Single bond |
| P1-554 | O | A2 | A12 | O | O |
| P1-555 | O | A2 | A12 | O | L1 |
| P1-556 | O | A2 | A12 | O | L5 |
| P1-557 | O | A2 | A12 | O | L9 |
| P1-558 | O | A2 | A12 | O | L18 |
| P1-559 | O | A2 | A12 | O | L22 |
| P1-560 | O | A2 | A12 | O | L27 |
| P1-561 | O | A2 | A12 | O | L29 |
| P1-562 | O | A2 | A12 | O | L36 |
| P1-563 | O | A2 | A12 | O | L40 |
| P1-564 | O | A2 | A12 | O | L53 |
| P1-565 | O | A2 | A12 | O | L59 |
| P1-566 | O | A2 | A12 | O | L65 |
| P1-567 | O | A2 | A12 | O | L68 |
| P1-568 | O | A2 | A12 | O | L119 |
| P1-569 | O | A2 | A12 | O | L128 |
| P1-570 | O | A2 | A12 | O | L148 |
| P1-571 | O | B4 | A12 | O | Single bond |
| P1-572 | O | B4 | A12 | O | O |
| P1-573 | O | B4 | A12 | O | L1 |
| P1-574 | O | B4 | A12 | O | L5 |
| P1-575 | O | B4 | A12 | O | L9 |
| P1-576 | O | B4 | A12 | O | L18 |
| P1-577 | O | B4 | A12 | O | L22 |
| P1-578 | O | B4 | A12 | O | L27 |
| P1-579 | O | B4 | A12 | O | L29 |
| P1-580 | O | B4 | A12 | O | L36 |
| P1-581 | O | B4 | A12 | O | L40 |
| P1-582 | O | B4 | A12 | O | L53 |
| P1-583 | O | B4 | A12 | O | L59 |
| P1-584 | O | B4 | A12 | O | L65 |
| P1-585 | O | B4 | A12 | O | L68 |
| P1-586 | O | B4 | A12 | O | L119 |
| P1-587 | O | B4 | A12 | O | L128 |
| P1-588 | O | B4 | A12 | O | L148 |
| P1-589 | O | B23 | A12 | O | Single bond |
| P1-590 | O | B23 | A12 | O | O |
| P1-591 | O | B23 | A12 | O | L1 |
| P1-592 | O | B23 | A12 | O | L5 |
| P1-593 | O | B23 | A12 | O | L9 |
| P1-594 | O | B23 | A12 | O | L18 |
| P1-595 | O | B23 | A12 | O | L22 |
| P1-596 | O | B23 | A12 | O | L27 |
| P1-597 | O | B23 | A12 | O | L29 |
| P1-598 | O | B23 | A12 | O | L36 |
| P1-599 | O | B23 | A12 | O | L40 |
| P1-600 | O | B23 | A12 | O | L53 |
| P1-601 | O | B23 | A12 | O | L59 |
| P1-602 | O | B23 | A12 | O | L65 |
| P1-603 | O | B23 | A12 | O | L68 |
| P1-604 | O | B23 | A12 | O | L119 |
| P1-605 | O | B23 | A12 | O | L128 |
| P1-606 | O | B23 | A12 | O | L148 |
| P1-607 | O | B36 | A12 | O | Single bond |
| P1-608 | O | B36 | A12 | O | O |
| P1-609 | O | B36 | A12 | O | L1 |
| P1-610 | O | B36 | A12 | O | L5 |
| P1-611 | O | B36 | A12 | O | L9 |
| P1-612 | O | B36 | A12 | O | L18 |
| P1-613 | O | B36 | A12 | O | L22 |
| P1-614 | O | B36 | A12 | O | L27 |
| P1-615 | O | B36 | A12 | O | L29 |
| P1-616 | O | B36 | A12 | O | L36 |
| P1-617 | O | B36 | A12 | O | L40 |
| P1-618 | O | B36 | A12 | O | L53 |
| P1-619 | O | B36 | A12 | O | L59 |
| P1-620 | O | B36 | A12 | O | L65 |
| P1-621 | O | B36 | A12 | O | L68 |
| P1-622 | O | B36 | A12 | O | L119 |
| P1-623 | O | B36 | A12 | O | L128 |
| P1-624 | O | B36 | A12 | O | L148 |
| P1-625 | O | B44 | A12 | O | Single bond |
| P1-626 | O | B44 | A12 | O | O |
| P1-627 | O | B44 | A12 | O | L1 |
| P1-628 | O | B44 | A12 | O | L5 |
| P1-629 | O | B44 | A12 | O | L9 |
| P1-630 | O | B44 | A12 | O | L18 |
| P1-631 | O | B44 | A12 | O | L22 |
| P1-632 | O | B44 | A12 | O | L27 |
| P1-633 | O | B44 | A12 | O | L29 |
| P1-634 | O | B44 | A12 | O | L36 |
| P1-635 | O | B44 | A12 | O | L40 |
| P1-636 | O | B44 | A12 | O | L53 |
| P1-637 | O | B44 | A12 | O | L59 |
| P1-638 | O | B44 | A12 | O | L65 |
| P1-639 | O | B44 | A12 | O | L68 |
| P1-640 | O | B44 | A12 | O | L119 |
| P1-641 | O | B44 | A12 | O | L128 |
| P1-642 | O | B44 | A12 | O | L148 |
| P1-643 | O | B92 | A12 | O | Single bond |
| P1-644 | O | B92 | A12 | O | O |
| P1-645 | O | B92 | A12 | O | L1 |
| P1-646 | O | B92 | A12 | O | L5 |
| P1-647 | O | B92 | A12 | O | L9 |
| P1-648 | O | B92 | A12 | O | L18 |
| P1-649 | O | B92 | A12 | O | L22 |
| P1-650 | O | B92 | A12 | O | L27 |
| P1-651 | O | B92 | A12 | O | L29 |
| P1-652 | O | B92 | A12 | O | L36 |
| P1-653 | O | B92 | A12 | O | L40 |
| P1-654 | O | B92 | A12 | O | L53 |
| P1-655 | O | B92 | A12 | O | L59 |
| P1-656 | O | B92 | A12 | O | L65 |
| P1-657 | O | B92 | A12 | O | L68 |
| P1-658 | O | B92 | A12 | O | L119 |
| P1-659 | O | B92 | A12 | O | L128 |
| P1-660 | O | B92 | A12 | O | L148 |
| P1-661 | O | A2 | B41 | O | Single bond |
| P1-662 | O | A2 | B41 | O | O |
| P1-663 | O | A2 | B41 | O | L1 |
| P1-664 | O | A2 | B41 | O | L5 |
| P1-665 | O | A2 | B41 | O | L9 |
| P1-666 | O | A2 | B41 | O | L18 |
| P1-667 | O | A2 | B41 | O | L22 |
| P1-668 | O | A2 | B41 | O | L27 |
| P1-669 | O | A2 | B41 | O | L29 |
| P1-670 | O | A2 | B41 | O | L36 |
| P1-671 | O | A2 | B41 | O | L40 |
| P1-672 | O | A2 | B41 | O | L53 |
| P1-673 | O | A2 | B41 | O | L59 |
| P1-674 | O | A2 | B41 | O | L65 |
| P1-675 | O | A2 | B41 | O | L68 |
| P1-676 | O | A2 | B41 | O | L119 |
| P1-677 | O | A2 | B41 | O | L128 |
| P1-678 | O | A2 | B41 | O | L148 |
| P1-679 | O | B4 | B41 | O | Single bond |
| P1-680 | O | B4 | B41 | O | O |
| P1-681 | O | B4 | B41 | O | L1 |
| P1-682 | O | B4 | B41 | O | L5 |
| P1-683 | O | B4 | B41 | O | L9 |
| P1-684 | O | B4 | B41 | O | L18 |
| P1-685 | O | B4 | B41 | O | L22 |
| P1-686 | O | B4 | B41 | O | L27 |
| P1-687 | O | B4 | B41 | O | L29 |
| P1-688 | O | B4 | B41 | O | L36 |
| P1-689 | O | B4 | B41 | O | L40 |
| P1-690 | O | B4 | B41 | O | L53 |
| P1-691 | O | B4 | B41 | O | L59 |
| P1-692 | O | B4 | B41 | O | L65 |
| P1-693 | O | B4 | B41 | O | L68 |
| P1-694 | O | B4 | B41 | O | L119 |
| P1-695 | O | B4 | B41 | O | L128 |
| P1-696 | O | B4 | B41 | O | L148 |
| P1-697 | O | B23 | B41 | O | Single bond |
| P1-698 | O | B23 | B41 | O | O |
| P1-699 | O | B23 | B41 | O | L1 |
| P1-700 | O | B23 | B41 | O | L5 |
| P1-701 | O | B23 | B41 | O | L9 |
| P1-702 | O | B23 | B41 | O | L18 |
| P1-703 | O | B23 | B41 | O | L22 |
| P1-704 | O | B23 | B41 | O | L27 |
| P1-705 | O | B23 | B41 | O | L29 |
| P1-706 | O | B23 | B41 | O | L36 |
| P1-707 | O | B23 | B41 | O | L40 |
| P1-708 | O | B23 | B41 | O | L53 |
| P1-709 | O | B23 | B41 | O | L59 |
| P1-710 | O | B23 | B41 | O | L65 |
| P1-711 | O | B23 | B41 | O | L68 |
| P1-712 | O | B23 | B41 | O | L119 |
| P1-713 | O | B23 | B41 | O | L128 |
| P1-714 | O | B23 | B41 | O | L148 |
| P1-715 | O | B36 | B41 | O | Single bond |
| P1-716 | O | B36 | B41 | O | O |
| P1-717 | O | B36 | B41 | O | L1 |
| P1-718 | O | B36 | B41 | O | L5 |
| P1-719 | O | B36 | B41 | O | L9 |
| P1-720 | O | B36 | B41 | O | L18 |
| P1-721 | O | B36 | B41 | O | L22 |
| P1-722 | O | B36 | B41 | O | L27 |
| P1-723 | O | B36 | B41 | O | L29 |
| P1-724 | O | B36 | B41 | O | L36 |
| P1-725 | O | B36 | B41 | O | L40 |
| P1-726 | O | B36 | B41 | O | L53 |
| P1-727 | O | B36 | B41 | O | L59 |
| P1-728 | O | B36 | B41 | O | L65 |
| P1-729 | O | B36 | B41 | O | L68 |
| P1-730 | O | B36 | B41 | O | L119 |
| P1-731 | O | B36 | B41 | O | L128 |
| P1-732 | O | B36 | B41 | O | L148 |
| P1-733 | O | B44 | B41 | O | Single bond |
| P1-734 | O | B44 | B41 | O | O |
| P1-735 | O | B44 | B41 | O | L1 |
| P1-736 | O | B44 | B41 | O | L5 |
| P1-737 | O | B44 | B41 | O | L9 |
| P1-738 | O | B44 | B41 | O | L18 |
| P1-739 | O | B44 | B41 | O | L22 |
| P1-740 | O | B44 | B41 | O | L27 |
| P1-741 | O | B44 | B41 | O | L29 |
| P1-742 | O | B44 | B41 | O | L36 |
| P1-743 | O | B44 | B41 | O | L40 |
| P1-744 | O | B44 | B41 | O | L53 |
| P1-745 | O | B44 | B41 | O | L59 |
| P1-746 | O | B44 | B41 | O | L65 |
| P1-747 | O | B44 | B41 | O | L68 |
| P1-748 | O | B44 | B41 | O | L119 |
| P1-749 | O | B44 | B41 | O | L128 |
| P1-750 | O | B44 | B41 | O | L148 |
| P1-751 | O | B92 | B41 | O | Single bond |
| P1-752 | O | B92 | B41 | O | O |
| P1-753 | O | B92 | B41 | O | L1 |
| P1-754 | O | B92 | B41 | O | L5 |
| P1-755 | O | B92 | B41 | O | L9 |
| P1-756 | O | B92 | B41 | O | L18 |
| P1-757 | O | B92 | B41 | O | L22 |
| P1-758 | O | B92 | B41 | O | L27 |
| P1-759 | O | B92 | B41 | O | L29 |
| P1-760 | O | B92 | B41 | O | L36 |
| P1-761 | O | B92 | B41 | O | L40 |
| P1-762 | O | B92 | B41 | O | L53 |
| P1-763 | O | B92 | B41 | O | L59 |
| P1-764 | O | B92 | B41 | O | L65 |
| P1-765 | O | B92 | B41 | O | L68 |
| P1-766 | O | B92 | B41 | O | L119 |
| P1-767 | O | B92 | B41 | O | L128 |
| P1-768 | O | B92 | B41 | O | L148 |
| P1-769 | O | A2 | B56 | O | Single bond |
| P1-770 | O | A2 | B56 | O | O |
| P1-771 | O | A2 | B56 | O | L1 |
| P1-772 | O | A2 | B56 | O | L5 |
| P1-773 | O | A2 | B56 | O | L9 |
| P1-774 | O | A2 | B56 | O | L18 |
| P1-775 | O | A2 | B56 | O | L22 |
| P1-776 | O | A2 | B56 | O | L27 |
| P1-777 | O | A2 | B56 | O | L29 |
| P1-778 | O | A2 | B56 | O | L36 |
| P1-779 | O | A2 | B56 | O | L40 |
| P1-780 | O | A2 | B56 | O | L53 |
| P1-781 | O | A2 | B56 | O | L59 |
| P1-782 | O | A2 | B56 | O | L65 |
| P1-783 | O | A2 | B56 | O | L68 |
| P1-784 | O | A2 | B56 | O | L119 |
| P1-785 | O | A2 | B56 | O | L128 |
| P1-786 | O | A2 | B56 | O | L148 |
| P1-787 | O | B4 | B56 | O | Single bond |
| P1-788 | O | B4 | B56 | O | O |
| P1-789 | O | B4 | B56 | O | L1 |
| P1-790 | O | B4 | B56 | O | L5 |
| P1-791 | O | B4 | B56 | O | L9 |
| P1-792 | O | B4 | B56 | O | L18 |
| P1-793 | O | B4 | B56 | O | L22 |
| P1-794 | O | B4 | B56 | O | L27 |
| P1-795 | O | B4 | B56 | O | L29 |
| P1-796 | O | B4 | B56 | O | L36 |
| P1-797 | O | B4 | B56 | O | L40 |
| P1-798 | O | B4 | B56 | O | L53 |
| P1-799 | O | B4 | B56 | O | L59 |
| P1-800 | O | B4 | B56 | O | L65 |
| P1-801 | O | B4 | B56 | O | L68 |
| P1-802 | O | B4 | B56 | O | L119 |
| P1-803 | O | B4 | B56 | O | L128 |
| P1-804 | O | B4 | B56 | O | L148 |
| P1-805 | O | B23 | B56 | O | Single bond |
| P1-806 | O | B23 | B56 | O | O |
| P1-807 | O | B23 | B56 | O | L1 |
| P1-808 | O | B23 | B56 | O | L5 |
| P1-809 | O | B23 | B56 | O | L9 |
| P1-810 | O | B23 | B56 | O | L18 |
| P1-811 | O | B23 | B56 | O | L22 |
| P1-812 | O | B23 | B56 | O | L27 |
| P1-813 | O | B23 | B56 | O | L29 |
| P1-814 | O | B23 | B56 | O | L36 |
| P1-815 | O | B23 | B56 | O | L40 |
| P1-816 | O | B23 | B56 | O | L53 |
| P1-817 | O | B23 | B56 | O | L59 |
| P1-818 | O | B23 | B56 | O | L65 |
| P1-819 | O | B23 | B56 | O | L68 |
| P1-820 | O | B23 | B56 | O | L119 |
| P1-821 | O | B23 | B56 | O | L128 |
| P1-822 | O | B23 | B56 | O | L148 |
| P1-823 | O | B36 | B56 | O | Single bond |
| P1-824 | O | B36 | B56 | O | O |
| P1-825 | O | B36 | B56 | O | L1 |
| P1-826 | O | B36 | B56 | O | L5 |
| P1-827 | O | B36 | B56 | O | L9 |
| P1-828 | O | B36 | B56 | O | L18 |
| P1-829 | O | B36 | B56 | O | L22 |
| P1-830 | O | B36 | B56 | O | L27 |
| P1-831 | O | B36 | B56 | O | L29 |
| P1-832 | O | B36 | B56 | O | L36 |
| P1-833 | O | B36 | B56 | O | L40 |
| P1-834 | O | B36 | B56 | O | L53 |
| P1-835 | O | B36 | B56 | O | L59 |
| P1-836 | O | B36 | B56 | O | L65 |
| P1-837 | O | B36 | B56 | O | L68 |
| P1-838 | O | B36 | B56 | O | L119 |
| P1-839 | O | B36 | B56 | O | L128 |
| P1-840 | O | B36 | B56 | O | L148 |
| P1-841 | O | B44 | B56 | O | Single bond |
| P1-842 | O | B44 | B56 | O | O |
| P1-843 | O | B44 | B56 | O | L1 |
| P1-844 | O | B44 | B56 | O | L5 |
| P1-845 | O | B44 | B56 | O | L9 |
| P1-846 | O | B44 | B56 | O | L18 |
| P1-847 | O | B44 | B56 | O | L22 |
| P1-848 | O | B44 | B56 | O | L27 |
| P1-849 | O | B44 | B56 | O | L29 |
| P1-850 | O | B44 | B56 | O | L36 |
| P1-851 | O | B44 | B56 | O | L40 |
| P1-852 | O | B44 | B56 | O | L53 |
| P1-853 | O | B44 | B56 | O | L59 |
| P1-854 | O | B44 | B56 | O | L65 |
| P1-855 | O | B44 | B56 | O | L68 |
| P1-856 | O | B44 | B56 | O | L119 |
| P1-857 | O | B44 | B56 | O | L128 |
| P1-858 | O | B44 | B56 | O | L148 |
| P1-859 | O | B92 | B56 | O | Single bond |
| P1-860 | O | B92 | B56 | O | O |
| P1-861 | O | B92 | B56 | O | L1 |
| P1-862 | O | B92 | B56 | O | L5 |
| P1-863 | O | B92 | B56 | O | L9 |
| P1-864 | O | B92 | B56 | O | L18 |
| P1-865 | O | B92 | B56 | O | L22 |
| P1-866 | O | B92 | B56 | O | L27 |
| P1-867 | O | B92 | B56 | O | L29 |
| P1-868 | O | B92 | B56 | O | L36 |
| P1-869 | O | B92 | B56 | O | L40 |
| P1-870 | O | B92 | B56 | O | L53 |
| P1-871 | O | B92 | B56 | O | L59 |
| P1-872 | O | B92 | B56 | O | L65 |
| P1-873 | O | B92 | B56 | O | L68 |
| P1-874 | O | B92 | B56 | O | L119 |
| P1-875 | O | B92 | B56 | O | L128 |
| P1-876 | O | B92 | B56 | O | L148 |
| P1-877 | O | B44 | A2 | S | Single bond |
| P1-878 | O | B44 | A2 | S | L5 |
| P1-879 | O | B44 | A2 | S | L6 |
| P1-880 | O | B44 | A2 | S | L27 |
| P1-881 | O | B44 | A2 | S | L29 |
| P1-882 | O | B44 | A2 | S | L36 |
| P1-883 | O | B92 | A2 | S | Single bond |
| P1-884 | O | B92 | A2 | S | L5 |
| P1-885 | O | B92 | A2 | S | L6 |
| P1-886 | O | B92 | A2 | S | L27 |
| P1-887 | O | B92 | A2 | S | L29 |
| P1-888 | O | B92 | A2 | S | L36 |
| P1-889 | O | B44 | A3 | S | Single bond |
| P1-890 | O | B44 | A3 | S | L5 |
| P1-891 | O | B44 | A3 | S | L6 |
| P1-892 | O | B44 | A3 | S | L27 |
| P1-893 | O | B44 | A3 | S | L29 |
| P1-894 | O | B44 | A3 | S | L36 |
| P1-895 | O | B92 | A3 | S | Single bond |
| P1-896 | O | B92 | A3 | S | L5 |
| P1-897 | O | B92 | A3 | S | L6 |
| P1-898 | O | B92 | A3 | S | L27 |
| P1-899 | O | B92 | A3 | S | L29 |
| P1-900 | O | B92 | A3 | S | L36 |
| P1-901 | O | B44 | A2 | Se | L6 |
| P1-902 | O | B44 | A2 | Se | L36 |
| P1-903 | O | B92 | A2 | Se | L6 |
| P1-904 | O | B92 | A2 | Se | L36 |
| P1-905 | O | B44 | A3 | Se | L6 |
| P1-906 | O | B44 | A3 | Se | L36 |
| P1-907 | O | B92 | A3 | Se | L6 |
| P1-908 | O | B92 | A3 | Se | L36 |
| P1-909 | O | B44 | A2 | S═O | L6 |
| P1-910 | O | B44 | A2 | S═O | L36 |
| P1-911 | O | B92 | A2 | S═O | L6 |
| P1-912 | O | B92 | A2 | S═O | L36 |
| P1-913 | O | B44 | A3 | S═O | L6 |
| P1-914 | O | B44 | A3 | S═O | L36 |
| P1-915 | O | B92 | A3 | S═O | L6 |
| P1-916 | O | B92 | A3 | S═O | L36 |
| P1-917 | O | B44 | A2 | O═S═O | Single bond |
| P1-918 | O | B44 | A2 | O═S═O | L5 |
| P1-919 | O | B44 | A2 | O═S═O | L6 |
| P1-920 | O | B44 | A2 | O═S═O | L27 |
| P1-921 | O | B44 | A2 | O═S═O | L29 |
| P1-922 | O | B44 | A2 | O═S═O | L36 |
| P1-923 | O | B92 | A2 | O═S═O | Single bond |
| P1-924 | O | B92 | A2 | O═S═O | L5 |
| P1-925 | O | B92 | A2 | O═S═O | L6 |
| P1-926 | O | B92 | A2 | O═S═O | L27 |
| P1-927 | O | B92 | A2 | O═S═O | L29 |
| P1-928 | O | B92 | A2 | O═S═O | L36 |
| P1-929 | O | B44 | A3 | O═S═O | Single bond |
| P1-930 | O | B44 | A3 | O═S═O | L5 |
| P1-931 | O | B44 | A3 | O═S═O | L6 |
| P1-932 | O | B44 | A3 | O═S═O | L27 |
| P1-933 | O | B44 | A3 | O═S═O | L29 |
| P1-934 | O | B44 | A3 | O═S═O | L36 |
| P1-935 | O | B92 | A3 | O═S═O | Single bond |
| P1-936 | O | B92 | A3 | O═S═O | L5 |
| P1-937 | O | B92 | A3 | O═S═O | L6 |
| P1-938 | O | B92 | A3 | O═S═O | L27 |
| P1-939 | O | B92 | A3 | O═S═O | L29 |
| P1-940 | O | B92 | A3 | O═S═O | L36 |
| P1-941 | S | B44 | A2 | O | Single bond |
| P1-942 | S | B44 | A2 | O | L5 |
| P1-943 | S | B44 | A2 | O | L6 |
| P1-944 | S | B44 | A2 | O | L27 |
| P1-945 | S | B44 | A2 | O | L29 |
| P1-946 | S | B44 | A2 | O | L36 |
| P1-947 | S | B92 | A2 | O | Single bond |
| P1-948 | S | B92 | A2 | O | L5 |
| P1-949 | S | B92 | A2 | O | L6 |
| P1-950 | S | B92 | A2 | O | L27 |
| P1-951 | S | B92 | A2 | O | L29 |
| P1-952 | S | B92 | A2 | O | L36 |
| P1-953 | S | B44 | A3 | O | Single bond |
| P1-954 | S | B44 | A3 | O | L5 |
| P1-955 | S | B44 | A3 | O | L6 |
| P1-956 | S | B44 | A3 | O | L27 |
| P1-957 | S | B44 | A3 | O | L29 |
| P1-958 | S | B44 | A3 | O | L36 |
| P1-959 | S | B92 | A3 | O | Single bond |
| P1-960 | S | B92 | A3 | O | L5 |
| P1-961 | S | B92 | A3 | O | L6 |
| P1-962 | S | B92 | A3 | O | L27 |
| P1-963 | S | B92 | A3 | O | L29 |
| P1-964 | S | B92 | A3 | O | L36 |
| P1-965 | Se | B44 | A2 | O | L5 |
| P1-966 | Se | B44 | A2 | O | L6 |
| P1-967 | Se | B44 | A2 | O | L27 |
| P1-968 | Se | B44 | A2 | O | L29 |
| P1-969 | Se | B44 | A2 | O | L36 |
| P1-970 | Se | B92 | A2 | O | L5 |
| P1-971 | Se | B92 | A2 | O | L6 |
| P1-972 | Se | B92 | A2 | O | L27 |
| P1-973 | Se | B92 | A2 | O | L29 |
| P1-974 | Se | B92 | A2 | O | L36 |
| P1-975 | Se | B44 | A3 | O | L5 |
| P1-976 | Se | B44 | A3 | O | L6 |
| P1-977 | Se | B44 | A3 | O | L27 |
| P1-978 | Se | B44 | A3 | O | L29 |
| P1-979 | Se | B44 | A3 | O | L36 |
| P1-980 | Se | B92 | A3 | O | L5 |
| P1-981 | Se | B92 | A3 | O | L6 |
| P1-982 | Se | B92 | A3 | O | L27 |
| P1-983 | Se | B92 | A3 | O | L29 |
| P1-984 | Se | B92 | A3 | O | L36 |
| P1-985 | S | B44 | A2 | O═S═O | Single bond |
| P1-986 | S | B44 | A2 | O═S═O | L5 |
| P1-987 | S | B44 | A2 | O═S═O | L6 |
| P1-988 | S | B44 | A2 | O═S═O | L27 |
| P1-989 | S | B44 | A2 | O═S═O | L29 |
| P1-990 | S | B44 | A2 | O═S═O | L36 |
| P1-991 | S | B92 | A2 | O═S═O | Single bond |
| P1-992 | S | B92 | A2 | O═S═O | L5 |
| P1-993 | S | B92 | A2 | O═S═O | L6 |
| P1-994 | S | B92 | A2 | O═S═O | L27 |
| P1-995 | S | B92 | A2 | O═S═O | L29 |
| P1-996 | S | B92 | A2 | O═S═O | L36 |
| P1-997 | S | B44 | A3 | O═S═O | Single bond |
| P1-998 | S | B44 | A3 | O═S═O | L5 |
| P1-999 | S | B44 | A3 | O═S═O | L6 |
| P1-1000 | S | B44 | A3 | O═S═O | L27 |
| P1-1001 | S | B44 | A3 | O═S═O | L29 |
| P1-1002 | S | B44 | A3 | O═S═O | L36 |
| P1-1003 | S | B92 | A3 | O═S═O | Single bond |
| P1-1004 | S | B92 | A3 | O═S═O | L5 |
| P1-1005 | S | B92 | A3 | O═S═O | L6 |
| P1-1006 | S | B92 | A3 | O═S═O | L27 |
| P1-1007 | S | B92 | A3 | O═S═O | L29 |
| P1-1008 | S | B92 | A3 | O═S═O | L36 |
| P1-1009 | Se | B44 | A2 | O═S═O | L5 |
| P1-1010 | Se | B44 | A2 | O═S═O | L6 |
| P1-1011 | Se | B44 | A2 | O═S═O | L27 |
| P1-1012 | Se | B44 | A2 | O═S═O | L29 |
| P1-1013 | Se | B44 | A2 | O═S═O | L36 |
| P1-1014 | Se | B92 | A2 | O═S═O | L5 |
| P1-1015 | Se | B92 | A2 | O═S═O | L6 |
| P1-1016 | Se | B92 | A2 | O═S═O | L27 |
| P1-1017 | Se | B92 | A2 | O═S═O | L29 |
| P1-1018 | Se | B92 | A2 | O═S═O | L36 |
| P1-1019 | Se | B44 | A3 | O═S═O | L5 |
| P1-1020 | Se | B44 | A3 | O═S═O | L6 |
| P1-1021 | Se | B44 | A3 | O═S═O | L27 |
| P1-1022 | Se | B44 | A3 | O═S═O | L29 |
| P1-1023 | Se | B44 | A3 | O═S═O | L36 |
| P1-1024 | Se | B92 | A3 | O═S═O | L5 |
| P1-1025 | Se | B92 | A3 | O═S═O | L6 |
| P1-1026 | Se | B92 | A3 | O═S═O | L27 |
| P1-1027 | Se | B92 | A3 | O═S═O | L29 |
| P1-1028 | Se | B92 | A3 | O═S═O | L36 |

The compounds P2- to P2-1028 have structures represented by formula I-1-2; wherein R¹ and R² are the same, and W Ar¹, R¹, R², X, and L correspond to atoms or groups selected from in the following table 2, respectively:

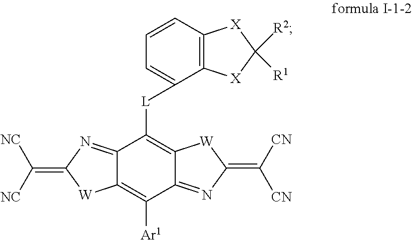

TABLE 2

| Compound No. | W | Ar¹ | R¹ and R² | X | L |
|---|---|---|---|---|---|
| P2-1 | O | A2 | A1 | O | Single bond |
| P2-2 | O | A2 | A1 | O | O |
| P2-3 | O | A2 | A1 | O | L1 |
| P2-4 | O | A2 | A1 | O | L5 |
| P2-5 | O | A2 | A1 | O | L9 |
| P2-6 | O | A2 | A1 | O | L18 |
| P2-7 | O | A2 | A1 | O | L22 |
| P2-8 | O | A2 | A1 | O | L27 |
| P2-9 | O | A2 | A1 | O | L29 |
| P2-10 | O | A2 | A1 | O | L36 |
| P2-11 | O | A2 | A1 | O | L40 |
| P2-12 | O | A2 | A1 | O | L53 |
| P2-13 | O | A2 | A1 | O | L59 |
| P2-14 | O | A2 | A1 | O | L65 |
| P2-15 | O | A2 | A1 | O | L68 |
| P2-16 | O | A2 | A1 | O | L119 |
| P2-17 | O | A2 | A1 | O | L128 |
| P2-18 | O | A2 | A1 | O | L148 |
| P2-19 | O | B4 | A1 | O | Single bond |
| P2-20 | O | B4 | A1 | O | O |
| P2-21 | O | B4 | A1 | O | L1 |
| P2-22 | O | B4 | A1 | O | L5 |
| P2-23 | O | B4 | A1 | O | L9 |
| P2-24 | O | B4 | A1 | O | L18 |
| P2-25 | O | B4 | A1 | O | L22 |
| P2-26 | O | B4 | A1 | O | L27 |
| P2-27 | O | B4 | A1 | O | L29 |
| P2-28 | O | B4 | A1 | O | L36 |
| P2-29 | O | B4 | A1 | O | L40 |
| P2-30 | O | B4 | A1 | O | L53 |
| P2-31 | O | B4 | A1 | O | L59 |
| P2-32 | O | B4 | A1 | O | L65 |
| P2-33 | O | B4 | A1 | O | L68 |
| P2-34 | O | B4 | A1 | O | L119 |
| P2-35 | O | B4 | A1 | O | L128 |
| P2-36 | O | B4 | A1 | O | L148 |
| P2-37 | O | B23 | A1 | O | Single bond |
| P2-38 | O | B23 | A1 | O | O |
| P2-39 | O | B23 | A1 | O | L1 |
| P2-40 | O | B23 | A1 | O | L5 |
| P2-41 | O | B23 | A1 | O | L9 |
| P2-42 | O | B23 | A1 | O | L18 |
| P2-43 | O | B23 | A1 | O | L22 |
| P2-44 | O | B23 | A1 | O | L27 |
| P2-45 | O | B23 | A1 | O | L29 |
| P2-46 | O | B23 | A1 | O | L36 |
| P2-47 | O | B23 | A1 | O | L40 |
| P2-48 | O | B23 | A1 | O | L53 |
| P2-49 | O | B23 | A1 | O | L59 |
| P2-50 | O | B23 | A1 | O | L65 |
| P2-51 | O | B23 | A1 | O | L68 |
| P2-52 | O | B23 | A1 | O | L119 |
| P2-53 | O | B23 | A1 | O | L128 |
| P2-54 | O | B23 | A1 | O | L148 |
| P2-55 | O | B36 | A1 | O | Single bond |
| P2-56 | O | B36 | A1 | O | O |
| P2-57 | O | B36 | A1 | O | L1 |
| P2-58 | O | B36 | A1 | O | L5 |
| P2-59 | O | B36 | A1 | O | L9 |
| P2-60 | O | B36 | A1 | O | L18 |
| P2-61 | O | B36 | A1 | O | L22 |
| P2-62 | O | B36 | A1 | O | L27 |
| P2-63 | O | B36 | A1 | O | L29 |
| P2-64 | O | B36 | A1 | O | L36 |
| P2-65 | O | B36 | A1 | O | L40 |
| P2-66 | O | B36 | A1 | O | L53 |
| P2-67 | O | B36 | A1 | O | L59 |
| P2-68 | O | B36 | A1 | O | L65 |
| P2-69 | O | B36 | A1 | O | L68 |
| P2-70 | O | B36 | A1 | O | L119 |
| P2-71 | O | B36 | A1 | O | L128 |
| P2-72 | O | B36 | A1 | O | L148 |
| P2-73 | O | B44 | A1 | O | Single bond |
| P2-74 | O | B44 | A1 | O | O |
| P2-75 | O | B44 | A1 | O | L1 |
| P2-76 | O | B44 | A1 | O | L5 |
| P2-77 | O | B44 | A1 | O | L9 |
| P2-78 | O | B44 | A1 | O | L18 |
| P2-79 | O | B44 | A1 | O | L22 |
| P2-80 | O | B44 | A1 | O | L27 |
| P2-81 | O | B44 | A1 | O | L29 |
| P2-82 | O | B44 | A1 | O | L36 |
| P2-83 | O | B44 | A1 | O | L40 |
| P2-84 | O | B44 | A1 | O | L53 |
| P2-85 | O | B44 | A1 | O | L59 |
| P2-86 | O | B44 | A1 | O | L65 |
| P2-87 | O | B44 | A1 | O | L68 |
| P2-88 | O | B44 | A1 | O | L119 |
| P2-89 | O | B44 | A1 | O | L128 |
| P2-90 | O | B44 | A1 | O | L148 |
| P2-91 | O | B92 | A1 | O | Single bond |
| P2-92 | O | B92 | A1 | O | O |
| P2-93 | O | B92 | A1 | O | L1 |
| P2-94 | O | B92 | A1 | O | L5 |
| P2-95 | O | B92 | A1 | O | L9 |
| P2-96 | O | B92 | A1 | O | L18 |
| P2-97 | O | B92 | A1 | O | L22 |
| P2-98 | O | B92 | A1 | O | L27 |
| P2-99 | O | B92 | A1 | O | L29 |
| P2-100 | O | B92 | A1 | O | L36 |
| P2-101 | O | B92 | A1 | O | L40 |
| P2-102 | O | B92 | A1 | O | L53 |
| P2-103 | O | B92 | A1 | O | L59 |
| P2-104 | O | B92 | A1 | O | L65 |
| P2-105 | O | B92 | A1 | O | L68 |
| P2-106 | O | B92 | A1 | O | L119 |
| P2-107 | O | B92 | A1 | O | L128 |
| P2-108 | O | B92 | A1 | O | L148 |
| P2-109 | O | A2 | A2 | O | Single bond |
| P2-110 | O | A2 | A2 | O | O |
| P2-111 | O | A2 | A2 | O | L1 |
| P2-112 | O | A2 | A2 | O | L2 |
| P2-113 | O | A2 | A2 | O | L3 |
| P2-114 | O | A2 | A2 | O | L4 |
| P2-115 | O | A2 | A2 | O | L5 |
| P2-116 | O | A2 | A2 | O | L6 |
| P2-117 | O | A2 | A2 | O | L7 |
| P2-118 | O | A2 | A2 | O | L9 |
| P2-119 | O | A2 | A2 | O | L11 |
| P2-120 | O | A2 | A2 | O | L14 |
| P2-121 | O | A2 | A2 | O | L18 |
| P2-122 | O | A2 | A2 | O | L22 |
| P2-123 | O | A2 | A2 | O | L27 |
| P2-124 | O | A2 | A2 | O | L28 |
| P2-125 | O | A2 | A2 | O | L29 |
| P2-126 | O | A2 | A2 | O | L30 |
| P2-127 | O | A2 | A2 | O | L32 |
| P2-128 | O | A2 | A2 | O | L36 |
| P2-129 | O | A2 | A2 | O | L40 |
| P2-130 | O | A2 | A2 | O | L48 |
| P2-131 | O | A2 | A2 | O | L53 |
| P2-132 | O | A2 | A2 | O | L59 |
| P2-133 | O | A2 | A2 | O | L65 |
| P2-134 | O | A2 | A2 | O | L68 |
| P2-135 | O | A2 | A2 | O | L79 |
| P2-136 | O | A2 | A2 | O | L89 |
| P2-137 | O | A2 | A2 | O | L102 |
| P2-138 | O | A2 | A2 | O | L111 |
| P2-139 | O | A2 | A2 | O | L119 |
| P2-140 | O | A2 | A2 | O | L128 |
| P2-141 | O | A2 | A2 | O | L148 |
| P2-142 | O | A2 | A2 | O | L156 |
| P2-143 | O | A2 | A2 | O | L167 |
| P2-144 | O | A2 | A2 | O | L169 |
| P2-145 | O | A2 | A2 | O | L172 |
| P2-146 | O | B4 | A2 | O | Single bond |
| P2-147 | O | B4 | A2 | O | O |
| P2-148 | O | B4 | A2 | O | L1 |
| P2-149 | O | B4 | A2 | O | L2 |
| P2-150 | O | B4 | A2 | O | L3 |
| P2-151 | O | B4 | A2 | O | L4 |
| P2-152 | O | B4 | A2 | O | L5 |
| P2-153 | O | B4 | A2 | O | L6 |
| P2-154 | O | B4 | A2 | O | L7 |
| P2-155 | O | B4 | A2 | O | L9 |
| P2-156 | O | B4 | A2 | O | L11 |
| P2-157 | O | B4 | A2 | O | L14 |
| P2-158 | O | B4 | A2 | O | L18 |
| P2-159 | O | B4 | A2 | O | L22 |
| P2-160 | O | B4 | A2 | O | L27 |
| P2-161 | O | B4 | A2 | O | L28 |
| P2-162 | O | B4 | A2 | O | L29 |
| P2-163 | O | B4 | A2 | O | L30 |
| P2-164 | O | B4 | A2 | O | L32 |
| P2-165 | O | B4 | A2 | O | L36 |
| P2-166 | O | B4 | A2 | O | L40 |
| P2-167 | O | B4 | A2 | O | L48 |
| P2-168 | O | B4 | A2 | O | L53 |
| P2-169 | O | B4 | A2 | O | L59 |
| P2-170 | O | B4 | A2 | O | L65 |
| P2-171 | O | B4 | A2 | O | L68 |
| P2-172 | O | B4 | A2 | O | L79 |
| P2-173 | O | B4 | A2 | O | L89 |
| P2-174 | O | B4 | A2 | O | L102 |
| P2-175 | O | B4 | A2 | O | L111 |
| P2-176 | O | B4 | A2 | O | L119 |
| P2-177 | O | B4 | A2 | O | L128 |
| P2-178 | O | B4 | A2 | O | L148 |
| P2-179 | O | B4 | A2 | O | L156 |
| P2-180 | O | B4 | A2 | O | L167 |
| P2-181 | O | B4 | A2 | O | L169 |
| P2-182 | O | B4 | A2 | O | L172 |
| P2-183 | O | B23 | A2 | O | Single bond |
| P2-184 | O | B23 | A2 | O | O |
| P2-185 | O | B23 | A2 | O | L1 |
| P2-186 | O | B23 | A2 | O | L2 |
| P2-187 | O | B23 | A2 | O | L3 |
| P2-188 | O | B23 | A2 | O | L4 |
| P2-189 | O | B23 | A2 | O | L5 |
| P2-190 | O | B23 | A2 | O | L6 |
| P2-191 | O | B23 | A2 | O | L7 |
| P2-192 | O | B23 | A2 | O | L9 |
| P2-193 | O | B23 | A2 | O | L11 |
| P2-194 | O | B23 | A2 | O | L14 |
| P2-195 | O | B23 | A2 | O | L18 |
| P2-196 | O | B23 | A2 | O | L22 |
| P2-197 | O | B23 | A2 | O | L27 |
| P2-198 | O | B23 | A2 | O | L28 |
| P2-199 | O | B23 | A2 | O | L29 |
| P2-200 | O | B23 | A2 | O | L30 |
| P2-201 | O | B23 | A2 | O | L32 |
| P2-202 | O | B23 | A2 | O | L36 |
| P2-203 | O | B23 | A2 | O | L40 |
| P2-204 | O | B23 | A2 | O | L48 |
| P2-205 | O | B23 | A2 | O | L53 |
| P2-206 | O | B23 | A2 | O | L59 |
| P2-207 | O | B23 | A2 | O | L65 |
| P2-208 | O | B23 | A2 | O | L68 |
| P2-209 | O | B23 | A2 | O | L79 |
| P2-210 | O | B23 | A2 | O | L89 |
| P2-211 | O | B23 | A2 | O | L102 |
| P2-212 | O | B23 | A2 | O | L111 |
| P2-213 | O | B23 | A2 | O | L119 |
| P2-214 | O | B23 | A2 | O | L128 |
| P2-215 | O | B23 | A2 | O | L148 |
| P2-216 | O | B23 | A2 | O | L156 |
| P2-217 | O | B23 | A2 | O | L167 |
| P2-218 | O | B23 | A2 | O | L169 |
| P2-219 | O | B23 | A2 | O | L172 |
| P2-220 | O | B36 | A2 | O | Single bond |
| P2-221 | O | B36 | A2 | O | O |
| P2-222 | O | B36 | A2 | O | L1 |
| P2-223 | O | B36 | A2 | O | L2 |
| P2-224 | O | B36 | A2 | O | L3 |
| P2-225 | O | B36 | A2 | O | L4 |
| P2-226 | O | B36 | A2 | O | L5 |
| P2-227 | O | B36 | A2 | O | L6 |
| P2-228 | O | B36 | A2 | O | L7 |
| P2-229 | O | B36 | A2 | O | L9 |
| P2-230 | O | B36 | A2 | O | L11 |
| P2-231 | O | B36 | A2 | O | L14 |
| P2-232 | O | B36 | A2 | O | L18 |
| P2-233 | O | B36 | A2 | O | L22 |
| P2-234 | O | B36 | A2 | O | L27 |
| P2-235 | O | B36 | A2 | O | L28 |
| P2-236 | O | B36 | A2 | O | L29 |
| P2-237 | O | B36 | A2 | O | L30 |
| P2-238 | O | B36 | A2 | O | L32 |
| P2-239 | O | B36 | A2 | O | L36 |
| P2-240 | O | B36 | A2 | O | L40 |
| P2-241 | O | B36 | A2 | O | L48 |
| P2-242 | O | B36 | A2 | O | L53 |
| P2-243 | O | B36 | A2 | O | L59 |
| P2-244 | O | B36 | A2 | O | L65 |
| P2-245 | O | B36 | A2 | O | L68 |
| P2-246 | O | B36 | A2 | O | L79 |
| P2-247 | O | B36 | A2 | O | L89 |
| P2-248 | O | B36 | A2 | O | L102 |
| P2-249 | O | B36 | A2 | O | L111 |
| P2-250 | O | B36 | A2 | O | L119 |
| P2-251 | O | B36 | A2 | O | L128 |
| P2-252 | O | B36 | A2 | O | L148 |
| P2-253 | O | B36 | A2 | O | L156 |
| P2-254 | O | B36 | A2 | O | L167 |
| P2-255 | O | B36 | A2 | O | L169 |
| P2-256 | O | B36 | A2 | O | L172 |
| P2-257 | O | B44 | A2 | O | Single bond |
| P2-258 | O | B44 | A2 | O | O |
| P2-259 | O | B44 | A2 | O | L1 |
| P2-260 | O | B44 | A2 | O | L2 |
| P2-261 | O | B44 | A2 | O | L3 |
| P2-262 | O | B44 | A2 | O | L4 |
| P2-263 | O | B44 | A2 | O | L5 |
| P2-264 | O | B44 | A2 | O | L6 |
| P2-265 | O | B44 | A2 | O | L7 |
| P2-266 | O | B44 | A2 | O | L9 |
| P2-267 | O | B44 | A2 | O | L11 |
| P2-268 | O | B44 | A2 | O | L14 |
| P2-269 | O | B44 | A2 | O | L18 |
| P2-270 | O | B44 | A2 | O | L22 |
| P2-271 | O | B56 | A2 | O | L27 |
| P2-272 | O | B44 | A2 | O | L28 |
| P2-273 | O | B44 | A2 | O | L29 |
| P2-274 | O | B44 | A2 | O | L30 |
| P2-275 | O | B44 | A2 | O | L32 |
| P2-276 | O | B44 | A2 | O | L36 |
| P2-277 | O | B44 | A2 | O | L40 |
| P2-278 | O | B44 | A2 | O | L48 |
| P2-279 | O | B44 | A2 | O | L53 |
| P2-280 | O | B44 | A2 | O | L59 |
| P2-281 | O | B44 | A2 | O | L65 |
| P2-282 | O | B44 | A2 | O | L68 |
| P2-283 | O | B44 | A2 | O | L79 |
| P2-284 | O | B44 | A2 | O | L89 |
| P2-285 | O | B44 | A2 | O | L102 |
| P2-286 | O | B44 | A2 | | L111 |
| P2-287 | O | B44 | A2 | O | L119 |
| P2-288 | O | B44 | A2 | O | L128 |
| P2-289 | O | B44 | A2 | O | L148 |
| P2-290 | O | B44 | A2 | O | L156 |
| P2-291 | O | B44 | A2 | O | L167 |
| P2-292 | O | B44 | A2 | O | L169 |
| P2-293 | O | B44 | A2 | O | L172 |
| P2-294 | O | B126 | A2 | O | Single bond |
| P2-295 | O | B92 | A2 | O | O |
| P2-296 | O | B92 | A2 | O | L1 |
| P2-297 | O | B92 | A2 | O | L2 |
| P2-298 | O | B92 | A2 | O | L3 |
| P2-299 | O | B92 | A2 | O | L4 |
| P2-300 | O | B92 | A2 | O | L5 |
| P2-301 | O | B92 | A2 | O | L6 |
| P2-302 | O | B92 | A2 | O | L7 |
| P2-303 | O | B92 | A2 | O | L9 |
| P2-304 | O | B92 | A2 | O | L11 |
| P2-305 | O | B92 | A2 | O | L14 |
| P2-306 | O | B92 | A2 | O | L18 |
| P2-307 | O | B92 | A2 | O | L22 |
| P2-308 | O | B92 | A2 | O | L27 |
| P2-309 | O | B92 | A2 | O | L28 |
| P2-310 | O | B92 | A2 | O | L29 |
| P2-311 | O | B92 | A2 | O | L30 |
| P2-312 | O | B92 | A2 | O | L32 |
| P2-313 | O | B92 | A2 | O | L36 |
| P2-314 | O | B92 | A2 | O | L40 |
| P2-315 | O | B92 | A2 | O | L48 |
| P2-316 | O | B92 | A2 | O | L53 |
| P2-317 | O | B92 | A2 | O | L59 |
| P2-318 | O | B92 | A2 | O | L65 |
| P2-319 | O | B92 | A2 | O | L68 |
| P2-320 | O | B92 | A2 | O | L79 |
| P2-321 | O | B92 | A2 | O | L89 |
| P2-322 | O | B92 | A2 | O | L102 |
| P2-323 | O | B92 | A2 | O | L111 |
| P2-324 | O | B92 | A2 | O | L119 |
| P2-325 | O | B92 | A2 | O | L128 |
| P2-326 | O | B92 | A2 | O | L148 |
| P2-327 | O | B92 | A2 | O | L156 |
| P2-328 | O | B92 | A2 | O | L167 |
| P2-329 | O | B92 | A2 | O | L169 |
| P2-330 | O | B92 | A2 | O | L172 |
| P2-331 | O | A2 | A3 | O | Single bond |
| P2-332 | O | A2 | A3 | O | O |
| P2-333 | O | A2 | A3 | O | L1 |
| P2-334 | O | A2 | A3 | O | L2 |
| P2-335 | O | A2 | A3 | O | L3 |
| P2-336 | O | A2 | A3 | O | L4 |
| P2-337 | O | A2 | A3 | O | L5 |
| P2-338 | O | A2 | A3 | O | L6 |
| P2-339 | O | A2 | A3 | O | L7 |
| P2-340 | O | A2 | A3 | O | L9 |
| P2-341 | O | A2 | A3 | O | L11 |
| P2-342 | O | A2 | A3 | O | L14 |
| P2-343 | O | A2 | A3 | O | L18 |
| P2-344 | O | A2 | A3 | O | L22 |
| P2-345 | O | A2 | A3 | O | L27 |
| P2-346 | O | A2 | A3 | O | L28 |
| P2-347 | O | A2 | A3 | O | L29 |
| P2-348 | O | A2 | A3 | O | L30 |
| P2-349 | O | A2 | A3 | O | L32 |
| P2-350 | O | A2 | A3 | O | L36 |
| P2-351 | O | A2 | A3 | O | L40 |
| P2-352 | O | A2 | A3 | O | L48 |
| P2-353 | O | A2 | A3 | O | L53 |
| P2-354 | O | A2 | A3 | O | L59 |
| P2-355 | O | A2 | A3 | O | L65 |
| P2-356 | O | A2 | A3 | O | L68 |
| P2-357 | O | A2 | A3 | O | L79 |
| P2-358 | O | A2 | A3 | O | L89 |
| P2-359 | O | A2 | A3 | O | L102 |
| P2-360 | O | A2 | A3 | O | L111 |
| P2-361 | O | A2 | A3 | O | L119 |
| P2-362 | O | A2 | A3 | O | L128 |
| P2-363 | O | A2 | A3 | O | L148 |
| P2-364 | O | A2 | A3 | O | L156 |
| P2-365 | O | A2 | A3 | O | L167 |
| P2-366 | O | A2 | A3 | O | L169 |
| P2-367 | O | A2 | A3 | O | L172 |
| P2-368 | O | B4 | A3 | O | Single bond |
| P2-369 | O | B4 | A3 | O | O |
| P2-370 | O | B4 | A3 | O | L1 |
| P2-371 | O | B4 | A3 | O | L2 |
| P2-372 | O | B4 | A3 | O | L3 |
| P2-373 | O | B4 | A3 | O | L4 |
| P2-374 | O | B4 | A3 | O | L5 |
| P2-375 | O | B4 | A3 | O | L6 |
| P2-376 | O | B4 | A3 | O | L7 |
| P2-377 | O | B4 | A3 | O | L9 |
| P2-378 | O | B4 | A3 | O | L11 |
| P2-379 | O | B4 | A3 | O | L14 |
| P2-380 | O | B4 | A3 | O | L18 |
| P2-381 | O | B4 | A3 | O | L22 |
| P2-382 | O | B4 | A3 | O | L27 |
| P2-383 | O | B4 | A3 | O | L28 |
| P2-384 | O | B4 | A3 | O | L29 |
| P2-385 | O | B4 | A3 | O | L30 |
| P2-386 | O | B4 | A3 | O | L32 |
| P2-387 | O | B4 | A3 | O | L36 |
| P2-388 | O | B4 | A3 | O | L40 |
| P2-389 | O | B4 | A3 | O | L48 |
| P2-390 | O | B4 | A3 | O | L53 |
| P2-391 | O | B4 | A3 | O | L59 |
| P2-392 | O | B4 | A3 | O | L65 |
| P2-393 | O | B4 | A3 | O | L68 |
| P2-394 | O | B4 | A3 | O | L79 |
| P2-395 | O | B4 | A3 | O | L89 |
| P2-396 | O | B4 | A3 | O | L102 |
| P2-397 | O | B4 | A3 | O | L111 |
| P2-398 | O | B4 | A3 | O | L119 |
| P2-399 | O | B4 | A3 | O | L128 |
| P2-400 | O | B4 | A3 | O | L148 |
| P2-401 | O | B4 | A3 | O | L156 |
| P2-402 | O | B4 | A3 | O | L167 |
| P2-403 | O | B4 | A3 | O | L169 |
| P2-404 | O | B4 | A3 | O | L172 |
| P2-405 | O | B23 | A3 | O | Single bond |
| P2-406 | O | B23 | A3 | O | O |
| P2-407 | O | B23 | A3 | O | L1 |
| P2-408 | O | B23 | A3 | O | L2 |
| P2-409 | O | B23 | A3 | O | L3 |
| P2-410 | O | B23 | A3 | O | L4 |
| P2-411 | O | B23 | A3 | O | L5 |
| P2-412 | O | B23 | A3 | O | L6 |
| P2-413 | O | B23 | A3 | O | L7 |
| P2-414 | O | B23 | A3 | O | L9 |
| P2-415 | O | B23 | A3 | O | L11 |
| P2-416 | O | B23 | A3 | O | L14 |
| P2-417 | O | B23 | A3 | O | L18 |
| P2-418 | O | B23 | A3 | O | L22 |
| P2-419 | O | B23 | A3 | O | L27 |
| P2-420 | O | B23 | A3 | O | L28 |
| P2-421 | O | B23 | A3 | O | L29 |
| P2-422 | O | B23 | A3 | O | L30 |
| P2-423 | O | B23 | A3 | O | L32 |
| P2-424 | O | B23 | A3 | O | L36 |
| P2-425 | O | B23 | A3 | O | L40 |
| P2-426 | O | B23 | A3 | O | L48 |
| P2-427 | O | B23 | A3 | O | L53 |
| P2-428 | O | B23 | A3 | O | L59 |
| P2-429 | O | B23 | A3 | O | L65 |
| P2-430 | O | B23 | A3 | O | L68 |
| P2-431 | O | B23 | A3 | O | L79 |
| P2-432 | O | B23 | A3 | O | L89 |
| P2-433 | O | B23 | A3 | O | L102 |
| P2-434 | O | B23 | A3 | O | L111 |
| P2-435 | O | B23 | A3 | O | L119 |
| P2-436 | O | B23 | A3 | O | L128 |
| P2-437 | O | B23 | A3 | O | L148 |
| P2-438 | O | B23 | A3 | O | L156 |
| P2-439 | O | B23 | A3 | O | L167 |
| P2-440 | O | B23 | A3 | O | L169 |
| P2-441 | O | B23 | A3 | O | L172 |
| P2-442 | O | B36 | A3 | O | Single bond |
| P2-443 | O | B36 | A3 | O | O |
| P2-444 | O | B36 | A3 | O | L1 |
| P2-445 | O | B36 | A3 | O | L2 |
| P2-446 | O | B36 | A3 | O | L3 |
| P2-447 | O | B36 | A3 | O | L4 |
| P2-448 | O | B36 | A3 | O | L5 |
| P2-449 | O | B36 | A3 | O | L6 |
| P2-450 | O | B36 | A3 | O | L7 |
| P2-451 | O | B36 | A3 | O | L9 |
| P2-452 | O | B36 | A3 | O | L11 |
| P2-453 | O | B36 | A3 | O | L14 |
| P2-454 | O | B36 | A3 | O | L18 |
| P2-455 | O | B36 | A3 | O | L22 |
| P2-456 | O | B36 | A3 | O | L27 |
| P2-457 | O | B36 | A3 | O | L28 |
| P2-458 | O | B36 | A3 | O | L29 |
| P2-459 | O | B36 | A3 | O | L30 |
| P2-460 | O | B36 | A3 | O | L32 |
| P2-461 | O | B36 | A3 | O | L36 |
| P2-462 | O | B36 | A3 | O | L40 |
| P2-463 | O | B36 | A3 | O | L48 |
| P2-464 | O | B36 | A3 | O | L53 |
| P2-465 | O | B36 | A3 | O | L59 |
| P2-466 | O | B36 | A3 | O | L65 |
| P2-467 | O | B36 | A3 | O | L68 |
| P2-468 | O | B36 | A3 | O | L79 |
| P2-469 | O | B36 | A3 | O | L89 |
| P2-470 | O | B36 | A3 | O | L102 |
| P2-471 | O | B36 | A3 | O | L111 |
| P2-472 | O | B36 | A3 | O | L119 |
| P2-473 | O | B36 | A3 | O | L128 |
| P2-474 | O | B36 | A3 | O | L148 |
| P2-475 | O | B36 | A3 | O | L156 |
| P2-476 | O | B36 | A3 | O | L167 |
| P2-477 | O | B36 | A3 | O | L169 |
| P2-478 | O | B36 | A3 | O | L172 |
| P2-479 | O | B44 | A3 | O | Single bond |
| P2-480 | O | B44 | A3 | O | O |
| P2-481 | O | B44 | A3 | O | L1 |
| P2-482 | O | B44 | A3 | O | L2 |
| P2-483 | O | B44 | A3 | O | L3 |
| P2-484 | O | B44 | A3 | O | L4 |
| P2-485 | O | B44 | A3 | O | L5 |
| P2-486 | O | B44 | A3 | O | L6 |
| P2-487 | O | B44 | A3 | O | L7 |
| P2-488 | O | B44 | A3 | O | L9 |
| P2-489 | O | B44 | A3 | O | L11 |
| P2-490 | O | B44 | A3 | O | L14 |
| P2-491 | O | B44 | A3 | O | L18 |
| P2-492 | O | B44 | A3 | O | L22 |
| P2-493 | O | B44 | A3 | O | L27 |
| P2-494 | O | B44 | A3 | O | L28 |
| P2-495 | O | B44 | A3 | O | L29 |
| P2-496 | O | B44 | A3 | O | L30 |
| P2-497 | O | B44 | A3 | O | L32 |
| P2-498 | O | B44 | A3 | O | L36 |
| P2-499 | O | B44 | A3 | O | L40 |
| P2-500 | O | B44 | A3 | O | L48 |
| P2-501 | O | B44 | A3 | O | L53 |
| P2-502 | O | B44 | A3 | O | L59 |
| P2-503 | O | B44 | A3 | O | L65 |
| P2-504 | O | B44 | A3 | O | L68 |
| P2-505 | O | B44 | A3 | O | L79 |
| P2-506 | O | B44 | A3 | O | L89 |
| P2-507 | O | B44 | A3 | O | L102 |
| P2-508 | O | B44 | A3 | O | L111 |
| P2-509 | O | B44 | A3 | O | L119 |
| P2-510 | O | B44 | A3 | O | L128 |
| P2-511 | O | B44 | A3 | O | L148 |
| P2-512 | O | B44 | A3 | O | L156 |
| P2-513 | O | B44 | A3 | O | L167 |
| P2-514 | O | B44 | A3 | O | L169 |
| P2-515 | O | B44 | A3 | O | L172 |
| P2-516 | O | B92 | A3 | O | Single bond |
| P2-517 | O | B92 | A3 | O | O |
| P2-518 | O | B92 | A3 | O | L1 |
| P2-519 | O | B92 | A3 | O | L2 |
| P2-520 | O | B92 | A3 | O | L3 |
| P2-521 | O | B92 | A3 | O | L4 |
| P2-522 | O | B92 | A3 | O | L5 |
| P2-523 | O | B92 | A3 | O | L6 |
| P2-524 | O | B92 | A3 | O | L7 |
| P2-525 | O | B92 | A3 | O | L9 |
| P2-526 | O | B92 | A3 | O | L11 |
| P2-527 | O | B92 | A3 | O | L14 |
| P2-528 | O | B92 | A3 | O | L18 |
| P2-529 | O | B92 | A3 | O | L22 |
| P2-530 | O | B92 | A3 | O | L27 |
| P2-531 | O | B92 | A3 | O | L28 |
| P2-532 | O | B92 | A3 | O | L29 |
| P2-533 | O | B92 | A3 | O | L30 |
| P2-534 | O | B92 | A3 | O | L32 |
| P2-535 | O | B92 | A3 | O | L36 |
| P2-536 | O | B92 | A3 | O | L40 |
| P2-537 | O | B92 | A3 | O | L48 |
| P2-538 | O | B92 | A3 | O | L53 |
| P2-539 | O | B92 | A3 | O | L59 |
| P2-540 | O | B92 | A3 | O | L65 |
| P2-541 | O | B92 | A3 | O | L68 |
| P2-542 | O | B92 | A3 | O | L79 |
| P2-543 | O | B92 | A3 | O | L89 |
| P2-544 | O | B92 | A3 | O | L102 |
| P2-545 | O | B92 | A3 | O | L111 |
| P2-546 | O | B92 | A3 | O | L119 |
| P2-547 | O | B92 | A3 | O | L128 |
| P2-548 | O | B92 | A3 | O | L148 |
| P2-549 | O | B92 | A3 | O | L156 |
| P2-550 | O | B92 | A3 | O | L167 |
| P2-551 | O | B92 | A3 | O | L169 |
| P2-552 | O | B92 | A3 | O | L172 |
| P2-553 | O | A2 | A12 | O | Single bond |
| P2-554 | O | A2 | A12 | O | O |
| P2-555 | O | A2 | A12 | O | L1 |
| P2-556 | O | A2 | A12 | O | L5 |
| P2-557 | O | A2 | A12 | O | L9 |
| P2-558 | O | A2 | A12 | O | L18 |
| P2-559 | O | A2 | A12 | O | L22 |
| P2-560 | O | A2 | A12 | O | L27 |
| P2-561 | O | A2 | A12 | O | L29 |
| P2-562 | O | A2 | A12 | O | L36 |
| P2-563 | O | A2 | A12 | O | L40 |
| P2-564 | O | A2 | A12 | O | L53 |
| P2-565 | O | A2 | A12 | O | L59 |
| P2-566 | O | A2 | A12 | O | L65 |
| P2-567 | O | A2 | A12 | O | L68 |
| P2-568 | O | A2 | A12 | O | L119 |
| P2-569 | O | A2 | A12 | O | L128 |
| P2-570 | O | A2 | A12 | O | L148 |
| P2-571 | O | B4 | A12 | O | Single bond |
| P2-572 | O | B4 | A12 | O | O |
| P2-573 | O | B4 | A12 | O | L1 |
| P2-574 | O | B4 | A12 | O | L5 |
| P2-575 | O | B4 | A12 | O | L9 |
| P2-576 | O | B4 | A12 | O | L18 |
| P2-577 | O | B4 | A12 | O | L22 |
| P2-578 | O | B4 | A12 | O | L27 |
| P2-579 | O | B4 | A12 | O | L29 |
| P2-580 | O | B4 | A12 | O | L36 |
| P2-581 | O | B4 | A12 | O | L40 |
| P2-582 | O | B4 | A12 | O | L53 |
| P2-583 | O | B4 | A12 | O | L59 |
| P2-584 | O | B4 | A12 | O | L65 |
| P2-585 | O | B4 | A12 | O | L68 |
| P2-586 | O | B4 | A12 | O | L119 |
| P2-587 | O | B4 | A12 | O | L128 |
| P2-588 | O | B4 | A12 | O | L148 |
| P2-589 | O | B23 | A12 | O | Single bond |
| P2-590 | O | B23 | A12 | O | O |
| P2-591 | O | B23 | A12 | O | L1 |
| P2-592 | O | B23 | A12 | O | L5 |
| P2-593 | O | B23 | A12 | O | L9 |
| P2-594 | O | B23 | A12 | O | L18 |
| P2-595 | O | B23 | A12 | O | L22 |
| P2-596 | O | B23 | A12 | O | L27 |
| P2-597 | O | B23 | A12 | O | L29 |
| P2-598 | O | B23 | A12 | O | L36 |
| P2-599 | O | B23 | A12 | O | L40 |
| P2-600 | O | B23 | A12 | O | L53 |
| P2-601 | O | B23 | A12 | O | L59 |
| P2-602 | O | B23 | A12 | O | L65 |
| P2-603 | O | B23 | A12 | O | L68 |
| P2-604 | O | B23 | A12 | O | L119 |
| P2-605 | O | B23 | A12 | O | L128 |
| P2-606 | O | B23 | A12 | O | L148 |
| P2-607 | O | B36 | A12 | O | Single bond |
| P2-608 | O | B36 | A12 | O | O |
| P2-609 | O | B36 | A12 | O | L1 |
| P2-610 | O | B36 | A12 | O | L5 |
| P2-611 | O | B36 | A12 | O | L9 |
| P2-612 | O | B36 | A12 | O | L18 |
| P2-613 | O | B36 | A12 | O | L22 |
| P2-614 | O | B36 | A12 | O | L27 |
| P2-615 | O | B36 | A12 | O | L29 |
| P2-616 | O | B36 | A12 | O | L36 |
| P2-617 | O | B36 | A12 | O | L40 |
| P2-618 | O | B36 | A12 | O | L53 |
| P2-619 | O | B36 | A12 | O | L59 |
| P2-620 | O | B36 | A12 | O | L65 |
| P2-621 | O | B36 | A12 | O | L68 |
| P2-622 | O | B36 | A12 | O | L119 |
| P2-623 | O | B36 | A12 | O | L128 |
| P2-624 | O | B36 | A12 | O | L148 |
| P2-625 | O | B44 | A12 | O | Single bond |
| P2-626 | O | B44 | A12 | O | O |
| P2-627 | O | B44 | A12 | O | L1 |
| P2-628 | O | B44 | A12 | O | L5 |
| P2-629 | O | B44 | A12 | O | L9 |
| P2-630 | O | B44 | A12 | O | L18 |
| P2-631 | O | B44 | A12 | O | L22 |
| P2-632 | O | B44 | A12 | O | L27 |
| P2-633 | O | B44 | A12 | O | L29 |
| P2-634 | O | B44 | A12 | O | L36 |
| P2-635 | O | B44 | A12 | O | L40 |
| P2-636 | O | B44 | A12 | O | L53 |
| P2-637 | O | B44 | A12 | O | L59 |
| P2-638 | O | B44 | A12 | O | L65 |
| P2-639 | O | B44 | A12 | O | L68 |
| P2-640 | O | B44 | A12 | O | L119 |
| P2-641 | O | B44 | A12 | O | L128 |
| P2-642 | O | B44 | A12 | O | L148 |
| P2-643 | O | B92 | A12 | O | Single bond |
| P2-644 | O | B92 | A12 | O | O |
| P2-645 | O | B92 | A12 | O | L1 |
| P2-646 | O | B92 | A12 | O | L5 |
| P2-647 | O | B92 | A12 | O | L9 |
| P2-648 | O | B92 | A12 | O | L18 |
| P2-649 | O | B92 | A12 | O | L22 |
| P2-650 | O | B92 | A12 | O | L27 |
| P2-651 | O | B92 | A12 | O | L29 |
| P2-652 | O | B92 | A12 | O | L36 |
| P2-653 | O | B92 | A12 | O | L40 |
| P2-654 | O | B92 | A12 | O | L53 |
| P2-655 | O | B92 | A12 | O | L59 |
| P2-656 | O | B92 | A12 | O | L65 |
| P2-657 | O | B92 | A12 | O | L68 |
| P2-658 | O | B92 | A12 | O | L119 |
| P2-659 | O | B92 | A12 | O | L128 |
| P2-660 | O | B92 | A12 | O | L148 |
| P2-661 | O | A2 | B41 | O | Single bond |
| P2-662 | O | A2 | B41 | O | O |
| P2-663 | O | A2 | B41 | O | L1 |
| P2-664 | O | A2 | B41 | O | L5 |
| P2-665 | O | A2 | B41 | O | L9 |
| P2-666 | O | A2 | B41 | O | L18 |
| P2-667 | O | A2 | B41 | O | L22 |
| P2-668 | O | A2 | B41 | O | L27 |
| P2-669 | O | A2 | B41 | O | L29 |
| P2-670 | O | A2 | B41 | O | L36 |
| P2-671 | O | A2 | B41 | O | L40 |
| P2-672 | O | A2 | B41 | O | L53 |
| P2-673 | O | A2 | B41 | O | L59 |
| P2-674 | O | A2 | B41 | O | L65 |
| P2-675 | O | A2 | B41 | O | L68 |
| P2-676 | O | A2 | B41 | O | L119 |
| P2-677 | O | A2 | B41 | O | L128 |
| P2-678 | O | A2 | B41 | O | L148 |
| P2-679 | O | B4 | B41 | O | Single bond |
| P2-680 | O | B4 | B41 | O | O |
| P2-681 | O | B4 | B41 | O | L1 |
| P2-682 | O | B4 | B41 | O | L5 |
| P2-683 | O | B4 | B41 | O | L9 |
| P2-684 | O | B4 | B41 | O | L18 |
| P2-685 | O | B4 | B41 | O | L22 |
| P2-686 | O | B4 | B41 | O | L27 |
| P2-687 | O | B4 | B41 | O | L29 |
| P2-688 | O | B4 | B41 | O | L36 |
| P2-689 | O | B4 | B41 | O | L40 |
| P2-690 | O | B4 | B41 | O | L53 |
| P2-691 | O | B4 | B41 | O | L59 |
| P2-692 | O | B4 | B41 | O | L65 |
| P2-693 | O | B4 | B41 | O | L68 |
| P2-694 | O | B4 | B41 | O | L119 |
| P2-695 | O | B4 | B41 | O | L128 |
| P2-696 | O | B4 | B41 | O | L148 |
| P2-697 | O | B23 | B41 | O | Single bond |
| P2-698 | O | B23 | B41 | O | O |
| P2-699 | O | B23 | B41 | O | L1 |
| P2-700 | O | B23 | B41 | O | L5 |
| P2-701 | O | B23 | B41 | O | L9 |
| P2-702 | O | B23 | B41 | O | L18 |
| P2-703 | O | B23 | B41 | O | L22 |
| P2-704 | O | B23 | B41 | O | L27 |
| P2-705 | O | B23 | B41 | O | L29 |
| P2-706 | O | B23 | B41 | O | L36 |
| P2-707 | O | B23 | B41 | O | L40 |
| P2-708 | O | B23 | B41 | O | L53 |
| P2-709 | O | B23 | B41 | O | L59 |
| P2-710 | O | B23 | B41 | O | L65 |
| P2-711 | O | B23 | B41 | O | L68 |
| P2-712 | O | B23 | B41 | O | L119 |
| P2-713 | O | B23 | B41 | O | L128 |
| P2-714 | O | B23 | B41 | O | L148 |
| P2-715 | O | B36 | B41 | O | Single bond |
| P2-716 | O | B36 | B41 | O | O |
| P2-717 | O | B36 | B41 | O | L1 |
| P2-718 | O | B36 | B41 | O | L5 |
| P2-719 | O | B36 | B41 | O | L9 |
| P2-720 | O | B36 | B41 | O | L18 |
| P2-721 | O | B36 | B41 | O | L22 |
| P2-722 | O | B36 | B41 | O | L27 |
| P2-723 | O | B36 | B41 | O | L29 |
| P2-724 | O | B36 | B41 | O | L36 |
| P2-725 | O | B36 | B41 | O | L40 |
| P2-726 | O | B36 | B41 | O | L53 |
| P2-727 | O | B36 | B41 | O | L59 |
| P2-728 | O | B36 | B41 | O | L65 |
| P2-729 | O | B36 | B41 | O | L68 |
| P2-730 | O | B36 | B41 | O | L119 |
| P2-731 | O | B36 | B41 | O | L128 |
| P2-732 | O | B36 | B41 | O | L148 |
| P2-733 | O | B44 | B41 | O | Single bond |
| P2-734 | O | B44 | B41 | O | O |
| P2-735 | O | B44 | B41 | O | L1 |
| P2-736 | O | B44 | B41 | O | L5 |
| P2-737 | O | B44 | B41 | O | L9 |
| P2-738 | O | B44 | B41 | O | L18 |
| P2-739 | O | B44 | B41 | O | L22 |
| P2-740 | O | B44 | B41 | O | L27 |
| P2-741 | O | B44 | B41 | O | L29 |
| P2-742 | O | B44 | B41 | O | L36 |
| P2-743 | O | B44 | B41 | O | L40 |
| P2-744 | O | B44 | B41 | O | L53 |
| P2-745 | O | B44 | B41 | O | L59 |
| P2-746 | O | B44 | B41 | O | L65 |
| P2-747 | O | B44 | B41 | O | L68 |
| P2-748 | O | B44 | B41 | O | L119 |
| P2-749 | O | B44 | B41 | O | L128 |
| P2-750 | O | B44 | B41 | O | L148 |
| P2-751 | O | B92 | B41 | O | Single bond |
| P2-752 | O | B92 | B41 | O | O |
| P2-753 | O | B92 | B41 | O | L1 |
| P2-754 | O | B92 | B41 | O | L5 |
| P2-755 | O | B92 | B41 | O | L9 |
| P2-756 | O | B92 | B41 | O | L18 |
| P2-757 | O | B92 | B41 | O | L22 |
| P2-758 | O | B92 | B41 | O | L27 |
| P2-759 | O | B92 | B41 | O | L29 |
| P2-760 | O | B92 | B41 | O | L36 |
| P2-761 | O | B92 | B41 | O | L40 |
| P2-762 | O | B92 | B41 | O | L53 |
| P2-763 | O | B92 | B41 | O | L59 |
| P2-764 | O | B92 | B41 | O | L65 |
| P2-765 | O | B92 | B41 | O | L68 |
| P2-766 | O | B92 | B41 | O | L119 |
| P2-767 | O | B92 | B41 | O | L128 |
| P2-768 | O | B92 | B41 | O | L148 |
| P2-769 | O | A2 | B56 | O | Single bond |
| P2-770 | O | A2 | B56 | O | O |
| P2-771 | O | A2 | B56 | O | L1 |
| P2-772 | O | A2 | B56 | O | L5 |
| P2-773 | O | A2 | B56 | O | L9 |
| P2-774 | O | A2 | B56 | O | L18 |
| P2-775 | O | A2 | B56 | O | L22 |
| P2-776 | O | A2 | B56 | O | L27 |
| P2-777 | O | A2 | B56 | O | L29 |
| P2-778 | O | A2 | B56 | O | L36 |
| P2-779 | O | A2 | B56 | O | L40 |
| P2-780 | O | A2 | B56 | O | L53 |
| P2-781 | O | A2 | B56 | O | L59 |
| P2-782 | O | A2 | B56 | O | L65 |
| P2-783 | O | A2 | B56 | O | L68 |
| P2-784 | O | A2 | B56 | O | L119 |
| P2-785 | O | A2 | B56 | O | L128 |
| P2-786 | O | A2 | B56 | O | L148 |
| P2-787 | O | B4 | B56 | O | Single bond |
| P2-788 | O | B4 | B56 | O | O |
| P2-789 | O | B4 | B56 | O | L1 |
| P2-790 | O | B4 | B56 | O | L5 |
| P2-791 | O | B4 | B56 | O | L9 |
| P2-792 | O | B4 | B56 | O | L18 |
| P2-793 | O | B4 | B56 | O | L22 |
| P2-794 | O | B4 | B56 | O | L27 |
| P2-795 | O | B4 | B56 | O | L29 |
| P2-796 | O | B4 | B56 | O | L36 |
| P2-797 | O | B4 | B56 | O | L40 |
| P2-798 | O | B4 | B56 | O | L53 |
| P2-799 | O | B4 | B56 | O | L59 |
| P2-800 | O | B4 | B56 | O | L65 |
| P2-801 | O | B4 | B56 | O | L68 |
| P2-802 | O | B4 | B56 | O | L119 |
| P2-803 | O | B4 | B56 | O | L128 |
| P2-804 | O | B4 | B56 | O | L148 |
| P2-805 | O | B23 | B56 | O | Single bond |
| P2-806 | O | B23 | B56 | O | O |
| P2-807 | O | B23 | B56 | O | L1 |
| P2-808 | O | B23 | B56 | O | L5 |
| P2-809 | O | B23 | B56 | O | L9 |
| P2-810 | O | B23 | B56 | O | L18 |
| P2-811 | O | B23 | B56 | O | L22 |
| P2-812 | O | B23 | B56 | O | L27 |
| P2-813 | O | B23 | B56 | O | L29 |
| P2-814 | O | B23 | B56 | O | L36 |
| P2-815 | O | B23 | B56 | O | L40 |
| P2-816 | O | B23 | B56 | O | L53 |
| P2-817 | O | B23 | B56 | O | L59 |
| P2-818 | O | B23 | B56 | O | L65 |
| P2-819 | O | B23 | B56 | O | L68 |
| P2-820 | O | B23 | B56 | O | L119 |
| P2-821 | O | B23 | B56 | O | L128 |
| P2-822 | O | B23 | B56 | O | L148 |
| P2-823 | O | B36 | B56 | O | Single bond |
| P2-824 | O | B36 | B56 | O | O |
| P2-825 | O | B36 | B56 | O | L1 |
| P2-826 | O | B36 | B56 | O | L5 |
| P2-827 | O | B36 | B56 | O | L9 |
| P2-828 | O | B36 | B56 | O | L18 |
| P2-829 | O | B36 | B56 | O | L22 |
| P2-830 | O | B36 | B56 | O | L27 |
| P2-831 | O | B36 | B56 | O | L29 |
| P2-832 | O | B36 | B56 | O | L36 |
| P2-833 | O | B36 | B56 | O | L40 |
| P2-834 | O | B36 | B56 | O | L53 |
| P2-835 | O | B36 | B56 | O | L59 |
| P2-836 | O | B36 | B56 | O | L65 |
| P2-837 | O | B36 | B56 | O | L68 |
| P2-838 | O | B36 | B56 | O | L119 |
| P2-839 | O | B36 | B56 | O | L128 |
| P2-840 | O | B36 | B56 | O | L148 |
| P2-841 | O | B44 | B56 | O | Single bond |
| P2-842 | O | B44 | B56 | O | O |
| P2-843 | O | B44 | B56 | O | L1 |
| P2-844 | O | B44 | B56 | O | L5 |
| P2-845 | O | B44 | B56 | O | L9 |
| P2-846 | O | B44 | B56 | O | L18 |
| P2-847 | O | B44 | B56 | O | L22 |
| P2-848 | O | B44 | B56 | O | L27 |
| P2-849 | O | B44 | B56 | O | L29 |
| P2-850 | O | B44 | B56 | O | L36 |
| P2-851 | O | B44 | B56 | O | L40 |
| P2-852 | O | B44 | B56 | O | L53 |
| P2-853 | O | B44 | B56 | O | L59 |
| P2-854 | O | B44 | B56 | O | L65 |
| P2-855 | O | B44 | B56 | O | L68 |
| P2-856 | O | B44 | B56 | O | L119 |
| P2-857 | O | B44 | B56 | O | L128 |
| P2-858 | O | B44 | B56 | O | L148 |
| P2-859 | O | B92 | B56 | O | Single bond |
| P2-860 | O | B92 | B56 | O | O |
| P2-861 | O | B92 | B56 | O | L1 |
| P2-862 | O | B92 | B56 | O | L5 |
| P2-863 | O | B92 | B56 | O | L9 |
| P2-864 | O | B92 | B56 | O | L18 |
| P2-865 | O | B92 | B56 | O | L22 |
| P2-866 | O | B92 | B56 | O | L27 |
| P2-867 | O | B92 | B56 | O | L29 |
| P2-868 | O | B92 | B56 | O | L36 |
| P2-869 | O | B92 | B56 | O | L40 |
| P2-870 | O | B92 | B56 | O | L53 |
| P2-871 | O | B92 | B56 | O | L59 |
| P2-872 | O | B92 | B56 | O | L65 |
| P2-873 | O | B92 | B56 | O | L68 |
| P2-874 | O | B92 | B56 | O | L119 |
| P2-875 | O | B92 | B56 | O | L128 |
| P2-876 | O | B92 | B56 | O | L148 |
| P2-877 | O | B44 | A2 | S | Single bond |
| P2-878 | O | B44 | A2 | S | L5 |
| P2-879 | O | B44 | A2 | S | L6 |
| P2-880 | O | B44 | A2 | S | L27 |
| P2-881 | O | B44 | A2 | S | L29 |
| P2-882 | O | B44 | A2 | S | L36 |
| P2-883 | O | B92 | A2 | S | Single bond |
| P2-884 | O | B92 | A2 | S | L5 |
| P2-885 | O | B92 | A2 | S | L6 |
| P2-886 | O | B92 | A2 | S | L27 |
| P2-887 | O | B92 | A2 | S | L29 |
| P2-888 | O | B92 | A2 | S | L36 |
| P2-889 | O | B44 | A3 | S | Single bond |
| P2-890 | O | B44 | A3 | S | L5 |
| P2-891 | O | B44 | A3 | S | L6 |
| P2-892 | O | B44 | A3 | S | L27 |
| P2-893 | O | B44 | A3 | S | L29 |
| P2-894 | O | B44 | A3 | S | L36 |
| P2-895 | O | B92 | A3 | S | Single bond |
| P2-896 | O | B92 | A3 | S | L5 |
| P2-897 | O | B92 | A3 | S | L6 |
| P2-898 | O | B92 | A3 | S | L27 |
| P2-899 | O | B92 | A3 | S | L29 |
| P2-900 | O | B92 | A3 | S | L36 |
| P2-901 | O | B44 | A2 | Se | L6 |
| P2-902 | O | B44 | A2 | Se | L36 |
| P2-903 | O | B92 | A2 | Se | L6 |
| P2-904 | O | B92 | A2 | Se | L36 |
| P2-905 | O | B44 | A3 | Se | L6 |
| P2-906 | O | B44 | A3 | Se | L36 |
| P2-907 | O | B92 | A3 | Se | L6 |
| P2-908 | O | B92 | A3 | Se | L36 |
| P2-909 | O | B44 | A2 | S═O | L6 |
| P2-910 | O | B44 | A2 | S═O | L36 |
| P2-911 | O | B92 | A2 | S═O | L6 |
| P2-912 | O | B92 | A2 | S═O | L36 |
| P2-913 | O | B44 | A3 | S═O | L6 |
| P2-914 | O | B44 | A3 | S═O | L36 |
| P2-915 | O | B92 | A3 | S═O | L6 |
| P2-916 | O | B92 | A3 | S═O | L36 |
| P2-917 | O | B44 | A2 | O═S═O | Single bond |
| P2-918 | O | B44 | A2 | O═S═O | L5 |
| P2-919 | O | B44 | A2 | O═S═O | L6 |
| P2-920 | O | B44 | A2 | O═S═O | L27 |
| P2-921 | O | B44 | A2 | O═S═O | L29 |
| P2-922 | O | B44 | A2 | O═S═O | L36 |
| P2-923 | O | B92 | A2 | O═S═O | Single bond |
| P2-924 | O | B92 | A2 | O═S═O | L5 |
| P2-925 | O | B92 | A2 | O═S═O | L6 |
| P2-926 | O | B92 | A2 | O═S═O | L27 |
| P2-927 | O | B92 | A2 | O═S═O | L29 |
| P2-928 | O | B92 | A2 | O═S═O | L36 |
| P2-929 | O | B44 | A3 | O═S═O | Single bond |
| P2-930 | O | B44 | A3 | O═S═O | L5 |
| P2-931 | O | B44 | A3 | O═S═O | L6 |
| P2-932 | O | B44 | A3 | O═S═O | L27 |
| P2-933 | O | B44 | A3 | O═S═O | L29 |
| P2-934 | O | B44 | A3 | O═S═O | L36 |
| P2-935 | O | B92 | A3 | O═S═O | Single bond |
| P2-936 | O | B92 | A3 | O═S═O | L5 |
| P2-937 | O | B92 | A3 | O═S═O | L6 |
| P2-938 | O | B92 | A3 | O═S═O | L27 |
| P2-939 | O | B92 | A3 | O═S═O | L29 |
| P2-940 | O | B92 | A3 | O═S═O | L36 |
| P2-941 | S | B44 | A2 | O | Single bond |
| P2-942 | S | B44 | A2 | O | L5 |
| P2-943 | S | B44 | A2 | O | L6 |
| P2-944 | S | B44 | A2 | O | L27 |
| P2-945 | S | B44 | A2 | O | L29 |
| P2-946 | S | B44 | A2 | O | L36 |
| P2-947 | S | B92 | A2 | O | Single bond |
| P2-948 | S | B92 | A2 | O | L5 |
| P2-949 | S | B92 | A2 | O | L6 |
| P2-950 | S | B92 | A2 | O | L27 |
| P2-951 | S | B92 | A2 | O | L29 |
| P2-952 | S | B92 | A2 | O | L36 |
| P2-953 | S | B44 | A3 | O | Single bond |
| P2-954 | S | B44 | A3 | O | L5 |
| P2-955 | S | B44 | A3 | O | L6 |
| P2-956 | S | B44 | A3 | O | L27 |
| P2-957 | S | B44 | A3 | O | L29 |
| P2-958 | S | B44 | A3 | O | L36 |
| P2-959 | S | B92 | A3 | O | Single bond |
| P2-960 | S | B92 | A3 | O | L5 |
| P2-961 | S | B92 | A3 | O | L6 |
| P2-962 | S | B92 | A3 | O | L27 |
| P2-963 | S | B92 | A3 | O | L29 |
| P2-964 | S | B92 | A3 | O | L36 |
| P2-965 | Se | B44 | A2 | O | L5 |
| P2-966 | Se | B44 | A2 | O | L6 |
| P2-967 | Se | B44 | A2 | O | L27 |
| P2-968 | Se | B44 | A2 | O | L29 |
| P2-969 | Se | B44 | A2 | O | L36 |
| P2-970 | Se | B92 | A2 | O | L5 |
| P2-971 | Se | B92 | A2 | O | L6 |
| P2-972 | Se | B92 | A2 | O | L27 |
| P2-973 | Se | B92 | A2 | O | L29 |
| P2-974 | Se | B92 | A2 | O | L36 |
| P2-975 | Se | B44 | A3 | O | L5 |
| P2-976 | Se | B44 | A3 | O | L6 |
| P2-977 | Se | B44 | A3 | O | L27 |
| P2-978 | Se | B44 | A3 | O | L29 |
| P2-979 | Se | B44 | A3 | O | L36 |
| P2-980 | Se | B92 | A3 | O | L5 |
| P2-981 | Se | B92 | A3 | O | L6 |
| P2-982 | Se | B92 | A3 | O | L27 |
| P2-983 | Se | B92 | A3 | O | L29 |
| P2-984 | Se | B92 | A3 | O | L36 |
| P2-985 | S | B44 | A2 | O═S═O | Single bond |
| P2-986 | S | B44 | A2 | O═S═O | L5 |
| P2-987 | S | B44 | A2 | O═S═O | L6 |
| P2-988 | S | B44 | A2 | O═S═O | L27 |
| P2-989 | S | B44 | A2 | O═S═O | L29 |
| P2-990 | S | B44 | A2 | O═S═O | L36 |
| P2-991 | S | B92 | A2 | O═S═O | Single bond |
| P2-992 | S | B92 | A2 | O═S═O | L5 |
| P2-993 | S | B92 | A2 | O═S═O | L6 |
| P2-994 | S | B92 | A2 | O═S═O | L27 |
| P2-995 | S | B92 | A2 | O═S═O | L29 |
| P2-996 | S | B92 | A2 | O═S═O | L36 |
| P2-997 | S | B44 | A3 | O═S═O | Single bond |
| P2-998 | S | B44 | A3 | O═S═O | L5 |
| P2-999 | S | B44 | A3 | O═S═O | L6 |
| P2-1000 | S | B44 | A3 | O═S═O | L27 |
| P2-1001 | S | B44 | A3 | O═S═O | L29 |
| P2-1002 | S | B44 | A3 | O═S═O | L36 |
| P2-1003 | S | B92 | A3 | O═S═O | Single bond |
| P2-1004 | S | B92 | A3 | O═S═O | L5 |
| P2-1005 | S | B92 | A3 | O═S═O | L6 |
| P2-1006 | S | B92 | A3 | O═S═O | L27 |
| P2-1007 | S | B92 | A3 | O═S═O | L29 |
| P2-1008 | S | B92 | A3 | O═S═O | L36 |
| P2-1009 | Se | B44 | A2 | O═S═O | L5 |
| P2-1010 | Se | B44 | A2 | O═S═O | L6 |
| P2-1011 | Se | B44 | A2 | O═S═O | L27 |
| P2-1012 | Se | B44 | A2 | O═S═O | L29 |
| P2-1013 | Se | B44 | A2 | O═S═O | L36 |
| P2-1014 | Se | B92 | A2 | O═S═O | L5 |
| P2-1015 | Se | B92 | A2 | O═S═O | L6 |
| P2-1016 | Se | B92 | A2 | O═S═O | L27 |
| P2-1017 | Se | B92 | A2 | O═S═O | L29 |
| P2-1018 | Se | B92 | A2 | O═S═O | L36 |
| P2-1019 | Se | B44 | A3 | O═S═O | L5 |
| P2-1020 | Se | B44 | A3 | O═S═O | L6 |
| P2-1021 | Se | B44 | A3 | O═S═O | L27 |
| P2-1022 | Se | B44 | A3 | O═S═O | L29 |
| P2-1023 | Se | B44 | A3 | O═S═O | L36 |
| P2-1024 | Se | B92 | A3 | O═S═O | L5 |
| P2-1025 | Se | B92 | A3 | O═S═O | L6 |
| P2-1026 | Se | B92 | A3 | O═S═O | L27 |
| P2-1027 | Se | B92 | A3 | O═S═O | L29 |
| P2-1028 | Se | B92 | A3 | O═S═O | L36 |

The compounds P3-1 to P3-1028 have structures represented by formula I-2-1; wherein R¹ and R² are the same, and W, Z, R¹, R², X, and L correspond to atoms or groups selected from the following table 3, respectively:

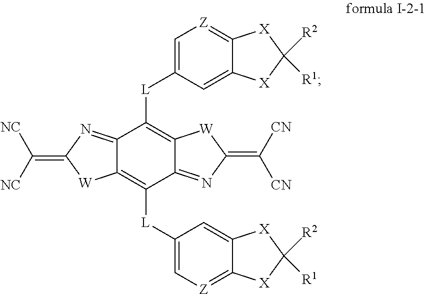

TABLE 3

| Compound No. | W | Z | R¹ and R² | X | L |
|---|---|---|---|---|---|
| P3-1 | O | C-A1 | A1 | O | Single bond |
| P3-2 | O | C-A1 | A1 | O | O |
| P3-3 | O | C-A1 | A1 | O | L1 |
| P3-4 | O | C-A1 | A1 | O | L5 |
| P3-5 | O | C-A1 | A1 | O | L9 |
| P3-6 | O | C-A1 | A1 | O | L18 |
| P3-7 | O | C-A1 | A1 | O | L22 |
| P3-8 | O | C-A1 | A1 | O | L27 |
| P3-9 | O | C-A1 | A1 | O | L29 |
| P3-10 | O | C-A1 | A1 | O | L36 |
| P3-11 | O | C-A1 | A1 | O | L40 |
| P3-12 | O | C-A1 | A1 | O | L53 |
| P3-13 | O | C-A1 | A1 | O | L59 |
| P3-14 | O | C-A1 | A1 | O | L65 |
| P3-15 | O | C-A1 | A1 | O | L68 |
| P3-16 | O | C-A1 | A1 | O | L119 |
| P3-17 | O | C-A1 | A1 | O | L128 |
| P3-18 | O | C-A1 | A1 | O | L148 |
| P3-19 | O | C-A2 | A1 | O | Single bond |
| P3-20 | O | C-A2 | A1 | O | O |
| P3-21 | O | C-A2 | A1 | O | L1 |
| P3-22 | O | C-A2 | A1 | O | L5 |
| P3-23 | O | C-A2 | A1 | O | L9 |
| P3-24 | O | C-A2 | A1 | O | L18 |
| P3-25 | O | C-A2 | A1 | O | L22 |
| P3-26 | O | C-A2 | A1 | O | L27 |
| P3-27 | O | C-A2 | A1 | O | L29 |
| P3-28 | O | C-A2 | A1 | O | L36 |
| P3-29 | O | C-A2 | A1 | O | L40 |
| P3-30 | O | C-A2 | A1 | O | L53 |
| P3-31 | O | C-A2 | A1 | O | L59 |
| P3-32 | O | C-A2 | A1 | O | L65 |
| P3-33 | O | C-A2 | A1 | O | L68 |
| P3-34 | O | C-A2 | A1 | O | L119 |
| P3-35 | O | C-A2 | A1 | O | L128 |
| P3-36 | O | C-A2 | A1 | O | L148 |
| P3-37 | O | C-A3 | A1 | O | Single bond |
| P3-38 | O | C-A3 | A1 | O | O |
| P3-39 | O | C-A3 | A1 | O | L1 |
| P3-40 | O | C-A3 | A1 | O | L5 |
| P3-41 | O | C-A3 | A1 | O | L9 |
| P3-42 | O | C-A3 | A1 | O | L18 |
| P3-43 | O | C-A3 | A1 | O | L22 |
| P3-44 | O | C-A3 | A1 | O | L27 |
| P3-45 | O | C-A3 | A1 | O | L29 |
| P3-46 | O | C-A3 | A1 | O | L36 |
| P3-47 | O | C-A3 | A1 | O | L40 |
| P3-48 | O | C-A3 | A1 | O | L53 |
| P3-49 | O | C-A3 | A1 | O | L59 |
| P3-50 | O | C-A3 | A1 | O | L65 |
| P3-51 | O | C-A3 | A1 | O | L68 |
| P3-52 | O | C-A3 | A1 | O | L119 |
| P3-53 | O | C-A3 | A1 | O | L128 |
| P3-54 | O | C-A3 | A1 | O | L148 |
| P3-55 | O | C-B41 | A1 | O | Single bond |
| P3-56 | O | C-B41 | A1 | O | O |
| P3-57 | O | C-B41 | A1 | O | L1 |
| P3-58 | O | C-B41 | A1 | O | L5 |
| P3-59 | O | C-B41 | A1 | O | L9 |
| P3-60 | O | C-B41 | A1 | O | L18 |
| P3-61 | O | C-B41 | A1 | O | L22 |
| P3-62 | O | C-B41 | A1 | O | L27 |
| P3-63 | O | C-B41 | A1 | O | L29 |
| P3-64 | O | C-B41 | A1 | O | L36 |
| P3-65 | O | C-B41 | A1 | O | L40 |
| P3-66 | O | C-B41 | A1 | O | L53 |
| P3-67 | O | C-B41 | A1 | O | L59 |
| P3-68 | O | C-B41 | A1 | O | L65 |
| P3-69 | O | C-B41 | A1 | O | L68 |
| P3-70 | O | C-B41 | A1 | O | L119 |
| P3-71 | O | C-B41 | A1 | O | L128 |
| P3-72 | O | C-B41 | A1 | O | L148 |
| P3-73 | O | C-B56 | A1 | O | Single bond |
| P3-74 | O | C-B56 | A1 | O | O |
| P3-75 | O | C-B56 | A1 | O | L1 |
| P3-76 | O | C-B56 | A1 | O | L5 |
| P3-77 | O | C-B56 | A1 | O | L9 |
| P3-78 | O | C-B56 | A1 | O | L18 |
| P3-79 | O | C-B56 | A1 | O | L22 |
| P3-80 | O | C-B56 | A1 | O | L27 |
| P3-81 | O | C-B56 | A1 | O | L29 |
| P3-82 | O | C-B56 | A1 | O | L36 |
| P3-83 | O | C-B56 | A1 | O | L40 |
| P3-84 | O | C-B56 | A1 | O | L53 |
| P3-85 | O | C-B56 | A1 | O | L59 |
| P3-86 | O | C-B56 | A1 | O | L65 |
| P3-87 | O | C-B56 | A1 | O | L68 |
| P3-88 | O | C-B56 | A1 | O | L119 |
| P3-89 | O | C-B56 | A1 | O | L128 |
| P3-90 | O | C-B56 | A1 | O | L148 |
| P3-91 | O | N | A1 | O | Single bond |
| P3-92 | O | N | A1 | O | O |
| P3-93 | O | N | A1 | O | L1 |
| P3-94 | O | N | A1 | O | L5 |
| P3-95 | O | N | A1 | O | L9 |
| P3-96 | O | N | A1 | O | L18 |
| P3-97 | O | N | A1 | O | L22 |
| P3-98 | O | N | A1 | O | L27 |
| P3-99 | O | N | A1 | O | L29 |
| P3-100 | O | N | A1 | O | L36 |
| P3-101 | O | N | A1 | O | L40 |
| P3-102 | O | N | A1 | O | L53 |
| P3-103 | O | N | A1 | O | L59 |
| P3-104 | O | N | A1 | O | L65 |
| P3-105 | O | N | A1 | O | L68 |
| P3-106 | O | N | A1 | O | L119 |
| P3-107 | O | N | A1 | O | L128 |
| P3-108 | O | N | A1 | O | L148 |
| P3-109 | O | C-A1 | A2 | O | Single bond |
| P3-110 | O | C-A1 | A2 | O | O |
| P3-111 | O | C-A1 | A2 | O | L1 |
| P3-112 | O | C-A1 | A2 | O | L2 |
| P3-113 | O | C-A1 | A2 | O | L3 |
| P3-114 | O | C-A1 | A2 | O | L4 |
| P3-115 | O | C-A1 | A2 | O | L5 |
| P3-116 | O | C-A1 | A2 | O | L6 |
| P3-117 | O | C-A1 | A2 | O | L7 |
| P3-118 | O | C-A1 | A2 | O | L9 |
| P3-119 | O | C-A1 | A2 | O | L11 |
| P3-120 | O | C-A1 | A2 | O | L14 |
| P3-121 | O | C-A1 | A2 | O | L18 |
| P3-122 | O | C-A1 | A2 | O | L22 |
| P3-123 | O | C-A1 | A2 | O | L27 |
| P3-124 | O | C-A1 | A2 | O | L28 |
| P3-125 | O | C-A1 | A2 | O | L29 |
| P3-126 | O | C-A1 | A2 | O | L30 |
| P3-127 | O | C-A1 | A2 | O | L32 |
| P3-128 | O | C-A1 | A2 | O | L36 |
| P3-129 | O | C-A1 | A2 | O | L40 |
| P3-130 | O | C-A1 | A2 | O | L48 |
| P3-131 | O | C-A1 | A2 | O | L53 |
| P3-132 | O | C-A1 | A2 | O | L59 |
| P3-133 | O | C-A1 | A2 | O | L65 |
| P3-134 | O | C-A1 | A2 | O | L68 |
| P3-135 | O | C-A1 | A2 | O | L79 |
| P3-136 | O | C-A1 | A2 | O | L89 |
| P3-137 | O | C-A1 | A2 | O | L102 |
| P3-138 | O | C-A1 | A2 | O | L111 |
| P3-139 | O | C-A1 | A2 | O | L119 |
| P3-140 | O | C-A1 | A2 | O | L128 |
| P3-141 | O | C-A1 | A2 | O | L148 |
| P3-142 | O | C-A1 | A2 | O | L156 |
| P3-143 | O | C-A1 | A2 | O | L167 |
| P3-144 | O | C-A1 | A2 | O | L169 |
| P3-145 | O | C-A1 | A2 | O | L172 |
| P3-146 | O | C-A2 | A2 | O | Single bond |
| P3-147 | O | C-A2 | A2 | O | O |
| P3-148 | O | C-A2 | A2 | O | L1 |
| P3-149 | O | C-A2 | A2 | O | L2 |
| P3-150 | O | C-A2 | A2 | O | L3 |
| P3-151 | O | C-A2 | A2 | O | L4 |
| P3-152 | O | C-A2 | A2 | O | L5 |
| P3-153 | O | C-A2 | A2 | O | L6 |
| P3-154 | O | C-A2 | A2 | O | L7 |
| P3-155 | O | C-A2 | A2 | O | L9 |
| P3-156 | O | C-A2 | A2 | O | L11 |
| P3-157 | O | C-A2 | A2 | O | L14 |
| P3-158 | O | C-A2 | A2 | O | L18 |
| P3-159 | O | C-A2 | A2 | O | L22 |
| P3-160 | O | C-A2 | A2 | O | L27 |
| P3-161 | O | C-A2 | A2 | O | L28 |
| P3-162 | O | C-A2 | A2 | O | L29 |
| P3-163 | O | C-A2 | A2 | O | L30 |
| P3-164 | O | C-A2 | A2 | O | L32 |
| P3-165 | O | C-A2 | A2 | O | L36 |
| P3-166 | O | C-A2 | A2 | O | L40 |
| P3-167 | O | C-A2 | A2 | O | L48 |
| P3-168 | O | C-A2 | A2 | O | L53 |
| P3-169 | O | C-A2 | A2 | O | L59 |
| P3-170 | O | C-A2 | A2 | O | L65 |
| P3-171 | O | C-A2 | A2 | O | L68 |
| P3-172 | O | C-A2 | A2 | O | L79 |
| P3-173 | O | C-A2 | A2 | O | L89 |
| P3-174 | O | C-A2 | A2 | O | L102 |
| P3-175 | O | C-A2 | A2 | O | L111 |
| P3-176 | O | C-A2 | A2 | O | L119 |
| P3-177 | O | C-A2 | A2 | O | L128 |
| P3-178 | O | C-A2 | A2 | O | L148 |
| P3-179 | O | C-A2 | A2 | O | L156 |
| P3-180 | O | C-A2 | A2 | O | L167 |
| P3-181 | O | C-A2 | A2 | O | L169 |
| P3-182 | O | C-A2 | A2 | O | L172 |
| P3-183 | O | C-A3 | A2 | O | Single bond |
| P3-184 | O | C-A3 | A2 | O | O |
| P3-185 | O | C-A3 | A2 | O | L1 |
| P3-186 | O | C-A3 | A2 | O | L2 |
| P3-187 | O | C-A3 | A2 | O | L3 |
| P3-188 | O | C-A3 | A2 | O | L4 |
| P3-189 | O | C-A3 | A2 | O | L5 |
| P3-190 | O | C-A3 | A2 | O | L6 |
| P3-191 | O | C-A3 | A2 | O | L7 |
| P3-192 | O | C-A3 | A2 | O | L9 |
| P3-193 | O | C-A3 | A2 | O | L11 |
| P3-194 | O | C-A3 | A2 | O | L14 |
| P3-195 | O | C-A3 | A2 | O | L18 |
| P3-196 | O | C-A3 | A2 | O | L22 |
| P3-197 | O | C-A3 | A2 | O | L27 |
| P3-198 | O | C-A3 | A2 | O | L28 |
| P3-199 | O | C-A3 | A2 | O | L29 |
| P3-200 | O | C-A3 | A2 | O | L30 |
| P3-201 | O | C-A3 | A2 | O | L32 |
| P3-202 | O | C-A3 | A2 | O | L36 |
| P3-203 | O | C-A3 | A2 | O | L40 |
| P3-204 | O | C-A3 | A2 | O | L48 |
| P3-205 | O | C-A3 | A2 | O | L53 |
| P3-206 | O | C-A3 | A2 | O | L59 |
| P3-207 | O | C-A3 | A2 | O | L65 |
| P3-208 | O | C-A3 | A2 | O | L68 |
| P3-209 | O | C-A3 | A2 | O | L79 |
| P3-210 | O | C-A3 | A2 | O | L89 |
| P3-211 | O | C-A3 | A2 | O | L102 |
| P3-212 | O | C-A3 | A2 | O | L111 |
| P3-213 | O | C-A3 | A2 | O | L119 |
| P3-214 | O | C-A3 | A2 | O | L128 |
| P3-215 | O | C-A3 | A2 | O | L148 |
| P3-216 | O | C-A3 | A2 | O | L156 |
| P3-217 | O | C-A3 | A2 | O | L167 |
| P3-218 | O | C-A3 | A2 | O | L169 |
| P3-219 | O | C-A3 | A2 | O | L172 |
| P3-220 | O | C-B41 | A2 | O | Single bond |
| P3-221 | O | C-B41 | A2 | O | O |
| P3-222 | O | C-B41 | A2 | O | L1 |
| P3-223 | O | C-B41 | A2 | O | L2 |
| P3-224 | O | C-B41 | A2 | O | L3 |
| P3-225 | O | C-B41 | A2 | O | L4 |
| P3-226 | O | C-B41 | A2 | O | L5 |
| P3-227 | O | C-B41 | A2 | O | L6 |
| P3-228 | O | C-B41 | A2 | O | L7 |
| P3-229 | O | C-B41 | A2 | O | L9 |
| P3-230 | O | C-B41 | A2 | O | L11 |
| P3-231 | O | C-B41 | A2 | O | L14 |
| P3-232 | O | C-B41 | A2 | O | L18 |
| P3-233 | O | C-B41 | A2 | O | L22 |
| P3-234 | O | C-B41 | A2 | O | L27 |
| P3-235 | O | C-B41 | A2 | O | L28 |
| P3-236 | O | C-B41 | A2 | O | L29 |
| P3-237 | O | C-B41 | A2 | O | L30 |
| P3-238 | O | C-B41 | A2 | O | L32 |
| P3-239 | O | C-B41 | A2 | O | L36 |
| P3-240 | O | C-B41 | A2 | O | L40 |
| P3-241 | O | C-B41 | A2 | O | L48 |
| P3-242 | O | C-B41 | A2 | O | L53 |
| P3-243 | O | C-B41 | A2 | O | L59 |
| P3-244 | O | C-B41 | A2 | O | L65 |
| P3-245 | O | C-B41 | A2 | O | L68 |
| P3-246 | O | C-B41 | A2 | O | L79 |
| P3-247 | O | C-B41 | A2 | O | L89 |
| P3-248 | O | C-B41 | A2 | O | L102 |
| P3-249 | O | C-B41 | A2 | O | L111 |
| P3-250 | O | C-B41 | A2 | O | L119 |
| P3-251 | O | C-B41 | A2 | O | L128 |
| P3-252 | O | C-B41 | A2 | O | L148 |
| P3-253 | O | C-B41 | A2 | O | L156 |
| P3-254 | O | C-B41 | A2 | O | L167 |
| P3-255 | O | C-B41 | A2 | O | L169 |
| P3-256 | O | C-B41 | A2 | O | L172 |
| P3-257 | O | C-B56 | A2 | O | Single bond |
| P3-258 | O | C-B56 | A2 | O | O |
| P3-259 | O | C-B56 | A2 | O | L1 |
| P3-260 | O | C-B56 | A2 | O | L2 |
| P3-261 | O | C-B56 | A2 | O | L3 |
| P3-262 | O | C-B56 | A2 | O | L4 |
| P3-263 | O | C-B56 | A2 | O | L5 |
| P3-264 | O | C-B56 | A2 | O | L6 |
| P3-265 | O | C-B56 | A2 | O | L7 |
| P3-266 | O | C-B56 | A2 | O | L9 |
| P3-267 | O | C-B56 | A2 | O | L11 |
| P3-268 | O | C-B56 | A2 | O | L14 |
| P3-269 | O | C-B56 | A2 | O | L18 |
| P3-270 | O | C-B56 | A2 | O | L22 |
| P3-271 | O | C-B56 | A2 | O | L27 |
| P3-272 | O | C-B56 | A2 | O | L28 |
| P3-273 | O | C-B56 | A2 | O | L29 |
| P3-274 | O | C-B56 | A2 | O | L30 |
| P3-275 | O | C-B56 | A2 | O | L32 |
| P3-276 | O | C-B56 | A2 | O | L36 |
| P3-277 | O | C-B56 | A2 | O | L40 |
| P3-278 | O | C-B56 | A2 | O | L48 |
| P3-279 | O | C-B56 | A2 | O | L53 |
| P3-280 | O | C-B56 | A2 | O | L59 |
| P3-281 | O | C-B56 | A2 | O | L65 |
| P3-282 | O | C-B56 | A2 | O | L68 |
| P3-283 | O | C-B56 | A2 | O | L79 |
| P3-284 | O | C-B56 | A2 | O | L89 |
| P3-285 | O | C-B56 | A2 | O | L102 |
| P3-286 | O | C-B56 | A2 | O | L111 |
| P3-287 | O | C-B56 | A2 | O | L119 |
| P3-288 | O | C-B56 | A2 | O | L128 |
| P3-289 | O | C-B56 | A2 | O | L148 |
| P3-290 | O | C-B56 | A2 | O | L156 |
| P3-291 | O | C-B56 | A2 | O | L167 |
| P3-292 | O | C-B56 | A2 | O | L169 |
| P3-293 | O | C-B56 | A2 | O | L172 |
| P3-294 | O | N | A2 | O | Single bond |
| P3-295 | O | N | A2 | O | O |
| P3-296 | O | N | A2 | O | L1 |
| P3-297 | O | N | A2 | O | L2 |
| P3-298 | O | N | A2 | O | L3 |
| P3-299 | O | N | A2 | O | L4 |
| P3-300 | O | N | A2 | O | L5 |
| P3-301 | O | N | A2 | O | L6 |
| P3-302 | O | N | A2 | O | L7 |
| P3-303 | O | N | A2 | O | L9 |
| P3-304 | O | N | A2 | O | L11 |
| P3-305 | O | N | A2 | O | L14 |
| P3-306 | O | N | A2 | O | L18 |
| P3-307 | O | N | A2 | O | L22 |
| P3-308 | O | N | A2 | O | L27 |
| P3-309 | O | N | A2 | O | L28 |
| P3-310 | O | N | A2 | O | L29 |
| P3-311 | O | N | A2 | O | L30 |
| P3-312 | O | N | A2 | O | L32 |
| P3-313 | O | N | A2 | O | L36 |
| P3-314 | O | N | A2 | O | L40 |
| P3-315 | O | N | A2 | O | L48 |
| P3-316 | O | N | A2 | O | L53 |
| P3-317 | O | N | A2 | O | L59 |
| P3-318 | O | N | A2 | O | L65 |
| P3-319 | O | N | A2 | O | L68 |
| P3-320 | O | N | A2 | O | L79 |
| P3-321 | O | N | A2 | O | L89 |
| P3-322 | O | N | A2 | O | L102 |
| P3-323 | O | N | A2 | O | L111 |
| P3-324 | O | N | A2 | O | L119 |
| P3-325 | O | N | A2 | O | L128 |
| P3-326 | O | N | A2 | O | L148 |
| P3-327 | O | N | A2 | O | L156 |
| P3-328 | O | N | A2 | O | L167 |
| P3-329 | O | N | A2 | O | L169 |
| P3-330 | O | N | A2 | O | L172 |
| P3-331 | O | C-A1 | A3 | O | Single bond |
| P3-332 | O | C-A1 | A3 | O | O |
| P3-333 | O | C-A1 | A3 | O | L1 |
| P3-334 | O | C-A1 | A3 | O | L2 |
| P3-335 | O | C-A1 | A3 | O | L3 |
| P3-336 | O | C-A1 | A3 | O | L4 |
| P3-337 | O | C-A1 | A3 | O | L5 |
| P3-338 | O | C-A1 | A3 | O | L6 |
| P3-339 | O | C-A1 | A3 | O | L7 |
| P3-340 | O | C-A1 | A3 | O | L9 |
| P3-341 | O | C-A1 | A3 | O | L11 |
| P3-342 | O | C-A1 | A3 | O | L14 |
| P3-343 | O | C-A1 | A3 | O | L18 |
| P3-344 | O | C-A1 | A3 | O | L22 |
| P3-345 | O | C-A1 | A3 | O | L27 |
| P3-346 | O | C-A1 | A3 | O | L28 |
| P3-347 | O | C-A1 | A3 | O | L29 |
| P3-348 | O | C-A1 | A3 | O | L30 |
| P3-349 | O | C-A1 | A3 | O | L32 |
| P3-350 | O | C-A1 | A3 | O | L36 |
| P3-351 | O | C-A1 | A3 | O | L40 |
| P3-352 | O | C-A1 | A3 | O | L48 |
| P3-353 | O | C-A1 | A3 | O | L53 |
| P3-354 | O | C-A1 | A3 | O | L59 |
| P3-355 | O | C-A1 | A3 | O | L65 |
| P3-356 | O | C-A1 | A3 | O | L68 |
| P3-357 | O | C-A1 | A3 | O | L79 |
| P3-358 | O | C-A1 | A3 | O | L89 |
| P3-359 | O | C-A1 | A3 | O | L102 |
| P3-360 | O | C-A1 | A3 | O | L111 |
| P3-361 | O | C-A1 | A3 | O | L119 |
| P3-362 | O | C-A1 | A3 | O | L128 |
| P3-363 | O | C-A1 | A3 | O | L148 |
| P3-364 | O | C-A1 | A3 | O | L156 |
| P3-365 | O | C-A1 | A3 | O | L167 |
| P3-366 | O | C-A1 | A3 | O | L169 |
| P3-367 | O | C-A1 | A3 | O | L172 |
| P3-368 | O | C-A2 | A3 | O | Single bond |
| P3-369 | O | C-A2 | A3 | O | O |
| P3-370 | O | C-A2 | A3 | O | L1 |
| P3-371 | O | C-A2 | A3 | O | L2 |
| P3-372 | O | C-A2 | A3 | O | L3 |
| P3-373 | O | C-A2 | A3 | O | L4 |
| P3-374 | O | C-A2 | A3 | O | L5 |
| P3-375 | O | C-A2 | A3 | O | L6 |
| P3-376 | O | C-A2 | A3 | O | L7 |
| P3-377 | O | C-A2 | A3 | O | L9 |
| P3-378 | O | C-A2 | A3 | O | L11 |
| P3-379 | O | C-A2 | A3 | O | L14 |
| P3-380 | O | C-A2 | A3 | O | L18 |
| P3-381 | O | C-A2 | A3 | O | L22 |
| P3-382 | O | C-A2 | A3 | O | L27 |
| P3-383 | O | C-A2 | A3 | O | L28 |
| P3-384 | O | C-A2 | A3 | O | L29 |
| P3-385 | O | C-A2 | A3 | O | L30 |
| P3-386 | O | C-A2 | A3 | O | L32 |
| P3-387 | O | C-A2 | A3 | O | L36 |
| P3-388 | O | C-A2 | A3 | O | L40 |
| P3-389 | O | C-A2 | A3 | O | L48 |
| P3-390 | O | C-A2 | A3 | O | L53 |
| P3-391 | O | C-A2 | A3 | O | L59 |
| P3-392 | O | C-A2 | A3 | O | L65 |
| P3-393 | O | C-A2 | A3 | O | L68 |
| P3-394 | O | C-A2 | A3 | O | L79 |
| P3-395 | O | C-A2 | A3 | O | L89 |
| P3-396 | O | C-A2 | A3 | O | L102 |
| P3-397 | O | C-A2 | A3 | O | L111 |
| P3-398 | O | C-A2 | A3 | O | L119 |
| P3-399 | O | C-A2 | A3 | O | L128 |
| P3-400 | O | C-A2 | A3 | O | L148 |
| P3-401 | O | C-A2 | A3 | O | L156 |
| P3-402 | O | C-A2 | A3 | O | L167 |
| P3-403 | O | C-A2 | A3 | O | L169 |
| P3-404 | O | C-A2 | A3 | O | L172 |
| P3-405 | O | C-A3 | A3 | O | Single bond |
| P3-406 | O | C-A3 | A3 | O | O |
| P3-407 | O | C-A3 | A3 | O | L1 |
| P3-408 | O | C-A3 | A3 | O | L2 |
| P3-409 | O | C-A3 | A3 | O | L3 |
| P3-410 | O | C-A3 | A3 | O | L4 |
| P3-411 | O | C-A3 | A3 | O | L5 |
| P3-412 | O | C-A3 | A3 | O | L6 |
| P3-413 | O | C-A3 | A3 | O | L7 |
| P3-414 | O | C-A3 | A3 | O | L9 |
| P3-415 | O | C-A3 | A3 | O | L11 |
| P3-416 | O | C-A3 | A3 | O | L14 |
| P3-417 | O | C-A3 | A3 | O | L18 |
| P3-418 | O | C-A3 | A3 | O | L22 |
| P3-419 | O | C-A3 | A3 | O | L27 |
| P3-420 | O | C-A3 | A3 | O | L28 |
| P3-421 | O | C-A3 | A3 | O | L29 |
| P3-422 | O | C-A3 | A3 | O | L30 |
| P3-423 | O | C-A3 | A3 | O | L32 |
| P3-424 | O | C-A3 | A3 | O | L36 |
| P3-425 | O | C-A3 | A3 | O | L40 |
| P3-426 | O | C-A3 | A3 | O | L48 |
| P3-427 | O | C-A3 | A3 | O | L53 |
| P3-428 | O | C-A3 | A3 | O | L59 |
| P3-429 | O | C-A3 | A3 | O | L65 |
| P3-430 | O | C-A3 | A3 | O | L68 |
| P3-431 | O | C-A3 | A3 | O | L79 |
| P3-432 | O | C-A3 | A3 | O | L89 |
| P3-433 | O | C-A3 | A3 | O | L102 |
| P3-434 | O | C-A3 | A3 | O | L111 |
| P3-435 | O | C-A3 | A3 | O | L119 |
| P3-436 | O | C-A3 | A3 | O | L128 |
| P3-437 | O | C-A3 | A3 | O | L148 |
| P3-438 | O | C-A3 | A3 | O | L156 |
| P3-439 | O | C-A3 | A3 | O | L167 |
| P3-440 | O | C-A3 | A3 | O | L169 |
| P3-441 | O | C-A3 | A3 | O | L172 |
| P3-442 | O | C-B41 | A3 | O | Single bond |
| P3-443 | O | C-B41 | A3 | O | O |
| P3-444 | O | C-B41 | A3 | O | L1 |
| P3-445 | O | C-B41 | A3 | O | L2 |
| P3-446 | O | C-B41 | A3 | O | L3 |
| P3-447 | O | C-B41 | A3 | O | L4 |
| P3-448 | O | C-B41 | A3 | O | L5 |
| P3-449 | O | C-B41 | A3 | O | L6 |
| P3-450 | O | C-B41 | A3 | O | L7 |
| P3-451 | O | C-B41 | A3 | O | L9 |
| P3-452 | O | C-B41 | A3 | O | L11 |
| P3-453 | O | C-B41 | A3 | O | L14 |
| P3-454 | O | C-B41 | A3 | O | L18 |
| P3-455 | O | C-B41 | A3 | O | L22 |
| P3-456 | O | C-B41 | A3 | O | L27 |
| P3-457 | O | C-B41 | A3 | O | L28 |
| P3-458 | O | C-B41 | A3 | O | L29 |
| P3-459 | O | C-B41 | A3 | O | L30 |
| P3-460 | O | C-B41 | A3 | O | L32 |
| P3-461 | O | C-B41 | A3 | O | L36 |
| P3-462 | O | C-B41 | A3 | O | L40 |
| P3-463 | O | C-B41 | A3 | O | L48 |
| P3-464 | O | C-B41 | A3 | O | L53 |
| P3-465 | O | C-B41 | A3 | O | L59 |
| P3-466 | O | C-B41 | A3 | O | L65 |
| P3-467 | O | C-B41 | A3 | O | L68 |
| P3-468 | O | C-B41 | A3 | O | L79 |
| P3-469 | O | C-B41 | A3 | O | L89 |
| P3-470 | O | C-B41 | A3 | O | L102 |
| P3-471 | O | C-B41 | A3 | O | L111 |
| P3-472 | O | C-B41 | A3 | O | L119 |
| P3-473 | O | C-B41 | A3 | O | L128 |
| P3-474 | O | C-B41 | A3 | O | L148 |
| P3-475 | O | C-B41 | A3 | O | L156 |
| P3-476 | O | C-B41 | A3 | O | L167 |
| P3-477 | O | C-B41 | A3 | O | L169 |
| P3-478 | O | C-B41 | A3 | O | L172 |
| P3-479 | O | C-B56 | A3 | O | Single bond |
| P3-480 | O | C-B56 | A3 | O | O |
| P3-481 | O | C-B56 | A3 | O | L1 |
| P3-482 | O | C-B56 | A3 | O | L2 |
| P3-483 | O | C-B56 | A3 | O | L3 |
| P3-484 | O | C-B56 | A3 | O | L4 |
| P3-485 | O | C-B56 | A3 | O | L5 |
| P3-486 | O | C-B56 | A3 | O | L6 |
| P3-487 | O | C-B56 | A3 | O | L7 |
| P3-488 | O | C-B56 | A3 | O | L9 |
| P3-489 | O | C-B56 | A3 | O | L11 |
| P3-490 | O | C-B56 | A3 | O | L14 |
| P3-491 | O | C-B56 | A3 | O | L18 |
| P3-492 | O | C-B56 | A3 | O | L22 |
| P3-493 | O | C-B56 | A3 | O | L27 |
| P3-494 | O | C-B56 | A3 | O | L28 |
| P3-495 | O | C-B56 | A3 | O | L29 |
| P3-496 | O | C-B56 | A3 | O | L30 |
| P3-497 | O | C-B56 | A3 | O | L32 |
| P3-498 | O | C-B56 | A3 | O | L36 |
| P3-499 | O | C-B56 | A3 | O | L40 |
| P3-500 | O | C-B56 | A3 | O | L48 |
| P3-501 | O | C-B56 | A3 | O | L53 |
| P3-502 | O | C-B56 | A3 | O | L59 |
| P3-503 | O | C-B56 | A3 | O | L65 |
| P3-504 | O | C-B56 | A3 | O | L68 |
| P3-505 | O | C-B56 | A3 | O | L79 |
| P3-506 | O | C-B56 | A3 | O | L89 |
| P3-507 | O | C-B56 | A3 | O | L102 |
| P3-508 | O | C-B56 | A3 | O | L111 |
| P3-509 | O | C-B56 | A3 | O | L119 |
| P3-510 | O | C-B56 | A3 | O | L128 |
| P3-511 | O | C-B56 | A3 | O | L148 |
| P3-512 | O | C-B56 | A3 | O | L156 |
| P3-513 | O | C-B56 | A3 | O | L167 |
| P3-514 | O | C-B56 | A3 | O | L169 |
| P3-515 | O | C-B56 | A3 | O | L172 |
| P3-516 | O | N | A3 | O | Single bond |
| P3-517 | O | N | A3 | O | O |
| P3-518 | O | N | A3 | O | L1 |
| P3-519 | O | N | A3 | O | L2 |
| P3-520 | O | N | A3 | O | L3 |
| P3-521 | O | N | A3 | O | L4 |
| P3-522 | O | N | A3 | O | L5 |
| P3-523 | O | N | A3 | O | L6 |
| P3-524 | O | N | A3 | O | L7 |
| P3-525 | O | N | A3 | O | L9 |
| P3-526 | O | N | A3 | O | L11 |
| P3-527 | O | N | A3 | O | L14 |
| P3-528 | O | N | A3 | O | L18 |
| P3-529 | O | N | A3 | O | L22 |
| P3-530 | O | N | A3 | O | L27 |
| P3-531 | O | N | A3 | O | L28 |
| P3-532 | O | N | A3 | O | L29 |
| P3-533 | O | N | A3 | O | L30 |
| P3-534 | O | N | A3 | O | L32 |
| P3-535 | O | N | A3 | O | L36 |
| P3-536 | O | N | A3 | O | L40 |
| P3-537 | O | N | A3 | O | L48 |
| P3-538 | O | N | A3 | O | L53 |
| P3-539 | O | N | A3 | O | L59 |
| P3-540 | O | N | A3 | O | L65 |
| P3-541 | O | N | A3 | O | L68 |
| P3-542 | O | N | A3 | O | L79 |
| P3-543 | O | N | A3 | O | L89 |
| P3-544 | O | N | A3 | O | L102 |
| P3-545 | O | N | A3 | O | L111 |
| P3-546 | O | N | A3 | O | L119 |
| P3-547 | O | N | A3 | O | L128 |
| P3-548 | O | N | A3 | O | L148 |
| P3-549 | O | N | A3 | O | L156 |
| P3-550 | O | N | A3 | O | L167 |
| P3-551 | O | N | A3 | O | L169 |
| P3-552 | O | N | A3 | O | L172 |
| P3-553 | O | C-A1 | A12 | O | Single bond |
| P3-554 | O | C-A1 | A12 | O | O |
| P3-555 | O | C-A1 | A12 | O | L1 |
| P3-556 | O | C-A1 | A12 | O | L5 |
| P3-557 | O | C-A1 | A12 | O | L9 |
| P3-558 | O | C-A1 | A12 | O | L18 |
| P3-559 | O | C-A1 | A12 | O | L22 |
| P3-560 | O | C-A1 | A12 | O | L27 |
| P3-561 | O | C-A1 | A12 | O | L29 |
| P3-562 | O | C-A1 | A12 | O | L36 |
| P3-563 | O | C-A1 | A12 | O | L40 |
| P3-564 | O | C-A1 | A12 | O | L53 |
| P3-565 | O | C-A1 | A12 | O | L59 |
| P3-566 | O | C-A1 | A12 | O | L65 |
| P3-567 | O | C-A1 | A12 | O | L68 |
| P3-568 | O | C-A1 | A12 | O | L119 |
| P3-569 | O | C-A1 | A12 | O | L128 |
| P3-570 | O | C-A1 | A12 | O | L148 |
| P3-571 | O | C-A2 | A12 | O | Single bond |
| P3-572 | O | C-A2 | A12 | O | O |
| P3-573 | O | C-A2 | A12 | O | L1 |
| P3-574 | O | C-A2 | A12 | O | L5 |
| P3-575 | O | C-A2 | A12 | O | L9 |
| P3-576 | O | C-A2 | A12 | O | L18 |
| P3-577 | O | C-A2 | A12 | O | L22 |
| P3-578 | O | C-A2 | A12 | O | L27 |
| P3-579 | O | C-A2 | A12 | O | L29 |
| P3-580 | O | C-A2 | A12 | O | L36 |
| P3-581 | O | C-A2 | A12 | O | L40 |
| P3-582 | O | C-A2 | A12 | O | L53 |
| P3-583 | O | C-A2 | A12 | O | L59 |
| P3-584 | O | C-A2 | A12 | O | L65 |
| P3-585 | O | C-A2 | A12 | O | L68 |
| P3-586 | O | C-A2 | A12 | O | L119 |
| P3-587 | O | C-A2 | A12 | O | L128 |
| P3-588 | O | C-A2 | A12 | O | L148 |
| P3-589 | O | C-A3 | A12 | O | Single bond |
| P3-590 | O | C-A3 | A12 | O | O |
| P3-591 | O | C-A3 | A12 | O | L1 |
| P3-592 | O | C-A3 | A12 | O | L5 |
| P3-593 | O | C-A3 | A12 | O | L9 |
| P3-594 | O | C-A3 | A12 | O | L18 |
| P3-595 | O | C-A3 | A12 | O | L22 |
| P3-596 | O | C-A3 | A12 | O | L27 |
| P3-597 | O | C-A3 | A12 | O | L29 |
| P3-598 | O | C-A3 | A12 | O | L36 |
| P3-599 | O | C-A3 | A12 | O | L40 |
| P3-600 | O | C-A3 | A12 | O | L53 |
| P3-601 | O | C-A3 | A12 | O | L59 |
| P3-602 | O | C-A3 | A12 | O | L65 |
| P3-603 | O | C-A3 | A12 | O | L68 |
| P3-604 | O | C-A3 | A12 | O | L119 |
| P3-605 | O | C-A3 | A12 | O | L128 |
| P3-606 | O | C-A3 | A12 | O | L148 |
| P3-607 | O | C-B41 | A12 | O | Single bond |
| P3-608 | O | C-B41 | A12 | O | O |
| P3-609 | O | C-B41 | A12 | O | L1 |
| P3-610 | O | C-B41 | A12 | O | L5 |
| P3-611 | O | C-B41 | A12 | O | L9 |
| P3-612 | O | C-B41 | A12 | O | L18 |
| P3-613 | O | C-B41 | A12 | O | L22 |
| P3-614 | O | C-B41 | A12 | O | L27 |
| P3-615 | O | C-B41 | A12 | O | L29 |
| P3-616 | O | C-B41 | A12 | O | L36 |
| P3-617 | O | C-B41 | A12 | O | L40 |
| P3-618 | O | C-B41 | A12 | O | L53 |
| P3-619 | O | C-B41 | A12 | O | L59 |
| P3-620 | O | C-B41 | A12 | O | L65 |
| P3-621 | O | C-B41 | A12 | O | L68 |
| P3-622 | O | C-B41 | A12 | O | L119 |
| P3-623 | O | C-B41 | A12 | O | L128 |
| P3-624 | O | C-B41 | A12 | O | L148 |
| P3-625 | O | C-B56 | A12 | O | Single bond |
| P3-626 | O | C-B56 | A12 | O | O |
| P3-627 | O | C-B56 | A12 | O | L1 |
| P3-628 | O | C-B56 | A12 | O | L5 |
| P3-629 | O | C-B56 | A12 | O | L9 |
| P3-630 | O | C-B56 | A12 | O | L18 |
| P3-631 | O | C-B56 | A12 | O | L22 |
| P3-632 | O | C-B56 | A12 | O | L27 |
| P3-633 | O | C-B56 | A12 | O | L29 |
| P3-634 | O | C-B56 | A12 | O | L36 |
| P3-635 | O | C-B56 | A12 | O | L40 |
| P3-636 | O | C-B56 | A12 | O | L53 |
| P3-637 | O | C-B56 | A12 | O | L59 |
| P3-638 | O | C-B56 | A12 | O | L65 |
| P3-639 | O | C-B56 | A12 | O | L68 |
| P3-640 | O | C-B56 | A12 | O | L119 |
| P3-641 | O | C-B56 | A12 | O | L128 |
| P3-642 | O | C-B56 | A12 | O | L148 |
| P3-643 | O | N | A12 | O | Single bond |
| P3-644 | O | N | A12 | O | O |
| P3-645 | O | N | A12 | O | L1 |
| P3-646 | O | N | A12 | O | L5 |
| P3-647 | O | N | A12 | O | L9 |
| P3-648 | O | N | A12 | O | L18 |
| P3-649 | O | N | A12 | O | L22 |
| P3-650 | O | N | A12 | O | L27 |
| P3-651 | O | N | A12 | O | L29 |
| P3-652 | O | N | A12 | O | L36 |
| P3-653 | O | N | A12 | O | L40 |
| P3-654 | O | N | A12 | O | L53 |
| P3-655 | O | N | A12 | O | L59 |
| P3-656 | O | N | A12 | O | L65 |
| P3-657 | O | N | A12 | O | L68 |
| P3-658 | O | N | A12 | O | L119 |
| P3-659 | O | N | A12 | O | L128 |
| P3-660 | O | N | A12 | O | L148 |
| P3-661 | O | C-A1 | B41 | O | Single bond |
| P3-662 | O | C-A1 | B41 | O | O |
| P3-663 | O | C-A1 | B41 | O | L1 |
| P3-664 | O | C-A1 | B41 | O | L5 |
| P3-665 | O | C-A1 | B41 | O | L9 |
| P3-666 | O | C-A1 | B41 | O | L18 |
| P3-667 | O | C-A1 | B41 | O | L22 |
| P3-668 | O | C-A1 | B41 | O | L27 |
| P3-669 | O | C-A1 | B41 | O | L29 |
| P3-670 | O | C-A1 | B41 | O | L36 |
| P3-671 | O | C-A1 | B41 | O | L40 |
| P3-672 | O | C-A1 | B41 | O | L53 |
| P3-673 | O | C-A1 | B41 | O | L59 |
| P3-674 | O | C-A1 | B41 | O | L65 |
| P3-675 | O | C-A1 | B41 | O | L68 |
| P3-676 | O | C-A1 | B41 | O | L119 |
| P3-677 | O | C-A1 | B41 | O | L128 |
| P3-678 | O | C-A1 | B41 | O | L148 |
| P3-679 | O | C-A2 | B41 | O | Single bond |
| P3-680 | O | C-A2 | B41 | O | O |
| P3-681 | O | C-A2 | B41 | O | L1 |
| P3-682 | O | C-A2 | B41 | O | L5 |
| P3-683 | O | C-A2 | B41 | O | L9 |
| P3-684 | O | C-A2 | B41 | O | L18 |
| P3-685 | O | C-A2 | B41 | O | L22 |
| P3-686 | O | C-A2 | B41 | O | L27 |
| P3-687 | O | C-A2 | B41 | O | L29 |
| P3-688 | O | C-A2 | B41 | O | L36 |
| P3-689 | O | C-A2 | B41 | O | L40 |
| P3-690 | O | C-A2 | B41 | O | L53 |
| P3-691 | O | C-A2 | B41 | O | L59 |
| P3-692 | O | C-A2 | B41 | O | L65 |
| P3-693 | O | C-A2 | B41 | O | L68 |
| P3-694 | O | C-A2 | B41 | O | L119 |
| P3-695 | O | C-A2 | B41 | O | L128 |
| P3-696 | O | C-A2 | B41 | O | L148 |
| P3-697 | O | C-A3 | B41 | O | Single bond |
| P3-698 | O | C-A3 | B41 | O | O |
| P3-699 | O | C-A3 | B41 | O | L1 |
| P3-700 | O | C-A3 | B41 | O | L5 |
| P3-701 | O | C-A3 | B41 | O | L9 |
| P3-702 | O | C-A3 | B41 | O | L18 |
| P3-703 | O | C-A3 | B41 | O | L22 |
| P3-704 | O | C-A3 | B41 | O | L27 |
| P3-705 | O | C-A3 | B41 | O | L29 |
| P3-706 | O | C-A3 | B41 | O | L36 |
| P3-707 | O | C-A3 | B41 | O | L40 |
| P3-708 | O | C-A3 | B41 | O | L53 |
| P3-709 | O | C-A3 | B41 | O | L59 |
| P3-710 | O | C-A3 | B41 | O | L65 |
| P3-711 | O | C-A3 | B41 | O | L68 |
| P3-712 | O | C-A3 | B41 | O | L119 |
| P3-713 | O | C-A3 | B41 | O | L128 |
| P3-714 | O | C-A3 | B41 | O | L148 |
| P3-715 | O | C-B41 | B41 | O | Single bond |
| P3-716 | O | C-B41 | B41 | O | O |
| P3-717 | O | C-B41 | B41 | O | L1 |
| P3-718 | O | C-B41 | B41 | O | L5 |
| P3-719 | O | C-B41 | B41 | O | L9 |
| P3-720 | O | C-B41 | B41 | O | L18 |
| P3-721 | O | C-B41 | B41 | O | L22 |
| P3-722 | O | C-B41 | B41 | O | L27 |
| P3-723 | O | C-B41 | B41 | O | L29 |
| P3-724 | O | C-B41 | B41 | O | L36 |
| P3-725 | O | C-B41 | B41 | O | L40 |
| P3-726 | O | C-B41 | B41 | O | L53 |
| P3-727 | O | C-B41 | B41 | O | L59 |
| P3-728 | O | C-B41 | B41 | O | L65 |
| P3-729 | O | C-B41 | B41 | O | L68 |
| P3-730 | O | C-B41 | B41 | O | L119 |
| P3-731 | O | C-B41 | B41 | O | L128 |
| P3-732 | O | C-B41 | B41 | O | L148 |
| P3-733 | O | C-B56 | B41 | O | Single bond |
| P3-734 | O | C-B56 | B41 | O | O |
| P3-735 | O | C-B56 | B41 | O | L1 |
| P3-736 | O | C-B56 | B41 | O | L5 |
| P3-737 | O | C-B56 | B41 | O | L9 |
| P3-738 | O | C-B56 | B41 | O | L18 |
| P3-739 | O | C-B56 | B41 | O | L22 |
| P3-740 | O | C-B56 | B41 | O | L27 |
| P3-741 | O | C-B56 | B41 | O | L29 |
| P3-742 | O | C-B56 | B41 | O | L36 |
| P3-743 | O | C-B56 | B41 | O | L40 |
| P3-744 | O | C-B56 | B41 | O | L53 |
| P3-745 | O | C-B56 | B41 | O | L59 |
| P3-746 | O | C-B56 | B41 | O | L65 |
| P3-747 | O | C-B56 | B41 | O | L68 |
| P3-748 | O | C-B56 | B41 | O | L119 |
| P3-749 | O | C-B56 | B41 | O | L128 |
| P3-750 | O | C-B56 | B41 | O | L148 |
| P3-751 | O | N | B41 | O | Single bond |
| P3-752 | O | N | B41 | O | O |
| P3-753 | O | N | B41 | O | L1 |
| P3-754 | O | N | B41 | O | L5 |
| P3-755 | O | N | B41 | O | L9 |
| P3-756 | O | N | B41 | O | L18 |
| P3-757 | O | N | B41 | O | L22 |
| P3-758 | O | N | B41 | O | L27 |
| P3-759 | O | N | B41 | O | L29 |
| P3-760 | O | N | B41 | O | L36 |
| P3-761 | O | N | B41 | O | L40 |
| P3-762 | O | N | B41 | O | L53 |
| P3-763 | O | N | B41 | O | L59 |
| P3-764 | O | N | B41 | O | L65 |
| P3-765 | O | N | B41 | O | L68 |
| P3-766 | O | N | B41 | O | L119 |
| P3-767 | O | N | B41 | O | L128 |
| P3-768 | O | N | B41 | O | L148 |
| P3-769 | O | C-A1 | B56 | O | Single bond |
| P3-770 | O | C-A1 | B56 | O | O |
| P3-771 | O | C-A1 | B56 | O | L1 |
| P3-772 | O | C-A1 | B56 | O | L5 |
| P3-773 | O | C-A1 | B56 | O | L9 |
| P3-774 | O | C-A1 | B56 | O | L18 |
| P3-775 | O | C-A1 | B56 | O | L22 |
| P3-776 | O | C-A1 | B56 | O | L27 |
| P3-777 | O | C-A1 | B56 | O | L29 |
| P3-778 | O | C-A1 | B56 | O | L36 |
| P3-779 | O | C-A1 | B56 | O | L40 |
| P3-780 | O | C-A1 | B56 | O | L53 |
| P3-781 | O | C-A1 | B56 | O | L59 |
| P3-782 | O | C-A1 | B56 | O | L65 |
| P3-783 | O | C-A1 | B56 | O | L68 |
| P3-784 | O | C-A1 | B56 | O | L119 |
| P3-785 | O | C-A1 | B56 | O | L128 |
| P3-786 | O | C-A1 | B56 | O | L148 |
| P3-787 | O | C-A2 | B56 | O | Single bond |
| P3-788 | O | C-A2 | B56 | O | O |
| P3-789 | O | C-A2 | B56 | O | L1 |
| P3-790 | O | C-A2 | B56 | O | L5 |
| P3-791 | O | C-A2 | B56 | O | L9 |
| P3-792 | O | C-A2 | B56 | O | L18 |
| P3-793 | O | C-A2 | B56 | O | L22 |
| P3-794 | O | C-A2 | B56 | O | L27 |
| P3-795 | O | C-A2 | B56 | O | L29 |
| P3-796 | O | C-A2 | B56 | O | L36 |
| P3-797 | O | C-A2 | B56 | O | L40 |
| P3-798 | O | C-A2 | B56 | O | L53 |
| P3-799 | O | C-A2 | B56 | O | L59 |
| P3-800 | O | C-A2 | B56 | O | L65 |
| P3-801 | O | C-A2 | B56 | O | L68 |
| P3-802 | O | C-A2 | B56 | O | L119 |
| P3-803 | O | C-A2 | B56 | O | L128 |
| P3-804 | O | C-A2 | B56 | O | L148 |
| P3-805 | O | C-A3 | B56 | O | Single bond |
| P3-806 | O | C-A3 | B56 | O | O |
| P3-807 | O | C-A3 | B56 | O | L1 |
| P3-808 | O | C-A3 | B56 | O | L5 |
| P3-809 | O | C-A3 | B56 | O | L9 |
| P3-810 | O | C-A3 | B56 | O | L18 |
| P3-811 | O | C-A3 | B56 | O | L22 |
| P3-812 | O | C-A3 | B56 | O | L27 |
| P3-813 | O | C-A3 | B56 | O | L29 |
| P3-814 | O | C-A3 | B56 | O | L36 |
| P3-815 | O | C-A3 | B56 | O | L40 |
| P3-816 | O | C-A3 | B56 | O | L53 |
| P3-817 | O | C-A3 | B56 | O | L59 |
| P3-818 | O | C-A3 | B56 | O | L65 |
| P3-819 | O | C-A3 | B56 | O | L68 |
| P3-820 | O | C-A3 | B56 | O | L119 |
| P3-821 | O | C-A3 | B56 | O | L128 |
| P3-822 | O | C-A3 | B56 | O | L148 |
| P3-823 | O | C-B41 | B56 | O | Single bond |
| P3-824 | O | C-B41 | B56 | O | O |
| P3-825 | O | C-B41 | B56 | O | L1 |
| P3-826 | O | C-B41 | B56 | O | L5 |
| P3-827 | O | C-B41 | B56 | O | L9 |
| P3-828 | O | C-B41 | B56 | O | L18 |
| P3-829 | O | C-B41 | B56 | O | L22 |
| P3-830 | O | C-B41 | B56 | O | L27 |
| P3-831 | O | C-B41 | B56 | O | L29 |
| P3-832 | O | C-B41 | B56 | O | L36 |
| P3-833 | O | C-B41 | B56 | O | L40 |
| P3-834 | O | C-B41 | B56 | O | L53 |
| P3-835 | O | C-B41 | B56 | O | L59 |
| P3-836 | O | C-B41 | B56 | O | L65 |
| P3-837 | O | C-B41 | B56 | O | L68 |
| P3-838 | O | C-B41 | B56 | O | L119 |
| P3-839 | O | C-B41 | B56 | O | L128 |
| P3-840 | O | C-B41 | B56 | O | L148 |
| P3-841 | O | C-B56 | B56 | O | Single bond |
| P3-842 | O | C-B56 | B56 | O | O |
| P3-843 | O | C-B56 | B56 | O | L1 |
| P3-844 | O | C-B56 | B56 | O | L5 |
| P3-845 | O | C-B56 | B56 | O | L9 |
| P3-846 | O | C-B56 | B56 | O | L18 |
| P3-847 | O | C-B56 | B56 | O | L22 |
| P3-848 | O | C-B56 | B56 | O | L27 |
| P3-849 | O | C-B56 | B56 | O | L29 |
| P3-850 | O | C-B56 | B56 | O | L36 |
| P3-851 | O | C-B56 | B56 | O | L40 |
| P3-852 | O | C-B56 | B56 | O | L53 |
| P3-853 | O | C-B56 | B56 | O | L59 |
| P3-854 | O | C-B56 | B56 | O | L65 |
| P3-855 | O | C-B56 | B56 | O | L68 |
| P3-856 | O | C-B56 | B56 | O | L119 |
| P3-857 | O | C-B56 | B56 | O | L128 |
| P3-858 | O | C-B56 | B56 | O | L148 |
| P3-859 | O | N | B56 | O | Single bond |
| P3-860 | O | N | B56 | O | O |
| P3-861 | O | N | B56 | O | L1 |
| P3-862 | O | N | B56 | O | L5 |
| P3-863 | O | N | B56 | O | L9 |
| P3-864 | O | N | B56 | O | L18 |
| P3-865 | O | N | B56 | O | L22 |
| P3-866 | O | N | B56 | O | L27 |
| P3-867 | O | N | B56 | O | L29 |
| P3-868 | O | N | B56 | O | L36 |
| P3-869 | O | N | B56 | O | L40 |
| P3-870 | O | N | B56 | O | L53 |
| P3-871 | O | N | B56 | O | L59 |
| P3-872 | O | N | B56 | O | L65 |
| P3-873 | O | N | B56 | O | L68 |
| P3-874 | O | N | B56 | O | L119 |
| P3-875 | O | N | B56 | O | L128 |
| P3-876 | O | N | B56 | O | L148 |
| P3-877 | O | C-A1 | A2 | S | Single bond |
| P3-878 | O | C-A1 | A2 | S | L5 |
| P3-879 | O | C-A1 | A2 | S | L6 |
| P3-880 | O | C-A1 | A2 | S | L27 |
| P3-881 | O | C-A1 | A2 | S | L29 |
| P3-882 | O | C-A1 | A2 | S | L36 |
| P3-883 | O | C-B41 | A2 | S | Single bond |
| P3-884 | O | C-B41 | A2 | S | L5 |
| P3-885 | O | C-B41 | A2 | S | L6 |
| P3-886 | O | C-B41 | A2 | S | L27 |
| P3-887 | O | C-B41 | A2 | S | L29 |
| P3-888 | O | C-B41 | A2 | S | L36 |
| P3-889 | O | C-A1 | A3 | S | Single bond |
| P3-890 | O | C-A1 | A3 | S | L5 |
| P3-891 | O | C-A1 | A3 | S | L6 |
| P3-892 | O | C-A1 | A3 | S | L27 |
| P3-893 | O | C-A1 | A3 | S | L29 |
| P3-894 | O | C-A1 | A3 | S | L36 |
| P3-895 | O | C-B41 | A3 | S | Single bond |
| P3-896 | O | C-B41 | A3 | S | L5 |
| P3-897 | O | C-B41 | A3 | S | L6 |
| P3-898 | O | C-B41 | A3 | S | L27 |
| P3-899 | O | C-B41 | A3 | S | L29 |
| P3-900 | O | C-B41 | A3 | S | L36 |
| P3-901 | O | C-A1 | A2 | Se | L6 |
| P3-902 | O | C-A1 | A2 | Se | L36 |
| P3-903 | O | C-B41 | A2 | Se | L6 |
| P3-904 | O | C-B41 | A2 | Se | L36 |
| P3-905 | O | C-A1 | A3 | Se | L6 |
| P3-906 | O | C-A1 | A3 | Se | L36 |
| P3-907 | O | C-B41 | A3 | Se | L6 |
| P3-908 | O | C-B41 | A3 | Se | L36 |
| P3-909 | O | C-A1 | A2 | S═O | L6 |
| P3-910 | O | C-A1 | A2 | S═O | L36 |
| P3-911 | O | C-B41 | A2 | S═O | L6 |
| P3-912 | O | C-B41 | A2 | S═O | L36 |
| P3-913 | O | C-A1 | A3 | S═O | L6 |
| P3-914 | O | C-A1 | A3 | S═O | L36 |
| P3-915 | O | C-B41 | A3 | S═O | L6 |
| P3-916 | O | C-B41 | A3 | S═O | L36 |
| P3-917 | O | C-A1 | A2 | O═S═O | Single bond |
| P3-918 | O | C-A1 | A2 | O═S═O | L5 |
| P3-919 | O | C-A1 | A2 | O═S═O | L6 |
| P3-920 | O | C-A1 | A2 | O═S═O | L27 |
| P3-921 | O | C-A1 | A2 | O═S═O | L29 |
| P3-922 | O | C-A1 | A2 | O═S═O | L36 |
| P3-923 | O | C-B41 | A2 | O═S═O | Single bond |
| P3-924 | O | C-B41 | A2 | O═S═O | L5 |
| P3-925 | O | C-B41 | A2 | O═S═O | L6 |
| P3-926 | O | C-B41 | A2 | O═S═O | L27 |
| P3-927 | O | C-B41 | A2 | O═S═O | L29 |
| P3-928 | O | C-B41 | A2 | O═S═O | L36 |
| P3-929 | O | C-A1 | A3 | O═S═O | Single bond |
| P3-930 | O | C-A1 | A3 | O═S═O | L5 |
| P3-931 | O | C-A1 | A3 | O═S═O | L6 |
| P3-932 | O | C-A1 | A3 | O═S═O | L27 |
| P3-933 | O | C-A1 | A3 | O═S═O | L29 |
| P3-934 | O | C-A1 | A3 | O═S═O | L36 |
| P3-935 | O | C-B41 | A3 | O═S═O | Single bond |
| P3-936 | O | C-B41 | A3 | O═S═O | L5 |
| P3-937 | O | C-B41 | A3 | O═S═O | L6 |
| P3-938 | O | C-B41 | A3 | O═S═O | L27 |
| P3-939 | O | C-B41 | A3 | O═S═O | L29 |
| P3-940 | O | C-B41 | A3 | O═S═O | L36 |
| P3-941 | S | C-A1 | A2 | O | Single bond |
| P3-942 | S | C-A1 | A2 | O | L5 |
| P3-943 | S | C-A1 | A2 | O | L6 |
| P3-944 | S | C-A1 | A2 | O | L27 |
| P3-945 | S | C-A1 | A2 | O | L29 |
| P3-946 | S | C-A1 | A2 | O | L36 |
| P3-947 | S | C-B41 | A2 | O | Single bond |
| P3-948 | S | C-B41 | A2 | O | L5 |
| P3-949 | S | C-B41 | A2 | O | L6 |
| P3-950 | S | C-B41 | A2 | O | L27 |
| P3-951 | S | C-B41 | A2 | O | L29 |
| P3-952 | S | C-B41 | A2 | O | L36 |
| P3-953 | S | C-A1 | A3 | O | Single bond |
| P3-954 | S | C-A1 | A3 | O | L5 |
| P3-955 | S | C-A1 | A3 | O | L6 |
| P3-956 | S | C-A1 | A3 | O | L27 |
| P3-957 | S | C-A1 | A3 | O | L29 |
| P3-958 | S | C-A1 | A3 | O | L36 |
| P3-959 | S | C-B41 | A3 | O | Single bond |
| P3-960 | S | C-B41 | A3 | O | L5 |
| P3-961 | S | C-B41 | A3 | O | L6 |
| P3-962 | S | C-B41 | A3 | O | L27 |
| P3-963 | S | C-B41 | A3 | O | L29 |
| P3-964 | S | C-B41 | A3 | O | L36 |
| P3-965 | Se | C-A1 | A2 | O | L5 |
| P3-966 | Se | C-A1 | A2 | O | L6 |
| P3-967 | Se | C-A1 | A2 | O | L27 |
| P3-968 | Se | C-A1 | A2 | O | L29 |
| P3-969 | Se | C-A1 | A2 | O | L36 |
| P3-970 | Se | C-B41 | A2 | O | L5 |
| P3-971 | Se | C-B41 | A2 | O | L6 |
| P3-972 | Se | C-B41 | A2 | O | L27 |
| P3-973 | Se | C-B41 | A2 | O | L29 |
| P3-974 | Se | C-B41 | A2 | O | L36 |
| P3-975 | Se | C-A1 | A3 | O | L5 |
| P3-976 | Se | C-A1 | A3 | O | L6 |
| P3-977 | Se | C-A1 | A3 | O | L27 |
| P3-978 | Se | C-A1 | A3 | O | L29 |
| P3-979 | Se | C-A1 | A3 | O | L36 |
| P3-980 | Se | C-B41 | A3 | O | L5 |
| P3-981 | Se | C-B41 | A3 | O | L6 |
| P3-982 | Se | C-B41 | A3 | O | L27 |
| P3-983 | Se | C-B41 | A3 | O | L29 |
| P3-984 | Se | C-B41 | A3 | O | L36 |
| P3-985 | S | C-A1 | A2 | O═S═O | Single bond |
| P3-986 | S | C-A1 | A2 | O═S═O | L5 |
| P3-987 | S | C-A1 | A2 | O═S═O | L6 |
| P3-988 | S | C-A1 | A2 | O═S═O | L27 |
| P3-989 | S | C-A1 | A2 | O═S═O | L29 |
| P3-990 | S | C-A1 | A2 | O═S═O | L36 |
| P3-991 | S | C-B41 | A2 | O═S═O | Single bond |
| P3-992 | S | C-B41 | A2 | O═S═O | L5 |
| P3-993 | S | C-B41 | A2 | O═S═O | L6 |
| P3-994 | S | C-B41 | A2 | O═S═O | L27 |
| P3-995 | S | C-B41 | A2 | O═S═O | L29 |
| P3-996 | S | C-B41 | A2 | O═S═O | L36 |
| P3-997 | S | C-A1 | A3 | O═S═O | Single bond |
| P3-998 | S | C-A1 | A3 | O═S═O | L5 |
| P3-999 | S | C-A1 | A3 | O═S═O | L6 |
| P3-1000 | S | C-A1 | A3 | O═S═O | L27 |
| P3-1001 | S | C-A1 | A3 | O═S═O | L29 |
| P3-1002 | S | C-A1 | A3 | O═S═O | L36 |
| P3-1003 | S | C-B41 | A3 | O═S═O | Single bond |
| P3-1004 | S | C-B41 | A3 | O═S═O | L5 |
| P3-1005 | S | C-B41 | A3 | O═S═O | L6 |
| P3-1006 | S | C-B41 | A3 | O═S═O | L27 |
| P3-1007 | S | C-B41 | A3 | O═S═O | L29 |
| P3-1008 | S | C-B41 | A3 | O═S═O | L36 |
| P3-1009 | Se | C-A1 | A2 | O═S═O | L5 |
| P3-1010 | Se | C-A1 | A2 | O═S═O | L6 |
| P3-1011 | Se | C-A1 | A2 | O═S═O | L27 |
| P3-1012 | Se | C-A1 | A2 | O═S═O | L29 |
| P3-1013 | Se | C-A1 | A2 | O═S═O | L36 |
| P3-1014 | Se | C-B41 | A2 | O═S═O | L5 |
| P3-1015 | Se | C-B41 | A2 | O═S═O | L6 |
| P3-1016 | Se | C-B41 | A2 | O═S═O | L27 |
| P3-1017 | Se | C-B41 | A2 | O═S═O | L29 |
| P3-1018 | Se | C-B41 | A2 | O═S═O | L36 |
| P3-1019 | Se | C-A1 | A3 | O═S═O | L5 |
| P3-1020 | Se | C-A1 | A3 | O═S═O | L6 |
| P3-1021 | Se | C-A1 | A3 | O═S═O | L27 |
| P3-1022 | Se | C-A1 | A3 | O═S═O | L29 |
| P3-1023 | Se | C-A1 | A3 | O═S═O | L36 |
| P3-1024 | Se | C-B41 | A3 | O═S═O | L5 |
| P3-1025 | Se | C-B41 | A3 | O═S═O | L6 |
| P3-1026 | Se | C-B41 | A3 | O═S═O | L27 |
| P3-1027 | Se | C-B41 | A3 | O═S═O | L29 |
| P3-1028 | Se | C-B41 | A3 | O═S═O | L36 |

The compounds P4-1 to P4-1028 have structures represented by formula I-2-2; wherein R¹ and R² are the same, and W, Z, R¹, R², X, and L correspond to atoms or groups selected from the following table 4, respectively:

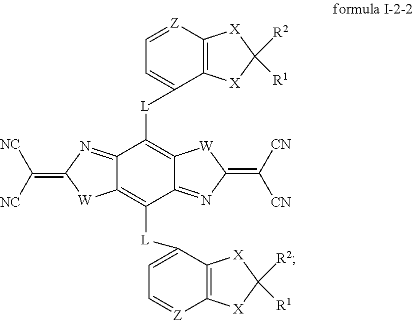

TABLE 4

| Compound No. | W | Z | R¹ and R² | X | L |
|---|---|---|---|---|---|
| P4-1 | O | C-A1 | A1 | O | Single bond |
| P4-2 | O | C-A1 | A1 | O | O |
| P4-3 | O | C-A1 | A1 | O | L1 |
| P4-4 | O | C-A1 | A1 | O | L5 |
| P4-5 | O | C-A1 | A1 | O | L9 |
| P4-6 | O | C-A1 | A1 | O | L18 |
| P4-7 | O | C-A1 | A1 | O | L22 |
| P4-8 | O | C-A1 | A1 | O | L27 |
| P4-9 | O | C-A1 | A1 | O | L29 |
| P4-10 | O | C-A1 | A1 | O | L36 |
| P4-11 | O | C-A1 | A1 | O | L40 |
| P4-12 | O | C-A1 | A1 | O | L53 |
| P4-13 | O | C-A1 | A1 | O | L59 |
| P4-14 | O | C-A1 | A1 | O | L65 |
| P4-15 | O | C-A1 | A1 | O | L68 |
| P4-16 | O | C-A1 | A1 | O | L119 |
| P4-17 | O | C-A1 | A1 | O | L128 |
| P4-18 | O | C-A1 | A1 | O | L148 |
| P4-19 | O | C-A2 | A1 | O | Single bond |
| P4-20 | O | C-A2 | A1 | O | O |
| P4-21 | O | C-A2 | A1 | O | L1 |
| P4-22 | O | C-A2 | A1 | O | L5 |
| P4-23 | O | C-A2 | A1 | O | L9 |
| P4-24 | O | C-A2 | A1 | O | L18 |
| P4-25 | O | C-A2 | A1 | O | L22 |
| P4-26 | O | C-A2 | A1 | O | L27 |
| P4-27 | O | C-A2 | A1 | O | L29 |
| P4-28 | O | C-A2 | A1 | O | L36 |
| P4-29 | O | C-A2 | A1 | O | L40 |
| P4-30 | O | C-A2 | A1 | O | L53 |
| P4-31 | O | C-A2 | A1 | O | L59 |
| P4-32 | O | C-A2 | A1 | O | L65 |
| P4-33 | O | C-A2 | A1 | O | L68 |
| P4-34 | O | C-A2 | A1 | O | L119 |
| P4-35 | O | C-A2 | A1 | O | L128 |
| P4-36 | O | C-A2 | A1 | O | L148 |
| P4-37 | O | C-A3 | A1 | O | Single bond |
| P4-38 | O | C-A3 | A1 | O | O |
| P4-39 | O | C-A3 | A1 | O | L1 |
| P4-40 | O | C-A3 | A1 | O | L5 |
| P4-41 | O | C-A3 | A1 | O | L9 |
| P4-42 | O | C-A3 | A1 | O | L18 |
| P4-43 | O | C-A3 | A1 | O | L22 |
| P4-44 | O | C-A3 | A1 | O | L27 |
| P4-45 | O | C-A3 | A1 | O | L29 |
| P4-46 | O | C-A3 | A1 | O | L36 |
| P4-47 | O | C-A3 | A1 | O | L40 |
| P4-48 | O | C-A3 | A1 | O | L53 |
| P4-49 | O | C-A3 | A1 | O | L59 |
| P4-50 | O | C-A3 | A1 | O | L65 |
| P4-51 | O | C-A3 | A1 | O | L68 |
| P4-52 | O | C-A3 | A1 | O | L119 |
| P4-53 | O | C-A3 | A1 | O | L128 |
| P4-54 | O | C-A3 | A1 | O | L148 |
| P4-55 | O | C-B41 | A1 | O | Single bond |
| P4-56 | O | C-B41 | A1 | O | O |
| P4-57 | O | C-B41 | A1 | O | L1 |
| P4-58 | O | C-B41 | A1 | O | L5 |
| P4-59 | O | C-B41 | A1 | O | L9 |
| P4-60 | O | C-B41 | A1 | O | L18 |
| P4-61 | O | C-B41 | A1 | O | L22 |
| P4-62 | O | C-B41 | A1 | O | L27 |
| P4-63 | O | C-B41 | A1 | O | L29 |
| P4-64 | O | C-B41 | A1 | O | L36 |
| P4-65 | O | C-B41 | A1 | O | L40 |
| P4-66 | O | C-B41 | A1 | O | L53 |
| P4-67 | O | C-B41 | A1 | O | L59 |
| P4-68 | O | C-B41 | A1 | O | L65 |
| P4-69 | O | C-B41 | A1 | O | L68 |
| P4-70 | O | C-B41 | A1 | O | L119 |
| P4-71 | O | C-B41 | A1 | O | L128 |
| P4-72 | O | C-B41 | A1 | O | L148 |
| P4-73 | O | C-B56 | A1 | O | Single bond |
| P4-74 | O | C-B56 | A1 | O | O |
| P4-75 | O | C-B56 | A1 | O | L1 |
| P4-76 | O | C-B56 | A1 | O | L5 |
| P4-77 | O | C-B56 | A1 | O | L9 |
| P4-78 | O | C-B56 | A1 | O | L18 |
| P4-79 | O | C-B56 | A1 | O | L22 |
| P4-80 | O | C-B56 | A1 | O | L27 |
| P4-81 | O | C-B56 | A1 | O | L29 |
| P4-82 | O | C-B56 | A1 | O | L36 |
| P4-83 | O | C-B56 | A1 | O | L40 |
| P4-84 | O | C-B56 | A1 | O | L53 |
| P4-85 | O | C-B56 | A1 | O | L59 |
| P4-86 | O | C-B56 | A1 | O | L65 |
| P4-87 | O | C-B56 | A1 | O | L68 |
| P4-88 | O | C-B56 | A1 | O | L119 |
| P4-89 | O | C-B56 | A1 | O | L128 |
| P4-90 | O | C-B56 | A1 | O | L148 |
| P4-91 | O | N | A1 | O | Single bond |
| P4-92 | O | N | A1 | O | O |
| P4-93 | O | N | A1 | O | L1 |
| P4-94 | O | N | A1 | O | L5 |
| P4-95 | O | N | A1 | O | L9 |
| P4-96 | O | N | A1 | O | L18 |
| P4-97 | O | N | A1 | O | L22 |
| P4-98 | O | N | A1 | O | L27 |
| P4-99 | O | N | A1 | O | L29 |
| P4-100 | O | N | A1 | O | L36 |
| P4-101 | O | N | A1 | O | L40 |
| P4-102 | O | N | A1 | O | L53 |
| P4-103 | O | N | A1 | O | L59 |
| P4-104 | O | N | A1 | O | L65 |
| P4-105 | O | N | A1 | O | L68 |
| P4-106 | O | N | A1 | O | L119 |
| P4-107 | O | N | A1 | O | L128 |
| P4-108 | O | N | A1 | O | L148 |
| P4-109 | O | C-A1 | A2 | O | Single bond |
| P4-110 | O | C-A1 | A2 | O | O |
| P4-111 | O | C-A1 | A2 | O | L1 |
| P4-112 | O | C-A1 | A2 | O | L2 |
| P4-113 | O | C-A1 | A2 | O | L3 |
| P4-114 | O | C-A1 | A2 | O | L4 |
| P4-115 | O | C-A1 | A2 | O | L5 |
| P4-116 | O | C-A1 | A2 | O | L6 |
| P4-117 | O | C-A1 | A2 | O | L7 |
| P4-118 | O | C-A1 | A2 | O | L9 |
| P4-119 | O | C-A1 | A2 | O | L11 |
| P4-120 | O | C-A1 | A2 | O | L14 |
| P4-121 | O | C-A1 | A2 | O | L18 |
| P4-122 | O | C-A1 | A2 | O | L22 |
| P4-123 | O | C-A1 | A2 | O | L27 |
| P4-124 | O | C-A1 | A2 | O | L28 |
| P4-125 | O | C-A1 | A2 | O | L29 |
| P4-126 | O | C-A1 | A2 | O | L30 |
| P4-127 | O | C-A1 | A2 | O | L32 |
| P4-128 | O | C-A1 | A2 | O | L36 |
| P4-129 | O | C-A1 | A2 | O | L40 |
| P4-130 | O | C-A1 | A2 | O | L48 |
| P4-131 | O | C-A1 | A2 | O | L53 |
| P4-132 | O | C-A1 | A2 | O | L59 |
| P4-133 | O | C-A1 | A2 | O | L65 |
| P4-134 | O | C-A1 | A2 | O | L68 |
| P4-135 | O | C-A1 | A2 | O | L79 |
| P4-136 | O | C-A1 | A2 | O | L88 |
| P4-137 | O | C-A1 | A2 | O | L102 |
| P4-138 | O | C-A1 | A2 | O | L111 |
| P4-139 | O | C-A1 | A2 | O | L119 |
| P4-140 | O | C-A1 | A2 | O | L128 |
| P4-141 | O | C-A1 | A2 | O | L148 |
| P4-142 | O | C-A1 | A2 | O | L156 |
| P4-143 | O | C-A1 | A2 | O | L167 |
| P4-144 | O | C-A1 | A2 | O | L169 |
| P4-145 | O | C-A1 | A2 | O | L172 |
| P4-146 | O | C-A2 | A2 | O | Single bond |
| P4-147 | O | C-A2 | A2 | O | O |
| P4-148 | O | C-A2 | A2 | O | L1 |
| P4-149 | O | C-A2 | A2 | O | L2 |
| P4-150 | O | C-A2 | A2 | O | L3 |
| P4-151 | O | C-A2 | A2 | O | L4 |
| P4-152 | O | C-A2 | A2 | O | L5 |
| P4-153 | O | C-A2 | A2 | O | L6 |
| P4-154 | O | C-A2 | A2 | O | L7 |
| P4-155 | O | C-A2 | A2 | O | L9 |
| P4-156 | O | C-A2 | A2 | O | L11 |
| P4-157 | O | C-A2 | A2 | O | L14 |
| P4-158 | O | C-A2 | A2 | O | L18 |
| P4-159 | O | C-A2 | A2 | O | L22 |
| P4-160 | O | C-A2 | A2 | O | L27 |
| P4-161 | O | C-A2 | A2 | O | L28 |
| P4-162 | O | C-A2 | A2 | O | L29 |
| P4-163 | O | C-A2 | A2 | O | L30 |
| P4-164 | O | C-A2 | A2 | O | L32 |
| P4-165 | O | C-A2 | A2 | O | L36 |
| P4-166 | O | C-A2 | A2 | O | L40 |
| P4-167 | O | C-A2 | A2 | O | L48 |
| P4-168 | O | C-A2 | A2 | O | L53 |
| P4-169 | O | C-A2 | A2 | O | L59 |
| P4-170 | O | C-A2 | A2 | O | L65 |
| P4-171 | O | C-A2 | A2 | O | L68 |
| P4-172 | O | C-A2 | A2 | O | L79 |
| P4-173 | O | C-A2 | A2 | O | L89 |
| P4-174 | O | C-A2 | A2 | O | L102 |
| P4-175 | O | C-A2 | A2 | O | L111 |
| P4-176 | O | C-A2 | A2 | O | L119 |
| P4-177 | O | C-A2 | A2 | O | L128 |
| P4-178 | O | C-A2 | A2 | O | L148 |
| P4-179 | O | C-A2 | A2 | O | L156 |
| P4-180 | O | C-A2 | A2 | O | L167 |
| P4-181 | O | C-A2 | A2 | O | L169 |
| P4-182 | O | C-A2 | A2 | O | L172 |
| P4-183 | O | C-A3 | A2 | O | Single bond |
| P4-184 | O | C-A3 | A2 | O | O |
| P4-185 | O | C-A3 | A2 | O | L1 |
| P4-186 | O | C-A3 | A2 | O | L2 |
| P4-187 | O | C-A3 | A2 | O | L3 |
| P4-188 | O | C-A3 | A2 | O | L4 |
| P4-189 | O | C-A3 | A2 | O | L5 |
| P4-190 | O | C-A3 | A2 | O | L6 |
| P4-191 | O | C-A3 | A2 | O | L7 |
| P4-192 | O | C-A3 | A2 | O | L9 |
| P4-193 | O | C-A3 | A2 | O | L11 |
| P4-194 | O | C-A3 | A2 | O | L14 |
| P4-195 | O | C-A3 | A2 | O | L18 |
| P4-196 | O | C-A3 | A2 | O | L22 |
| P4-197 | O | C-A3 | A2 | O | L27 |
| P4-198 | O | C-A3 | A2 | O | L28 |
| P4-199 | O | C-A3 | A2 | O | L29 |
| P4-200 | O | C-A3 | A2 | O | L30 |
| P4-201 | O | C-A3 | A2 | O | L32 |
| P4-202 | O | C-A3 | A2 | O | L36 |
| P4-203 | O | C-A3 | A2 | O | L40 |
| P4-204 | O | C-A3 | A2 | O | L48 |
| P4-205 | O | C-A3 | A2 | O | L53 |
| P4-206 | O | C-A3 | A2 | O | L59 |
| P4-207 | O | C-A3 | A2 | O | L65 |
| P4-208 | O | C-A3 | A2 | O | L68 |
| P4-209 | O | C-A3 | A2 | O | L79 |
| P4-210 | O | C-A3 | A2 | O | L89 |
| P4-211 | O | C-A3 | A2 | O | L102 |
| P4-212 | O | C-A3 | A2 | O | L111 |
| P4-213 | O | C-A3 | A2 | O | L119 |
| P4-214 | O | C-A3 | A2 | O | L128 |
| P4-215 | O | C-A3 | A2 | O | L148 |
| P4-216 | O | C-A3 | A2 | O | L156 |
| P4-217 | O | C-A3 | A2 | O | L167 |
| P4-218 | O | C-A3 | A2 | O | L169 |
| P4-219 | O | C-A3 | A2 | O | L172 |
| P4-220 | O | C-B41 | A2 | O | Single bond |
| P4-221 | O | C-B41 | A2 | O | O |
| P4-222 | O | C-B41 | A2 | O | L1 |
| P4-223 | O | C-B41 | A2 | O | L2 |
| P4-224 | O | C-B41 | A2 | O | L3 |
| P4-225 | O | C-B41 | A2 | O | L4 |
| P4-226 | O | C-B41 | A2 | O | L5 |
| P4-227 | O | C-B41 | A2 | O | L6 |
| P4-228 | O | C-B41 | A2 | O | L7 |
| P4-229 | O | C-B41 | A2 | O | L9 |
| P4-230 | O | C-B41 | A2 | O | L11 |
| P4-231 | O | C-B41 | A2 | O | L14 |
| P4-232 | O | C-B41 | A2 | O | L18 |
| P4-233 | O | C-B41 | A2 | O | L22 |
| P4-234 | O | C-B41 | A2 | O | L27 |
| P4-235 | O | C-B41 | A2 | O | L28 |
| P4-236 | O | C-B41 | A2 | O | L29 |
| P4-237 | O | C-B41 | A2 | O | L30 |
| P4-238 | O | C-B41 | A2 | O | L32 |
| P4-239 | O | C-B41 | A2 | O | L36 |
| P4-240 | O | C-B41 | A2 | O | L40 |
| P4-241 | O | C-B41 | A2 | O | L48 |
| P4-242 | O | C-B41 | A2 | O | L53 |
| P4-243 | O | C-B41 | A2 | O | L59 |
| P4-244 | O | C-B41 | A2 | O | L65 |
| P4-245 | O | C-B41 | A2 | O | L68 |
| P4-246 | O | C-B41 | A2 | O | L79 |
| P4-247 | O | C-B41 | A2 | O | L89 |
| P4-248 | O | C-B41 | A2 | O | L102 |
| P4-249 | O | C-B41 | A2 | O | L111 |
| P4-250 | O | C-B41 | A2 | O | L119 |
| P4-251 | O | C-B41 | A2 | O | L128 |
| P4-252 | O | C-B41 | A2 | O | L148 |
| P4-253 | O | C-B41 | A2 | O | L156 |
| P4-254 | O | C-B41 | A2 | O | L167 |
| P4-255 | O | C-B41 | A2 | O | L169 |
| P4-256 | O | C-B41 | A2 | O | L172 |
| P4-257 | O | C-B56 | A2 | O | Single bond |
| P4-258 | O | C-B56 | A2 | O | O |
| P4-259 | O | C-B56 | A2 | O | L1 |
| P4-260 | O | C-B56 | A2 | O | L2 |
| P4-261 | O | C-B56 | A2 | O | L3 |
| P4-262 | O | C-B56 | A2 | O | L4 |
| P4-263 | O | C-B56 | A2 | O | L5 |
| P4-264 | O | C-B56 | A2 | O | L6 |
| P4-265 | O | C-B56 | A2 | O | L7 |
| P4-266 | O | C-B56 | A2 | O | L9 |
| P4-267 | O | C-B56 | A2 | O | L11 |
| P4-268 | O | C-B56 | A2 | O | L14 |
| P4-269 | O | C-B56 | A2 | O | L18 |
| P4-270 | O | C-B56 | A2 | O | L22 |
| P4-271 | O | C-B56 | A2 | O | L27 |
| P4-272 | O | C-B56 | A2 | O | L28 |
| P4-273 | O | C-B56 | A2 | O | L29 |
| P4-274 | O | C-B56 | A2 | O | L30 |
| P4-275 | O | C-B56 | A2 | O | L32 |
| P4-276 | O | C-B56 | A2 | O | L36 |
| P4-277 | O | C-B56 | A2 | O | L40 |
| P4-278 | O | C-B56 | A2 | O | L48 |
| P4-279 | O | C-B56 | A2 | O | L53 |
| P4-280 | O | C-B56 | A2 | O | L59 |
| P4-281 | O | C-B56 | A2 | O | L65 |
| P4-282 | O | C-B56 | A2 | O | L68 |
| P4-283 | O | C-B56 | A2 | O | L79 |
| P4-284 | O | C-B56 | A2 | O | L89 |
| P4-285 | O | C-B56 | A2 | O | L102 |
| P4-286 | O | C-B56 | A2 | O | L111 |
| P4-287 | O | C-B56 | A2 | O | L119 |
| P4-288 | O | C-B56 | A2 | O | L128 |
| P4-289 | O | C-B56 | A2 | O | L148 |
| P4-290 | O | C-B56 | A2 | O | L156 |
| P4-291 | O | C-B56 | A2 | O | L167 |
| P4-292 | O | C-B56 | A2 | O | L169 |
| P4-293 | O | C-B56 | A2 | O | L172 |
| P4-294 | O | N | A2 | O | Single bond |
| P4-295 | O | N | A2 | O | O |
| P4-296 | O | N | A2 | O | L1 |
| P4-297 | O | N | A2 | O | L2 |
| P4-298 | O | N | A2 | O | L3 |
| P4-299 | O | N | A2 | O | L4 |
| P4-300 | O | N | A2 | O | L5 |
| P4-301 | O | N | A2 | O | L6 |
| P4-302 | O | N | A2 | O | L7 |
| P4-303 | O | N | A2 | O | L9 |
| P4-304 | O | N | A2 | O | L11 |
| P4-305 | O | N | A2 | O | L14 |
| P4-306 | O | N | A2 | O | L18 |
| P4-307 | O | N | A2 | O | L22 |
| P4-308 | O | N | A2 | O | L27 |
| P4-309 | O | N | A2 | O | L28 |
| P4-310 | O | N | A2 | O | L29 |
| P4-311 | O | N | A2 | O | L30 |
| P4-312 | O | N | A2 | O | L32 |
| P4-313 | O | N | A2 | O | L36 |
| P4-314 | O | N | A2 | O | L40 |
| P4-315 | O | N | A2 | O | L48 |
| P4-316 | O | N | A2 | O | L53 |
| P4-317 | O | N | A2 | O | L59 |
| P4-318 | O | N | A2 | O | L65 |
| P4-319 | O | N | A2 | O | L68 |
| P4-320 | O | N | A2 | O | L79 |
| P4-321 | O | N | A2 | O | L89 |
| P4-322 | O | N | A2 | O | L102 |
| P4-323 | O | N | A2 | O | L111 |
| P4-324 | O | N | A2 | O | L119 |
| P4-325 | O | N | A2 | O | L128 |
| P4-326 | O | N | A2 | O | L148 |
| P4-327 | O | N | A2 | O | L156 |
| P4-328 | O | N | A2 | O | L167 |
| P4-329 | O | N | A2 | O | L169 |
| P4-330 | O | N | A2 | O | L172 |
| P4-331 | O | C-A1 | A3 | O | Single bond |
| P4-332 | O | C-A1 | A3 | O | O |
| P4-333 | O | C-A1 | A3 | O | L1 |
| P4-334 | O | C-A1 | A3 | O | L2 |
| P4-335 | O | C-A1 | A3 | O | L3 |
| P4-336 | O | C-A1 | A3 | O | L4 |
| P4-337 | O | C-A1 | A3 | O | L5 |
| P4-338 | O | C-A1 | A3 | O | L6 |
| P4-339 | O | C-A1 | A3 | O | L7 |
| P4-340 | O | C-A1 | A3 | O | L9 |
| P4-341 | O | C-A1 | A3 | O | L11 |
| P4-342 | O | C-A1 | A3 | O | L14 |
| P4-343 | O | C-A1 | A3 | O | L18 |
| P4-344 | O | C-A1 | A3 | O | L22 |
| P4-345 | O | C-A1 | A3 | O | L27 |
| P4-346 | O | C-A1 | A3 | O | L28 |
| P4-347 | O | C-A1 | A3 | O | L29 |
| P4-348 | O | C-A1 | A3 | O | L30 |
| P4-349 | O | C-A1 | A3 | O | L32 |
| P4-350 | O | C-A1 | A3 | O | L36 |
| P4-351 | O | C-A1 | A3 | O | L40 |
| P4-352 | O | C-A1 | A3 | O | L48 |
| P4-353 | O | C-A1 | A3 | O | L53 |
| P4-354 | O | C-A1 | A3 | O | L59 |
| P4-355 | O | C-A1 | A3 | O | L65 |
| P4-356 | O | C-A1 | A3 | O | L68 |
| P4-357 | O | C-A1 | A3 | O | L79 |
| P4-358 | O | C-A1 | A3 | O | L89 |
| P4-359 | O | C-A1 | A3 | O | L102 |
| P4-360 | O | C-A1 | A3 | O | L111 |
| P4-361 | O | C-A1 | A3 | O | L119 |
| P4-362 | O | C-A1 | A3 | O | L128 |
| P4-363 | O | C-A1 | A3 | O | L148 |
| P4-364 | O | C-A1 | A3 | O | L156 |
| P4-365 | O | C-A1 | A3 | O | L167 |
| P4-366 | O | C-A1 | A3 | O | L169 |
| P4-367 | O | C-A1 | A3 | O | L172 |
| P4-368 | O | C-A2 | A3 | O | Single bond |
| P4-369 | O | C-A2 | A3 | O | O |
| P4-370 | O | C-A2 | A3 | O | L1 |
| P4-371 | O | C-A2 | A3 | O | L2 |
| P4-372 | O | C-A2 | A3 | O | L3 |
| P4-373 | O | C-A2 | A3 | O | L4 |
| P4-374 | O | C-A2 | A3 | O | L5 |
| P4-375 | O | C-A2 | A3 | O | L6 |
| P4-376 | O | C-A2 | A3 | O | L7 |
| P4-377 | O | C-A2 | A3 | O | L9 |
| P4-378 | O | C-A2 | A3 | O | L11 |
| P4-379 | O | C-A2 | A3 | O | L14 |
| P4-380 | O | C-A2 | A3 | O | L18 |
| P4-381 | O | C-A2 | A3 | O | L22 |
| P4-382 | O | C-A2 | A3 | O | L27 |
| P4-383 | O | C-A2 | A3 | O | L28 |
| P4-384 | O | C-A2 | A3 | O | L29 |
| P4-385 | O | C-A2 | A3 | O | L30 |
| P4-386 | O | C-A2 | A3 | O | L32 |
| P4-387 | O | C-A2 | A3 | O | L36 |
| P4-388 | O | C-A2 | A3 | O | L40 |
| P4-389 | O | C-A2 | A3 | O | L48 |
| P4-390 | O | C-A2 | A3 | O | L53 |
| P4-391 | O | C-A2 | A3 | O | L59 |
| P4-392 | O | C-A2 | A3 | O | L65 |
| P4-393 | O | C-A2 | A3 | O | L68 |
| P4-394 | O | C-A2 | A3 | O | L79 |
| P4-395 | O | C-A2 | A3 | O | L89 |
| P4-396 | O | C-A2 | A3 | O | L102 |
| P4-397 | O | C-A2 | A3 | O | L111 |
| P4-398 | O | C-A2 | A3 | O | L119 |
| P4-399 | O | C-A2 | A3 | O | L128 |
| P4-400 | O | C-A2 | A3 | O | L148 |
| P4-401 | O | C-A2 | A3 | O | L156 |
| P4-402 | O | C-A2 | A3 | O | L167 |
| P4-403 | O | C-A2 | A3 | O | L169 |
| P4-404 | O | C-A2 | A3 | O | L172 |
| P4-405 | O | C-A3 | A3 | O | Single bond |
| P4-406 | O | C-A3 | A3 | O | O |
| P4-407 | O | C-A3 | A3 | O | L1 |
| P4-408 | O | C-A3 | A3 | O | L2 |
| P4-409 | O | C-A3 | A3 | O | L3 |
| P4-410 | O | C-A3 | A3 | O | L4 |
| P4-411 | O | C-A3 | A3 | O | L5 |
| P4-412 | O | C-A3 | A3 | O | L6 |
| P4-413 | O | C-A3 | A3 | O | L7 |
| P4-414 | O | C-A3 | A3 | O | L9 |
| P4-415 | O | C-A3 | A3 | O | L11 |
| P4-416 | O | C-A3 | A3 | O | L14 |
| P4-417 | O | C-A3 | A3 | O | L18 |
| P4-418 | O | C-A3 | A3 | O | L22 |
| P4-419 | O | C-A3 | A3 | O | L27 |
| P4-420 | O | C-A3 | A3 | O | L28 |
| P4-421 | O | C-A3 | A3 | O | L29 |
| P4-422 | O | C-A3 | A3 | O | L30 |
| P4-423 | O | C-A3 | A3 | O | L32 |
| P4-424 | O | C-A3 | A3 | O | L36 |
| P4-425 | O | C-A3 | A3 | O | L40 |
| P4-426 | O | C-A3 | A3 | O | L48 |
| P4-427 | O | C-A3 | A3 | O | L53 |
| P4-428 | O | C-A3 | A3 | O | L59 |
| P4-429 | O | C-A3 | A3 | O | L65 |
| P4-430 | O | C-A3 | A3 | O | L68 |
| P4-431 | O | C-A3 | A3 | O | L79 |
| P4-432 | O | C-A3 | A3 | O | L89 |
| P4-433 | O | C-A3 | A3 | O | L102 |
| P4-434 | O | C-A3 | A3 | O | L111 |
| P4-435 | O | C-A3 | A3 | O | L119 |
| P4-436 | O | C-A3 | A3 | O | L128 |
| P4-437 | O | C-A3 | A3 | O | L148 |
| P4-438 | O | C-A3 | A3 | O | L156 |
| P4-439 | O | C-A3 | A3 | O | L167 |
| P4-440 | O | C-A3 | A3 | O | L169 |
| P4-441 | O | C-A3 | A3 | O | L172 |
| P4-442 | O | C-B41 | A3 | O | Single bond |
| P4-443 | O | C-B41 | A3 | O | O |
| P4-444 | O | C-B41 | A3 | O | L1 |
| P4-445 | O | C-B41 | A3 | O | L2 |
| P4-446 | O | C-B41 | A3 | O | L3 |
| P4-447 | O | C-B41 | A3 | O | L4 |
| P4-448 | O | C-B41 | A3 | O | L5 |
| P4-449 | O | C-B41 | A3 | O | L6 |
| P4-450 | O | C-B41 | A3 | O | L7 |
| P4-451 | O | C-B41 | A3 | O | L9 |
| P4-452 | O | C-B41 | A3 | O | L11 |
| P4-453 | O | C-B41 | A3 | O | L14 |
| P4-454 | O | C-B41 | A3 | O | L18 |
| P4-455 | O | C-B41 | A3 | O | L22 |
| P4-456 | O | C-B41 | A3 | O | L27 |
| P4-457 | O | C-B41 | A3 | O | L28 |
| P4-458 | O | C-B41 | A3 | O | L29 |
| P4-459 | O | C-B41 | A3 | O | L30 |
| P4-460 | O | C-B41 | A3 | O | L32 |
| P4-461 | O | C-B41 | A3 | O | L36 |
| P4-462 | O | C-B41 | A3 | O | L40 |
| P4-463 | O | C-B41 | A3 | O | L48 |
| P4-464 | O | C-B41 | A3 | O | L53 |
| P4-465 | O | C-B41 | A3 | O | L59 |
| P4-466 | O | C-B41 | A3 | O | L65 |
| P4-467 | O | C-B41 | A3 | O | L68 |
| P4-468 | O | C-B41 | A3 | O | L79 |
| P4-469 | O | C-B41 | A3 | O | L89 |
| P4-470 | O | C-B41 | A3 | O | L102 |
| P4-471 | O | C-B41 | A3 | O | L111 |
| P4-472 | O | C-B41 | A3 | O | L119 |
| P4-473 | O | C-B41 | A3 | O | L128 |
| P4-474 | O | C-B41 | A3 | O | L148 |
| P4-475 | O | C-B41 | A3 | O | L156 |
| P4-476 | O | C-B41 | A3 | O | L167 |
| P4-477 | O | C-B41 | A3 | O | L169 |
| P4-478 | O | C-B41 | A3 | O | L172 |
| P4-479 | O | C-B56 | A3 | O | Single bond |
| P4-480 | O | C-B56 | A3 | O | O |
| P4-481 | O | C-B56 | A3 | O | L1 |
| P4-482 | O | C-B56 | A3 | O | L2 |
| P4-483 | O | C-B56 | A3 | O | L3 |
| P4-484 | O | C-B56 | A3 | O | L4 |
| P4-485 | O | C-B56 | A3 | O | L5 |
| P4-486 | O | C-B56 | A3 | O | L6 |
| P4-487 | O | C-B56 | A3 | O | L7 |
| P4-488 | O | C-B56 | A3 | O | L9 |
| P4-489 | O | C-B56 | A3 | O | L11 |
| P4-490 | O | C-B56 | A3 | O | L14 |
| P4-491 | O | C-B56 | A3 | O | L18 |
| P4-492 | O | C-B56 | A3 | O | L22 |
| P4-493 | O | C-B56 | A3 | O | L27 |
| P4-494 | O | C-B56 | A3 | O | L28 |
| P4-495 | O | C-B56 | A3 | O | L29 |
| P4-496 | O | C-B56 | A3 | O | L30 |
| P4-497 | O | C-B56 | A3 | O | L32 |
| P4-498 | O | C-B56 | A3 | O | L36 |
| P4-499 | O | C-B56 | A3 | O | L40 |
| P4-500 | O | C-B56 | A3 | O | L48 |
| P4-501 | O | C-B56 | A3 | O | L53 |
| P4-502 | O | C-B56 | A3 | O | L59 |
| P4-503 | O | C-B56 | A3 | O | L65 |
| P4-504 | O | C-B56 | A3 | O | L68 |
| P4-505 | O | C-B56 | A3 | O | L79 |
| P4-506 | O | C-B56 | A3 | O | L89 |
| P4-507 | O | C-B56 | A3 | O | L102 |
| P4-508 | O | C-B56 | A3 | O | L111 |
| P4-509 | O | C-B56 | A3 | O | L119 |
| P4-510 | O | C-B56 | A3 | O | L128 |
| P4-511 | O | C-B56 | A3 | O | L148 |
| P4-512 | O | C-B56 | A3 | O | L156 |
| P4-513 | O | C-B56 | A3 | O | L167 |
| P4-514 | O | C-B56 | A3 | O | L169 |
| P4-515 | O | C-B56 | A3 | O | L172 |
| P4-516 | O | N | A3 | O | Single bond |
| P4-517 | O | N | A3 | O | O |
| P4-518 | O | N | A3 | O | L1 |
| P4-519 | O | N | A3 | O | L2 |
| P4-520 | O | N | A3 | O | L3 |
| P4-521 | O | N | A3 | O | L4 |
| P4-522 | O | N | A3 | O | L5 |
| P4-523 | O | N | A3 | O | L6 |
| P4-524 | O | N | A3 | O | L7 |
| P4-525 | O | N | A3 | O | L9 |
| P4-526 | O | N | A3 | O | L11 |
| P4-527 | O | N | A3 | O | L14 |
| P4-528 | O | N | A3 | O | L18 |
| P4-529 | O | N | A3 | O | L22 |
| P4-530 | O | N | A3 | O | L27 |
| P4-531 | O | N | A3 | O | L28 |
| P4-532 | O | N | A3 | O | L29 |
| P4-533 | O | N | A3 | O | L30 |
| P4-534 | O | N | A3 | O | L32 |
| P4-535 | O | N | A3 | O | L36 |
| P4-536 | O | N | A3 | O | L40 |
| P4-537 | O | N | A3 | O | L48 |
| P4-538 | O | N | A3 | O | L53 |
| P4-539 | O | N | A3 | O | L59 |
| P4-540 | O | N | A3 | O | L65 |
| P4-541 | O | N | A3 | O | L68 |
| P4-542 | O | N | A3 | O | L79 |
| P4-543 | O | N | A3 | O | L89 |
| P4-544 | O | N | A3 | O | L102 |
| P4-545 | O | N | A3 | O | L111 |
| P4-546 | O | N | A3 | O | L119 |
| P4-547 | O | N | A3 | O | L128 |
| P4-548 | O | N | A3 | O | L148 |
| P4-549 | O | N | A3 | O | L156 |
| P4-550 | O | N | A3 | O | L167 |
| P4-551 | O | N | A3 | O | L169 |
| P4-552 | O | N | A3 | O | L172 |
| P4-553 | O | C-A1 | A12 | O | Single bond |
| P4-554 | O | C-A1 | A12 | O | O |
| P4-555 | O | C-A1 | A12 | O | L1 |
| P4-556 | O | C-A1 | A12 | O | L5 |
| P4-557 | O | C-A1 | A12 | O | L9 |
| P4-558 | O | C-A1 | A12 | O | L18 |
| P4-559 | O | C-A1 | A12 | O | L22 |
| P4-560 | O | C-A1 | A12 | O | L27 |
| P4-561 | O | C-A1 | A12 | O | L29 |
| P4-562 | O | C-A1 | A12 | O | L36 |
| P4-563 | O | C-A1 | A12 | O | L40 |
| P4-564 | O | C-A1 | A12 | O | L53 |
| P4-565 | O | C-A1 | A12 | O | L59 |
| P4-566 | O | C-A1 | A12 | O | L65 |
| P4-567 | O | C-A1 | A12 | O | L68 |
| P4-568 | O | C-A1 | A12 | O | L119 |
| P4-569 | O | C-A1 | A12 | O | L128 |
| P4-570 | O | C-A1 | A12 | O | L148 |
| P4-571 | O | C-A2 | A12 | O | Single bond |
| P4-572 | O | C-A2 | A12 | O | O |
| P4-573 | O | C-A2 | A12 | O | L1 |
| P4-574 | O | C-A2 | A12 | O | L5 |
| P4-575 | O | C-A2 | A12 | O | L9 |
| P4-576 | O | C-A2 | A12 | O | L18 |
| P4-577 | O | C-A2 | A12 | O | L22 |
| P4-578 | O | C-A2 | A12 | O | L27 |
| P4-579 | O | C-A2 | A12 | O | L29 |
| P4-580 | O | C-A2 | A12 | O | L36 |
| P4-581 | O | C-A2 | A12 | O | L40 |
| P4-582 | O | C-A2 | A12 | O | L53 |
| P4-583 | O | C-A2 | A12 | O | L59 |
| P4-584 | O | C-A2 | A12 | O | L65 |
| P4-585 | O | C-A2 | A12 | O | L68 |
| P4-586 | O | C-A2 | A12 | O | L119 |
| P4-587 | O | C-A2 | A12 | O | L128 |
| P4-588 | O | C-A2 | A12 | O | L148 |
| P4-589 | O | C-A3 | A12 | O | Single bond |
| P4-590 | O | C-A3 | A12 | O | O |
| P4-591 | O | C-A3 | A12 | O | L1 |
| P4-592 | O | C-A3 | A12 | O | L5 |
| P4-593 | O | C-A3 | A12 | O | L9 |
| P4-594 | O | C-A3 | A12 | O | L18 |
| P4-595 | O | C-A3 | A12 | O | L22 |
| P4-596 | O | C-A3 | A12 | O | L27 |
| P4-597 | O | C-A3 | A12 | O | L29 |
| P4-598 | O | C-A3 | A12 | O | L36 |
| P4-599 | O | C-A3 | A12 | O | L40 |
| P4-600 | O | C-A3 | A12 | O | L53 |
| P4-601 | O | C-A3 | A12 | O | L59 |
| P4-602 | O | C-A3 | A12 | O | L65 |
| P4-603 | O | C-A3 | A12 | O | L68 |
| P4-604 | O | C-A3 | A12 | O | L119 |
| P4-605 | O | C-A3 | A12 | O | L128 |
| P4-606 | O | C-A3 | A12 | O | L148 |
| P4-607 | O | C-B41 | A12 | O | Single bond |
| P4-608 | O | C-B41 | A12 | O | O |
| P4-609 | O | C-B41 | A12 | O | L1 |
| P4-610 | O | C-B41 | A12 | O | L5 |
| P4-611 | O | C-B41 | A12 | O | L9 |
| P4-612 | O | C-B41 | A12 | O | L18 |
| P4-613 | O | C-B41 | A12 | O | L22 |
| P4-614 | O | C-B41 | A12 | O | L27 |
| P4-615 | O | C-B41 | A12 | O | L29 |
| P4-616 | O | C-B41 | A12 | O | L36 |
| P4-617 | O | C-B41 | A12 | O | L40 |
| P4-618 | O | C-B41 | A12 | O | L53 |
| P4-619 | O | C-B41 | A12 | O | L59 |
| P4-620 | O | C-B41 | A12 | O | L65 |
| P4-621 | O | C-B41 | A12 | O | L68 |
| P4-622 | O | C-B41 | A12 | O | L119 |
| P4-623 | O | C-B41 | A12 | O | L128 |
| P4-624 | O | C-B41 | A12 | O | L148 |
| P4-625 | O | C-B56 | A12 | O | Single bond |
| P4-626 | O | C-B56 | A12 | O | O |
| P4-627 | O | C-B56 | A12 | O | L1 |
| P4-628 | O | C-B56 | A12 | O | L5 |
| P4-629 | O | C-B56 | A12 | O | L9 |
| P4-630 | O | C-B56 | A12 | O | L18 |
| P4-631 | O | C-B56 | A12 | O | L22 |
| P4-632 | O | C-B56 | A12 | O | L27 |
| P4-633 | O | C-B56 | A12 | O | L29 |
| P4-634 | O | C-B56 | A12 | O | L36 |
| P4-635 | O | C-B56 | A12 | O | L40 |
| P4-636 | O | C-B56 | A12 | O | L53 |
| P4-637 | O | C-B56 | A12 | O | L59 |
| P4-638 | O | C-B56 | A12 | O | L65 |
| P4-639 | O | C-B56 | A12 | O | L68 |
| P4-640 | O | C-B56 | A12 | O | L119 |
| P4-641 | O | C-B56 | A12 | O | L128 |
| P4-642 | O | C-B56 | A12 | O | L148 |
| P4-643 | O | N | A12 | O | Single bond |
| P4-644 | O | N | A12 | O | O |
| P4-645 | O | N | A12 | O | L1 |
| P4-646 | O | N | A12 | O | L5 |
| P4-647 | O | N | A12 | O | L9 |
| P4-648 | O | N | A12 | O | L18 |
| P4-649 | O | N | A12 | O | L22 |
| P4-650 | O | N | A12 | O | L27 |
| P4-651 | O | N | A12 | O | L29 |
| P4-652 | O | N | A12 | O | L36 |
| P4-653 | O | N | A12 | O | L40 |
| P4-654 | O | N | A12 | O | L53 |
| P4-655 | O | N | A12 | O | L59 |
| P4-656 | O | N | A12 | O | L65 |
| P4-657 | O | N | A12 | O | L68 |
| P4-658 | O | N | A12 | O | L119 |
| P4-659 | O | N | A12 | O | L128 |
| P4-660 | O | N | A12 | O | L148 |
| P4-661 | O | C-A1 | B41 | O | Single bond |
| P4-662 | O | C-A1 | B41 | O | O |
| P4-663 | O | C-A1 | B41 | O | L1 |
| P4-664 | O | C-A1 | B41 | O | L5 |
| P4-665 | O | C-A1 | B41 | O | L9 |
| P4-666 | O | C-A1 | B41 | O | L18 |
| P4-667 | O | C-A1 | B41 | O | L22 |
| P4-668 | O | C-A1 | B41 | O | L27 |
| P4-669 | O | C-A1 | B41 | O | L29 |
| P4-670 | O | C-A1 | B41 | O | L36 |
| P4-671 | O | C-A1 | B41 | O | L40 |
| P4-672 | O | C-A1 | B41 | O | L53 |
| P4-673 | O | C-A1 | B41 | O | L59 |
| P4-674 | O | C-A1 | B41 | O | L65 |
| P4-675 | O | C-A1 | B41 | O | L68 |
| P4-676 | O | C-A1 | B41 | O | L119 |
| P4-677 | O | C-A1 | B41 | O | L128 |
| P4-678 | O | C-A1 | B41 | O | L148 |
| P4-679 | O | C-A2 | B41 | O | Single bond |
| P4-680 | O | C-A2 | B41 | O | O |
| P4-681 | O | C-A2 | B41 | O | L1 |
| P4-682 | O | C-A2 | B41 | O | L5 |
| P4-683 | O | C-A2 | B41 | O | L9 |
| P4-684 | O | C-A2 | B41 | O | L18 |
| P4-685 | O | C-A2 | B41 | O | L22 |
| P4-686 | O | C-A2 | B41 | O | L27 |
| P4-687 | O | C-A2 | B41 | O | L29 |
| P4-688 | O | C-A2 | B41 | O | L36 |
| P4-689 | O | C-A2 | B41 | O | L40 |
| P4-690 | O | C-A2 | B41 | O | L53 |
| P4-691 | O | C-A2 | B41 | O | L59 |
| P4-692 | O | C-A2 | B41 | O | L65 |
| P4-693 | O | C-A2 | B41 | O | L68 |
| P4-694 | O | C-A2 | B41 | O | L119 |
| P4-695 | O | C-A2 | B41 | O | L128 |
| P4-696 | O | C-A2 | B41 | O | L148 |
| P4-697 | O | C-A3 | B41 | O | Single bond |
| P4-698 | O | C-A3 | B41 | O | O |
| P4-699 | O | C-A3 | B41 | O | L1 |
| P4-700 | O | C-A3 | B41 | O | L5 |
| P4-701 | O | C-A3 | B41 | O | L9 |
| P4-702 | O | C-A3 | B41 | O | L18 |
| P4-703 | O | C-A3 | B41 | O | L22 |
| P4-704 | O | C-A3 | B41 | O | L27 |
| P4-705 | O | C-A3 | B41 | O | L29 |
| P4-706 | O | C-A3 | B41 | O | L36 |
| P4-707 | O | C-A3 | B41 | O | L40 |
| P4-708 | O | C-A3 | B41 | O | L53 |
| P4-709 | O | C-A3 | B41 | O | L59 |
| P4-710 | O | C-A3 | B41 | O | L65 |
| P4-711 | O | C-A3 | B41 | O | L68 |
| P4-712 | O | C-A3 | B41 | O | L119 |
| P4-713 | O | C-A3 | B41 | O | L128 |
| P4-714 | O | C-A3 | B41 | O | L148 |
| P4-715 | O | C-B41 | B41 | O | Single bond |
| P4-716 | O | C-B41 | B41 | O | O |
| P4-717 | O | C-B41 | B41 | O | L1 |
| P4-718 | O | C-B41 | B41 | O | L5 |
| P4-719 | O | C-B41 | B41 | O | L9 |
| P4-720 | O | C-B41 | B41 | O | L18 |
| P4-721 | O | C-B41 | B41 | O | L22 |
| P4-722 | O | C-B41 | B41 | O | L27 |
| P4-723 | O | C-B41 | B41 | O | L29 |
| P4-724 | O | C-B41 | B41 | O | L36 |
| P4-725 | O | C-B41 | B41 | O | L40 |
| P4-726 | O | C-B41 | B41 | O | L53 |
| P4-727 | O | C-B41 | B41 | O | L59 |
| P4-728 | O | C-B41 | B41 | O | L65 |
| P4-729 | O | C-B41 | B41 | O | L68 |
| P4-730 | O | C-B41 | B41 | O | L119 |
| P4-731 | O | C-B41 | B41 | O | L128 |
| P4-732 | O | C-B41 | B41 | O | L148 |
| P4-733 | O | C-B56 | B41 | O | Single bond |
| P4-734 | O | C-B56 | B41 | O | O |
| P4-735 | O | C-B56 | B41 | O | L1 |
| P4-736 | O | C-B56 | B41 | O | L5 |
| P4-737 | O | C-B56 | B41 | O | L9 |
| P4-738 | O | C-B56 | B41 | O | L18 |
| P4-739 | O | C-B56 | B41 | O | L22 |
| P4-740 | O | C-B56 | B41 | O | L27 |
| P4-741 | O | C-B56 | B41 | O | L29 |
| P4-742 | O | C-B56 | B41 | O | L36 |
| P4-743 | O | C-B56 | B41 | O | L40 |
| P4-744 | O | C-B56 | B41 | O | L53 |
| P4-745 | O | C-B56 | B41 | O | L59 |
| P4-746 | O | C-B56 | B41 | O | L65 |
| P4-747 | O | C-B56 | B41 | O | L68 |
| P4-748 | O | C-B56 | B41 | O | L119 |
| P4-749 | O | C-B56 | B41 | O | L128 |
| P4-750 | O | C-B56 | B41 | O | L148 |
| P4-751 | O | N | B41 | O | Single bond |
| P4-752 | O | N | B41 | O | O |
| P4-753 | O | N | B41 | O | L1 |
| P4-754 | O | N | B41 | O | L5 |
| P4-755 | O | N | B41 | O | L9 |
| P4-756 | O | N | B41 | O | L18 |
| P4-757 | O | N | B41 | O | L22 |
| P4-758 | O | N | B41 | O | L27 |
| P4-759 | O | N | B41 | O | L29 |
| P4-760 | O | N | B41 | O | L36 |
| P4-761 | O | N | B41 | O | L40 |
| P4-762 | O | N | B41 | O | L53 |
| P4-763 | O | N | B41 | O | L59 |
| P4-764 | O | N | B41 | O | L65 |
| P4-765 | O | N | B41 | O | L68 |
| P4-766 | O | N | B41 | O | L119 |
| P4-767 | O | N | B41 | O | L128 |
| P4-768 | O | N | B41 | O | L148 |
| P4-769 | O | C-A1 | B56 | O | Single bond |
| P4-770 | O | C-A1 | B56 | O | O |
| P4-771 | O | C-A1 | B56 | O | L1 |
| P4-772 | O | C-A1 | B56 | O | L5 |
| P4-773 | O | C-A1 | B56 | O | L9 |
| P4-774 | O | C-A1 | B56 | O | L18 |
| P4-775 | O | C-A1 | B56 | O | L22 |
| P4-776 | O | C-A1 | B56 | O | L27 |
| P4-777 | O | C-A1 | B56 | O | L29 |
| P4-778 | O | C-A1 | B56 | O | L36 |
| P4-779 | O | C-A1 | B56 | O | L40 |
| P4-780 | O | C-A1 | B56 | O | L53 |
| P4-781 | O | C-A1 | B56 | O | L59 |
| P4-782 | O | C-A1 | B56 | O | L65 |
| P4-783 | O | C-A1 | B56 | O | L68 |
| P4-784 | O | C-A1 | B56 | O | L119 |
| P4-785 | O | C-A1 | B56 | O | L128 |
| P4-786 | O | C-A1 | B56 | O | L148 |
| P4-787 | O | C-A2 | B56 | O | Single bond |
| P4-788 | O | C-A2 | B56 | O | O |
| P4-789 | O | C-A2 | B56 | O | L1 |
| P4-790 | O | C-A2 | B56 | O | L5 |
| P4-791 | O | C-A2 | B56 | O | L9 |
| P4-792 | O | C-A2 | B56 | O | L18 |
| P4-793 | O | C-A2 | B56 | O | L22 |
| P4-794 | O | C-A2 | B56 | O | L27 |
| P4-795 | O | C-A2 | B56 | O | L29 |
| P4-796 | O | C-A2 | B56 | O | L36 |
| P4-797 | O | C-A2 | B56 | O | L40 |
| P4-798 | O | C-A2 | B56 | O | L53 |
| P4-799 | O | C-A2 | B56 | O | L59 |
| P4-800 | O | C-A2 | B56 | O | L65 |
| P4-801 | O | C-A2 | B56 | O | L68 |
| P4-802 | O | C-A2 | B56 | O | L119 |
| P4-803 | O | C-A2 | B56 | O | L128 |
| P4-804 | O | C-A2 | B56 | O | L148 |
| P4-805 | O | C-A3 | B56 | O | Single bond |
| P4-806 | O | C-A3 | B56 | O | O |
| P4-807 | O | C-A3 | B56 | O | L1 |
| P4-808 | O | C-A3 | B56 | O | L5 |
| P4-809 | O | C-A3 | B56 | O | L9 |
| P4-810 | O | C-A3 | B56 | O | L18 |
| P4-811 | O | C-A3 | B56 | O | L22 |
| P4-812 | O | C-A3 | B56 | O | L27 |
| P4-813 | O | C-A3 | B56 | O | L29 |
| P4-814 | O | C-A3 | B56 | O | L36 |
| P4-815 | O | C-A3 | B56 | O | L40 |
| P4-816 | O | C-A3 | B56 | O | L53 |
| P4-817 | O | C-A3 | B56 | O | L59 |
| P4-818 | O | C-A3 | B56 | O | L65 |
| P4-819 | O | C-A3 | B56 | O | L68 |
| P4-820 | O | C-A3 | B56 | O | L119 |
| P4-821 | O | C-A3 | B56 | O | L128 |
| P4-822 | O | C-A3 | B56 | O | L148 |
| P4-823 | O | C-B41 | B56 | O | Single bond |
| P4-824 | O | C-B41 | B56 | O | O |
| P4-825 | O | C-B41 | B56 | O | L1 |
| P4-826 | O | C-B41 | B56 | O | L5 |
| P4-827 | O | C-B41 | B56 | O | L9 |
| P4-828 | O | C-B41 | B56 | O | L18 |
| P4-829 | O | C-B41 | B56 | O | L22 |
| P4-830 | O | C-B41 | B56 | O | L27 |
| P4-831 | O | C-B41 | B56 | O | L29 |
| P4-832 | O | C-B41 | B56 | O | L36 |
| P4-833 | O | C-B41 | B56 | O | L40 |
| P4-834 | O | C-B41 | B56 | O | L53 |
| P4-835 | O | C-B41 | B56 | O | L59 |
| P4-836 | O | C-B41 | B56 | O | L65 |
| P4-837 | O | C-B41 | B56 | O | L68 |
| P4-838 | O | C-B41 | B56 | O | L119 |
| P4-839 | O | C-B41 | B56 | O | L128 |
| P4-840 | O | C-B41 | B56 | O | L148 |
| P4-841 | O | C-B56 | B56 | O | Single bond |
| P4-842 | O | C-B56 | B56 | O | O |
| P4-843 | O | C-B56 | B56 | O | L1 |
| P4-844 | O | C-B56 | B56 | O | L5 |
| P4-845 | O | C-B56 | B56 | O | L9 |
| P4-846 | O | C-B56 | B56 | O | L18 |
| P4-847 | O | C-B56 | B56 | O | L22 |
| P4-848 | O | C-B56 | B56 | O | L27 |
| P4-849 | O | C-B56 | B56 | O | L29 |
| P4-850 | O | C-B56 | B56 | O | L36 |
| P4-851 | O | C-B56 | B56 | O | L40 |
| P4-852 | O | C-B56 | B56 | O | L53 |
| P4-853 | O | C-B56 | B56 | O | L59 |
| P4-854 | O | C-B56 | B56 | O | L65 |
| P4-855 | O | C-B56 | B56 | O | L68 |
| P4-856 | O | C-B56 | B56 | O | L119 |
| P4-857 | O | C-B56 | B56 | O | L128 |
| P4-858 | O | C-B56 | B56 | O | L148 |
| P4-859 | O | N | B56 | O | Single bond |
| P4-860 | O | N | B56 | O | O |
| P4-861 | O | N | B56 | O | L1 |
| P4-862 | O | N | B56 | O | L5 |
| P4-863 | O | N | B56 | O | L9 |
| P4-864 | O | N | B56 | O | L18 |
| P4-865 | O | N | B56 | O | L22 |
| P4-866 | O | N | B56 | O | L27 |
| P4-867 | O | N | B56 | O | L29 |
| P4-868 | O | N | B56 | O | L36 |
| P4-869 | O | N | B56 | O | L40 |
| P4-870 | O | N | B56 | O | L53 |
| P4-871 | O | N | B56 | O | L59 |
| P4-872 | O | N | B56 | O | L65 |
| P4-873 | O | N | B56 | O | L68 |
| P4-874 | O | N | B56 | O | L119 |
| P4-875 | O | N | B56 | O | L128 |
| P4-876 | O | N | B56 | O | L148 |
| P4-877 | O | C-A1 | A2 | S | Single bond |
| P4-878 | O | C-A1 | A2 | S | L5 |
| P4-879 | O | C-A1 | A2 | S | L6 |
| P4-880 | O | C-A1 | A2 | S | L27 |
| P4-881 | O | C-A1 | A2 | S | L29 |
| P4-882 | O | C-A1 | A2 | S | L36 |
| P4-883 | O | C-B41 | A2 | S | Single bond |
| P4-884 | O | C-B41 | A2 | S | L5 |
| P4-885 | O | C-B41 | A2 | S | L6 |
| P4-886 | O | C-B41 | A2 | S | L27 |
| P4-887 | O | C-B41 | A2 | S | L29 |
| P4-888 | O | C-B41 | A2 | S | L36 |
| P4-889 | O | C-A1 | A3 | S | Single bond |
| P4-890 | O | C-A1 | A3 | S | L5 |
| P4-891 | O | C-A1 | A3 | S | L6 |
| P4-892 | O | C-A1 | A3 | S | L27 |
| P4-893 | O | C-A1 | A3 | S | L29 |
| P4-894 | O | C-A1 | A3 | S | L36 |
| P4-895 | O | C-B41 | A3 | S | Single bond |
| P4-896 | O | C-B41 | A3 | S | L5 |
| P4-897 | O | C-B41 | A3 | S | L6 |
| P4-898 | O | C-B41 | A3 | S | L27 |
| P4-899 | O | C-B41 | A3 | S | L29 |
| P4-900 | O | C-B41 | A3 | S | L36 |
| P4-901 | O | C-A1 | A2 | Se | L6 |
| P4-902 | O | C-A1 | A2 | Se | L36 |
| P4-903 | O | C-B41 | A2 | Se | L6 |
| P4-904 | O | C-B41 | A2 | Se | L36 |
| P4-905 | O | C-A1 | A3 | Se | L6 |
| P4-906 | O | C-A1 | A3 | Se | L36 |
| P4-907 | O | C-B41 | A3 | Se | L6 |
| P4-908 | O | C-B41 | A3 | Se | L36 |
| P4-909 | O | C-A1 | A2 | S═O | L6 |
| P4-910 | O | C-A1 | A2 | S═O | L36 |
| P4-911 | O | C-B41 | A2 | S═O | L6 |
| P4-912 | O | C-B41 | A2 | S═O | L36 |
| P4-913 | O | C-A1 | A3 | S═O | L6 |
| P4-914 | O | C-A1 | A3 | S═O | L36 |
| P4-915 | O | C-B41 | A3 | S═O | L6 |
| P4-916 | O | C-B41 | A3 | S═O | L36 |
| P4-917 | O | C-A1 | A2 | O═S═O | Single bond |
| P4-918 | O | C-A1 | A2 | O═S═O | L5 |
| P4-919 | O | C-A1 | A2 | O═S═O | L6 |
| P4-920 | O | C-A1 | A2 | O═S═O | L27 |
| P4-921 | O | C-A1 | A2 | O═S═O | L29 |
| P4-922 | O | C-A1 | A2 | O═S═O | L36 |
| P4-923 | O | C-B41 | A2 | O═S═O | Single bond |
| P4-924 | O | C-B41 | A2 | O═S═O | L5 |
| P4-925 | O | C-B41 | A2 | O═S═O | L6 |
| P4-926 | O | C-B41 | A2 | O═S═O | L27 |
| P4-927 | O | C-B41 | A2 | O═S═O | L29 |
| P4-928 | O | C-B41 | A2 | O═S═O | L36 |
| P4-929 | O | C-A1 | A3 | O═S═O | Single bond |
| P4-930 | O | C-A1 | A3 | O═S═O | L5 |
| P4-931 | O | C-A1 | A3 | O═S═O | L6 |
| P4-932 | O | C-A1 | A3 | O═S═O | L27 |
| P4-933 | O | C-A1 | A3 | O═S═O | L29 |
| P4-934 | O | C-A1 | A3 | O═S═O | L36 |
| P4-935 | O | C-B41 | A3 | O═S═O | Single bond |
| P4-936 | O | C-B41 | A3 | O═S═O | L5 |
| P4-937 | O | C-B41 | A3 | O═S═O | L6 |
| P4-938 | O | C-B41 | A3 | O═S═O | L27 |
| P4-939 | O | C-B41 | A3 | O═S═O | L29 |
| P4-940 | O | C-B41 | A3 | O═S═O | L36 |
| P4-941 | S | C-A1 | A2 | O | Single bond |
| P4-942 | S | C-A1 | A2 | O | L5 |
| P4-943 | S | C-A1 | A2 | O | L6 |
| P4-944 | S | C-A1 | A2 | O | L27 |
| P4-945 | S | C-A1 | A2 | O | L29 |
| P4-946 | S | C-A1 | A2 | O | L36 |
| P4-947 | S | C-B41 | A2 | O | Single bond |
| P4-948 | S | C-B41 | A2 | O | L5 |
| P4-949 | S | C-B41 | A2 | O | L6 |
| P4-950 | S | C-B41 | A2 | O | L27 |
| P4-951 | S | C-B41 | A2 | O | L29 |
| P4-952 | S | C-B41 | A2 | O | L36 |
| P4-953 | S | C-A1 | A3 | O | Single bond |
| P4-954 | S | C-A1 | A3 | O | L5 |
| P4-955 | S | C-A1 | A3 | O | L6 |
| P4-956 | S | C-A1 | A3 | O | L27 |
| P4-957 | S | C-A1 | A3 | O | L29 |
| P4-958 | S | C-A1 | A3 | O | L36 |
| P4-959 | S | C-B41 | A3 | O | Single bond |
| P4-960 | S | C-B41 | A3 | O | L5 |
| P4-961 | S | C-B41 | A3 | O | L6 |
| P4-962 | S | C-B41 | A3 | O | L27 |
| P4-963 | S | C-B41 | A3 | O | L29 |
| P4-964 | S | C-B41 | A3 | O | L36 |
| P4-965 | Se | C-A1 | A2 | O | L5 |
| P4-966 | Se | C-A1 | A2 | O | L6 |
| P4-967 | Se | C-A1 | A2 | O | L27 |
| P4-968 | Se | C-A1 | A2 | O | L29 |
| P4-969 | Se | C-A1 | A2 | O | L36 |
| P4-970 | Se | C-B41 | A2 | O | L5 |
| P4-971 | Se | C-B41 | A2 | O | L6 |
| P4-972 | Se | C-B41 | A2 | O | L27 |
| P4-973 | Se | C-B41 | A2 | O | L29 |
| P4-974 | Se | C-B41 | A2 | O | L36 |
| P4-975 | Se | C-A1 | A3 | O | L5 |
| P4-976 | Se | C-A1 | A3 | O | L6 |
| P4-977 | Se | C-A1 | A3 | O | L27 |
| P4-978 | Se | C-A1 | A3 | O | L29 |
| P4-979 | Se | C-A1 | A3 | O | L36 |
| P4-980 | Se | C-B41 | A3 | O | L5 |
| P4-981 | Se | C-B41 | A3 | O | L6 |
| P4-982 | Se | C-B41 | A3 | O | L27 |
| P4-983 | Se | C-B41 | A3 | O | L29 |
| P4-984 | Se | C-B41 | A3 | O | L36 |
| P4-985 | S | C-A1 | A2 | O═S═O | Single bond |
| P4-986 | S | C-A1 | A2 | O═S═O | L5 |
| P4-987 | S | C-A1 | A2 | O═S═O | L6 |
| P4-988 | S | C-A1 | A2 | O═S═O | L27 |
| P4-989 | S | C-A1 | A2 | O═S═O | L29 |
| P4-990 | S | C-A1 | A2 | O═S═O | L36 |
| P4-991 | S | C-B41 | A2 | O═S═O | Single bond |
| P4-992 | S | C-B41 | A2 | O═S═O | L5 |
| P4-993 | S | C-B41 | A2 | O═S═O | L6 |
| P4-994 | S | C-B41 | A2 | O═S═O | L27 |
| P4-995 | S | C-B41 | A2 | O═S═O | L29 |
| P4-996 | S | C-B41 | A2 | O═S═O | L36 |
| P4-997 | S | C-A1 | A3 | O═S═O | Single bond |
| P4-998 | S | C-A1 | A3 | O═S═O | L5 |
| P4-999 | S | C-A1 | A3 | O═S═O | L6 |
| P4-1000 | S | C-A1 | A3 | O═S═O | L27 |
| P4-1001 | S | C-A1 | A3 | O═S═O | L29 |
| P4-1002 | S | C-A1 | A3 | O═S═O | L36 |
| P4-1003 | S | C-B41 | A3 | O═S═O | Single bond |
| P4-1004 | S | C-B41 | A3 | O═S═O | L5 |
| P4-1005 | S | C-B41 | A3 | O═S═O | L6 |
| P4-1006 | S | C-B41 | A3 | O═S═O | L27 |
| P4-1007 | S | C-B41 | A3 | O═S═O | L29 |
| P4-1008 | S | C-B41 | A3 | O═S═O | L36 |
| P4-1009 | Se | C-A1 | A2 | O═S═O | L5 |
| P4-1010 | Se | C-A1 | A2 | O═S═O | L6 |
| P4-1011 | Se | C-A1 | A2 | O═S═O | L27 |
| P4-1012 | Se | C-A1 | A2 | O═S═O | L29 |
| P4-1013 | Se | C-A1 | A2 | O═S═O | L36 |
| P4-1014 | Se | C-B41 | A2 | O═S═O | L5 |
| P4-1015 | Se | C-B41 | A2 | O═S═O | L6 |
| P4-1016 | Se | C-B41 | A2 | O═S═O | L27 |
| P4-1017 | Se | C-B41 | A2 | O═S═O | L29 |
| P4-1018 | Se | C-B41 | A2 | O═S═O | L36 |
| P4-1019 | Se | C-A1 | A3 | O═S═O | L5 |
| P4-1020 | Se | C-A1 | A3 | O═S═O | L6 |
| P4-1021 | Se | C-A1 | A3 | O═S═O | L27 |
| P4-1022 | Se | C-A1 | A3 | O═S═O | L29 |
| P4-1023 | Se | C-A1 | A3 | O═S═O | L36 |
| P4-1024 | Se | C-B41 | A3 | O═S═O | L5 |
| P4-1025 | Se | C-B41 | A3 | O═S═O | L6 |
| P4-1026 | Se | C-B41 | A3 | O═S═O | L27 |
| P4-1027 | Se | C-B41 | A3 | O═S═O | L29 |
| P4-1028 | Se | C-B41 | A3 | O═S═O | L36 |

hydrogens in the compounds with the above structures can be partially or completely replaced by deuterium.

A second aspect provides an organic electroluminescent scent device, comprising:
an anode,
a cathode,
and an organic layer arranged between the anode and the cathode, wherein the organic layer comprises the compound as described above.

Furthermore, the organic layer is a hole injection layer or a hole transport layer, and the hole injection layer or the hole transport layer is formed from the compound as described above alone.

Furthermore, the organic layer is a hole injection layer or a hole transport layer, and the hole injection layer or the hole transport layer further comprises at least one hole transport material, the mass doping ratio of the compound to the at least one hole transport material being 1000:1 to 1:1000, preferably 10:1 to 1:100.

Furthermore, the organic electroluminescent device comprises at least two light-emitting units, and the organic layer is a charge generation layer arranged between the at least two light-emitting units, wherein the charge generation layer comprises a p-type charge generation layer and an n-type charge generation layer;
the p-type charge generation layer comprises the compound as described above; and
the p-type charge generation layer further comprises at least one hole transport material, the mass doping ratio of the compound to the at least one hole transport material being 1000:1 to 1:1000, preferably 10:1 to 1:100.

Furthermore, the hole transport material is selected from compounds having triarylamine units, spirobifluorene compounds, pentacene compounds, oligothiophene compounds, oligophenyl compounds, oligo(phenylene vinylene) compounds, oligofluorene compounds, porphyrin complexes, and metal phthalocyanine complexes.

Furthermore, the charge generation layer further comprises a buffer layer arranged between the p-type charge generation layer and the n-type charge generation layer, and the buffer layer comprises the compound as described above.

A third aspect provides a display assembly, comprising the organic electroluminescent device as described above.

### Beneficial Effects of the Invention

The present invention provides an organic compound and an organic electroluminescent device comprising the organic compound. The organic compound has a structure represented by formula I. The organic compound is a compound having a multi-substituted dehydrogenated fused-ring structure with a plurality of substituents, wherein at least one of Ar₁ and Ar₂ has a structure represented by formula II. Formula II has a unique double-X five-membered ring structure. The unique double-X five-membered ring structure has not only good thermal stability but also stable electron-withdrawing ability. The unique double-X five-membered ring structure is connected to a multi-substituted dehydrogenated fused-ring parent nucleus, so that not only can the sublimation yield of the material be improved, but also superior doping ability is achieved. The organic compound is used for preparing an organic electroluminescent device, can improve the hole injection ability in the hole injection layer, reduce the voltage of the device, and improve the efficiency and lifetime of the device. Furthermore, in the formula II of the compound provided by the present invention, the double-X five-membered ring structure is connected to the multi-substituted dehydrogenated fused-ring parent nucleus via the spacer group L, so that the sublimation yield of the material can be significantly improved.

### BRIEF DESCRIPTION OF THE DRAWINGS

FIG. 1 is a schematic diagram of an organic electroluminescent device according to an embodiment in the description of the present invention, in which a device 100 comprises a substrate 101, an anode 110, a hole injection layer 120, a hole transport layer 130, an electron barrier layer 140, a light-emitting layer 150, a hole barrier layer 160, an electron transport layer 170, an electron injection layer 180, and a cathode 190.

FIG. 2 is a schematic diagram of an organic light-emitting device with an encapsulation layer according to an embodiment in the description of the present invention, in which a device 200 comprises a substrate 201, an anode 210, a hole injection layer 220, a hole transport layer 230, an electron barrier layer 240, a light-emitting layer 250, a hole barrier layer 260, an electron transport layer 270, an electron injection layer 280, a cathode 290, and an encapsulation layer 202.

FIG. 3 is a schematic diagram of a tandem organic light-emitting device with an encapsulation layer according to an embodiment of the description of the present invention, in which a device 300 comprises a substrate 301, an anode 310, a first hole injection layer 311, a first hole transport layer 312, a first electron barrier layer 313, a first light-emitting layer 314, a first hole barrier layer 315, a first electron transport layer 316, an N-type charge generation layer 320, a P-type charge generation layer 321, a second hole injection layer 330, a second hole transport layer 331, a second electron barrier layer 332, a second light-emitting layer 333, a second hole barrier layer 334, a second electron transport layer 335, a second electron injection layer 336, a cathode 337, and an encapsulation layer 302.

### DETAILED DESCRIPTION OF EMBODIMENTS

In order to explain the present invention more clearly, the present invention will be further explained below in conjunction with preferred examples and the accompanying drawings. A person skilled in the art should understand that the following detailed description is illustrative rather than restrictive, and should not limit the scope of protection of the present invention. The examples and comparative examples in the present description are provided to explain the present description more completely to those skilled in the art. The examples and comparative examples according to the present description can be transformed into various forms, and the scope of protection of the present invention should not be limited to the examples and comparative examples detailed below.

The organic compound of the present invention is suitable for light-emitting elements, display panels, and electronic devices, especially for organic electroluminescent devices. The electronic device of the present invention is a device comprising a layer of at least one organic compound, and the device may also comprise an inorganic material or a layer formed entirely of an inorganic material.

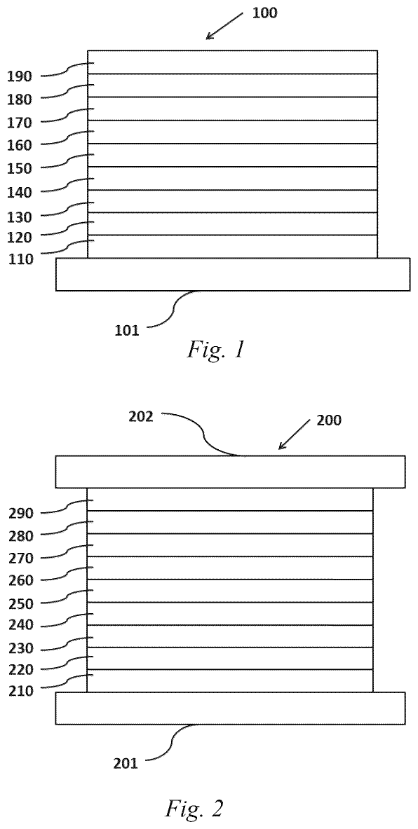

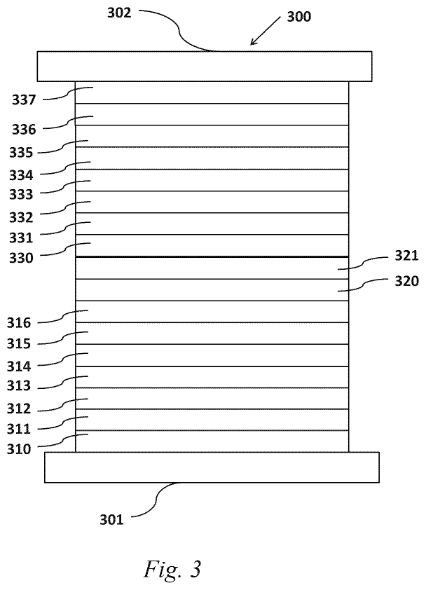

The electronic device is preferably an organic electroluminescent device (OLED), an organic integrated circuit (O-IC), an organic field effect transistor (O-FET), an organic thin film transistor (O-TFT), an organic light-emitting transistor (O-LET), an organic solar cell (O-SC), an organic dye-sensitized solar cell (O-DSSC), an organic optical detector, an organic photosensor, an organic field-quenching device (O-FQD), a luminescent electrochemical cell (LEC), an organic laser diode (O-laser), and an organic plasma emitting device. The electronic device is preferably an organic electroluminescent device (OLED), and the device structure may have the structure as shown in FIG. 1, FIG. 2, or FIG. 3.

### Definition of Substituent Terminology

Halogen or halide—As used herein, it includes fluoro, chloro, bromo, and iodo.

Alkyl—As used herein, it includes linear and branched alkyl groups. The alkyl can be an alkyl group with 1 to 20 carbon atoms, preferably an alkyl group with 1 to 12 carbon atoms, more preferably an alkyl group with 1 to 6 carbon atoms. Examples of alkyl include methyl, ethyl, propyl, isopropyl, n-butyl, sec-butyl, isobutyl, tert-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, neopentyl, 1-methylpentyl, 2-methylpentyl, 1-pentylhexyl, 1-butylpentyl, 1-heptyloctyl, and 3-methylpentyl. In the above description, methyl, ethyl, propyl, isopropyl, n-butyl, sec-butyl, isobutyl, tert-butyl, n-pentyl, neopentyl, and n-hexyl are preferred.

Cycloalkyl—As use herein, it includes a cyclic alkyl group. The cycloalkyl may be a cycloalkyl group with 3 to 20 ring carbon atoms, preferably a cycloalkyl group with 4 to 10 carbon atoms. Examples of cycloalkyl include cyclobutyl, cyclopentyl, cyclohexyl, 4-methylcyclohexyl, 4,4-dimethylcyclohexyl, 1-adamantyl, 2-adamantyl, 1-norbornyl, 2-norbornyl, etc. In the above description, cyclopentyl, cyclohexyl, 4-methylcyclohexyl, and 4,4-dimethylcyclohexyl are preferred.

Heteroalkyl—As used herein, the heteroalkyl is formed by replacing one or more carbons in the alkyl chain by heteroatom(s) selected from the group consisting of a nitrogen atom, an oxygen atom, a sulfur atom, a selenium atom, a phosphorus atom, a silicon atom, a germanium atom, and a boron atom. The heteroalkyl can be a heteroalkyl group with 1 to 20 carbon atoms, preferably a heteroalkyl group with 1 to 10 carbon atoms, more preferably a heteroalkyl group with 1 to 6 carbon atoms. Examples of heteroalkyl include methoxymethyl, ethoxymethyl, ethoxyethyl, methylthiomethyl, ethylthiomethyl, ethylthioethyl, methoxymethoxymethyl, ethoxymethoxymethyl, ethoxyethoxyethyl, hydroxymethyl, hydroxyethyl, hydroxypropyl, mercaptomethyl, mercaptoethyl, mercaptopropyl, aminomethyl, aminoethyl, aminopropyl, dimethylaminomethyl, trimethylgermylmethyl, trimethylgermylethyl, trimethylgermylisopropyl, dimethylethylgermylmethyl, dimethylisopropylgermylmethyl, tert-butyldimethylgermylmethyl, triethylgermylmethyl, triethylgermylethyl, triisopropylgermylmethyl, triisopropylgermylethyl, trimethylsilylmethyl, trimethylsilylethyl, trimethylsilylisopropyl, triisopropylsilylmethyl, and triisopropylsilylethyl.

Alkenyl—As used herein, it encompasses linear, branched, and cyclic olefin groups. The alkenyl group can be an alkenyl group containing 2 to 20 carbon atoms, preferably an alkenyl group with 2 to 10 carbon atoms. Examples of alkenyl include vinyl, propenyl, 1-butenyl, 2-butenyl, 3-butenyl, 1,3-butadienyl, 1-methylvinyl, styryl, 2,2-diphenylvinyl, 1,2-diphenylvinyl, 1-methylallyl, 1,1-dimethylallyl, 2-methylallyl, 1-phenylallyl, 2-phenylallyl, 3-phenylallyl, 3,3-diphenylallyl, 1,2-dimethylallyl, 1-phenyl-1-butenyl, 3-phenyl-1-butenyl, cyclopentenyl, cyclopentadienyl, cyclohexenyl, cycloheptenyl, cycloheptatrienyl, cyclooctenyl, cyclooctatetraenyl, and norbornenyl.

Alkynyl—As used herein, it encompasses linear alkynyl groups. The alkynyl group may be an alkynyl group containing 2 to 20 carbon atoms, preferably an alkynyl group with 2 to 10 carbon atoms. Examples of alkynyl include ethynyl, propynyl, propargyl, 1-butynyl, 2-butynyl, 3-butynyl, 1-pentynyl, 2-pentynyl, 3,3-dimethyl-1-butynyl, 3-ethyl-3-methyl-1-pentynyl, 3,3-diisopropyl1-pentynyl, phenylethynyl, phenylpropynyl, etc. In the above description, ethynyl, propynyl, propargyl, 1-butynyl, 2-butynyl, 3-butynyl, 1-pentynyl, and phenylethynyl are preferred. In addition, the alkynyl can be optionally substituted.

Aryl or aromatic group—As used herein, non-fused and fused systems are considered. The aryl can be an aryl group with 6 to 30 carbon atoms, preferably an aryl group with 6 to 20 carbon atoms, more preferably an aryl group with 6 to 12 carbon atoms. Examples of aryl include phenyl, biphenyl, terphenyl, terphenylene, tetraphenylene, naphthalene, anthracene, phenalene, phenanthrene, fluorene, pyrene, perylene, and azulene, preferably phenyl, biphenyl, terphenyl, triphenylene, fluorene, and naphthalene. Examples of non-fused aryl include phenyl, biphenyl-2-yl, biphenyl-3-yl, biphenyl-4-yl, p-terphenyl-4-yl, p-terphenyl-3-yl, p-terphenyl-2-yl, m-terphenyl-4-yl, m-terphenyl-3-yl, m-terphenyl-2-yl, o-tolyl, m-tolyl, p-tolyl, p-(2-phenylpropyl)phenyl, 4′-methylbiphenyl, 4″-tert-butyl-p-terphenyl-4-yl, o-cumyl, m-cumyl, p-cumyl, 2,3-xylyl, 3,4-xylyl, 2,5-xylyl, mesityl, and m-quaterphenyl. In addition, the aryl can be optionally substituted.

Arylene or divalent aromatic group—As used herein, it refers to a divalent group formed by further loss of a hydrogen atom from an aryl group.

Aromatic ring—As used herein, it can refer to an aromatic cyclic group and can be non-fused and fused aromatic cyclic groups.

Heterocyclic group or heterocyclic ring—As used herein, non-aromatic cyclic groups are considered. Non-aromatic heterocyclic groups include saturated heterocyclic groups with 3-20 ring atoms and unsaturated non-aromatic heterocyclic groups with 3-20 ring atoms, wherein at least one ring atom is selected from the group consisting of a nitrogen atom, an oxygen atom, a sulfur atom, a selenium atom, a silicon atom, a phosphorus atom, a germanium atom, and a boron atom, and preferred non-aromatic heterocyclic groups are those with 3 to 7 ring atoms, which comprise at least one heteroatom such as nitrogen, oxygen, silicon, or sulfur. Examples of non-aromatic heterocyclic groups include oxiranyl, oxetanyl, tetrahydrofuranyl, tetrahydropyranyl, dioxolanyl, dioxanyl, aziridinyl, dihydropyrrolyl, tetrahydropyrrolyl, piperidinyl, oxazolidinyl, morpholinyl, piperazinyl, oxepinyl, thiepinyl, azepinyl, and tetrahydrothienyl. In addition, the heterocyclic group can be optionally substituted.

Heteroaryl—As used herein, it can include non-fused and fused heteroaromatic groups containing 1 to 5 heteroatoms, at least one of which is selected from the group consisting of a nitrogen atom, an oxygen atom, a sulfur atom, a selenium atom, a silicon atom, a phosphorus atom, a germanium atom, and a boron atom. A heteroaromatic group also refers to heteroaryl. The heteroaryl can be a heteroaryl group with 3 to 30 carbon atoms, preferably a heteroaryl group with 3 to 20 carbon atoms, more preferably a heteroaryl group with 3 to 12 carbon atoms. Suitable heteroaryl groups include dibenzothiophene, dibenzofuran, dibenzoselenophene, furan, thiophene, benzofuran, benzothiophene, benzoselenophene, carbazole, indolocarbazole, pyridoindole, pyrrolopyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indenoazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, benzofuropyridine, furanodipyridine, benzothienopyridine, thienodipyridine, benzoselenophenopyridine, and selenophenodipyridine, preferably dibenzothiophene, dibenzofuran, dibenzoselenophene, carbazole, indolocarbazole, imidazole, pyridine, triazine, benzimidazole, 1,2-azaborane, 1,3-azaborane, 1,4-azaborane, borazole, and aza-analogues thereof. In addition, the heteroaryl can be optionally substituted.

Heteroarylene or divalent heteroaromatic group—As used herein, it refers to a divalent group formed by further loss of a hydrogen atom from a heteroaryl group.

Heteroaromatic ring—As used herein, it can refer to a heteroaromatic cyclic group and can be non-fused and fused heteroaromatic cyclic groups. Alkoxy—As used herein, it is represented by —O-alkyl, —O-cycloalkyl, —O-heteroalkyl, or —O— heterocyclyl. Examples and preferred examples of alkyl, cycloalkyl, heteroalkyl, and heterocyclyl are the same as above. The alkoxy can be an alkoxy group with 1 to 20 carbon atoms, preferably an alkoxy group with 1 to 6 carbon atoms. Examples of alkoxy include methoxy, ethoxy, propoxy, butoxy, pentoxy, hexyloxy, cyclopropyloxy, cyclobutoxy, cyclopentyloxy, cyclohexyloxy, tetrahydrofuranoxy, tetrahydropyranoxy, methoxypropyloxy, ethoxyethyloxy, methoxymethyloxy, and ethoxymethyloxy. In addition, the alkoxy can be optionally substituted.

Aryloxy—As used herein, it is represented by —O-aryl or —O-heteroaryl. Example and preferred example of aryl and heteroaryl are the same as described above. The aryloxy can be an aryloxy group with 6 to 30 carbon atoms, preferably an aryloxy group with 6 to 20 carbon atoms. Examples of aryloxy include phenoxy and biphenyloxy. In addition, the aryloxy can be optionally substituted.

Aralkyl—As used herein, it encompasses aryl-substituted alkyl groups. The aralkyl can be an aralkyl group with 7 to 30 carbon atoms, preferably an aralkyl group with 7 to 20 carbon atoms, more preferably an aralkyl group with 7 to 13 carbon atoms. Examples of aralkyl include benzyl, 1-phenylethyl, 2-phenylethyl, 1-phenylisopropyl, 2-phenylisopropyl, phenyltert-butyl, α-naphthylmethyl, 1-α-naphthyl-ethyl, 2-α-naphthylethyl, 1-α-naphthylisopropyl, 2-α-naphthylisopropyl, β-naphthylmethyl, 1-β-naphthyl-ethyl, 2-β-naphthyl-ethyl, 1-β-naphthylisopropyl, 2-β-naphthylisopropyl, p-methylbenzyl, m-methylbenzyl, o-methylbenzyl, p-chlorobenzyl, m-chlorobenzyl, o-chlorobenzyl, p-bromobenzyl, m-bromobenzyl, o-bromobenzyl, p-iodobenzyl, m-iodobenzyl, o-iodobenzyl, p-hydroxybenzyl, m-hydroxybenzyl, o-hydroxybenzyl, p-aminobenzyl, m-aminobenzyl, o-aminobenzyl, p-nitrobenzyl, m-nitrobenzyl, o-nitrobenzyl, p-cyanobenzyl, m-cyanobenzyl, o-cyanobenzyl, 1-hydroxy-2-phenylisopropyl, and 1-chloro-2-phenylisopropyl. In the above description, benzyl, p-cyanobenzyl, m-cyanobenzyl, o-cyanobenzyl, 1-phenylethyl, 2-phenylethyl, 1-phenylisopropyl, and 2-phenylisopropyl are preferred. In addition, the aralkyl can be optionally substituted.

Alkylsilyl—As used herein, it encompasses alkyl-substituted silyl groups. The alkylsilyl can be an alkylsilyl group with 3-20 carbon atoms, preferably an alkylsilyl group with 3 to 10 carbon atoms. Examples of alkylsilyl include trimethylsilyl, triethylsilyl, methyldiethylsilyl, ethyldimethylsilyl, tripropylsilyl, tributylsilyl, triisopropylsilyl, methyldiisopropylsilyl, dimethylisopropylsilyl, tri-tert-butylsilyl, triisobutylsilyl, dimethyltert-butylsilyl, and methyldi-tert-butylsilyl. In addition, the alkylsilyl can be optionally substituted.

Arylsilyl—As used herein, it encompasses silyl groups substituted with at least one aryl groups. The arylsilyl can be an arylsilyl group with 6 to 30 carbon atoms, preferably an arylsilyl group with 8 to 20 carbon atoms. Examples of arylsilyl include triphenylsilyl, phenylbiphenylsilyl, diphenylbiphenylsilyl, phenyldiethylsilyl, diphenylethylsilyl, phenyldimethylsilyl, diphenylmethylsilyl, phenyldiisopropylsilyl, diphenylisopropylsilyl, diphenylbutylsilyl, diphenylisobutylsilyl, and diphenyltert-butylsilyl. In addition, the arylsilyl can be optionally substituted.

Alkylgermyl—As used herein, it encompasses alkyl-substituted germyl groups. The alkylgermyl can be an alkylgermyl group with 3-20 carbon atoms, preferably an alkylgermyl group with 3 to 10 carbon atoms. Examples of alkylgermyl include trimethylgermyl, triethylgermyl, methyldiethylgermyl, ethyldimethylgermyl, tripropylgermyl, tributylgermyl, triisopropylgermyl, methyldiisopropylgermyl, dimethylisopropylgermyl, tri-tert-butylgermyl, triisobutylgermyl, dimethyltert-butylgermyl, and methyldi-tert-butylgermyl. In addition, the alkylgermyl can be optionally substituted.

Arylgermyl group—As used herein, it encompasses germyl groups substituted with at least one aryl or heteroaryl group. The arylgermyl can be an arylgermyl group with 6-30 carbon atoms, preferably an arylgermyl group with 8 to 20 carbon atoms. Examples of arylgermyl include triphenylgermyl, phenylbiphenylgermyl, diphenylbiphenylgermyl, phenyldiethylgermyl, diphenylethylgermyl, phenyldimethylgermyl, diphenylmethylgermyl, phenyldiisopropylgermyl, diphenylisopropylgermyl, diphenylbutylgermyl, diphenylisobutylgermyl, and diphenyltert-butylgermyl. In addition, the arylgermyl can be optionally substituted.

The term “aza” in azadibenzofuran, azadibenzothiophene, etc. means that one or more C—H groups in the corresponding aromatic fragments are replaced by nitrogen atoms. For example, azatriphenylene includes dibenzo[f,h]quinoxaline, dibenzo[f,h]quinoline, and other analogues having two or more nitrogen atoms in the ring system. Other nitrogen-based analogues of the above aza-derivatives are readily conceivable to those of ordinary skill in the art, and all these analogues are determined to be included in the terms described herein.

In the present disclosure, unless otherwise defined, when using any term selected from the group consisting of a substituted alkyl group, a substituted cycloalkyl group, a substituted heteroalkyl group, a substituted heterocyclyl group, a substituted aralkyl group, a substituted alkoxy group, a substituted aryloxy group, a substituted alkenyl group, a substituted alkynyl group, a substituted aryl group, a substituted heteroaryl group, a substituted alkylsilyl group, a substituted arylsilyl group, a substituted alkylgermyl group, a substituted arylgermyl group, a substituted amino group, a substituted acyl group, a substituted carbonyl group, a substituted carboxyl group, a substituted ester group, a substituted sulfinyl group, a substituted sulfonyl group, and a substituted phosphine oxide group, it means that any one of the groups alkyl, cycloalkyl, heteroalkyl, heterocyclyl, aralkyl, alkoxy, aryloxy, alkenyl, alkynyl, aryl, heteroaryl, alkylsilyl, arylsilyl, alkylgermyl, arylgermyl, amino, acyl, carbonyl, carboxyl, an ester group, sulfinyl, sulfonyl, and phosphoroso can be substituted with one or more groups selected from deuterium, halogen, an unsubstituted alkyl group with 1-20 carbon atoms, an unsubstituted cycloalkyl group with 3-20 ring carbon atoms, an unsubstituted heteroalkyl group with 1-20 carbon atoms, an unsubstituted heterocyclyl group with 3-20 ring atoms, an unsubstituted aralkyl group with 7-30 carbon atoms, an unsubstituted alkoxy group with 1-20 carbon atoms, an unsubstituted aryloxy group with 6-30 carbon atoms, an unsubstituted alkenyl group with 2-20 carbon atoms, an unsubstituted alkynyl group with 2-20 carbon atoms, an unsubstituted aryl group with 6-30 carbon atoms, an unsubstituted heteroaryl group with 3-30 carbon atoms, an unsubstituted alkylsilyl group with 3-20 carbon atoms, an unsubstituted arylsilyl group with 6-20 carbon atoms, an unsubstituted alkylgermyl group with 3-20 carbon atoms, an unsubstituted arylgermyl group with 6-20 carbon atoms, an unsubstituted amino group with 0-20 carbon atoms, hydroxy, mercapto, O, S, acyl, carbonyl, carboxyl, an ester group, cyano, isocyano, SCN, OCN, SF₅, nitro, nitroso, sulfinyl, sulfonyl, phosphoroso, and a combination thereof.

It should be understood that when a molecular fragment is described as a substituent or as being otherwise connected to another moiety, its name can be written according to whether it is a fragment (such as phenyl, phenylene, naphthyl, or dibenzofuranyl) or whether it is a whole molecule (such as benzene, naphthalene, or dibenzofuran). As used herein, these different ways of specifying substituents or linking fragments are considered equivalent.

In the compounds mentioned in the present disclosure, hydrogen atoms can be partially or completely replaced by deuterium. Other atoms such as carbon, nitrogen, oxygen, phosphorus, and boron can also be replaced by other stable isotopes thereof. Since it enhances the efficiency and stability of a device, replacement with other stable isotopes in the compounds can be preferred.

In the compounds mentioned in the present disclosure, multi-substitution refers to a range encompassing disubstitution up to the highest degree of substitution available. When a substituent in a compound mentioned in the present disclosure means polysubstitution (including disubstitution, trisubstitution, tetrasubstitution, etc.), it means that the substituent can exist in multiple available substitution positions on the structure to which it connects, and the substituents existing in the multiple available substitution positions can be either the same structure or different structures. In the compounds mentioned in the present disclosure, adjacent substituents in the compound cannot be connected to form a ring, unless explicitly defined, for example, adjacent substituents can be optionally connected to form a ring. In the compounds mentioned in the present disclosure, adjacent substituents can be optionally connected to form a ring, including both the case where adjacent substituents can be connected to form a ring and the case where adjacent substituents are not connected to form a ring. When adjacent substituents can be optionally connected to form a ring, the formed ring can be monocyclic or polycyclic (including spiro ring, bridged ring, fused ring, etc.), and alicyclic, heteroaliphatic, aromatic, or heteroaromatic rings. In this expression, adjacent substituents can refer to substituents bonded to the same atom, substituents bonded to carbon atoms that are directly bonded to each other, or substituents bonded to carbon atoms that are further away from each other. Preferably, adjacent substituents refer to substituents bonded to the same carbon atom and substituents bonded to carbon atoms that are directly bonded to each other.

The expression that adjacent substituents can be optionally connected to form a ring or bonded to form a ring is also intended to mean that the two substituents bonded to the same carbon atom are connected to each other via a chemical bond to form a ring, which can be exemplified by the following formula:

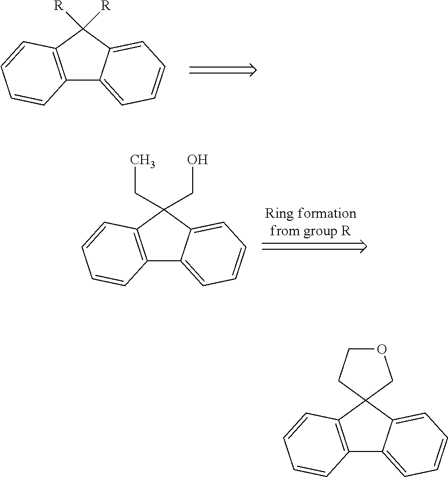

The expression that adjacent substituents can be optionally connected to form a ring or bonded to form a ring is also intended to mean that two substituents bonded to carbon atoms that are directly bonded to each other are connected to each other via a chemical bond to form a ring, which can be exemplified by the following formula:

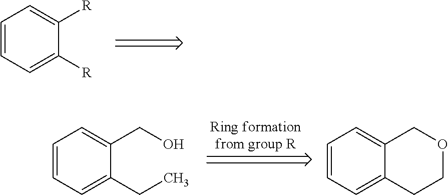

The expression that adjacent substituents can be optionally connected to form a ring or bonded to form a ring is also intended to mean that two substituents bonded to carbon atoms that are further away from each other are connected to each other via a chemical bond to form a ring, which can be exemplified by the following formula:

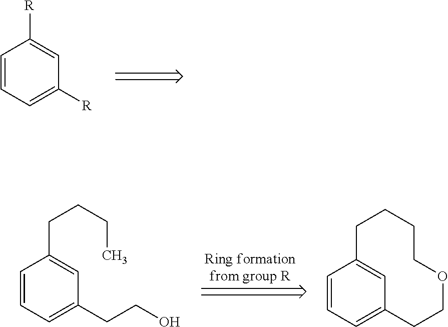

Furthermore, the expression that adjacent substituents can be optionally connected to form a ring or bonded to form a ring is also intended to mean that where one of two adjacent substituents represents hydrogen, the other substituent is bonded at the position to which the hydrogen atom is bonded, thereby forming a ring. This is exemplified by the following formula:

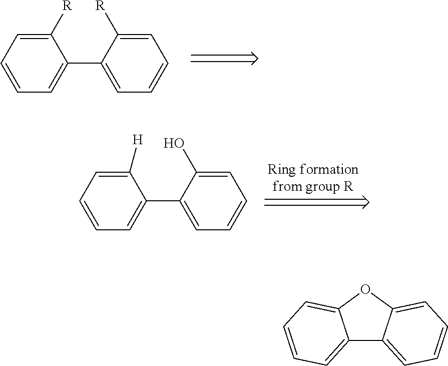

The first compound and the second compound used in the present invention can be obtained by reference to the preparation method in the prior art, and no more detailed description will be given here.

The preparation method for the organic electroluminescent device is not limited, and the preparation method of the following examples is only an example and should not be understood as a limitation. Those skilled in the art can rationally modify the preparation method of the following examples according to the prior art. By way of example, the ratio of various materials in each organic layer is not particularly limited, and those skilled in the art can make a rational selection in a certain range according to the prior art. In the device examples, the characteristics of the devices are also tested by using conventional equipment in the art and methods well known to those skilled in the art. Since those skilled in the art are all aware of related contents such as the use of the above equipment and test methods and can definitely obtain the inherent data of the sample without interference, the above related contents are no more repeated in the present patent.

### Synthesis Examples

The preparation method for the compound of the present invention is not limited, and the raw materials used can either be purchased or synthesized in-house.

The structural unit fragment

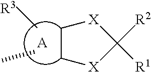

in formula II can be synthesized by the following route of the method:

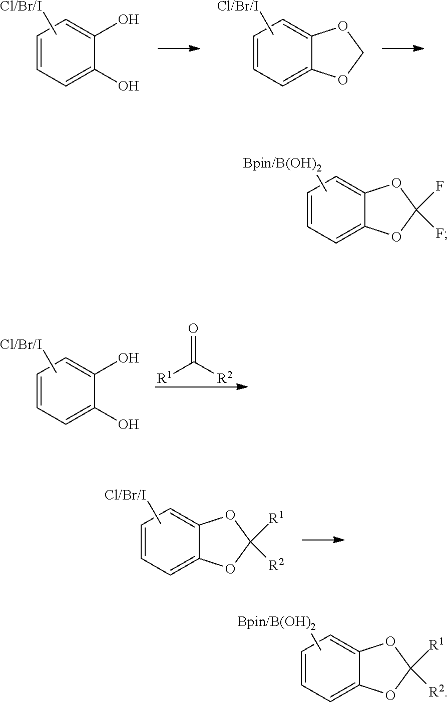

The obtained corresponding fragment can also be coupled with a halide, and finally, the obtained raw material compound is used to synthesize the compound of the present invention, as shown below:

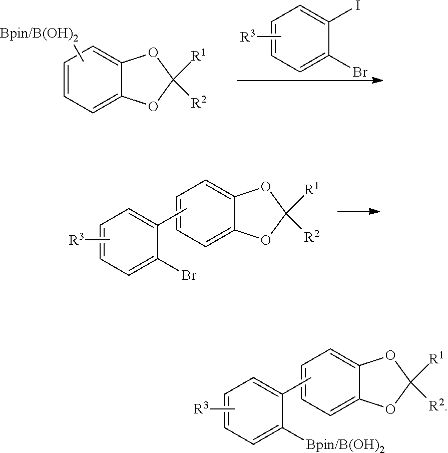

The above preparation method is not limiting and is provided for reference only. The atoms in the above structures can be replaced by other atoms or groups. The raw materials can be purchased. For the specific implementation method, reference can be made to publicly known methods in the art.

The preparation method for the compound of the present invention is not limited. Typically, but not by way of limitation, the following compound is provided as an example, and the synthesis route and preparation method thereof are as follows.

### Synthesis Example 1: Synthesis of Compound P1-493

### Step 1: Synthesis of Intermediate P1-493-A

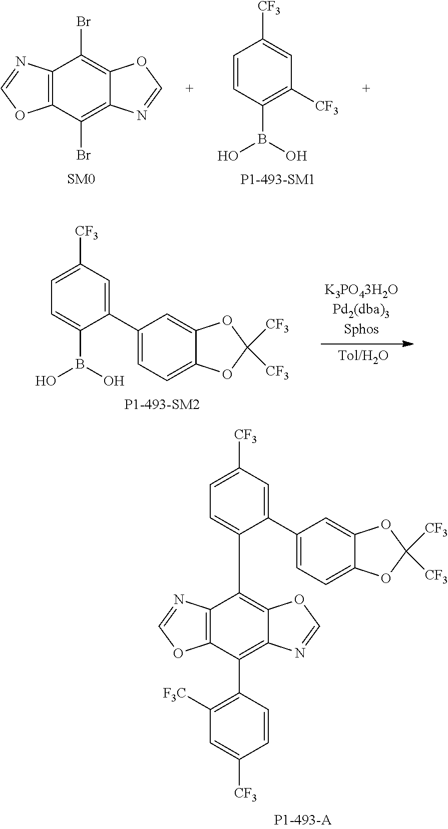 [SMILES: Brc1c2ncoc2c(Br)c2ncoc12 ; OB(O)c1ccc(C(F)(F)F)cc1C(F)(F)F ; OB(O)c1ccc(C(F)(F)F)cc1-c1ccc2c(c1)OC(C(F)(F)F)(C(F)(F)F)O2 ; FC(F)(F)c1ccc(-c2c3ncoc3c(-c3ccc(C(F)(F)F)cc3C(F)(F)F)c3ncoc23)c(-c2ccc3c(c2)OC(C(F)(F)F)(C(F)(F)F)O3)c1]

In a 2 L two-necked flask, intermediate SM0 (30.5 g, 95.9 mmol), intermediate P1-493-SM1 (30.2 g, 116.9 mmol), intermediate P1-493-SM2 (52.6 g, 117.9 mmol), potassium phosphate trihydrate (64.0 g, 240.5 mmol), Pd₂(dba)₃ (1.15 g, 1.252 mmol), and SPhos (2-dicyclohexylphosphino-2′,6′-dimethoxybiphenyl (2.16 g, 5.263 mmol) were successively added. After nitrogen displacement three times, 1000 mL of toluene and 250 mL of water were added under nitrogen protection, and the mixture was heated to 100 °C and reacted overnight. After the reaction was complete, the reaction liquid was cooled and extracted by adding an appropriate amount of tetrahydrofuran, and the extract liquor was dried over anhydrous magnesium sulfate, concentrated and then purified by column chromatography to obtain intermediate P1-493-A as a white solid (8.14 g, yield 11%).

### Step 2: Synthesis of Intermediate P1-493-B

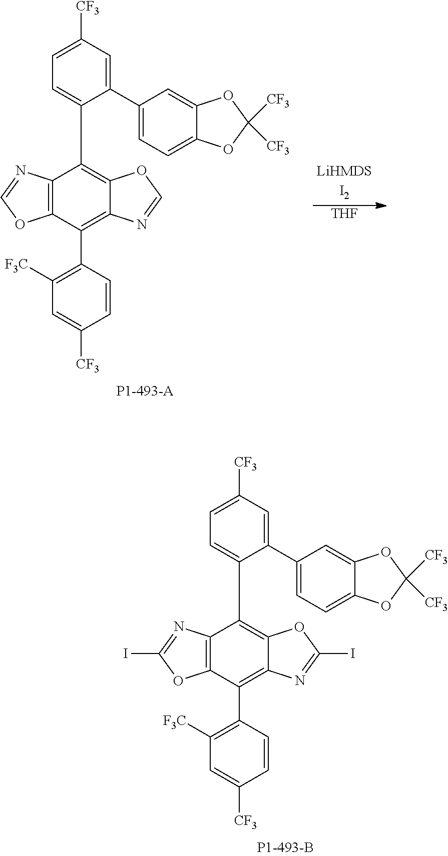 [SMILES: FC(F)(F)c1ccc(-c2c3ncoc3c(-c3ccc(C(F)(F)F)cc3C(F)(F)F)c3ncoc23)c(-c2ccc3c(c2)OC(C(F)(F)F)(C(F)(F)F)O3)c1 ; "THF" ; FC(F)(F)c1ccc(-c2c3nc(I)oc3c(-c3ccc(C(F)(F)F)cc3C(F)(F)F)c3nc(I)oc23)c(-c2ccc3c(c2)OC(C(F)(F)F)(C(F)(F)F)O3)c1]

In a 2 L two-necked flask, intermediate P1-493-A (8.14 g, 10.54 mmol) was added, 800 mL of ultradry THF was added under nitrogen protection, and the mixture was cooled to −30 °C. Lithium bis(trimethylsilyl)amide (LiHMDS, 30 mL, 30 mmol) was then dropwise added. The mixture was reacted at this temperature for 2 h. Elemental iodine (7.70 g, 30.3 mmol) was then added, and the mixture was heated to room temperature and reacted for 0.5 h. The reaction liquid was quenched with a saturated sodium sulfite solution, extracted by adding an appropriate amount of ethyl acetate, dried over anhydrous magnesium sulfate, concentrated and then purified by column chromatography to obtain intermediate P1-493-B as a white solid (10.3 g, yield 95%).

### Step 3: Synthesis of Intermediate P1-493-C

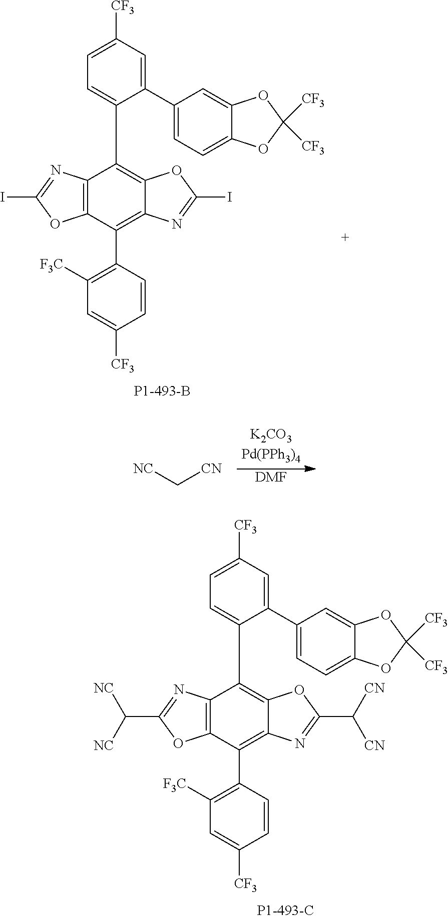

In a 2 L two-necked flask, intermediate P1-493-B (10.3 g, 10.0 mmol), malononitrile (3.21 g, 48.6 mmol), potassium carbonate (10.8 g, 78.1 mmol), and Pd(PPh₃)₄ (0.462 g, 0.400 mmol) were successively added, 350 mL of DMF was added under nitrogen protection, and the mixture was heated to 80 °C and reacted overnight. After the reaction was complete, the reaction liquid was cooled to room temperature and cooled in an ice-water bath. The reaction liquid was slowly dropwise added to 3N hydrochloric acid to precipitate a large amount of a yellow solid, which was filtered to obtain a crude product. After crystallization, intermediate P1-493-C as a white solid (7.20 g, yield 80%) was obtained.

### Step 4: Synthesis of Compound P1-493

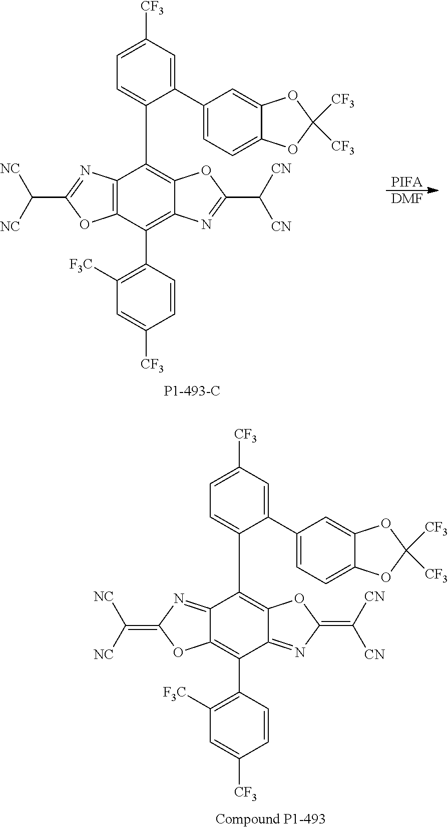

In a nitrogen atmosphere, to a 3 L single-necked flask, intermediate P1-493-C (7.20 g, 8.00 mmol) and then 1000 mL of DCM were added, and bis(trifluoroacetoxy)iodobenzene (PIFA, 6.35 g, 14.76 mmol) was added in portions at room temperature. After stirring at room temperature for 2 days, the reaction liquid was concentrated to an appropriate volume and filtered to obtain compound P1-493 as a black solid (5.60 g, yield 78%). The product was confirmed to be the target compound with a molecular weight of 898.0.

### Synthesis Example 2: Synthesis of Compound P2-257

### Step 1: Synthesis of Intermediate P2-257-A

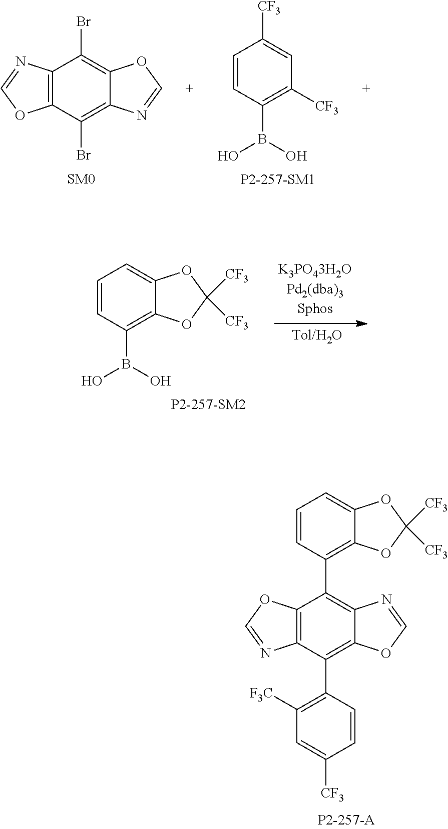 [SMILES: Brc1c2ncoc2c(Br)c2ncoc12 ; OB(O)c1ccc(C(F)(F)F)cc1C(F)(F)F ; OB(O)c1cccc2c1OC(C(F)(F)F)(C(F)(F)F)O2 ; FC(F)(F)c1ccc(-c2c3ncoc3c(-c3cccc4c3OC(C(F)(F)F)(C(F)(F)F)O4)c3ncoc23)c(C(F)(F)F)c1]

In a 2 L two-necked flask, intermediate SM0 (30.8 g, 96.9 mmol), intermediate P2-257-SM1 (29.7 g, 115.3 mmol), intermediate P2-257-SM2 (35.8 g, 118.6 mmol), potassium phosphate trihydrate (62.0 g, 232.8 mmol), Pd₂(dba)₃ (1.15 g, 1.252 mmol), and SPhos (2-dicyclohexylphosphino-2′,6′-dimethoxybiphenyl (2.16 g, 5.263 mmol) were successively added. After nitrogen displacement three times, 1000 mL of toluene and 250 mL of water were added under nitrogen protection, and the mixture was heated to 100 °C and reacted overnight. After the reaction was complete, the reaction liquid was cooled and extracted by adding an appropriate amount of tetrahydrofuran, and the extract liquor was dried over anhydrous magnesium sulfate, concentrated and then purified by column chromatography to obtain intermediate P2-257-A as a white solid (7.67 g, yield 15%).

### Step 2: Synthesis of Intermediate P2-257-B

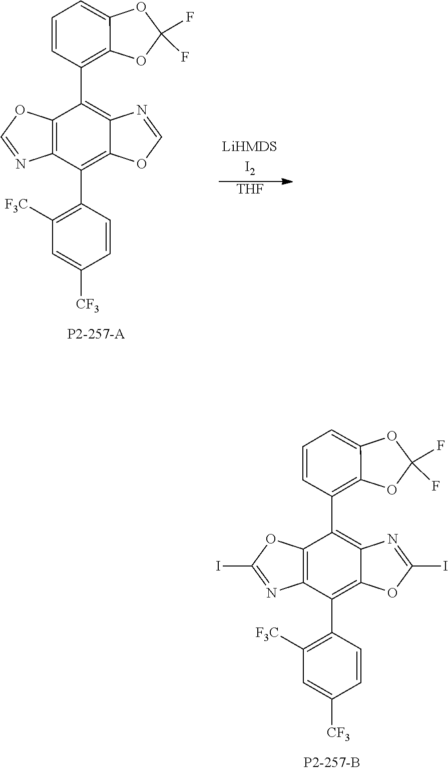

In a 2 L two-necked flask, intermediate P2-257-A (7.67 g, 14.54 mmol) was added, 800 mL of ultradry THF was added under nitrogen protection, and the mixture was cooled to −30 °C. Lithium bis(trimethylsilyl)amide (LiHMIDS, 45 mL, 45 mmol) was then dropwise added. The mixture was reacted at this temperature for 2 h. Elemental iodine (12.8 g, 50.3 mmol) was then added, and the mixture was heated to room temperature and reacted for 0.5 h. The reaction liquid was quenched with a saturated sodium sulfite solution and extracted by adding an appropriate amount of ethyl acetate, and the extract liquor was dried over anhydrous magnesium sulfate, concentrated and then purified by column chromatography to obtain intermediate P2-257-B as a white solid (10.5 g, yield 93%).

### Step 3: Synthesis of Intermediate P2-257-C

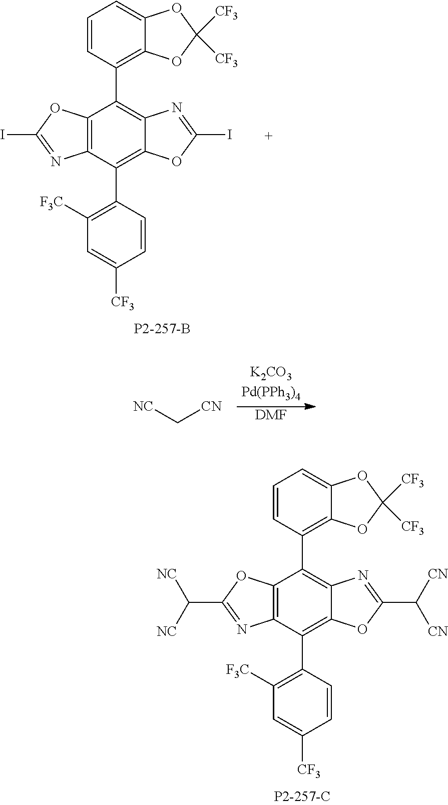

In a 2 L two-necked flask, intermediate P2-257-B (10.5 g, 13.5 mmol), malononitrile (3.60 g, 54.6 mmol), potassium carbonate (9.43 g, 68.2 mmol), and Pd(PPh₃)₄ (0.443 g, 0.382 mmol) were successively added, 350 mL of DMF was added under nitrogen protection, and the mixture was heated to 80 °C and reacted overnight. After the reaction was complete, the reaction liquid was cooled to room temperature and cooled in an ice-water bath. The reaction liquid was slowly dropwise added to 3N hydrochloric acid to precipitate a large amount of a yellow solid, which was filtered to obtain a crude product. After crystallization, intermediate P2-257-C as a white solid (7.44 g, yield 84%) was obtained.

### Step 4: Synthesis of Compound P2-257

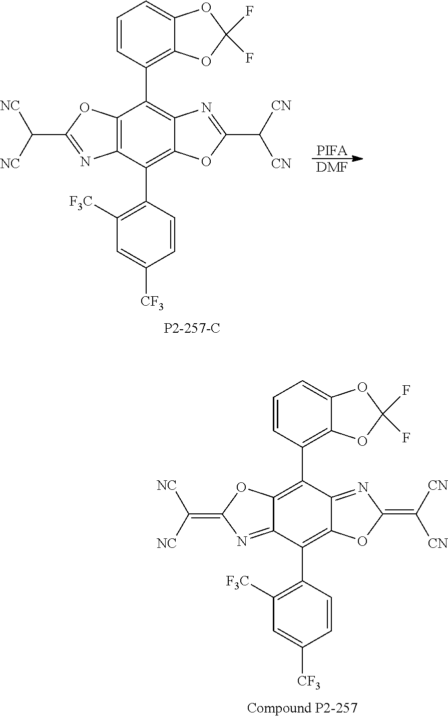

In a nitrogen atmosphere, to a 3 L single-necked flask, intermediate P2-257-C (7.44 g, 11.34 mmol) and then 1000 mL of DCM were added, and bis(trifluoroacetoxy)iodobenzene (PIFA, 7.40 g, 17.20 mmol) was added in portions at room temperature. After stirring at room temperature for 2 days, the reaction liquid was concentrated to an appropriate volume and filtered to obtain compound P2-257 as a black solid (6.38 g, yield 86%). The product was confirmed to be the target compound with a molecular weight of 654.0.

### Synthesis Example 3: Synthesis of Compound P2-271

### Step 1: Synthesis of Intermediate P2-271-A

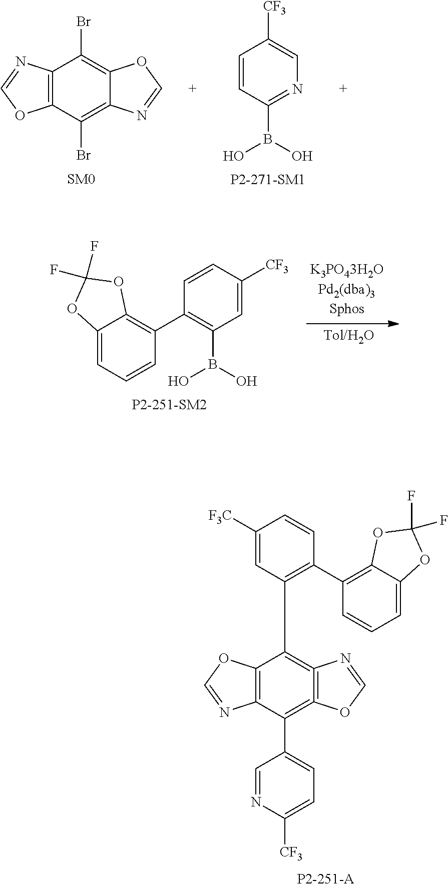

In a 2 L two-necked flask, intermediate SM0 (31.5 g, 98.9 mmol), intermediate P2-271-SM1 (22.6 g, 118.5 mmol), intermediate P2-271-SM2 (41.7 g, 120.4 mmol), potassium phosphate trihydrate (63.1 g, 236.8 mmol), Pd₂(dba)₃ (1.15 g, 1.252 mmol), were successively added. After nitrogen displacement three times, 1000 mL of toluene and 250 mL of water were added under nitrogen protection, and the mixture was heated to 100 °C and reacted overnight. After the reaction was complete, the reaction liquid was cooled and extracted by adding an appropriate amount of tetrahydrofuran, and the extract liquor was dried over anhydrous magnesium sulfate, concentrated and then purified by column chromatography to obtain intermediate P2-271-A as a white solid (8.38 g, yield 14%).

### Step 2: Synthesis of Intermediate P2-271-B

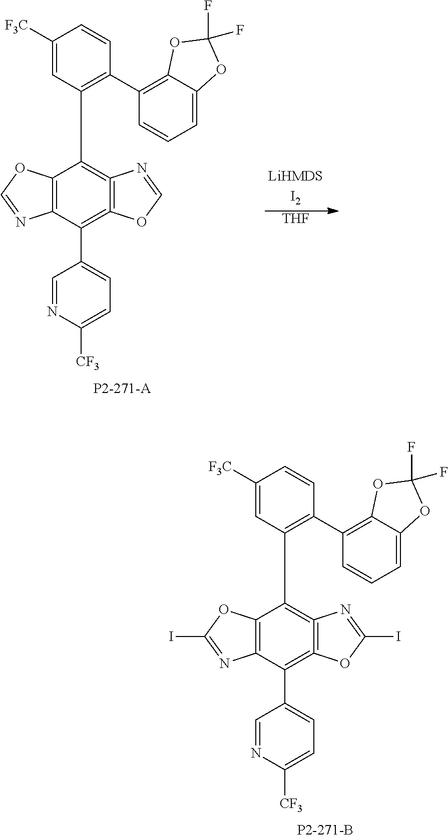

In a 2 L two-necked flask, intermediate P2-271-A(8.38 g, 13.8 mmol) was added, 800 mL of ultradry THF was added under nitrogen protection, and the mixture was cooled to −30 °C. Lithium bis(trimethylsilyl)amide (LiHMiDS, 45 mL, 45 mmol) was then dropwise added. The mixture was reacted at this temperature for 2 h. Elemental iodine (12.3 g, 48.3 mmol) was then added, and the mixture was heated to room temperature and reacted for 0.5 h. The reaction liquid was quenched with a saturated sodium sulfite solution and extracted by adding an appropriate amount of ethyl acetate, and the extract liquor was dried over anhydrous magnesium sulfate, concentrated and then purified by column chromatography to obtain intermediate P2-271-B as a white solid (10.8 g, yield 91%).

### Step 3: Synthesis of Intermediate P2-271-C

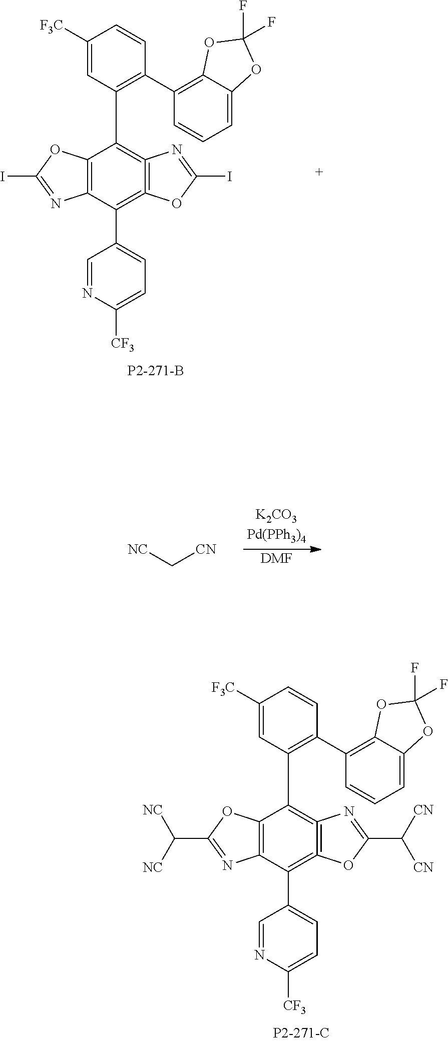

In a 2 L two-necked flask, intermediate P2-271-B (10.8 g, 12.6 mmol), malononitrile (3.34 g, 50.6 mmol), potassium carbonate (8.07 g, 58.4 mmol), and Pd(PPh₃)₄ (0.210 g, 0.182 mmol) were successively added, 350 mL of DMF was added under nitrogen protection, and the mixture was heated to 80 °C and reacted overnight. After the reaction was complete, the reaction liquid was cooled to room temperature and cooled in an ice-water bath. The reaction liquid was slowly dropwise added to 3N hydrochloric acid to precipitate a large amount of a yellow solid, which was filtered to obtain a crude product. After crystallization, intermediate P2-271-C as a white solid (7.67 g, yield 83%) was obtained.

### Step 4: Synthesis of Compound P2-271

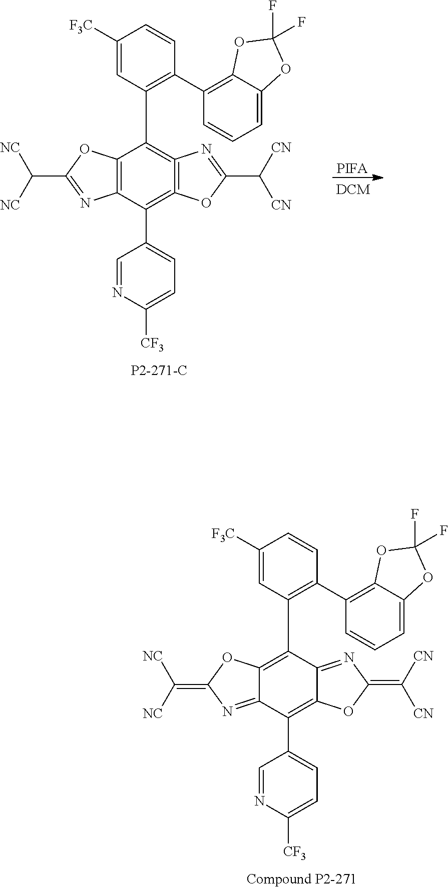

In a nitrogen atmosphere, to a 3 L single-necked flask, intermediate P2-271-C (7.67 g, 10.46 mmol) and then 1000 mL of DCM were added, and bis(trifluoroacetoxy)iodobenzene (PIFA, 7.40 g, 17.20 mmol) was added in portions at room temperature. After stirring at room temperature for 2 days, the reaction liquid was concentrated to an appropriate volume and filtered to obtain compound P2-271 as a black solid (6.81 g, yield 89%). The product was confirmed to be the target compound with a molecular weight of 731.1.

### Synthesis Example 4: Synthesis of Compound P3-115

### Step 1: Synthesis of Intermediate P3-115-A

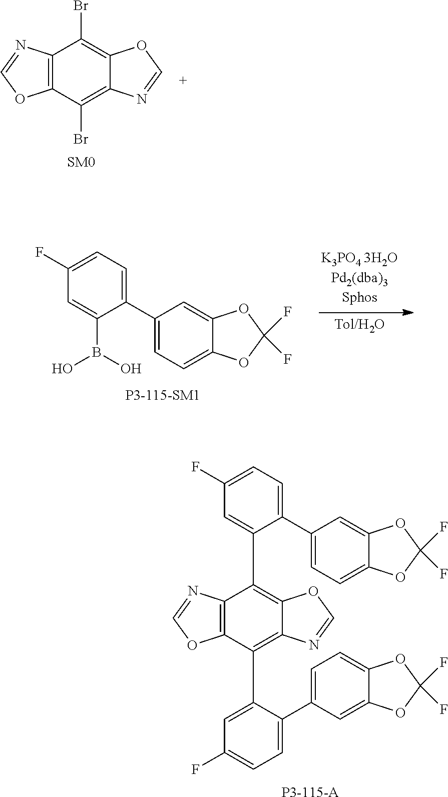

In a 2 L two-necked flask, intermediate SM0 (10.6 g, 33.3 mmol), intermediate P3-115-SM1 (34.5 g, 116.5 mmol), potassium phosphate trihydrate (36.2 g, 136.2 mmol), Pd₂(dba)₃ (1.46 g, 1.59 mmol), and SPhos (2-dicyclohexylphosphino-2′,6′-dimethoxybiphenyl (2.26 g, 5.50 mmol) were successively added. After nitrogen displacement three times, 800 mL of toluene and 200 mL of water were added under nitrogen protection, and the mixture was heated to 100 °C and reacted overnight. After the reaction was complete, the reaction liquid was cooled and extracted by adding an appropriate amount of tetrahydrofuran, and the extract liquor was dried over anhydrous magnesium sulfate, concentrated and then purified by column chromatography to obtain intermediate P3-115-A as a white solid (18.0 g, yield 82%).

### Step 2: Synthesis of Intermediate P3-115-B

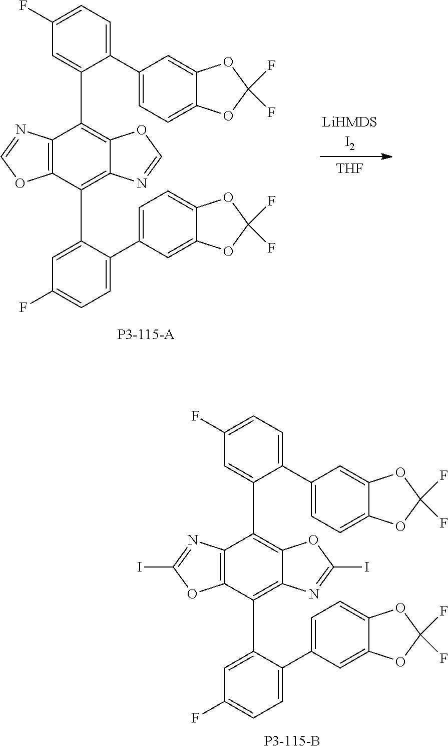

In a 2 L two-necked flask, intermediate P3-115-A (18.0 g, 27.3 mmol) was added, 800 mL of ultradry THF was added under nitrogen protection, and the mixture was cooled to −30 °C. Lithium bis(trimethylsilyl)amide (LiHMIDS, 65 mL, 65 mmol) was then dropwise added. The mixture was reacted at this temperature for 2 h. Elemental iodine (17.8 g, 70.2 mmol) was then added, and the mixture was heated to room temperature and reacted for 0.5 h. The reaction liquid was quenched with a saturated sodium sulfite solution and extracted by adding an appropriate amount of ethyl acetate, and the extract liquor was dried over anhydrous magnesium sulfate, concentrated and then purified by column chromatography to obtain intermediate P3-115-B as a white solid (22.4 g, yield 90%).

### Step 3: Synthesis of Intermediate P3-115-C

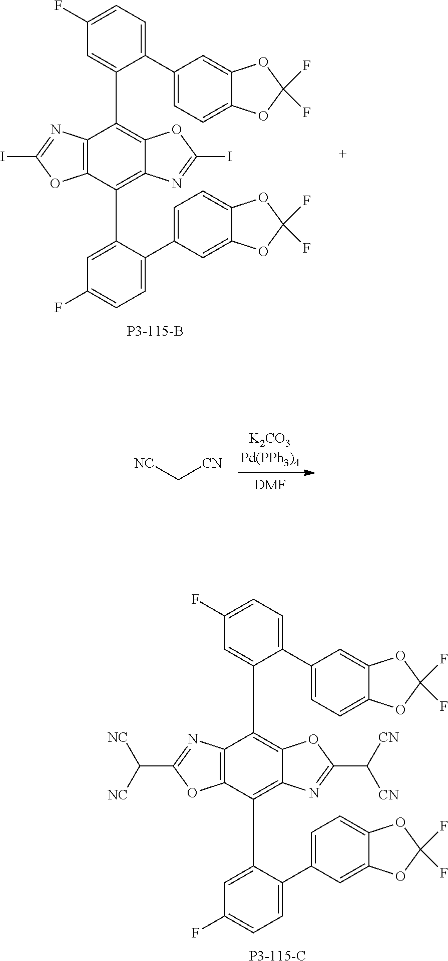

In a 2 L two-necked flask, intermediate P3-115-B (22.4 g, 24.6 mmol), malononitrile (6.31 g, 95.6 mmol), potassium carbonate (13.6 g, 98.4 mmol), and Pd(PPh₃)₄ (0.340 g, 0.294 mmol) were successively added, 350 mL of DMF was added under nitrogen protection, and the mixture was heated to 80 °C and reacted overnight. After the reaction was complete, the reaction liquid was cooled to room temperature and cooled in an ice-water bath. The reaction liquid was slowly dropwise added to 3N hydrochloric acid to precipitate a large amount of a yellow solid, which was filtered to obtain a crude product. After crystallization, intermediate P3-115-C as a white solid (15.7 g, yield 81%) was obtained.

### Step 4: Synthesis of Compound P3-115

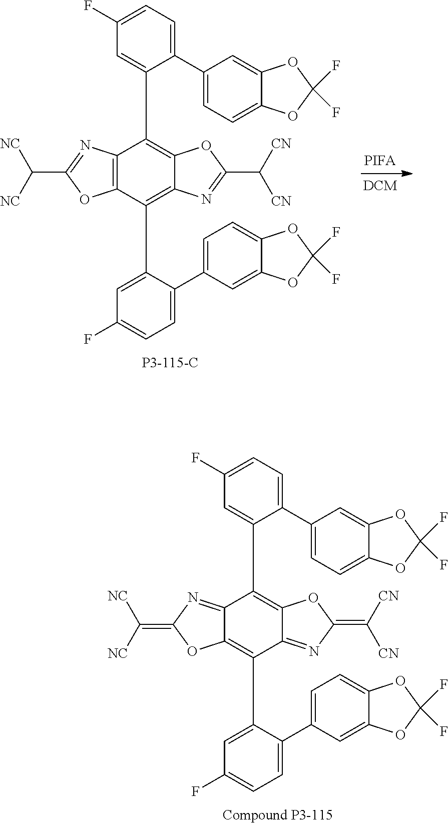

In a nitrogen atmosphere, to a 3 L single-necked flask, intermediate P3-115-C (15.7 g, 19.9 mmol) and then 1000 mL of DCM were added, and bis(trifluoroacetoxy)iodobenzene (PIFA, 16.7 g, 38.8 mmol) was added in portions at room temperature. After stirring at room temperature for 2 days, the reaction liquid was concentrated to an appropriate volume and filtered to obtain compound P3-115 as a black solid (13.0 g, yield 83%). The product was confirmed to be the target compound with a molecular weight of 786.0.

### Synthesis Example 5: Synthesis of Compound P3-124

### Step 1: Synthesis of Intermediate P3-124-A

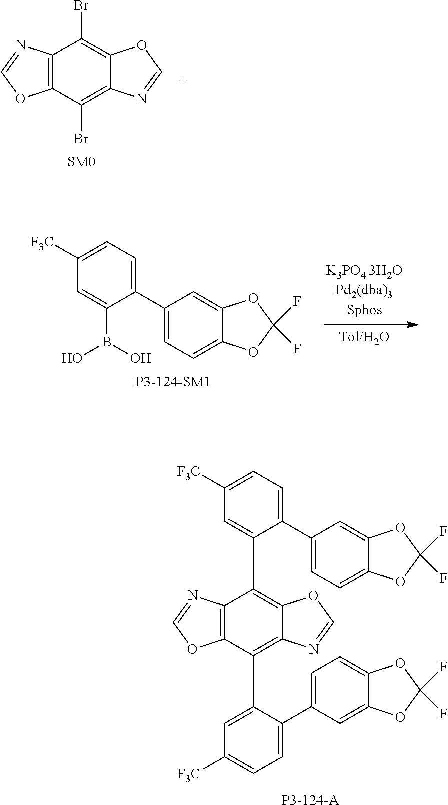

In a 2 L two-necked flask, intermediate SM0 (11.5 g, 36.2 mmol), intermediate P3-124-SM1 (41.7 g, 120.5 mmol), potassium phosphate trihydrate (36.2 g, 136.2 mmol), Pd₂(dba)₃ (1.68 g, 1.832 mmol), and SPhos (2-dicyclohexylphosphino-2′,6′-dimethoxybiphenyl (2.25 g, 5.48 mmol) were successively added. After nitrogen displacement three times, 800 mL of toluene and 200 mL of water were added under nitrogen protection, and the mixture was heated to 100 °C and reacted overnight. After the reaction was complete, the reaction liquid was cooled and extracted by adding an appropriate amount of tetrahydrofuran, and the extract liquor was dried over anhydrous magnesium sulfate, concentrated and then purified by column chromatography to obtain intermediate P3-124-A as a white solid (19.5 g, yield 71%).

### Step 2: Synthesis of Intermediate P3-124-B

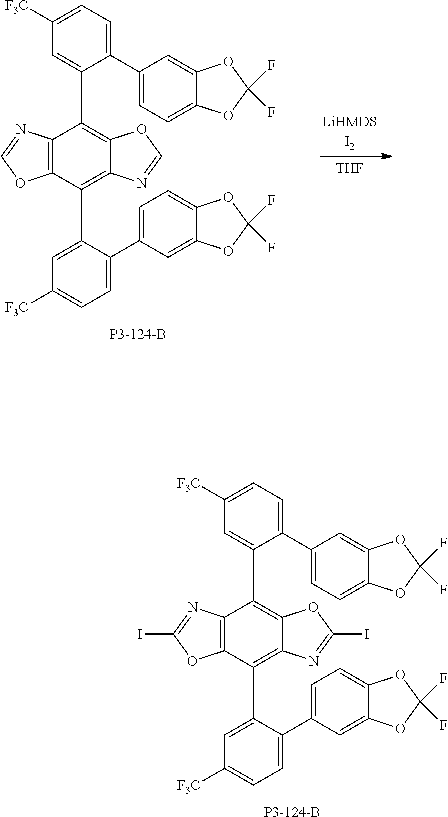

In a 2 L two-necked flask, intermediate P3-124-A(19.5 g, 25.7 mmol) was added, 800 mL of ultradry THF was added under nitrogen protection, and the mixture was cooled to −30 °C. Lithium bis(trimethylsilyl)amide (LiHMDS, 60 mL, 60 mmol) was then dropwise added. The mixture was reacted at this temperature for 2 h. Elemental iodine (18.4 g, 72.3 mmol) was then added, and the mixture was heated to room temperature and reacted for 0.5 h. The reaction liquid was quenched with a saturated sodium sulfite solution and extracted by adding an appropriate amount of ethyl acetate, and the extract liquor was dried over anhydrous magnesium sulfate, concentrated and then purified by column chromatography to obtain intermediate P3-124-B as a white solid (22.4 g, yield 86%).

### Step 3: Synthesis of Intermediate P3-124-C

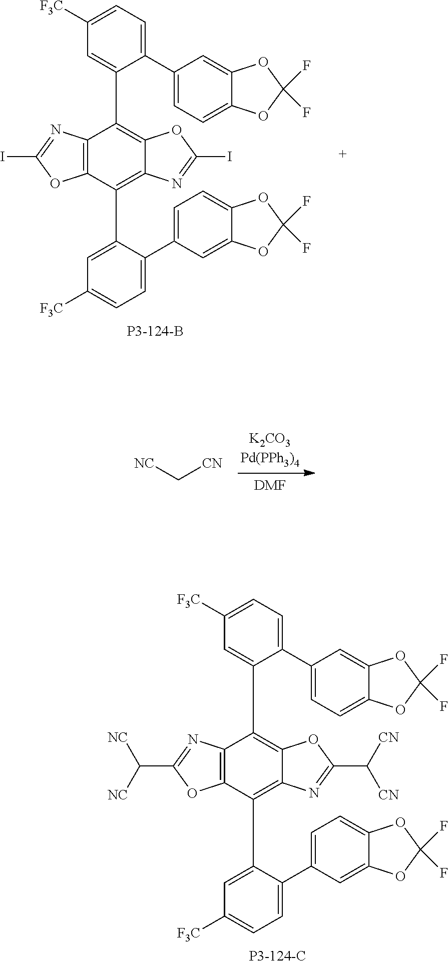

In a 2 L two-necked flask, intermediate P3-124-B (22.4 g, 22.1 mmol), malononitrile (5.85 g, 88.6 mmol), potassium carbonate (12.8 g, 92.4 mmol), and Pd(PPh₃)₄ (0.326 g, 0.282 mmol) were successively added, 350 mL of DMF was added under nitrogen protection, and the mixture was heated to 80 °C and reacted overnight. After the reaction was complete, the reaction liquid was cooled to room temperature and cooled in an ice-water bath. The reaction liquid was slowly dropwise added to 3N hydrochloric acid to precipitate a large amount of a yellow solid, which was filtered to obtain a crude product. After crystallization, intermediate P3-124-C as a white solid (16.1 g, yield 82%) was obtained.

### Step 4: Synthesis of Compound P3-124

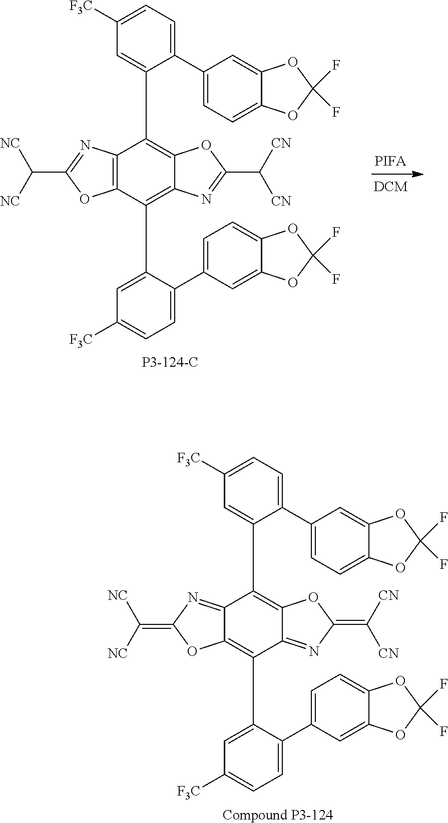

In a nitrogen atmosphere, to a 3 L single-necked flask, intermediate P3-124-C (16.1 g, 18.1 mmol) and then 1000 mL of DCM were added, and bis(trifluoroacetoxy)iodobenzene (PIFA, 15.8 g, 36.8 mmol) is added in portions at room temperature. After stirring at room temperature for 2 days, the reaction liquid was concentrated to an appropriate volume and filtered to obtain compound P3-124 as a black solid (12.0 g, yield 75%). The product was confirmed to be the target compound with a molecular weight of 886.0.

### Synthesis Example 6: Synthesis of Compound P3-128

### Step 1: Synthesis of Intermediate P3-128-A

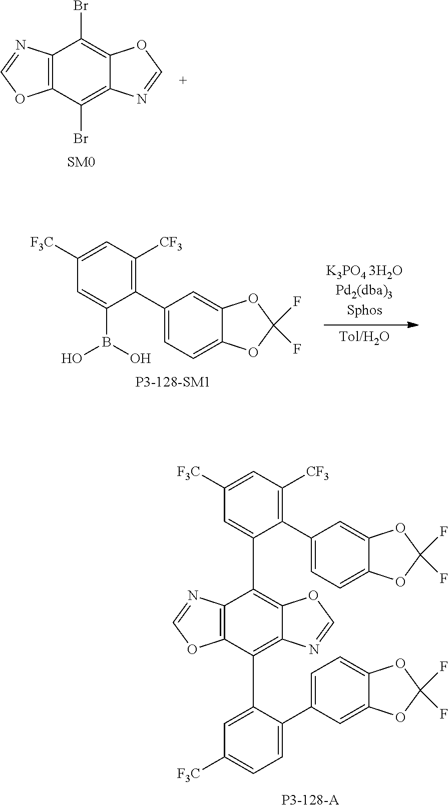

In a 2 L two-necked flask, intermediate SM0 (10.2 g, 32.2 mmol), intermediate P3-128-SM1 (38.0 g, 91.8 mmol), potassium phosphate trihydrate (28.0 g, 105.2 mmol), Pd₂(dba)₃ (1.52 g, 1.654 mmol), and SPhos (2-dicyclohexylphosphino-2′,6′-dimethoxybiphenyl (2.13 g, 5.19 mmol) were successively added. After nitrogen displacement three times, 800 mL of toluene and 200 mL of water were added under nitrogen protection, and the mixture was heated to 100 °C and reacted overnight. After the reaction was complete, the reaction liquid was cooled and extracted by adding an appropriate amount of tetrahydrofuran, and the extract liquor was dried over anhydrous magnesium sulfate, concentrated and then purified by column chromatography to obtain intermediate P3-128-A as a white solid (18.8 g, yield 65%).

### Step 2: Synthesis of Intermediate P3-128-B

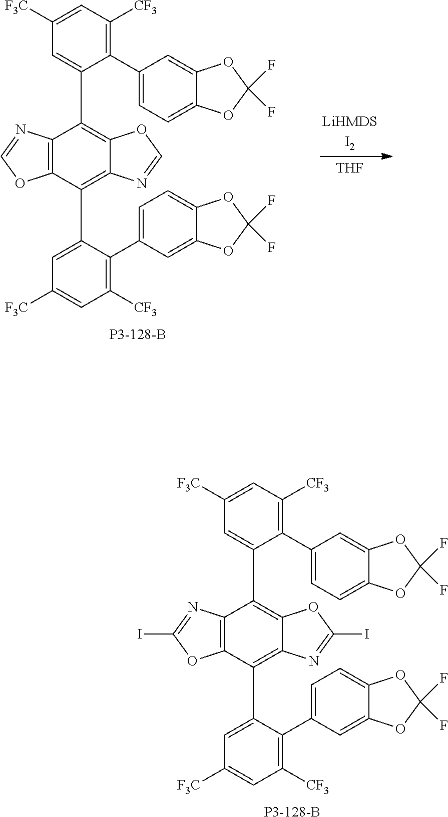

In a 2 L two-necked flask, intermediate P3-128-A (18.8 g, 20.9 mmol) was added, 800 mL of ultradry THF was added under nitrogen protection, and the mixture was cooled to −30 °C. Lithium bis(trimethylsilyl)amide (LiHMIDS, 60 mL, 60 mmol) was then dropwise added. The mixture was reacted at this temperature for 2 h. Elemental iodine (16.6 g, 65.3 mmol) was then added, and the mixture was heated to room temperature and reacted for 0.5 h. The reaction liquid was quenched with a saturated sodium sulfite solution and extracted by adding an appropriate amount of ethyl acetate, and the extract liquor was dried over anhydrous magnesium sulfate, concentrated and then purified by column chromatography to obtain intermediate P3-128-B as a white solid (21.1 g, yield 88%).

### Step 3: Synthesis of Intermediate P3-128-C

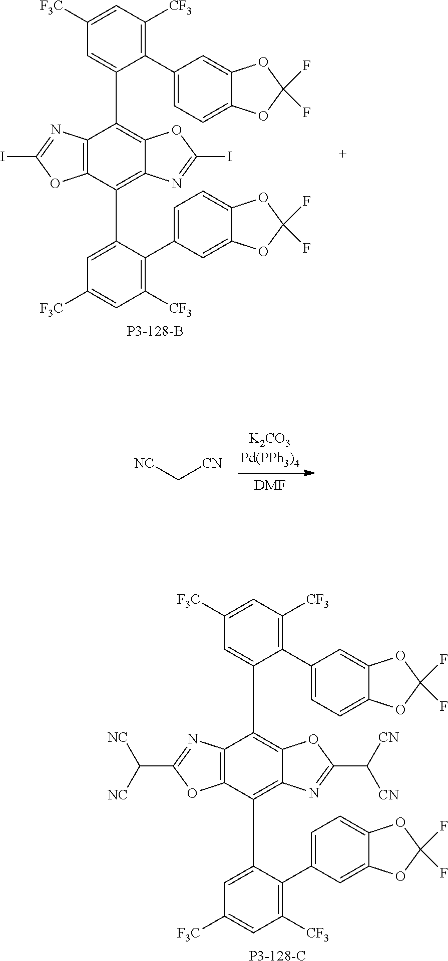

In a 2 L two-necked flask, intermediate P3-128-B (21.1 g, 18.4 mmol), malononitrile (5.00 g, 75.8 mmol), potassium carbonate (11.2 g, 81.2 mmol), and Pd(PPh₃)₄ (0.229 g, 0.198 mmol) were successively added, 350 mL of DMF was added under nitrogen protection, and the mixture was heated to 80 °C and reacted overnight. After the reaction was complete, the reaction liquid was cooled to room temperature and cooled in an ice-water bath. The reaction liquid was slowly dropwise added to 3N hydrochloric acid to precipitate a large amount of a yellow solid, which was filtered to obtain a crude product. After crystallization, intermediate P3-128-C as a white solid (13.6 g, yield 72%) was obtained.

### Step 4: Synthesis of Compound P3-128

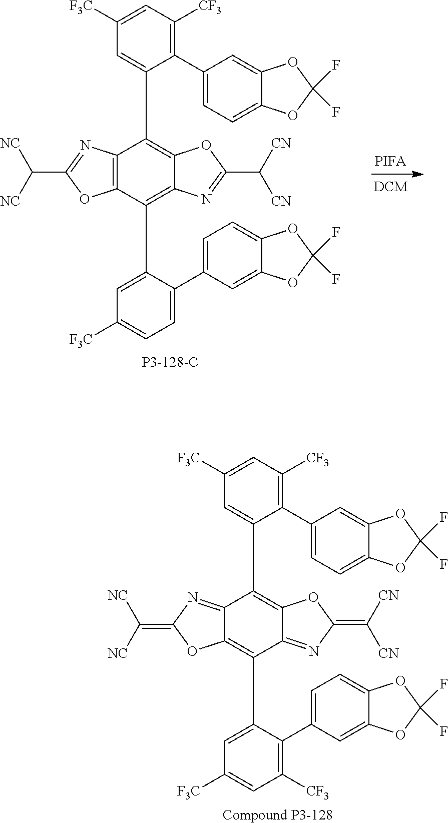

In a nitrogen atmosphere, to a 3 L single-necked flask, intermediate P3-128-C (13.6 g, 13.2 mmol) and then 1000 mL of DCM were added, and bis(trifluoroacetoxy)iodobenzene (PIFA, 11.0 g, 25.6 mmol) was added in portions at room temperature. After stirring at room temperature for 2 days, the reaction liquid was concentrated to an appropriate volume and filtered to obtain compound P3-128 as a black solid (10.3 g, yield 78%). The product was confirmed to be the target compound with a molecular weight of 1022.0.

### Synthesis Example 7: Synthesis of Compound P3-346

### Step 1: Synthesis of Intermediate P3-346-A

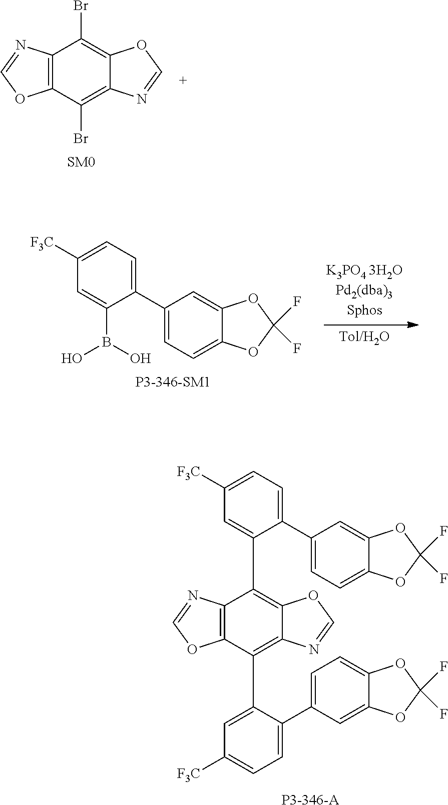

In a 2 L two-necked flask, intermediate SM0 (11.3 g, 35.4 mmol), intermediate P3-346-SM1 (45.1 g, 101.2 mmol), potassium phosphate trihydrate (31.4 g, 118.2 mmol), Pd₂(dba)₃ (1.72 g, 1.874 mmol), and SPhos (2-dicyclohexylphosphino-2′,6′-dimethoxybiphenyl (3.36 g, 8.19 mmol) were successively added. After nitrogen displacement three times, 800 mL of toluene and 200 mL of water were added under nitrogen protection, and the mixture was heated to 100 °C and reacted overnight. After the reaction was complete, the reaction liquid was cooled and extracted by adding an appropriate amount of tetrahydrofuran, and the extract liquor was dried over anhydrous magnesium sulfate, concentrated and then purified by column chromatography to obtain intermediate P3-346-A as a white solid (23.1 g, yield 68%).

### Step 2: Synthesis of Intermediate P3-346-B

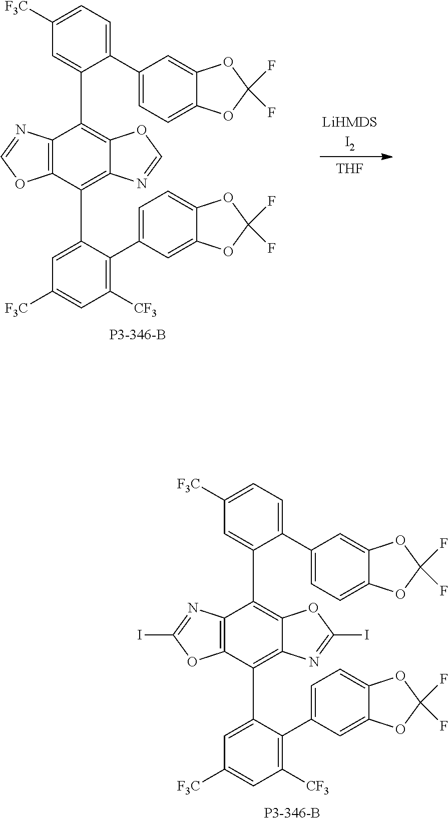

In a 2 L two-necked flask, intermediate P3-346-A (23.1 g, 24.1 mmol) was added, 800 mL of ultradry THF was added under nitrogen protection, and the mixture was cooled to −30 °C. Lithium bis(trimethylsilyl)amide (LiHMIDS, 60 mL, 60 mmol) was then dropwise added. The mixture was reacted at this temperature for 2 h. Elemental iodine (16.3 g, 64.2 mmol) was then added, and the mixture was heated to room temperature and reacted for 0.5 h. The reaction liquid was quenched with a saturated sodium sulfite solution and extracted by adding an appropriate amount of ethyl acetate, and the extract liquor was dried over anhydrous magnesium sulfate, concentrated and then purified by column chromatography to obtain intermediate P3-346-B as a white solid (24.5 g, yield 84%).

### Step 3: Synthesis of Intermediate P3-346-C

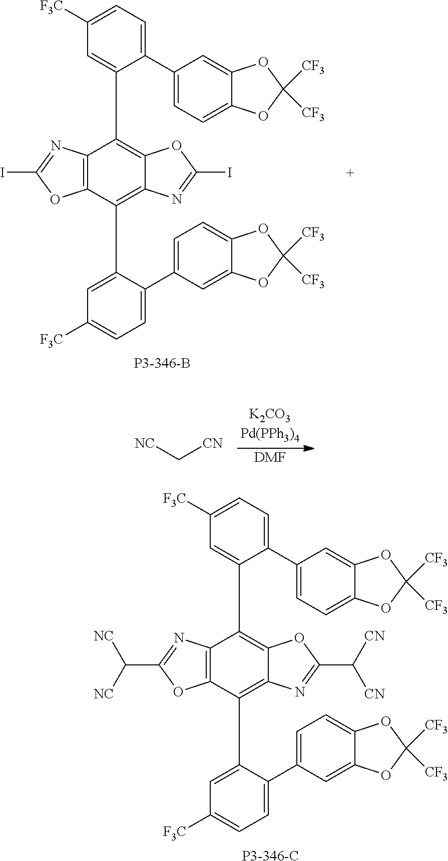

In a 2 L two-necked flask, intermediate P3-346-B (24.5 g, 20.2 mmol), malononitrile (5.18 g, 78.5 mmol), potassium carbonate (11.8 g, 85.2 mmol), and Pd(PPh₃)₄ (0.287 g, 0.248 mmol) were successively added, 350 mL of DMF was added under nitrogen protection, and the mixture was heated to 80 °C and reacted overnight. After the reaction was complete, the reaction liquid was cooled to room temperature and cooled in an ice-water bath. The reaction liquid was slowly dropwise added to 3N hydrochloric acid to precipitate a large amount of a yellow solid, which was filtered to obtain a crude product. After crystallization, intermediate P3-346-C as a white solid (16.7 g, yield 76%) was obtained.

### Step 4: Synthesis of Compound P3-346

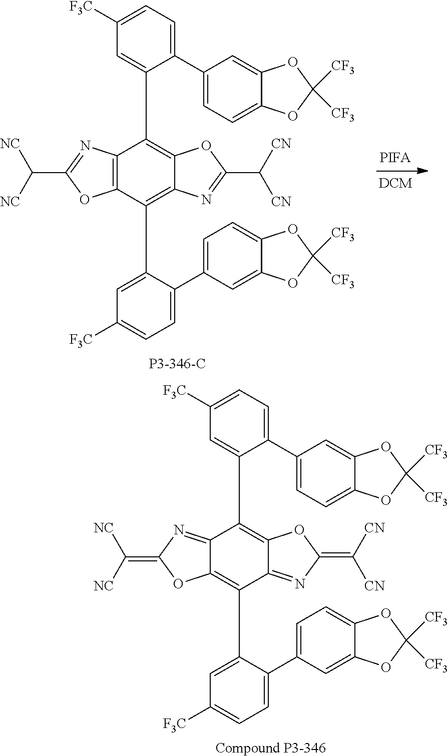

In a nitrogen atmosphere, to a 3 L single-necked flask, intermediate P3-346-C (16.7 g, 15.4 mmol) and then 1000 mL of DCM were added, and bis(trifluoroacetoxy)iodobenzene (PIFA, 13.7 g, 31.8 mmol) was added in portions at room temperature. After stirring at room temperature for 2 days, the reaction liquid was concentrated to an appropriate volume and filtered to obtain compound P3-128 as a black solid (11.4 g, yield 68%). The product was confirmed to be the target compound with a molecular weight of 1086.0.

### Synthesis Example 8: Synthesis of Compound P4-109

### Step 1: Synthesis of Intermediate P4-109-A

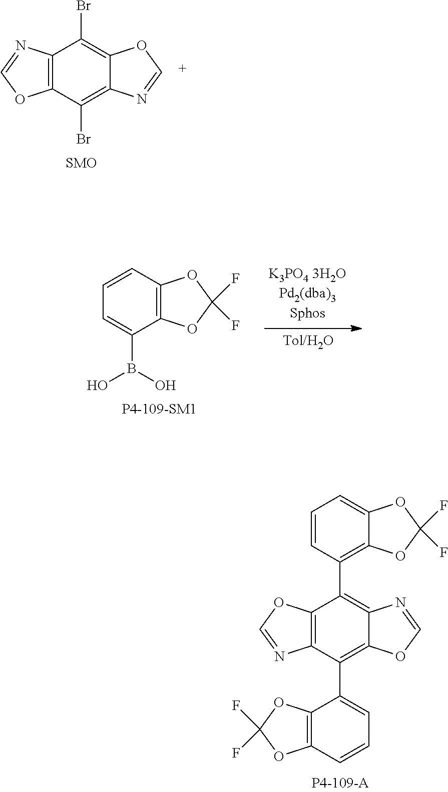 [SMILES: Brc1c2ncoc2c(Br)c2ncoc12 ; OB(O)c1cccc2c1OC(F)(F)O2 ; FC1(F)Oc2cccc(-c3c4ncoc4c(-c4cccc5c4OC(F)(F)O5)c4ncoc34)c2O1]

In a 2 L two-necked flask, intermediate SM0 (10.3 g, 32.3 mmol), intermediate P4-109-SM1 (21.5 g, 106.2 mmol), potassium phosphate trihydrate (29.3 g, 110.2 mmol), Pd₂(dba)₃ (1.62 g, 1.764 mmol), and SPhos (2-dicyclohexylphosphino-2′,6′-dimethoxybiphenyl (3.38 g, 8.24 mmol) were successively added. After nitrogen displacement three times, 800 mL of toluene and 200 mL of water were added under nitrogen protection, and the mixture was heated to 100 °C and reacted overnight. After the reaction was complete, the reaction liquid was cooled and extracted by adding an appropriate amount of tetrahydrofuran, and the extract liquor was dried over anhydrous magnesium sulfate, concentrated and then purified by column chromatography to obtain intermediate P4-109-A as a white solid (11.3 g, yield 74%).

### Step 2: Synthesis of Intermediate P4-109-B

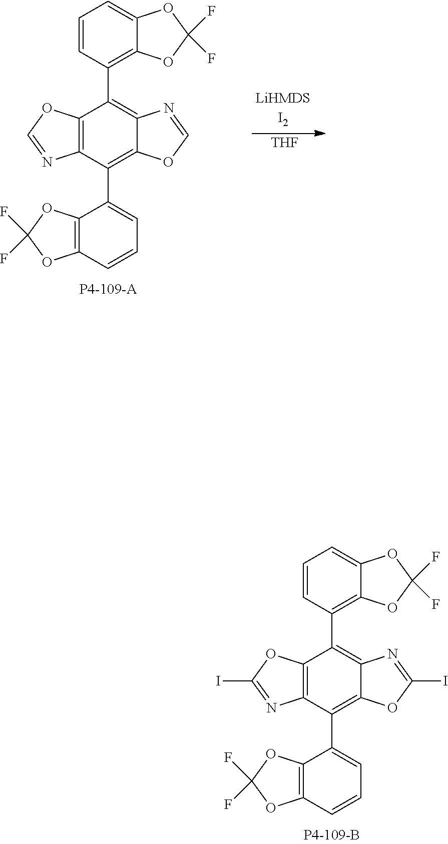 [SMILES: FC1(F)Oc2cccc(-c3c4ncoc4c(-c4cccc5c4OC(F)(F)O5)c4ncoc34)c2O1 ; "THF" ; FC1(F)Oc2cccc(-c3c4nc(I)oc4c(-c4cccc5c4OC(F)(F)O5)c4nc(I)oc34)c2O1]

In a 2 L two-necked flask, intermediate P4-109-A (11.3 g, 23.9 mmol) was added, 800 mL of ultradry THF was added under nitrogen protection, and the mixture was cooled to −30 °C. Lithium bis(trimethylsilyl)amide (LiHMIDS, 60 mL, 60 mmol) was then dropwise added. The mixture was reacted at this temperature for 2 h. Elemental iodine (16.3 g, 64.2 mmol) was then added, and the mixture was heated to room temperature and reacted for 0.5 h. The reaction liquid was quenched with a saturated sodium sulfite solution and extracted by adding an appropriate amount of ethyl acetate, and the extract liquor was dried over anhydrous magnesium sulfate, concentrated and then purified by column chromatography to obtain intermediate P4-109-B as a white solid (13.7 g, yield 79%).

### Step 3: Synthesis of Intermediate P4-109-C

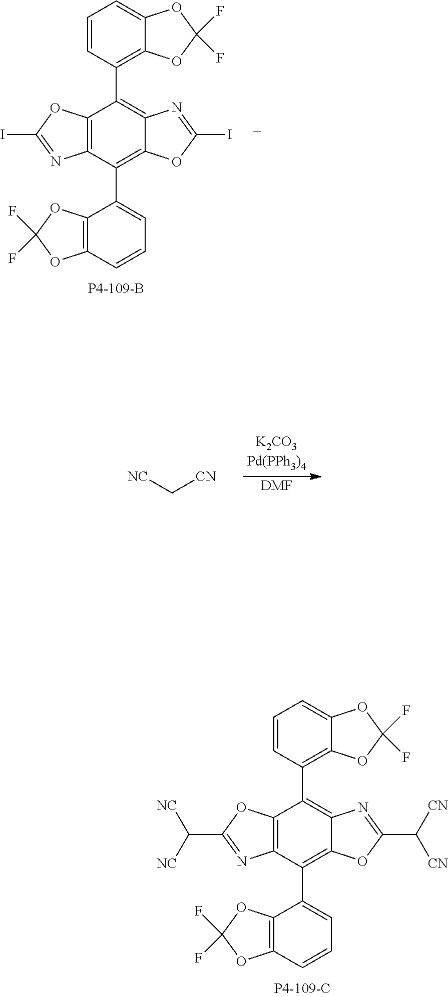

In a 2 L two-necked flask, intermediate P4-109-B (13.7 g, 18.9 mmol), malononitrile (5.05 g, 76.5 mmol), potassium carbonate (11.6 g, 83.8 mmol), and Pd(PPh₃)₄ (0.328 g, 0.284 mmol) were successively added, 350 mL of DMF was added under nitrogen protection, and the mixture was heated to 80 °C and reacted overnight. After the reaction was complete, the reaction liquid was cooled to room temperature and cooled in an ice-water bath. The reaction liquid was slowly dropwise added to 3N hydrochloric acid to precipitate a large amount of a yellow solid, which was filtered to obtain a crude product. After crystallization, intermediate P4-109-C as a white solid (8.40 g, yield 74%) was obtained.

### Step 4: Synthesis of Compound P4-109

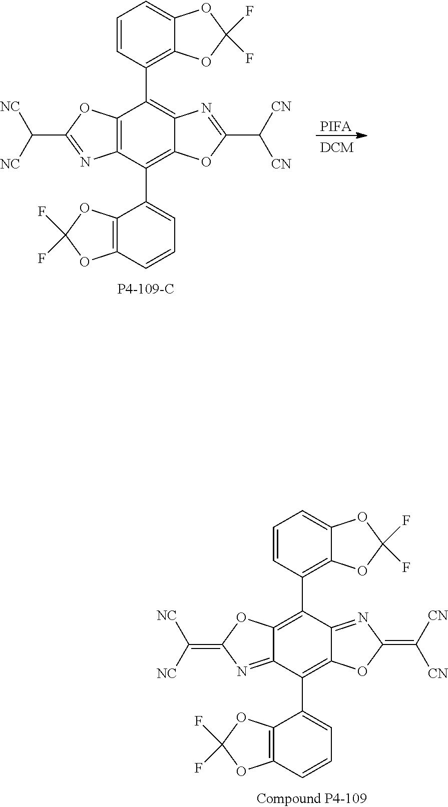

In a nitrogen atmosphere, to a 3 L single-necked flask, intermediate P4-109-C (8.40 g, 14.0 mmol) and then 1000 mL of DCM were added, and bis(trifluoroacetoxy)iodobenzene (PIFA, 11.5 g, 26.8 mmol) was added in portions at room temperature. After stirring at room temperature for 2 days, the reaction liquid was concentrated to an appropriate volume and filtered to obtain compound P4-109 as a black solid (7.6 g, yield 91%). The product was confirmed to be the target compound with a molecular weight of 598.0.

### Synthesis Example 9: Synthesis of Compound P4-128

Step 1. Synthesis of intermediate P4-128-A

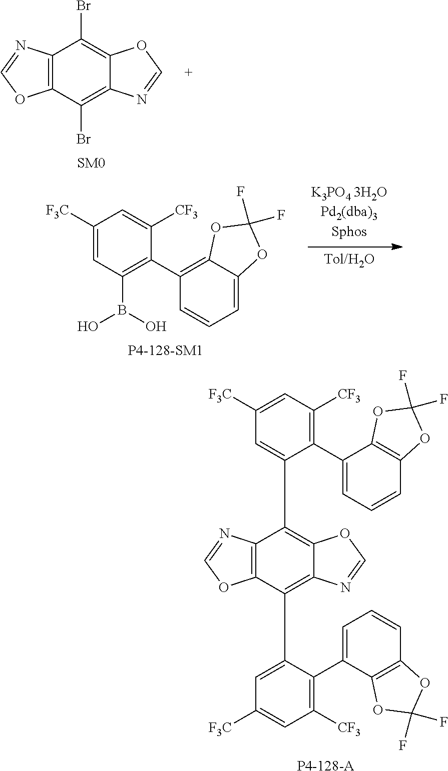

In a 2 L two-necked flask, intermediate SM0 (10.6 g, 33.4 mmol), intermediate P4-128-SM1 (40.8 g, 98.5 mmol), potassium phosphate trihydrate (29.2 g, 109.8 mmol), Pd₂(dba)₃ (1.76 g, 1.924 mmol), and SPhos (2-dicyclohexylphosphino-2′,6′-dimethoxybiphenyl (3.38 g, 8.24 mmol) were successively added. After nitrogen displacement three times, 800 mL of toluene and 200 mL of water were added under nitrogen protection, and the mixture was heated to 100 °C and reacted overnight. After the reaction was complete, the reaction liquid was cooled and extracted by adding an appropriate amount of tetrahydrofuran, and the extract liquor was dried over anhydrous magnesium sulfate, concentrated and then purified by column chromatography to obtain intermediate P4-128-A as a white solid (19.5 g, yield 65%).

### Step 2: Synthesis of Intermediate P4-128-B

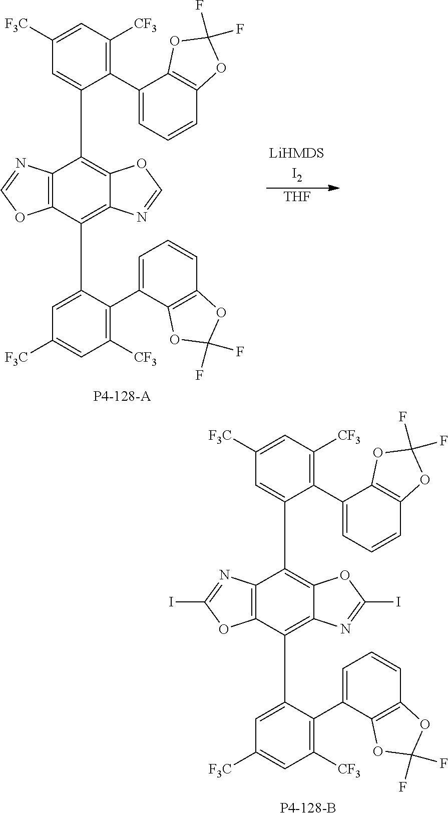

In a 2 L two-necked flask, intermediate P4-128-A (19.5 g, 21.7 mmol) was added, 800 mL of ultradry THF was added under nitrogen protection, and the mixture was cooed to −30 °C. Lithium bis(trimethylsilyl)amide (LiHMDS, 60 mL, 60 mmol) was then dropwise added. The mixture was reacted at this temperature for 2 h. Elemental iodine (16.8 g, 66.2 mmol) was then added, and the mixture was heated to room temperature and reacted for 0.5 h. The reaction liquid was quenched with a saturated sodium sulfite solution and extracted by adding an appropriate amount of ethyl acetate, and the extract liquor was dried over anhydrous magnesium sulfate, concentrated and then purified by column chromatography to obtain intermediate P4-128-B as a white solid (21.4 g, yield 86%).

### Step 3: Synthesis of Intermediate P4-128-C

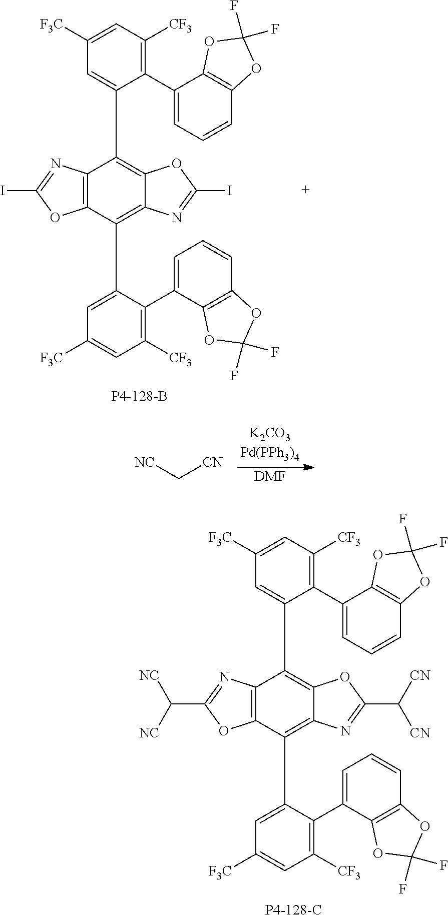

In a 2 L two-necked flask, intermediate P4-128-B (21.4 g, 18.7 mmol), malononitrile (5.17 g, 78.4 mmol), potassium carbonate (12.3 g, 88.8 mmol), and Pd(PPh₃)₄ (0.310 g, 0.268 mmol) were successively added, 350 mL of DMF was added under nitrogen protection, and the mixture was heated to 80 °C and reacted overnight. After the reaction was complete, the reaction liquid was cooled to room temperature and cooled in an ice-water bath. The reaction liquid was slowly dropwise added to 3N hydrochloric acid to precipitate a large amount of a yellow solid, which was filtered to obtain a crude product. After crystallization, intermediate P4-128-C as a white solid (13.8 g, yield 72%) was obtained.

### Step 4: Synthesis of Compound P4-128

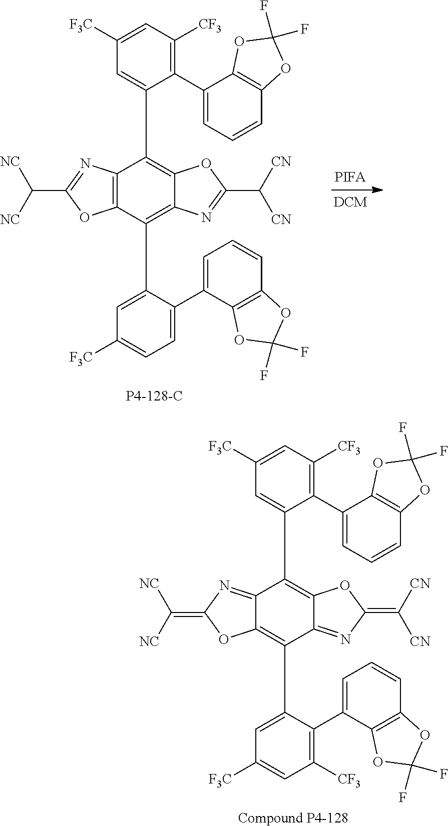

In a nitrogen atmosphere, to a 3 L single-necked flask, intermediate P4-128-C (13.8 g, 13.5 mmol) and then 1000 mL of DCM were added, and bis(trifluoroacetoxy)iodobenzene (PIFA, 11.0 g, 25.6 mmol) was added in portions at room temperature. After stirring at room temperature for 2 days, the reaction liquid was concentrated to an appropriate volume and filtered to obtain compound P4-128 as a black solid (10.5 g, yield 76%). The product was confirmed to be the target compound with a molecular weight of 1022.0.

### Synthesis Example 10: Synthesis of Compound P4-136

### Step 1: Synthesis of Intermediate P4-136-A

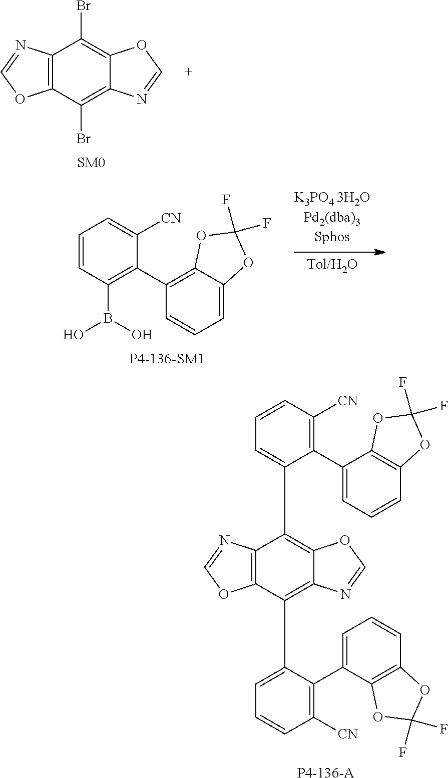

In a 2 L two-necked flask, intermediate SM0 (14.6 g, 45.9 mmol), intermediate P4-136-SM1 (50.8 g, 167.7 mmol), potassium phosphate trihydrate (42.7 g, 160.5 mmol), Pd₂(dba)₃ (1.85 g, 2.02 mmol), and SPhos (2-dicyclohexylphosphino-2′,6′-dimethoxybiphenyl (4.23 g, 10.3 mmol) were successively added. After nitrogen displacement three times, 800 mL of toluene and 200 mL of water were added under nitrogen protection, and the mixture was heated to 100 °C and reacted overnight. After the reaction was complete, the reaction liquid was cooled and extracted by adding an appropriate amount of tetrahydrofuran, and the extract liquor was dried over anhydrous magnesium sulfate, concentrated and then purified by column chromatography to obtain intermediate P4-136-A as a white solid (21.0 g, yield 68%).

### Step 2: Synthesis of Intermediate P4-136-B

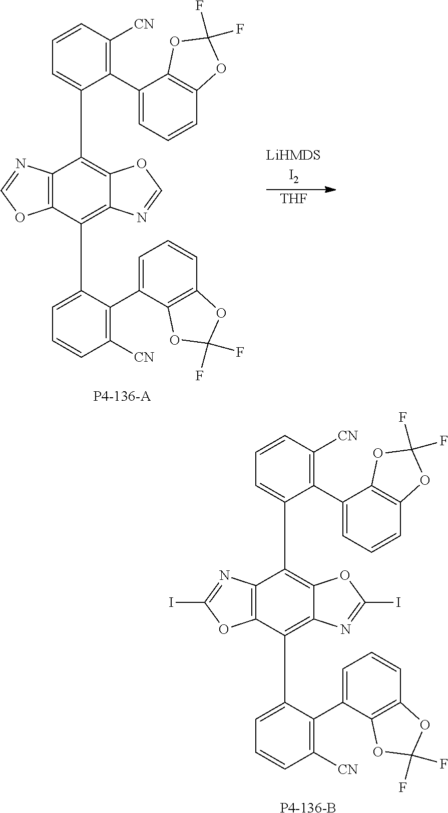

In a 2 L two-necked flask, intermediate P4-136-A (21.0 g, 31.2 mmol) was added, 1200 mL of ultradry THF was added under nitrogen protection, and the mixture was cooled to −20 °C. Lithium bis(trimethylsilyl)amide (LIHMIDS, 60 mL, 60 mmol) was then dropwise added. The mixture was reacted at this temperature for 2 h. Elemental iodine (16.2 g, 63.6 mmol) was then added, and the mixture was heated to room temperature and reacted for 0.5 h. The reaction liquid was quenched with a saturated sodium sulfite solution and extracted by adding an appropriate amount of ethyl acetate, and the extract liquor was dried over anhydrous magnesium sulfate, concentrated and then purified by column chromatography to obtain intermediate P4-136-B as a white solid (22.0 g, yield 76%).

Step 3. Synthesis of intermediate P4-136-C

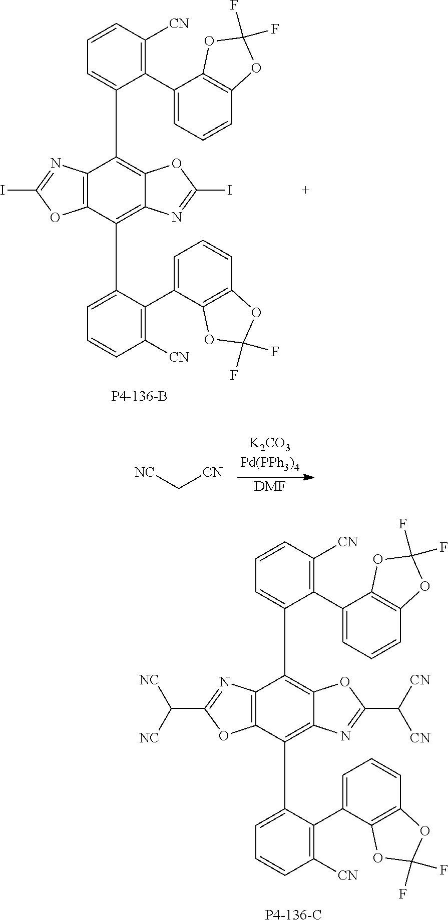

In a 2 L two-necked flask, intermediate P4-136-B (22.0 g, 23.8 mmol), malononitrile (6.34 g, 96.3 mmol), potassium carbonate (14.2 g, 102.8 mmol), and Pd(PPh₃)₄ (0.34 g, 0.294 mmol) were successively added, 500 mL of DMF was added under nitrogen protection, and the mixture was heated to 80 °C and reacted overnight. After the reaction was complete, the reaction liquid was cooled to room temperature and cooled in an ice-water bath. The reaction liquid was slowly dropwise added to 3N hydrochloric acid to precipitate a large amount of a yellow solid, which was filtered to obtain a crude product. After crystallization, intermediate P4-136-C as a white solid (11.8 g, yield 62%) was obtained.

### Step 4: Synthesis of Compound P4-136

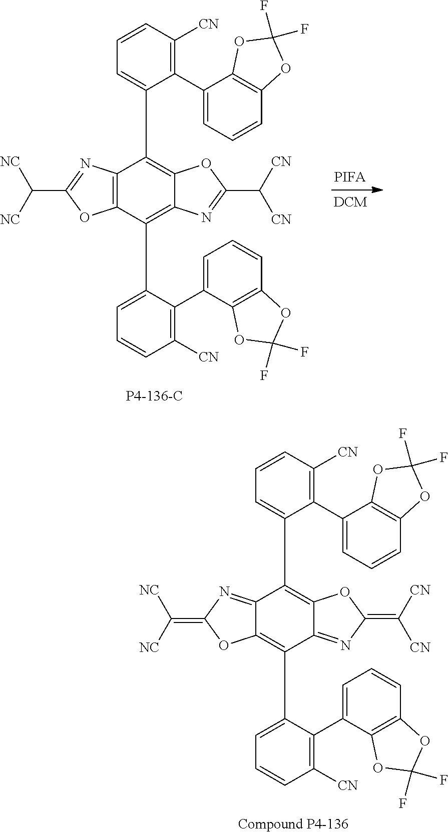

In a nitrogen atmosphere, to a 3 L single-necked flask, intermediate P4-136-C (11.8 g, 14.8 mmol) and then 1000 mL of DCM were added, and bis(trifluoroacetoxy)iodobenzene (PIFA, 8.77 g, 20.4 mmol) was added in portions at room temperature. After stirring at room temperature for 2 days, the reaction liquid was concentrated to an appropriate volume and filtered to obtain compound P4-136 as a black solid (10.1 g, yield 85%). The product was confirmed to be the target compound with a molecular weight of 800.1.

### Synthesis Example 11: Synthesis of Compound P4-206

### Step 1: Synthesis of Intermediate P4-206-A

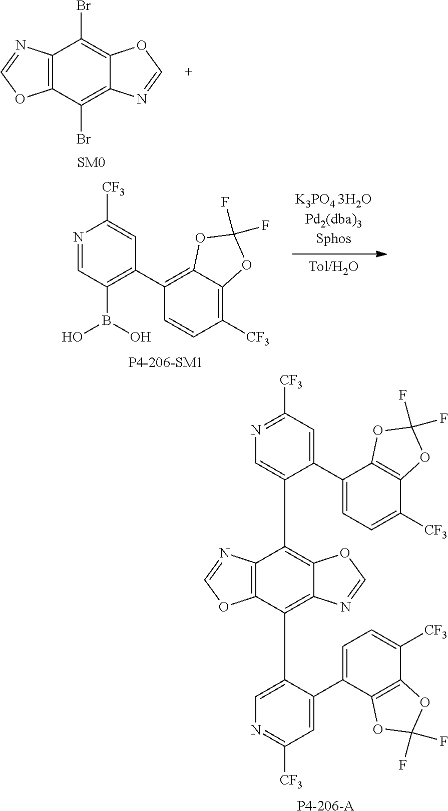

In a 2 L two-necked flask, intermediate SM0 (9.6 g, 30.2 mmol), intermediate P4-206-SM1 (52.5 g, 126.5 mmol), potassium phosphate trihydrate (31.3 g, 117.5 mmol), Pd₂(dba)₃ (1.45 g, 1.58 mmol), and SPhos (2-dicyclohexylphosphino-2′,6′-dimethoxybiphenyl (3.56 g, 8.67 mmol) were successively added. After nitrogen displacement three times, 800 mL of toluene and 200 mL of water were added under nitrogen protection, and the mixture was heated to 100 °C and reacted overnight. After the reaction was complete, the reaction liquid was cooled and extracted by adding an appropriate amount of tetrahydrofuran, and the extract liquor was dried over anhydrous magnesium sulfate, concentrated and then purified by column chromatography to obtain intermediate P4-206-A as a white solid (12.5 g, yield 46%).

### Step 2: Synthesis of Intermediate P4-206-B

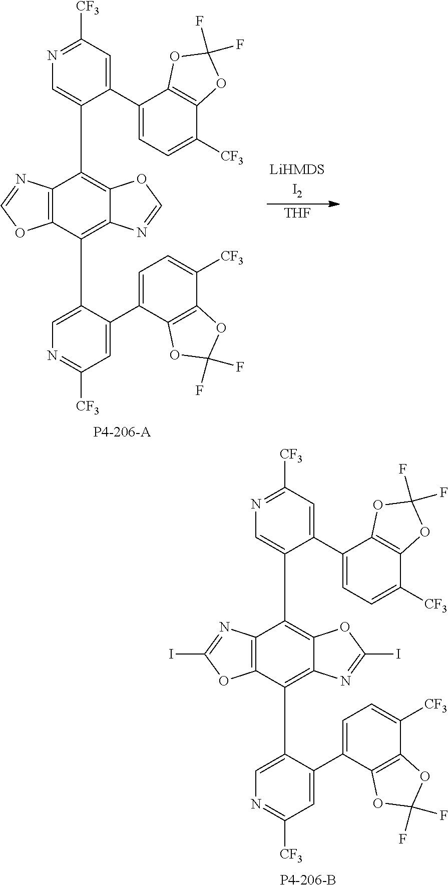

In a 2 L two-necked flask, intermediate P4-206-A (12.5 g, 13.9 mmol) was added, 800 mL of ultradry THF was added under nitrogen protection, and the mixture was cooled to −30 °C. Lithium bis(trimethylsilyl)amide (LiHMDS, 50 mL, 50 mmol) was then dropwise added. The mixture was reacted at this temperature for 2 h. Elemental iodine (14.9 g, 58.6 mmol) was then added, and the mixture was heated to room temperature and reacted for 0.5 h. The reaction liquid was quenched with a saturated sodium sulfite solution and extracted by adding an appropriate amount of ethyl acetate, and the extract liquor was dried over anhydrous magnesium sulfate, concentrated and then purified by column chromatography to obtain intermediate P4-206-B as a white solid (14.4 g, yield 90%).

### Step 3: Synthesis of Intermediate P4-206-C

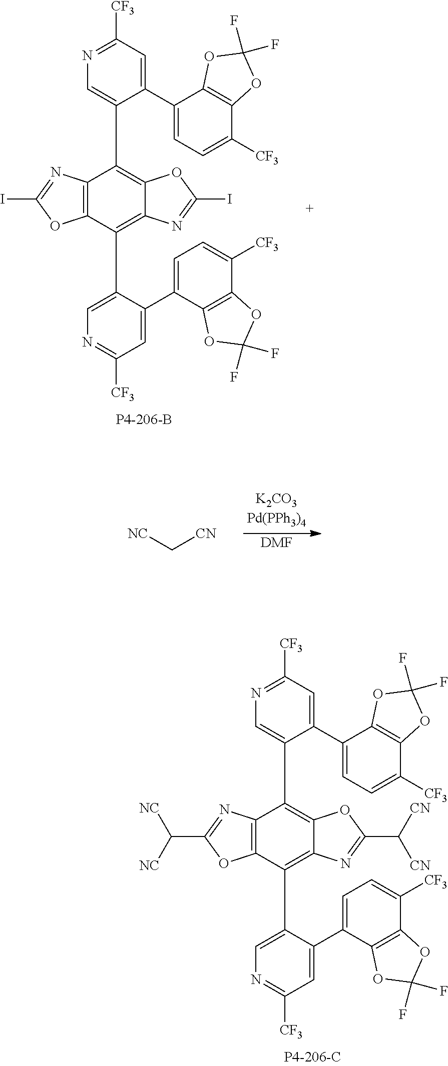

In a 2 L two-necked flask, intermediate P4-206-B (14.4 g, 12.5 mmol), malononitrile (3.19 g, 48.4 mmol), potassium carbonate (7.44 g, 53.8 mmol), and Pd(PPh₃)₄ (0.25 g, 0.216 mmol) were successively added, 350 mL of DMF was added under nitrogen protection, and the mixture was heated to 80 °C and reacted overnight. After the reaction was complete, the reaction liquid was cooled to room temperature and cooled in an ice-water bath. The reaction liquid was slowly dropwise added to 3N hydrochloric acid to precipitate a large amount of a yellow solid, which was filtered to obtain a crude product. After crystallization, intermediate P4-206-C as a white solid (10.4 g, yield 81%) was obtained.

### Step 4: Synthesis of Compound P4-206

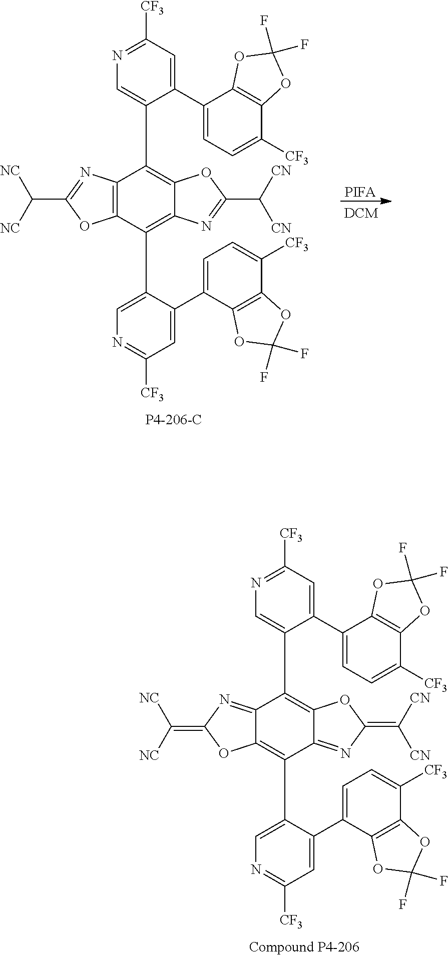

In a nitrogen atmosphere, to a 3 L single-necked flask, intermediate P4-206-C (10.4 g, 10.1 mmol) and then 1000 mL of DCM were added, and bis(trifluoroacetoxy)iodobenzene (PIFA, 7.14 g, 16.6 mmol) was added in portions at room temperature. After stirring at room temperature for 2 days, the reaction liquid was concentrated to an appropriate volume and filtered to obtain compound P4-206 as a black solid (5.80 g, yield 56%). The product was confirmed to be the target compound with a molecular weight of 1024.0.

### Comparative Compounds

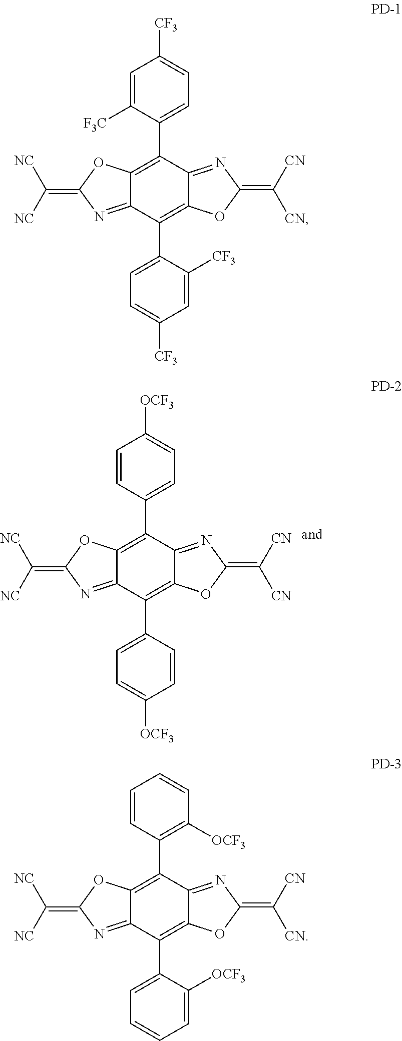

### Device Examples

### Device Example 1

This example provided an organic electroluminescent device. The preparation method therefor was as follows: an ITO substrate was patterned to achieve a light-emitting area of 2 mm×2 mm in size, followed by washing with isopropanol, UV, and ozone, respectively; subsequently, the ITO substrate was mounted on a substrate holder of a vacuum deposition device, and the pressure was adjusted to achieve a vacuum level of 1×10⁷ torr. Firstly, on an ITO layer (anode) formed on the substrate, compound P1-493 provided in Synthesis Example 1 of the present invention and compound HT-1 (the mass ratio of compound P1-493 to compound HT-1 was 2:98) were deposited under vacuum to a thickness of 10 nm to form a hole injection layer; secondly, on the above-mentioned hole injection layer, compound HT-1 was deposited under vacuum to a thickness of 110 nm to form a hole transport layer; thirdly, on the above-mentioned hole transport layer, compound EB-1 was deposited under vacuum to a thickness of 10 nm to form an electron barrier layer; fourthly, on the above-mentioned electron barrier layer, a mixture of compound BD-1 and compound BH was deposited under vacuum to a thickness of 20 nm to form a light-emitting layer (the mass ratio of BD-1 to BH was 2:98); next, compound HB-1 was deposited under vacuum to a thickness of 5 nm to form a hole barrier layer; subsequently, on the above-mentioned hole barrier layer, compound ET-1 and compound Liq (the mass ratio of ET-1 to Liq was 5:5) was deposited under vacuum to a thickness of 30 nm to form an electron transport layer; then, on the above-mentioned electron transport layer, LiF was deposited under vacuum to a thickness of 1 nm to form an electron injection layer; and finally, on the above-mentioned electron injection layer, aluminum (Al) was deposited under vacuum to a thickness of 150 nm to form a cathode, thereby preparing the organic electroluminescent device.

### Device Example 2

The preparation method was the same as that in Device Example 1, except that compound P1-493 in the hole injection layer was replaced by compound P2-257, and the mass ratio of compound P2-257 to HT-1 in the hole injection layer was 2:98.

### Device Example 3

The preparation method was the same as that in Device Example 1, except that compound P1-493 in the hole injection layer was replaced by compound P2-271, and the mass ratio of compound P2-271 to HT-1 in the hole injection layer was 2:98.

### Device Example 4

The preparation method was the same as that in Device Example 1, except that compound P1-493 in the hole injection layer was replaced by compound P3-115, and the mass ratio of compound P3-115 to HT-1 in the hole injection layer was 2:98.

### Device Example 5

The preparation method was the same as that in Device Example 1, except that compound P1-493 in the hole injection layer was replaced by compound P3-124, and the mass ratio of compound P3-124 to HT-1 in the hole injection layer was 2:98.

### Device Example 6

The preparation method was the same as that in Device Example 1, except that compound P1-493 in the hole injection layer was replaced by compound P3-128, and the mass ratio of compound P3-128 to HT-1 in the hole injection layer was 2:98.

### Device Example 7

The preparation method was the same as that in Device Example 1, except that compound P1-493 in the hole injection layer was replaced by compound P3-346, and the mass ratio of compound P3-346 to HT-1 in the hole injection layer was 2:98.

### Device Example 8

The preparation method was the same as that in Device Example 1, except that compound P1-493 in the hole injection layer was replaced by compound P4-109, and the mass ratio of compound P4-109 to HT-1 in the hole injection layer was 2:98.

### Device Example 9

The preparation method was the same as that in Device Example 1, except that compound P1-493 in the hole injection layer was replaced by compound P4-128, and the mass ratio of compound P4-128 to HT-1 in the hole injection layer was 2:98.

### Device Example 10

The preparation method was the same as that in Device Example 1, except that compound P1-493 in the hole injection layer was replaced by compound P4-136, and the mass ratio of compound P4-136 to HT-1 in the hole injection layer was 2:98.

### Device Example 11

The preparation method was the same as that in Device Example 1, except that compound P1-493 in the hole injection layer was replaced by compound P4-206, and the mass ratio of compound P4-206 to HT-1 in the hole injection layer was 2:98.

### Comparative Device Example 1

The preparation method was the same as that in Device Example 1, except that compound P1-493 in the hole injection layer was replaced by compound PD-1, and the mass ratio of compound PD-1 to HT-1 in the hole injection layer was 2:98.

### Comparative Device Example 2

The preparation method was the same as that in Device Example 1, except that compound P1-493 in the hole injection layer was replaced by compound PD-2, and the mass ratio of compound PD-2 to HT-1 in the hole injection layer was 2:98.

### Comparative Device Example 3

The preparation method was the same as that in Device Example 1, except that compound P1-493 in the hole injection layer was replaced by compound PD-3, and the mass ratio of compound PD-3 to HT-1 in the hole injection layer was 2:98.

The molecular structural formulas of the materials used in the present invention and the materials in the other layers of the devices were as follows:

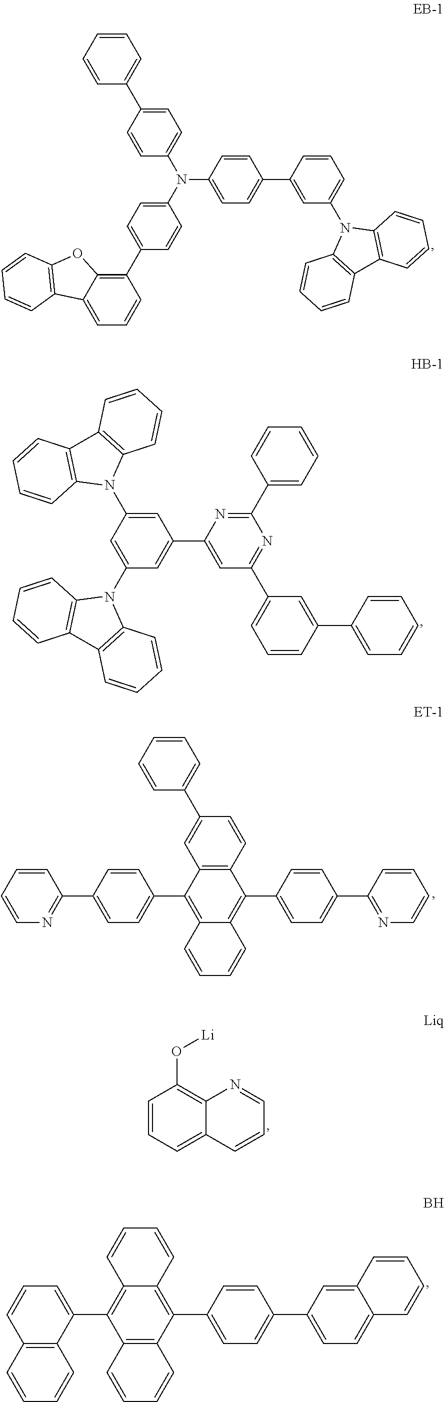

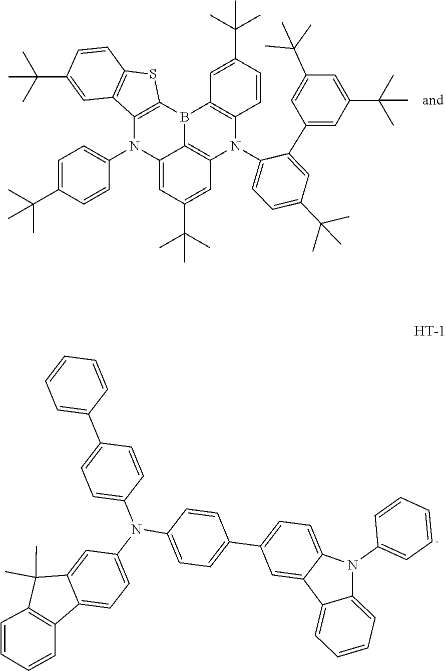 [SMILES: CC(C)(C)c1ccc(N2c3cc(C(C)(C)C)cc4c3B(c3cc(C(C)(C)C)ccc3N4c3ccc(C(C)(C)C)cc3-c3cc(C(C)(C)C)cc(C(C)(C)C)c3)c3sc4ccc(C(C)(C)C)cc4c32)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21]

The electrode preparation method and the deposition method for each functional layer in this embodiment were both conventional methods in the art, such as vacuum thermal evaporation or ink jet printing. No more detailed description would be given here.

### Device Performance

The organic electroluminescent devices provided by Device Examples 1-11 and Comparative Device Examples 1-3 were tested by standard methods. For this purpose, the driving voltage and current efficiency (CE) of the organic electroluminescent device were determined at a current density of J=10 mA/cm², while the lifetime (LT95) of the organic electroluminescent device was determined at a current density of J=25 mA/cm².

The test instruments and methods for testing the performance of the above OLED devices were as follows:
the brightness was tested by means of spectrum scanner PhotoResearch PR-635;
the current density and turn-on voltage were tested by digital SourceMeter Keithley 2400; and
lifetime test: on a silicon photoelectric OLED lifetime testing system was used.

The performance test results of the above devices were listed in Table 5.

TABLE 5
Device performance test results

| | | | | | Life- |
|---|---|---|---|---|---|
| | | | Driving | Current | time |
| | PD | Light | voltage | efficiency | LT95 |
| Device No. | material | color | (V) | CE (cd/A) | (h) |
| Device Example 1 | P1-493 | Blue light | 3.78 | 6.91 | 89.1 |
| Device Example 2 | P2-257 | Blue light | 3.65 | 6.89 | 85.8 |
| Device Example 3 | P2-271 | Blue light | 3.82 | 6.72 | 86.4 |
| Device Example 4 | P3-115 | Blue light | 3.91 | 6.74 | 86.9 |
| Device Example 5 | P3-124 | Blue light | 3.86 | 6.90 | 87.4 |
| Device Example 6 | P3-128 | Blue light | 3.73 | 6.98 | 89.2 |
| Device Example 7 | P3-346 | Blue light | 3.68 | 6.80 | 88.7 |
| Device Example 8 | P4-109 | Blue light | 3.88 | 6.70 | 85.5 |
| Device Example 9 | P4-128 | Blue light | 3.93 | 6.69 | 86.5 |
| Device Example 10 | P4-136 | Blue light | 3.80 | 6.83 | 88.0 |
| Device Example 11 | P4-206 | Blue light | 3.85 | 6.86 | 88.2 |
| Comparative Device | PD-1 | Blue light | 4.01 | 6.78 | 86.4 |
| Example 1 | | | | | |
| Comparative Device | PD-2 | Blue light | 4.29 | 6.28 | 62.5 |
| Example 2 | | | | | |
| Comparative Device | PD-3 | Blue light | 4.15 | 5.73 | 65.6 |
| Example 3 | | | | | |

As can be seen from the device performance test results in Table 5 above:
the organic electroluminescent devices of Examples 1-11 prepared by the organic compounds provided by the present invention have lower driving voltage than Comparative Device Example 1 and also have comparable current efficiency and lifetime, indicating that the organic compounds of the present invention have superior hole injection ability as doping materials in the hole injection layer.

In addition, as can be seen from the TGA test, the organic compounds provided by the present invention have better heat resistance and thermal stability as compared with PD-1. For example, the TGA test of P3-115 shows that the 5% weight loss temperature is 385 °C and the TGA test of P2-271 shows that the 5% weight loss temperature is 380 °C, whereas the TGA test of PD-1 shows that the 5% weight loss temperature is 346 °C. The higher the weight loss temperature, the better the heat resistance and thermal stability of the molecules.

Compared with the organic electroluminescent devices provided by Comparative Device Examples 2 and 3, the driving voltages, current efficiencies, and lifetimes of the organic electroluminescent devices of Examples 1-11 prepared from the organic compounds provided by the present invention are improved to different degrees. Compared with the organic electroluminescent device provided by Comparative Device Example 2, the driving voltages of the organic electroluminescent devices of Examples 1-11 prepared from the organic compounds provided by the present invention are reduced by 8.4% to 14.9%, the current efficiencies are increased by 6.5% to 11.1%, and the lifetimes are increased by 36.8% to 42.7%. Compared with the organic electroluminescent device provided by the Comparative Device Example 3, the driving voltages of the organic electroluminescent devices of Examples 1-11 prepared from the organic compounds provided by the present invention are reduced by 5.3% to 12.0%, the current efficiencies are increased by 16.8% to 21.8%, and the lifetimes are increased by 30.3% to 36.0%, indicating that the double-X ring of the formula II unit in the structures of the compounds of the present invention can better improve the doping ability of the material in the hole injection layer than a single OCF₃ group, leading to reduced voltage of the device and improved efficiency and lifetime of the device.

### Sublimation Thermal Stability Test:

The materials of the synthesis examples of the present invention and the materials of the comparative examples (PD-1 to PD-5) were uniformly spread in a vacuum sublimation apparatus, and the pressure was adjusted until the vacuum level was 1×10⁻⁵ torr or less; using a temperature-programmed method, the temperature was gradually increased until material deposition occurred in a collection zone of the vacuum sublimation apparatus, and the temperature was maintained constant; and after a predetermined period, confirmation was made that no additional material was accumulated in the collection zone of the vacuum sublimation apparatus, heating was stopped, and a sublimated material in the collection zone of the vacuum sublimation apparatus was collected.

The structures of the two new compounds PD-4 and PD-5 of the comparative examples were as follows:

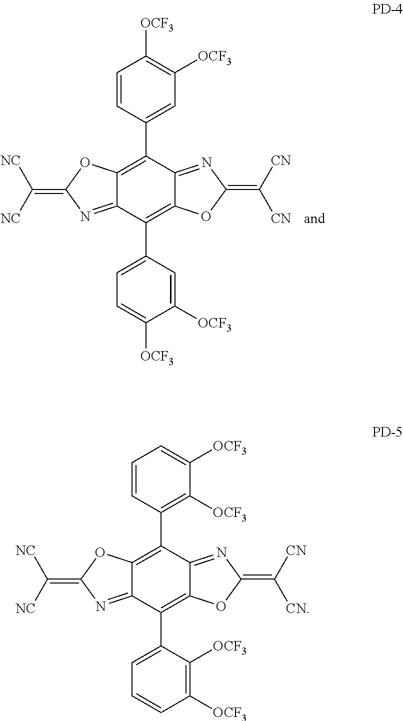 [SMILES: N#CC(C#N)=c1nc2c(-c3ccc(OC(F)(F)F)c(OC(F)(F)F)c3)c3oc(=C(C#N)C#N)nc3c(-c3ccc(OC(F)(F)F)c(OC(F)(F)F)c3)c2o1 ; "and" ; N#CC(C#N)=c1nc2c(-c3cccc(OC(F)(F)F)c3OC(F)(F)F)c3oc(=C(C#N)C#N)nc3c(-c3cccc(OC(F)(F)F)c3OC(F)(F)F)c2o1]

The sublimation performance test results of the above devices were listed in Table 6.

TABLE 6
Sublimation test results

| Sample No. | P1-493 | P2-257 | P2-271 | P3-115 | P3-124 | P3-128 | P3-346 | P4-109 |
|---|---|---|---|---|---|---|---|---|
| Purity before | 99.7 | 99.8 | 99.6 | 99.7 | 99.8 | 99.9 | 99.5 | 99.6 |
| sublimation (%) | | | | | | | | |
| Purity after | 99.9 | 99.8 | 99.9 | 99.8 | 99.7 | 99.8 | 99.5 | 99.5 |
| sublimation (%) | | | | | | | | |
| Purity change | 0.2 | 0 | 0.3 | 0.1 | −0.1 | −0.1 | 0 | −0.1 |
| before and after | | | | | | | | |
| sublimation (%) | | | | | | | | |
| Sublimation | 78 | 61 | 84 | 86 | 76 | 78 | 82 | 55 |
| yield (%) | | | | | | | | |
| Sample No. | P4-128 | P4-136 | P4-206 | PD-1 | PD-2 | PD-3 | PD-4 | PD-5 |
| Purity before | 99.8 | 99.7 | 99.5 | 99.9 | 99.8 | 99.9 | 99.8 | 99.7 |
| sublimation (%) | | | | | | | | |
| Purity after | 99.7 | 99.5 | 99.6 | 99.8 | 99.5 | 99.5 | 95.8 | 97.6 |
| sublimation (%) | | | | | | | | |
| Purity change | −0.1 | −0.2 | 0.1 | −0.1 | −0.3 | −0.4 | −4.0 | −2.1 |
| before and after | | | | | | | | |
| sublimation (%) | | | | | | | | |

From the above results, it can be seen that the purity of the organic compound material of the present invention changes by +/−0.3% before and after sublimation. The purity shows little to no change; in some cases, it is even increased rather than decreased. The change in the purity of the organic compound materials provided by the present invention before and after sublimation is basically equivalent to that of PD-1. In contrast, the purities of the materials PD-2 to PD-5 of the comparative examples all decrease to different degrees after sublimation. Although there are only two OCF₃ groups in the PD-2 and PD-3 structures, the purity decreases by 0.3% and 0.4% after sublimation. In addition, there are four OCF₃ groups in the PD-4 and PD-5 structures, and the purity is reduced by 4.0% and 2.1% after sublimation. The significant decrease in purity renders the material unsuitable for device evaluation. It can be seen that in the organic compounds of the present invention, the two X groups in the same ring in formula II make the compound more stable in structure and better in thermal resistance.

The sublimation yield of the organic compounds of the present invention is 55% to 86%, which is a superior sublimation yield. Compared with PD-5, the sublimation yield of the organic compound P4-109 of the present invention is increased by 32%, indicating that where the substitution sites on the benzene ring are the same, the cyclization of X in the structure of formula II of the compound of the present invention is beneficial to the stability of the material and the improvement of the sublimation yield. Compared with PD-3, the sublimation yield of the organic compound P4-109 of the present invention is higher by about 2%, and the compound of the present invention still has a relatively high sublimation yield. The organic compound P2-257 of the present invention has a comparable sublimation yield relative to the compound PD-1 of the Comparative Example. In addition, compared with the organic compounds P2-257 and P4-109, the sublimation yields of the other compounds of the present invention, especially those with the linker group L in formula II, are significantly increased by 15% to 31%.

In summary, the organic compounds provided by the present invention have superior doping ability for the hole injection layer, and the organic electroluminescent device prepared by using the organic compound of the present invention has excellent device performance. In addition, the unique ring-forming structure in the formula not only improves the thermal stability of the material, but also improves the sublimation yield of the material, and has wide application value.

The above description is only preferred embodiments of the present invention, and the scope of protection of the present invention is not limited thereto. Any changes, substitutions, etc. readily conceivable to any of those familiar with the technical field within the technical scope of the disclosure of the present invention should be included in the scope of protection of the present invention. Therefore, for the scope of protection of the present invention, the scope of protection of the claims shall prevail.

1. A compound for an organic electroluminescent device, characterized in that the general structural formula of the compound is as represented by formula I: 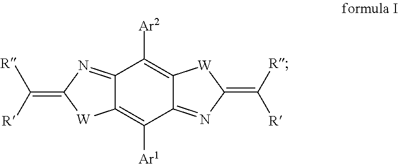
in formula I, each W is independently selected from O, S, or Se;
Ar¹ and Ar² are each independently selected from the group consisting of any one of or any combination of some of hydrogen, deuterium, halogen, carbonyl, cyano, sulfinyl, sulfonyl, phosphoroso, a substituted or unsubstituted alkyl group with 1-20 carbon atoms, a substituted or unsubstituted cycloalkyl group with 3-20 ring carbon atoms, a substituted or unsubstituted alkoxy group with 1-20 ring carbon atoms, a substituted or unsubstituted aryloxy group with 6-30 carbon atoms, a substituted or unsubstituted aryl group with 6-30 carbon atoms, and a substituted or unsubstituted heteroaryl group with 3-30 carbon atoms; and at least one of Ar¹ and Ar² has a structure as represented by the following formula II, 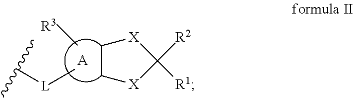
in formula II, each X is independently selected from any one of or any combination of some of O, S, Se, 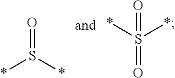
L is selected from a single bond, O, a substituted or unsubstituted arylene group with 6-30 carbon atoms, a substituted or unsubstituted heteroarylene group with 3-30 carbon atoms, and a combination thereof;
ring A is selected from any one of a substituted or unsubstituted aromatic ring with 6-30 carbon atoms and a substituted or unsubstituted heteroaromatic ring with 3-30 carbon atoms;
R³ represents a substituent on the ring A and is selected from monosubstitution, polysubstitution, or unsubstitution, and when R³ refers to polysubstitution, the plurality of substituents can be the same or different;
R¹, R², and R³ are, at each occurrence, each independently selected from the group consisting of any one of or any combination of some of hydrogen, deuterium, hydroxy, mercapto, halogen, nitroso, nitro, acyl, carbonyl, carboxy, an ester group, cyano, isocyano, SCN, OCN, SF₅, boranyl, sulfinyl, sulfonyl, phosphoroso, a substituted or unsubstituted alkyl group with 1-20 carbon atoms, a substituted or unsubstituted cycloalkyl group with 3-20 ring carbon atoms, a substituted or unsubstituted heteroalkyl group with 1-20 carbon atoms, a substituted or unsubstituted heterocyclic group with 3-20 ring atoms, a substituted or unsubstituted aralkyl group with 7-30 carbon atoms, a substituted or unsubstituted alkoxy group with 1-20 ring carbon atoms, a substituted or unsubstituted aryloxy group with 6-30 carbon atoms, a substituted or unsubstituted alkenyl group with 2-20 carbon atoms, a substituted or unsubstituted alkynyl group with 2-20 carbon atoms, a substituted or unsubstituted aryl group with 6-30 carbon atoms, a substituted or unsubstituted heteroaryl group with 3-30 carbon atoms, a substituted or unsubstituted alkylsilyl group with 3-20 carbon atoms, a substituted or unsubstituted arylsilyl group with 6-20 carbon atoms, a substituted or unsubstituted alkylgermyl group with 3-20 carbon atoms, a substituted or unsubstituted arylgermyl group with 6-20 carbon atoms, and a substituted or unsubstituted amino group with 0-20 carbon atoms;
R′ and R″ are each independently selected from the group consisting of any one of or any combination of some of hydroxy, mercapto, halogen, nitroso, nitro, acyl, carbonyl, carboxy, an ester group, cyano, isocyano, SCN, OCN, SF₅, sulfinyl, sulfonyl, phosphoroso, a substituted or unsubstituted alkyl group with 1-20 carbon atoms, a substituted or unsubstituted cycloalkyl group with 3-20 ring carbon atoms, a substituted or unsubstituted heteroalkyl group with 1-20 carbon atoms, a substituted or unsubstituted heterocyclic group with 3-20 ring atoms, a substituted or unsubstituted aralkyl group with 7-30 carbon atoms, a substituted or unsubstituted alkoxy group with 1-20 ring carbon atoms, a substituted or unsubstituted aryloxy group with 6-30 carbon atoms, a substituted or unsubstituted alkenyl group with 2-20 carbon atoms, a substituted or unsubstituted alkynyl group with 2-20 carbon atoms, a substituted or unsubstituted aryl group with 6-30 carbon atoms, and a substituted or unsubstituted heteroaryl group with 3-30 carbon atoms;
any one of Ar¹, Ar², R′, and R″ contains at least one electron-withdrawing group; and in Ar¹, Ar², R′, R″, R², R³, and L, any adjacent substituents can be bonded to each other to form a ring; and [symbol]
represents the position of connection of L to formula I, and * represents the position of connection of X to another group.

2. The compound according to claim 1, characterized in that the compound has a structure as represented by formula I-1: 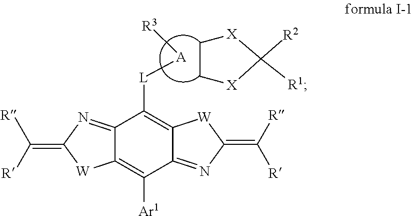
wherein in R′, R″, R¹, R², R³, and L, any adjacent substituents can be bonded to each other to form a ring.

3. The compound according to claim 1, characterized in that the compound has a structure as represented by formula I-2: 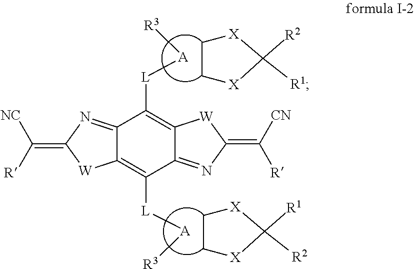
and
in R¹, R², R³, and L, any adjacent substituents can be bonded to each other to form a ring.

4. The compound according to claim 1, characterized in that the structure represented by formula II is selected from one of structures represented by formulas II-1 to II-6: 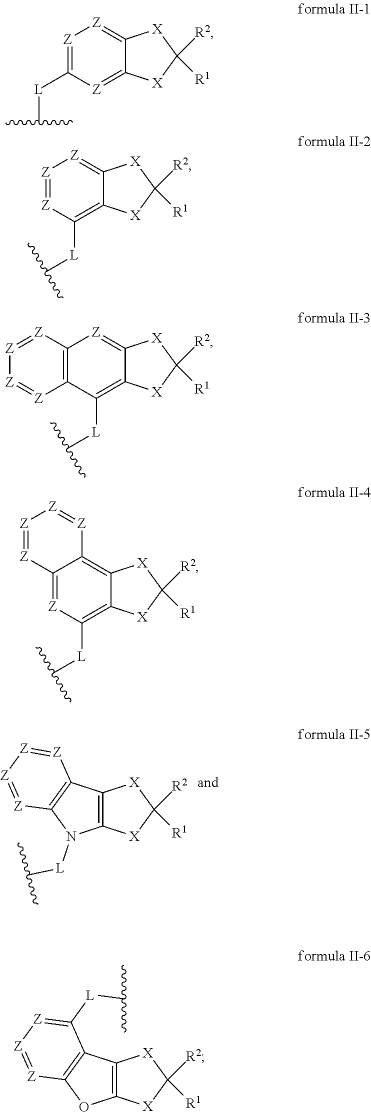
wherein Z is, at each occurrence, the same or different and is selected from CR³ or N;
in formulas II-1 to II-6, each X is independently selected from any one of or any combination of some of O, S, Se, 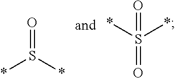
L is, at each occurrence, the same or different and is selected from a single bond, O, a substituted or unsubstituted arylene group with 6-30 carbon atoms, a substituted or unsubstituted heteroarylene group with 3-30 carbon atoms, and a combination thereof;
R¹, R², and R³ are, at each occurrence, the same or different and are selected from the group consisting of hydrogen, deuterium, hydroxy, mercapto, halogen, nitroso, nitro, acyl, carbonyl, carboxy, an ester group, cyano, isocyano, SCN, OCN, SF₅, boranyl, sulfinyl, sulfonyl, phosphoroso, a substituted or unsubstituted alkyl group with 1-20 carbon atoms, a substituted or unsubstituted cycloalkyl group with 3-20 ring carbon atoms, a substituted or unsubstituted heteroalkyl group with 1-20 carbon atoms, a substituted or unsubstituted heterocyclic group with 3-20 ring atoms, a substituted or unsubstituted aralkyl group with 7-30 carbon atoms, a substituted or unsubstituted alkoxy group with 1-20 ring carbon atoms, a substituted or unsubstituted aryloxy group with 6-30 carbon atoms, a substituted or unsubstituted alkenyl group with 2-20 carbon atoms, a substituted or unsubstituted alkynyl group with 2-20 carbon atoms, a substituted or unsubstituted aryl group with 6-30 carbon atoms, a substituted or unsubstituted heteroaryl group with 3-30 carbon atoms, a substituted or unsubstituted alkylsilyl group with 3-20 carbon atoms, a substituted or unsubstituted arylsilyl group with 6-20 carbon atoms, a substituted or unsubstituted alkylgermyl group with 3-20 carbon atoms, a substituted or unsubstituted arylgermyl group with 6-20 carbon atoms, a substituted or unsubstituted amino group with 0-20 carbon atoms, and a combination thereof;
in R¹, R², and R³, any adjacent substituents can be bonded to each other to form a ring; and [symbol]
represents the position of connection of L to formula I, and * represents the position of connection of X to another group.

5. The compound according to claim 4, characterized in that the structure represented by formula II is selected from structures represented by formula II-1 or II-2.

6. The compound according to claim 1, characterized in that R¹ and R² are each independently selected from the group consisting of any one of or any combination of some of hydrogen, deuterium, halogen, nitroso, nitro, carbonyl, cyano, SF₅, sulfinyl, sulfonyl, phosphoroso, a substituted or unsubstituted alkyl group with 1-20 carbon atoms, a substituted or unsubstituted cycloalkyl group with 3-20 ring carbon atoms, a substituted or unsubstituted heteroalkyl group with 1-20 carbon atoms, a substituted or unsubstituted heterocyclic group with 3-20 ring atoms, a substituted or unsubstituted aralkyl group with 7-30 carbon atoms, a substituted or unsubstituted alkoxy group with 1-20 ring carbon atoms, a substituted or unsubstituted aryloxy group with 6-30 carbon atoms, a substituted or unsubstituted alkenyl group with 2-20 carbon atoms, a substituted or unsubstituted alkynyl group with 2-20 carbon atoms, a substituted or unsubstituted aryl group with 6-30 carbon atoms, and a substituted or unsubstituted heteroaryl group with 3-30 carbon atoms; and
in R¹ and R², any adjacent substituents can be bonded to each other to form a ring.

7. The compound according to claim 1, characterized in that W is selected from O; and/or
R′ and R″ are, at each occurrence, the same or different and are selected from the group consisting of any one of or any combination of some of fluoro, cyano, SCN, sulfonyl, a substituted or unsubstituted alkyl group with 1-20 carbon atoms, a substituted or unsubstituted cycloalkyl group with 3-20 ring carbon atoms, a substituted or unsubstituted heteroalkyl group with 1-20 carbon atoms, a substituted or unsubstituted aralkyl group with 7-30 carbon atoms, a substituted or unsubstituted alkoxy group with 1-20 ring carbon atoms, a substituted or unsubstituted aryloxy group with 6-30 carbon atoms, a substituted or unsubstituted aryl group with 6-30 carbon atoms, and a substituted or unsubstituted heteroaryl group with 3-30 carbon atoms; and/or
R¹ and R² are, at each occurrence, the same or different and are selected from the group consisting of hydrogen, deuterium, fluoro, carbonyl, cyano, SF₅, a substituted or unsubstituted alkyl group with 1-20 carbon atoms, a substituted or unsubstituted aralkyl group with 7-30 carbon atoms, a substituted or unsubstituted alkoxy group with 1-20 ring carbon atoms, a substituted or unsubstituted aryloxy group with 6-30 carbon atoms, a substituted or unsubstituted aryl group with 6-30 carbon atoms, a substituted or unsubstituted heteroaryl group with 3-30 carbon atoms, and a combination thereof.

8. The compound according to claim 1, characterized in that X is selected from O; and/or
at least one of R′ and R″ is selected from cyano.

9. The compound according to claim 1, characterized in that when the ring A has a substituent, the ring A can be, at each occurrence, substituted with a single one or more R³, and when L has a substituent, L can be, at each occurrence, substituted with a single one or more R⁴; and
R³ and R⁴ are, at each occurrence, the same or different and are selected from the group consisting of any one of or any combination of some of hydrogen, deuterium, hydroxy, mercapto, halogen, carbonyl, cyano, SF₅, sulfonyl, phosphoroso, a substituted or unsubstituted alkyl group with 1-20 carbon atoms, a substituted or unsubstituted cycloalkyl group with 3-20 ring carbon atoms, a substituted or unsubstituted heteroalkyl group with 1-20 carbon atoms, a substituted or unsubstituted aralkyl group with 7-30 carbon atoms, a substituted or unsubstituted alkoxy group with 1-20 ring carbon atoms, a substituted or unsubstituted aryloxy group with 6-30 carbon atoms, a substituted or unsubstituted aryl group with 6-30 carbon atoms, and a substituted or unsubstituted heteroaryl group with 3-30 carbon atoms; and adjacent R³ and R⁴ can be optionally connected to form a ring.

10. The compound according to claim 1, characterized in that L is, at each occurrence, the same or different and is selected from any one of or any combination of some of a single bond, O, a substituted or unsubstituted phenylene group, a substituted or unsubstituted pyridinylene group, a substituted or unsubstituted biphenylene group, a substituted or unsubstituted terphenylene group, a substituted or unsubstituted naphthylene group, a substituted or unsubstituted naphthyridinylene group, a substituted or unsubstituted phenanthrenylene group, a substituted or unsubstituted phenanthrolinylene group, a substituted or unsubstituted anthracenylene group, a substituted or unsubstituted fluorenylidene group, a substituted or unsubstituted dibenzofuranylene group, a substituted or unsubstituted dibenzothienylene group, and a substituted or unsubstituted carbazolylene group.

11. The compound according to claim 9, characterized in that L is, at each occurrence, the same or different and is selected from a single bond, O, and the following structures, and a combination thereof: 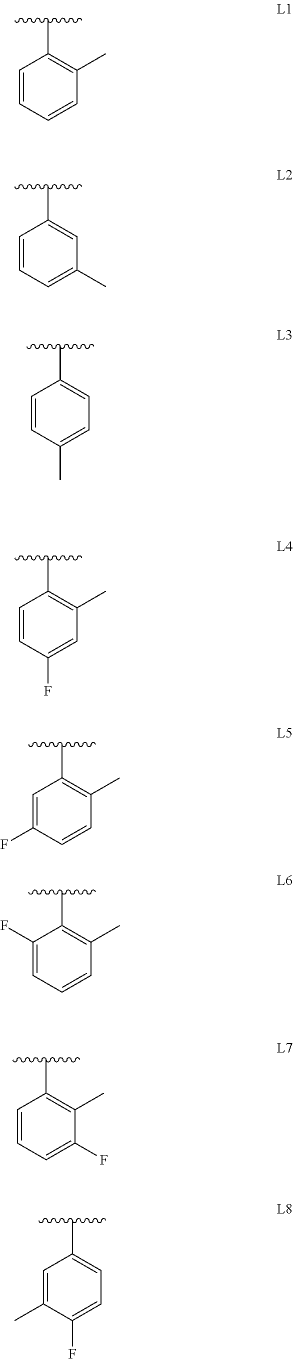 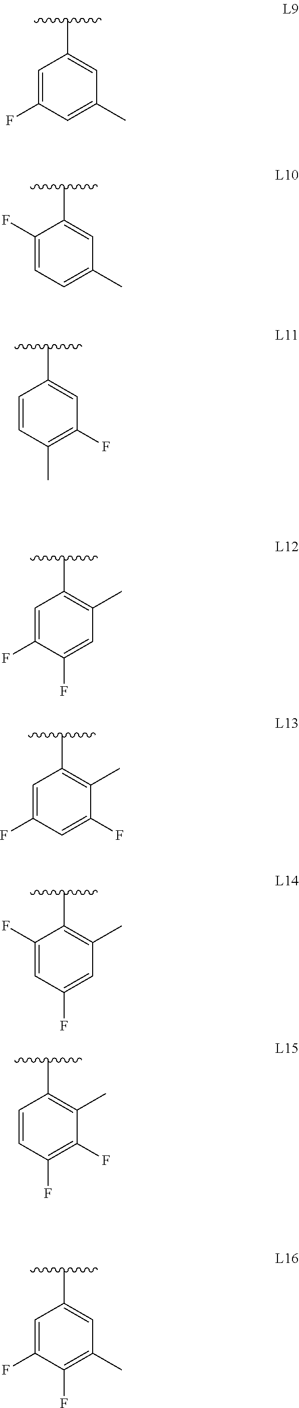 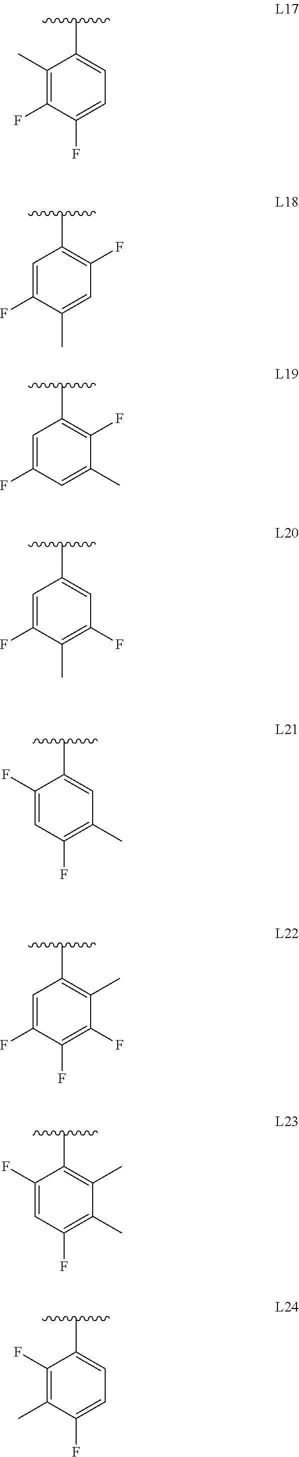 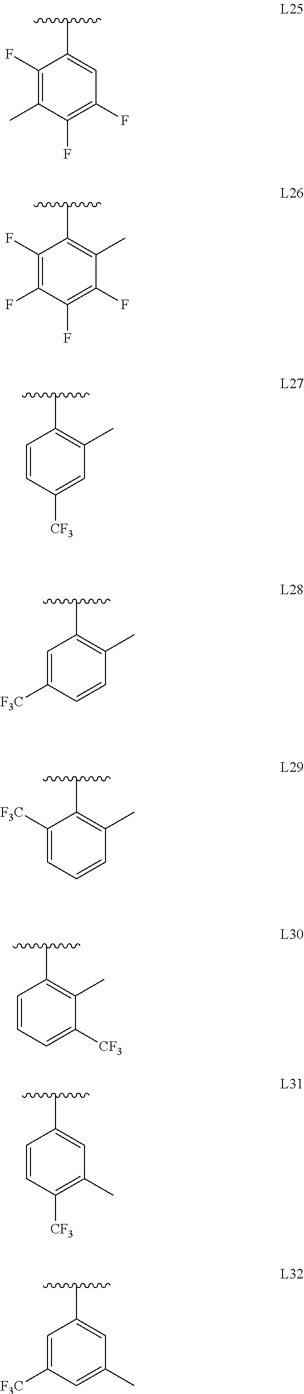 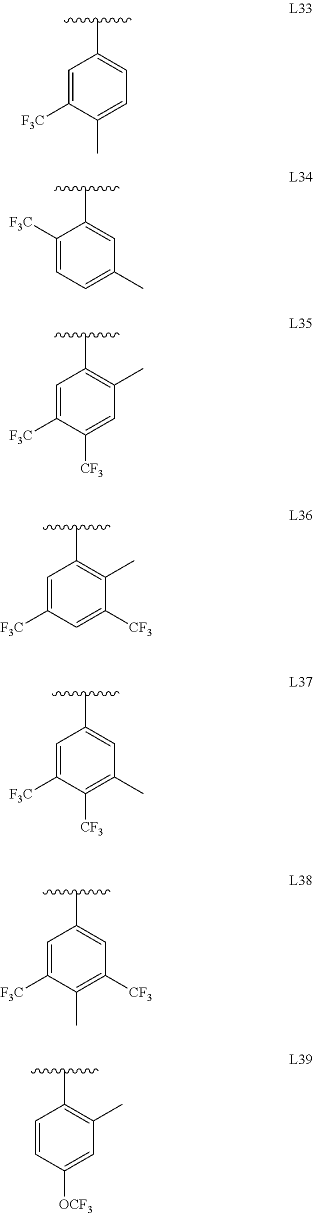 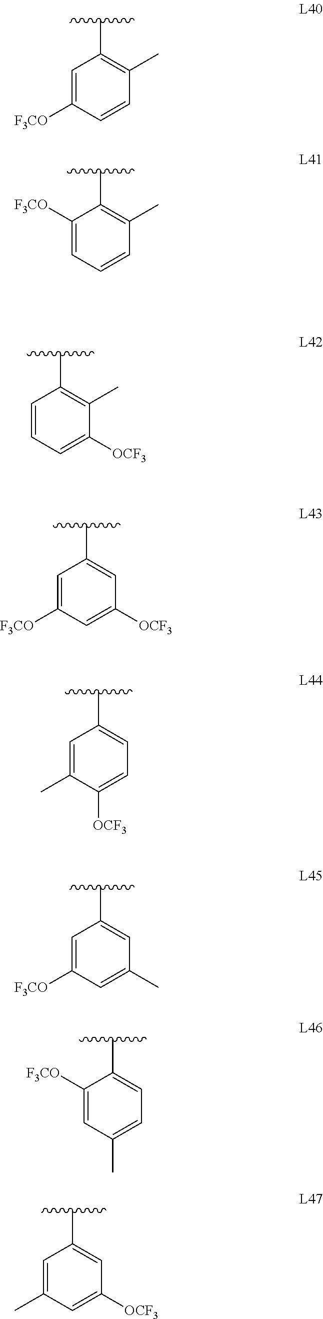 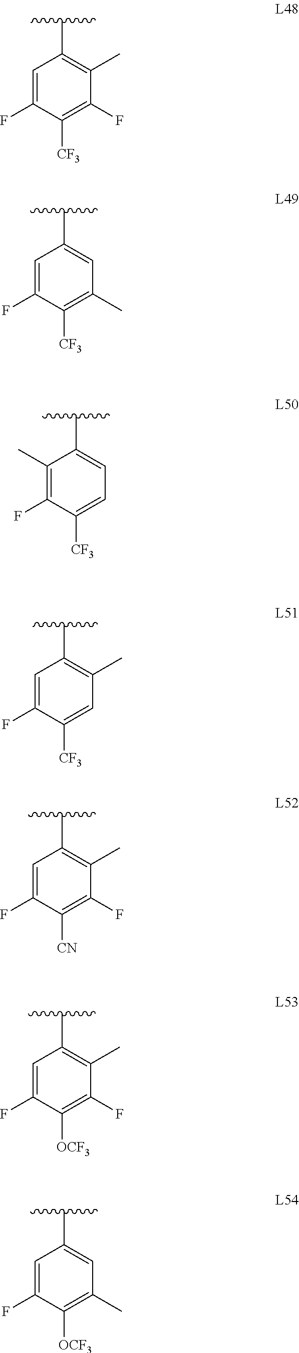 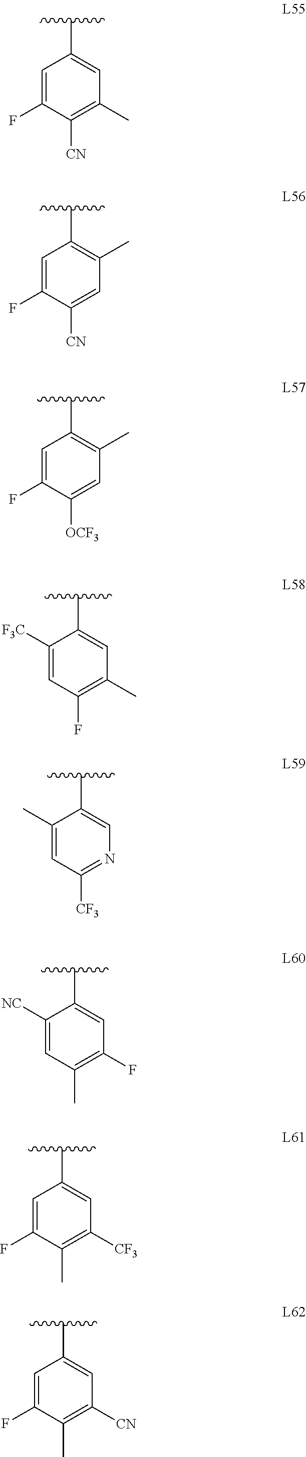 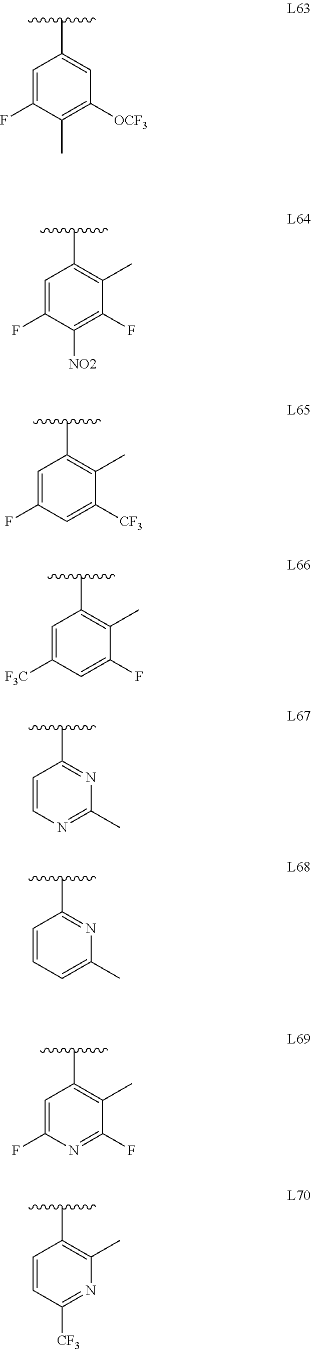 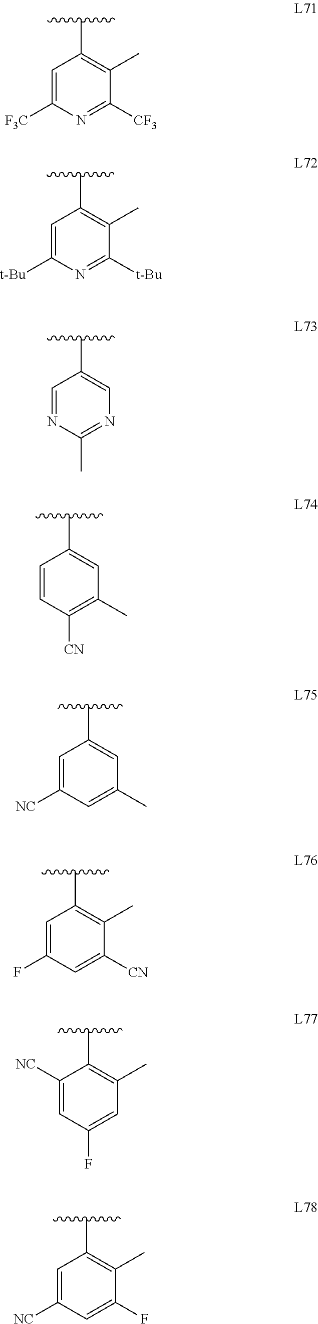 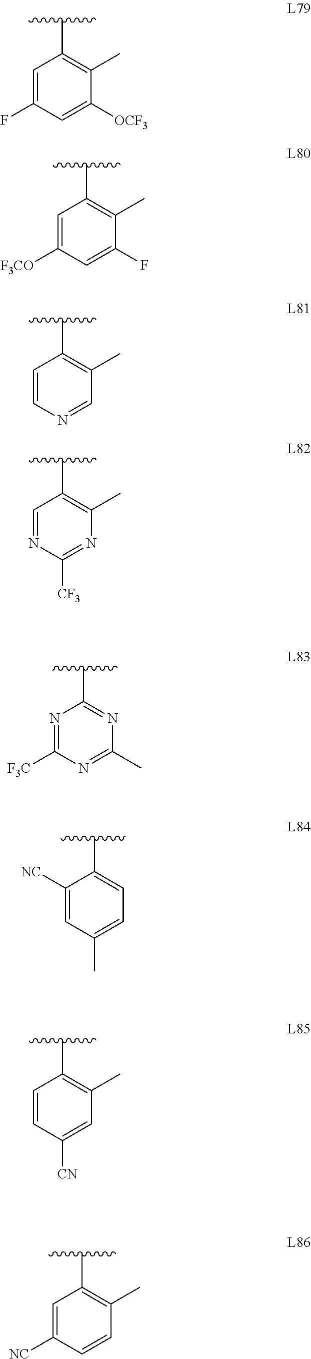 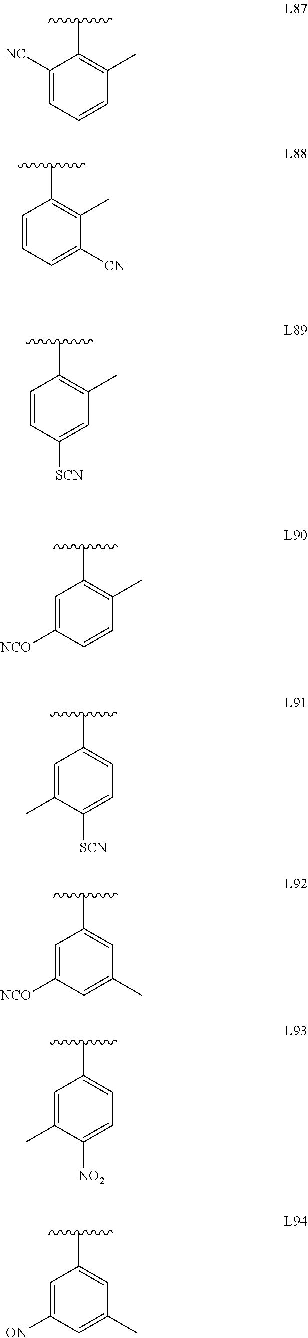 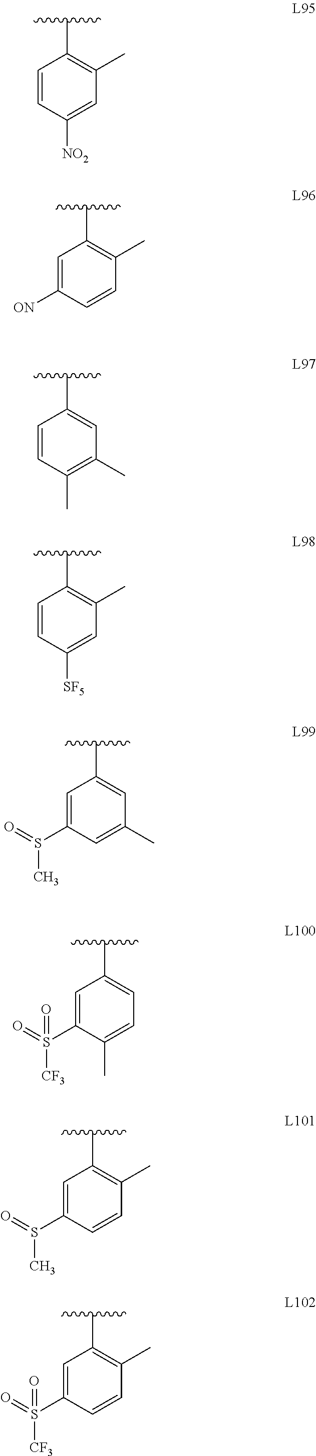 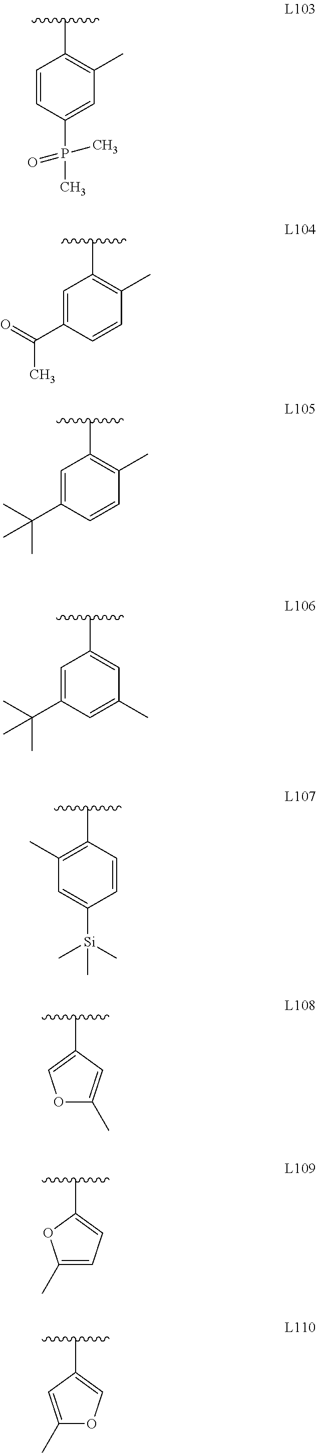 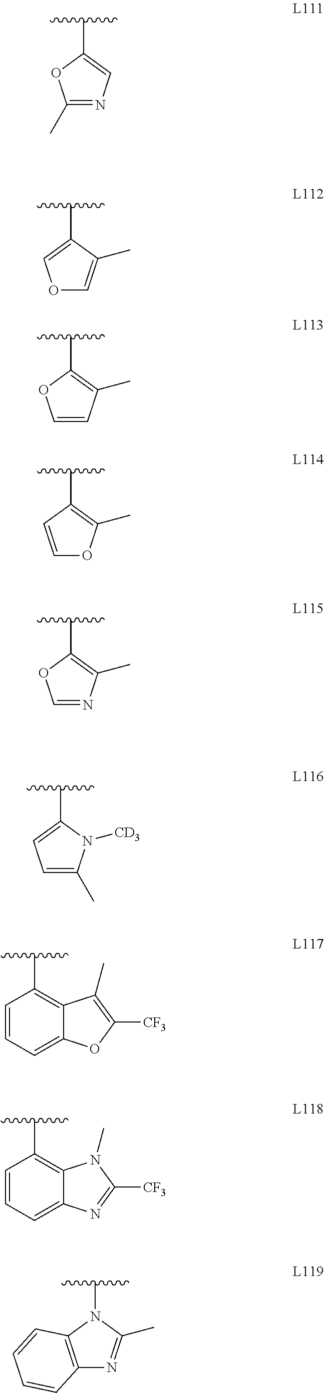 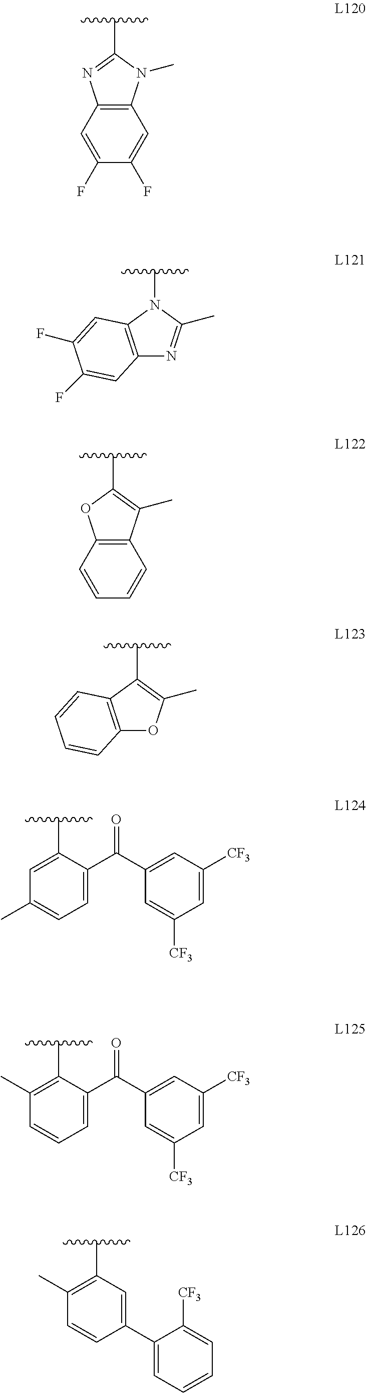 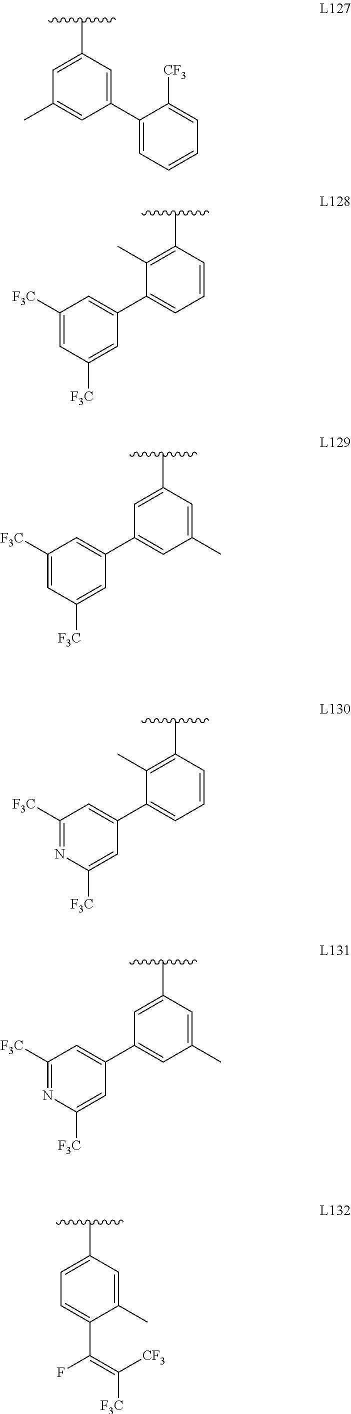 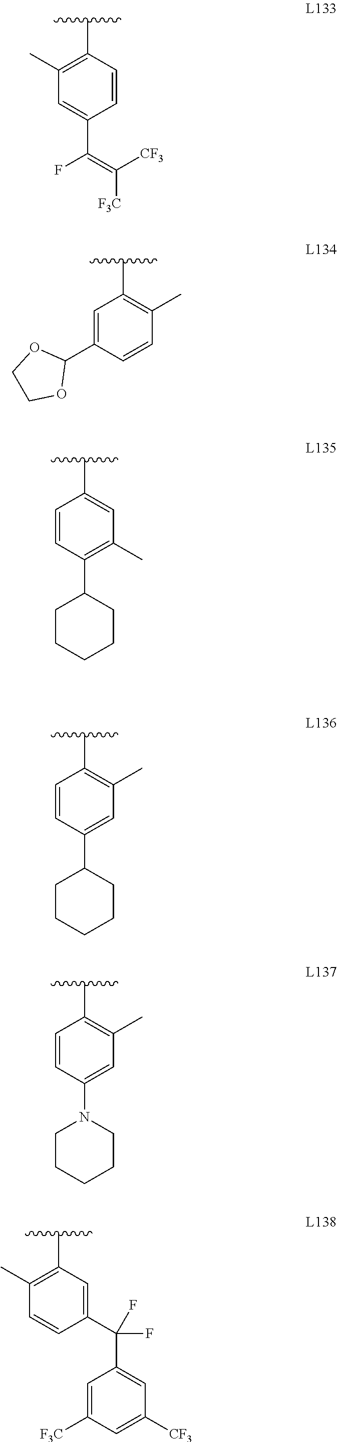 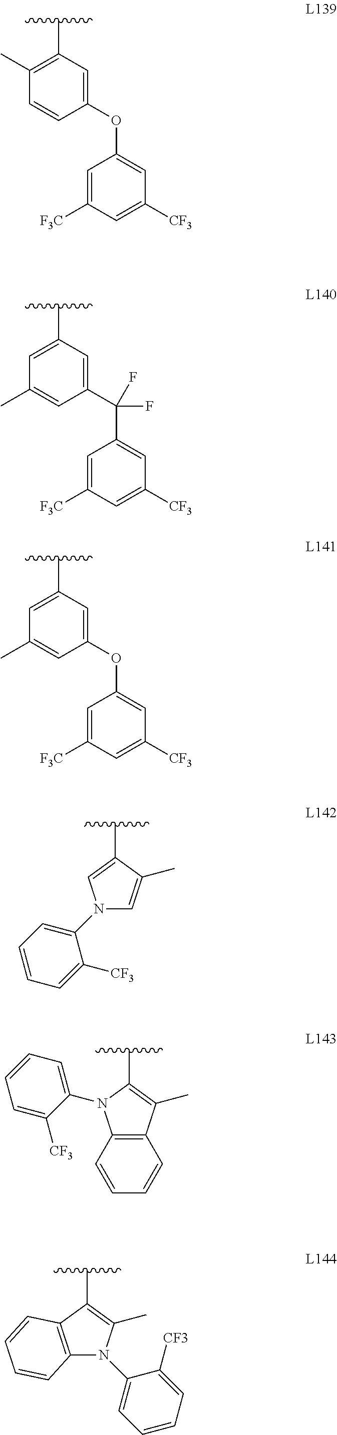 [SMILES: *c1cc(Oc2cc(C(F)(F)F)cc(C(F)(F)F)c2)ccc1C ; *c1cc(C)cc(C(F)(F)c2cc(C(F)(F)F)cc(C(F)(F)F)c2)c1 ; *c1cc(C)cc(Oc2cc(C(F)(F)F)cc(C(F)(F)F)c2)c1 ; *c1cn(-c2ccccc2C(F)(F)F)cc1C ; *c1c(C)c2ccccc2n1-c1ccccc1C(F)(F)F ; *c1c(C)n(-c2ccccc2C(F)(F)F)c2ccccc12] 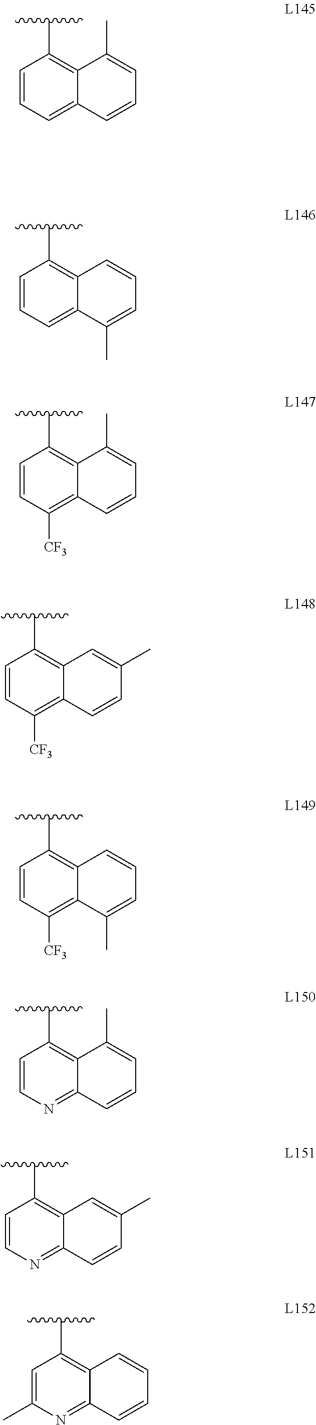 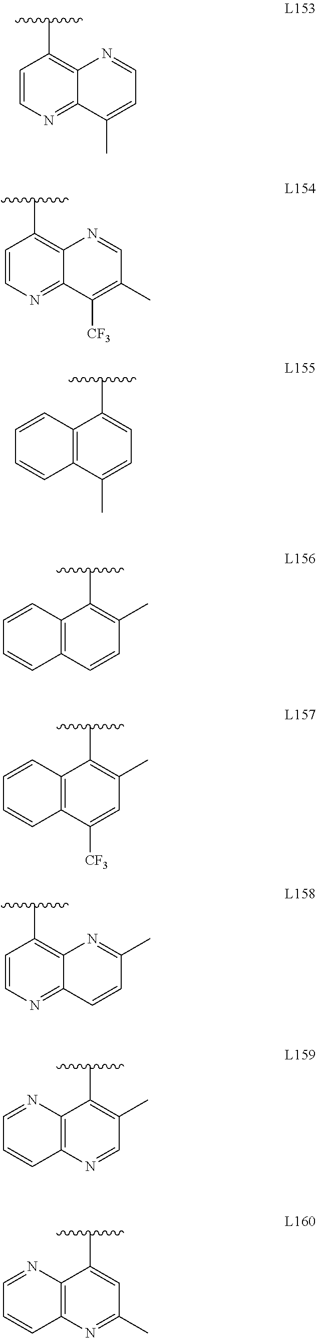 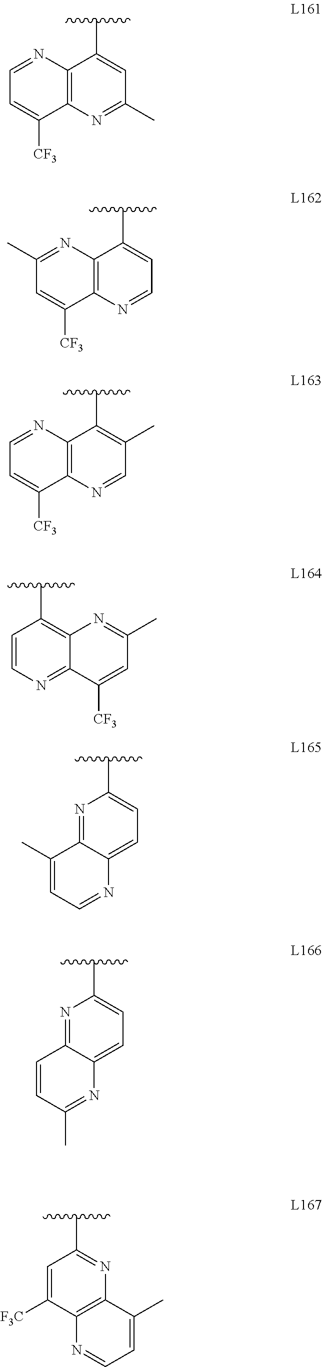 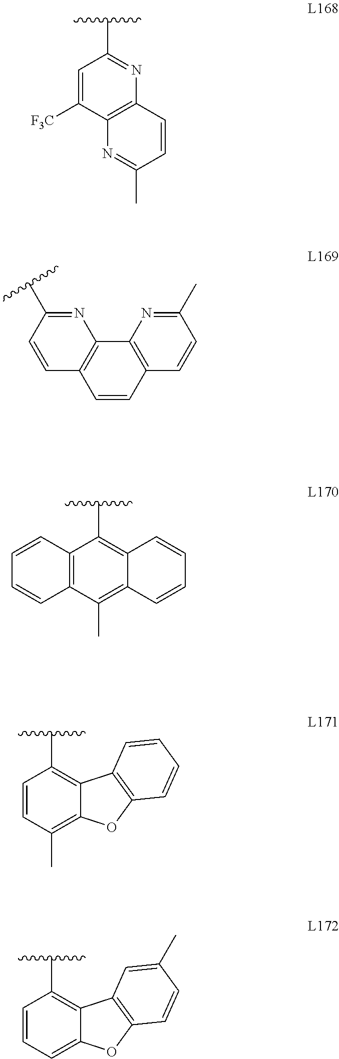 [SMILES: *c1cc(C(F)(F)F)c2nc(C)ccc2n1 ; *c1ccc2ccc3ccc(C)nc3c2n1 ; *c1c2ccccc2c(C)c2ccccc12 ; *c1ccc(C)c2oc3ccccc3c12 ; *c1cccc2oc3ccc(C)cc3c12]
hydrogens in the compounds with the above structures can be partially or completely replaced by deuterium; and
“ [symbol] ” represents the position of connection of L to formula I or another L, and “ [symbol] ” represents the position of connection of L to ring A or another L.

12. The compound according to claim 11, characterized in that Ar¹, Ar², R′, R″, R¹, R², R³, and R⁴ are, at each occurrence, the same or different and are selected from the group consisting of the following structures: [chemical structure] [chemical structure] 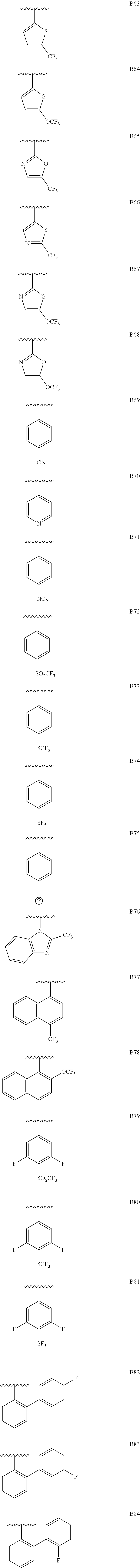 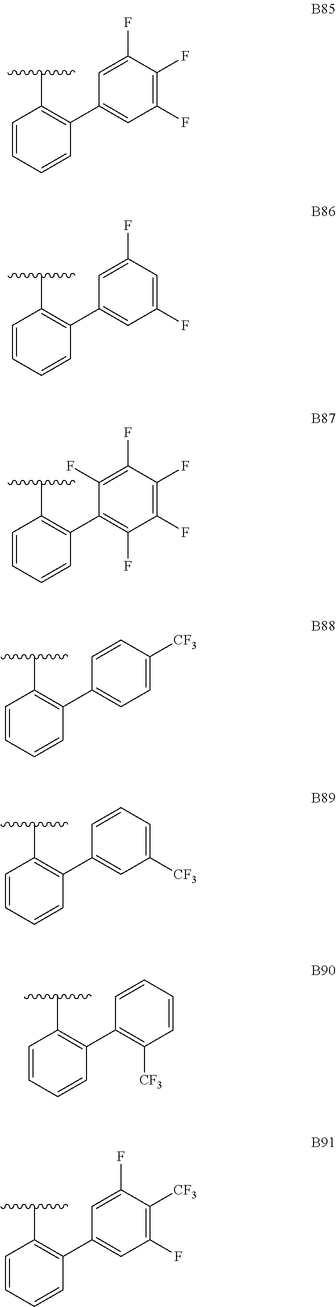 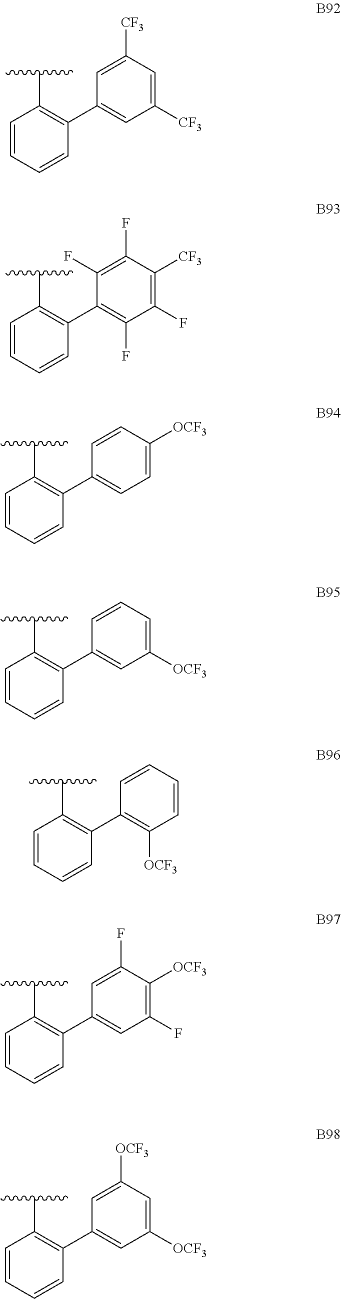 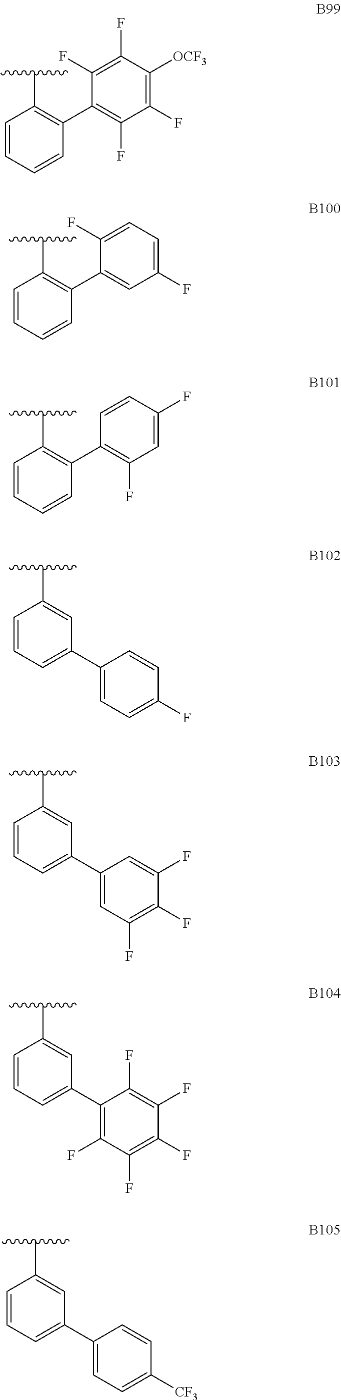 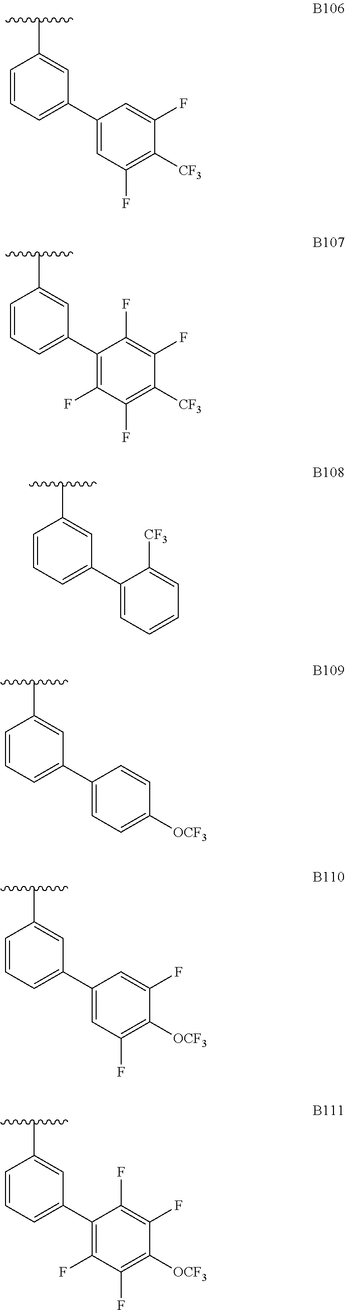 [SMILES: *c1cccc(-c2cc(F)c(C(F)(F)F)c(F)c2)c1 ; *c1cccc(-c2c(F)c(F)c(C(F)(F)F)c(F)c2F)c1 ; *c1cccc(-c2ccccc2C(F)(F)F)c1 ; *c1cccc(-c2ccc(OC(F)(F)F)cc2)c1 ; *c1cccc(-c2cc(F)c(OC(F)(F)F)c(F)c2)c1 ; *c1cccc(-c2c(F)c(F)c(OC(F)(F)F)c(F)c2F)c1] 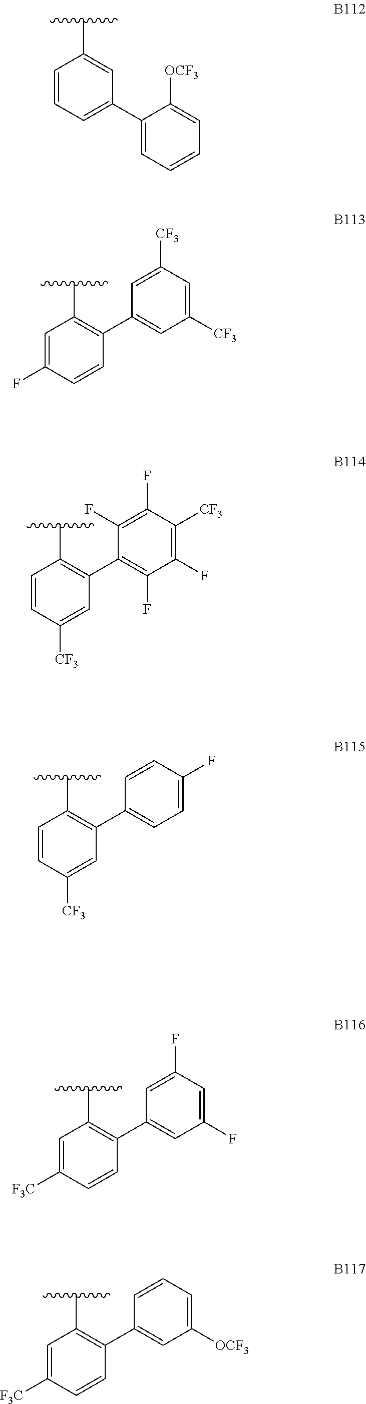 [SMILES: *c1cccc(-c2ccccc2OC(F)(F)F)c1 ; *c1cc(F)ccc1-c1cc(C(F)(F)F)cc(C(F)(F)F)c1 ; *c1ccc(C(F)(F)F)cc1-c1c(F)c(F)c(C(F)(F)F)c(F)c1F ; *c1ccc(C(F)(F)F)cc1-c1ccc(F)cc1 ; *c1cc(C(F)(F)F)ccc1-c1cc(F)cc(F)c1 ; *c1cc(C(F)(F)F)ccc1-c1cccc(OC(F)(F)F)c1] 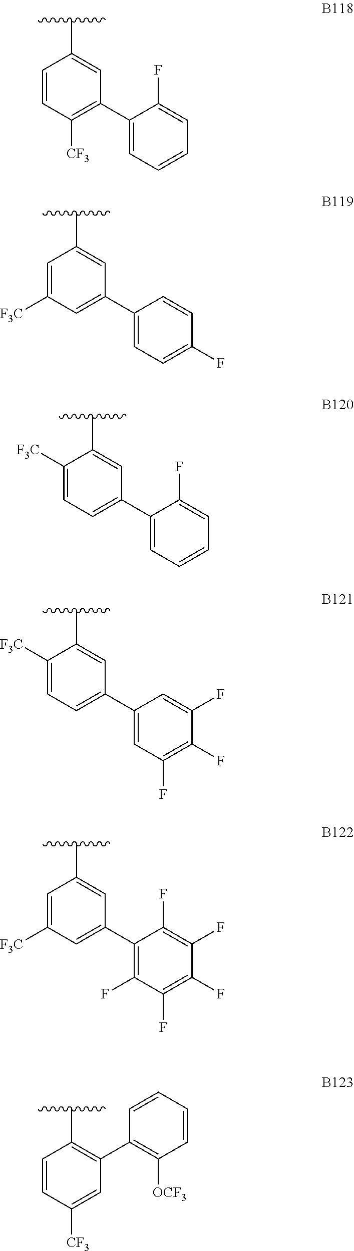 [SMILES: *c1ccc(C(F)(F)F)c(-c2ccccc2F)c1 ; *c1cc(-c2ccc(F)cc2)cc(C(F)(F)F)c1 ; *c1cc(-c2ccccc2F)ccc1C(F)(F)F ; *c1cc(-c2cc(F)c(F)c(F)c2)ccc1C(F)(F)F ; *c1cc(-c2c(F)c(F)c(F)c(F)c2F)cc(C(F)(F)F)c1 ; *c1ccc(C(F)(F)F)cc1-c1ccccc1OC(F)(F)F] 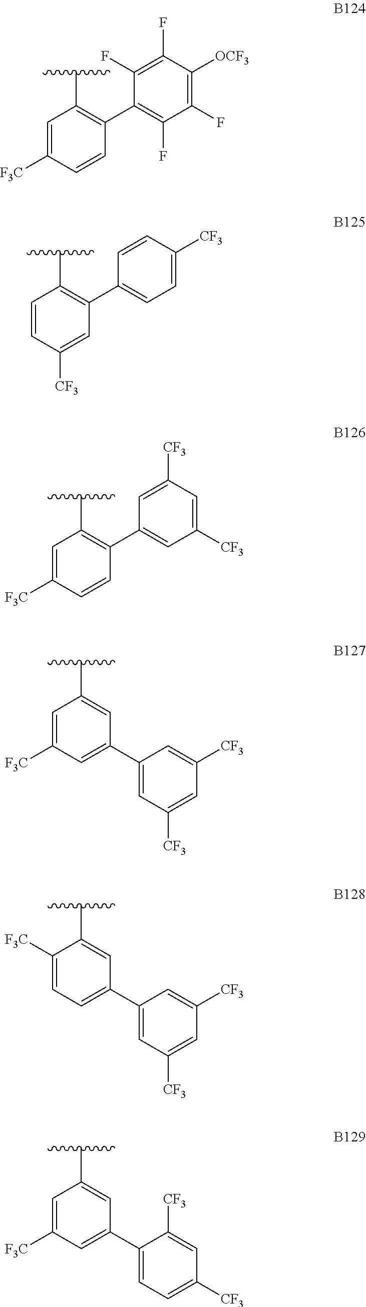 [SMILES: *c1cc(C(F)(F)F)ccc1-c1c(F)c(F)c(OC(F)(F)F)c(F)c1F ; *c1ccc(C(F)(F)F)cc1-c1ccc(C(F)(F)F)cc1 ; *c1cc(C(F)(F)F)ccc1-c1cc(C(F)(F)F)cc(C(F)(F)F)c1 ; *c1cc(-c2cc(C(F)(F)F)cc(C(F)(F)F)c2)cc(C(F)(F)F)c1 ; *c1cc(-c2cc(C(F)(F)F)cc(C(F)(F)F)c2)ccc1C(F)(F)F ; *c1cc(-c2ccc(C(F)(F)F)cc2C(F)(F)F)cc(C(F)(F)F)c1] 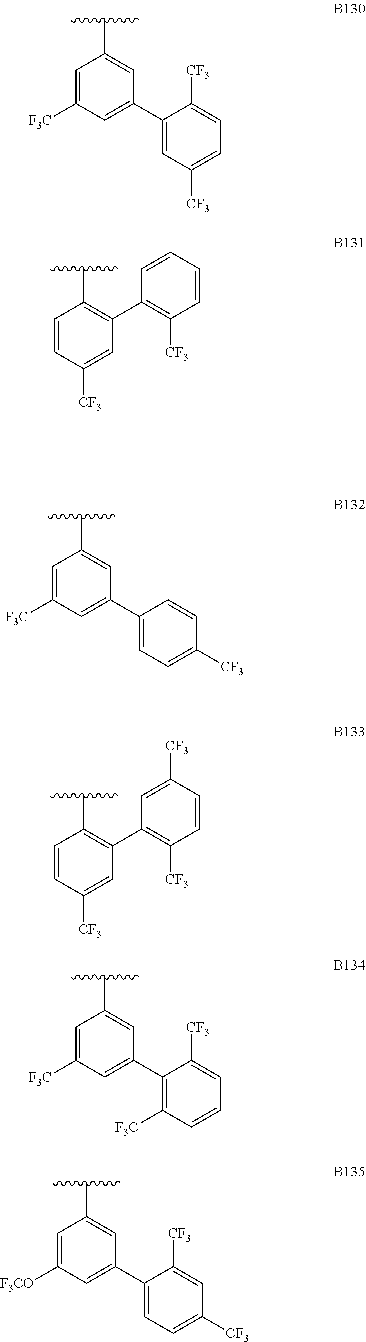 [SMILES: *c1cc(-c2cc(C(F)(F)F)ccc2C(F)(F)F)cc(C(F)(F)F)c1 ; *c1ccc(C(F)(F)F)cc1-c1ccccc1C(F)(F)F ; *c1cc(-c2ccc(C(F)(F)F)cc2)cc(C(F)(F)F)c1 ; *c1ccc(C(F)(F)F)cc1-c1cc(C(F)(F)F)ccc1C(F)(F)F ; *c1cc(-c2c(C(F)(F)F)cccc2C(F)(F)F)cc(C(F)(F)F)c1 ; *c1cc(OC(F)(F)F)cc(-c2ccc(C(F)(F)F)cc2C(F)(F)F)c1] 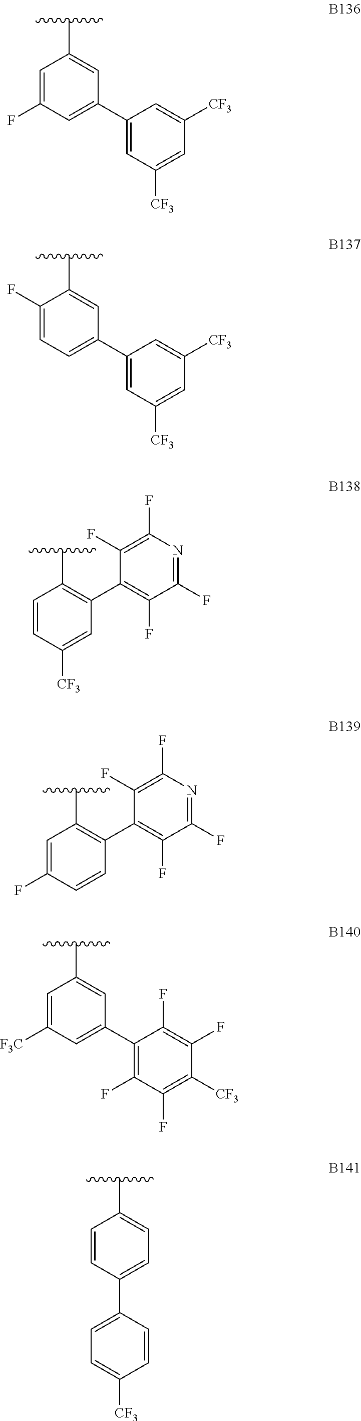 [SMILES: *c1cc(F)cc(-c2cc(C(F)(F)F)cc(C(F)(F)F)c2)c1 ; *c1cc(-c2cc(C(F)(F)F)cc(C(F)(F)F)c2)ccc1F ; *c1ccc(C(F)(F)F)cc1-c1c(F)c(F)nc(F)c1F ; *c1cc(F)ccc1-c1c(F)c(F)nc(F)c1F ; *c1cc(-c2c(F)c(F)c(C(F)(F)F)c(F)c2F)cc(C(F)(F)F)c1 ; *c1ccc(-c2ccc(C(F)(F)F)cc2)cc1] 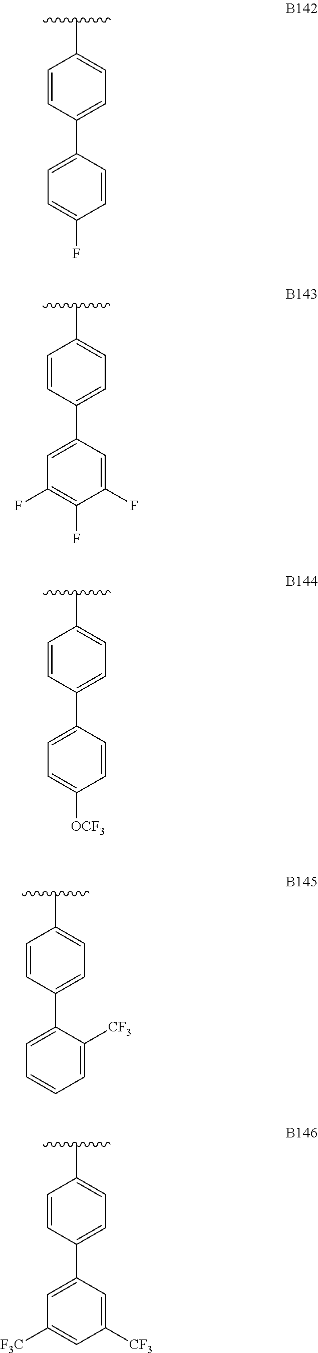 [SMILES: *c1ccc(-c2ccc(F)cc2)cc1 ; *c1ccc(-c2cc(F)c(F)c(F)c2)cc1 ; *c1ccc(-c2ccc(OC(F)(F)F)cc2)cc1 ; *c1ccc(-c2ccccc2C(F)(F)F)cc1 ; *c1ccc(-c2cc(C(F)(F)F)cc(C(F)(F)F)c2)cc1] 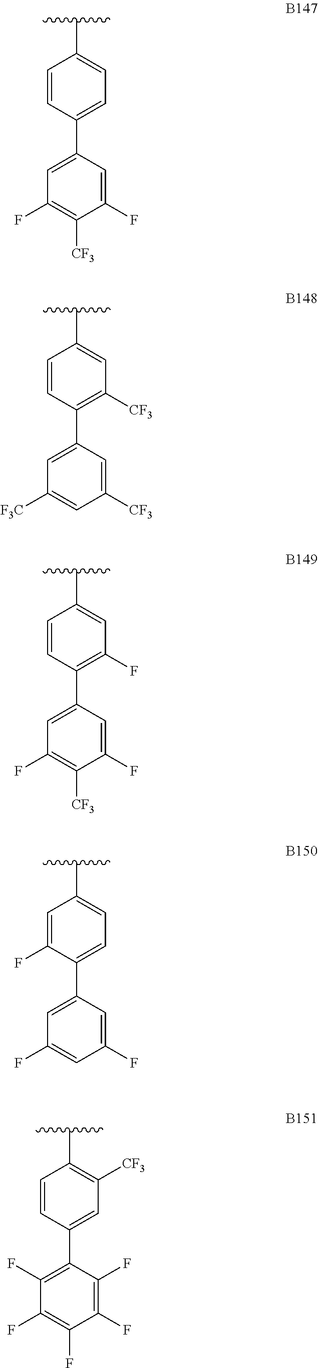 [SMILES: *c1ccc(-c2cc(F)c(C(F)(F)F)c(F)c2)cc1 ; *c1ccc(-c2cc(C(F)(F)F)cc(C(F)(F)F)c2)c(C(F)(F)F)c1 ; *c1ccc(-c2cc(F)c(C(F)(F)F)c(F)c2)c(F)c1 ; *c1ccc(-c2cc(F)cc(F)c2)c(F)c1 ; *c1ccc(-c2c(F)c(F)c(F)c(F)c2F)cc1C(F)(F)F] 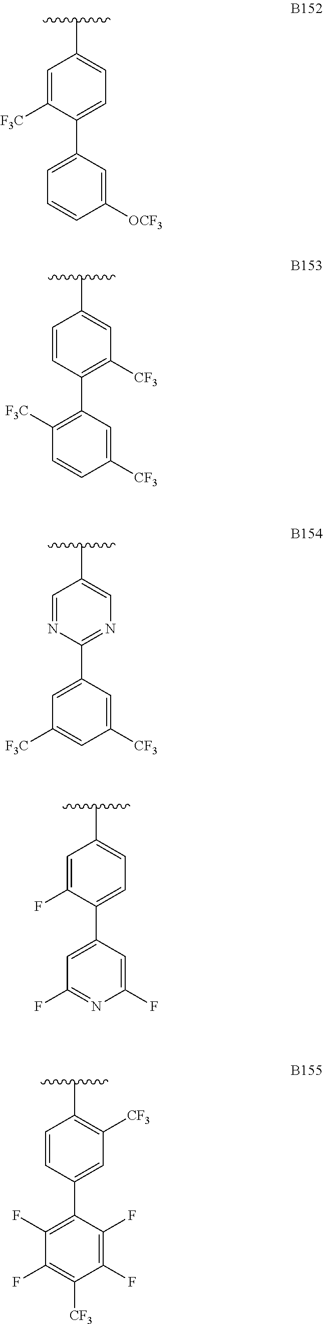 [SMILES: *c1ccc(-c2cccc(OC(F)(F)F)c2)c(C(F)(F)F)c1 ; *c1ccc(-c2cc(C(F)(F)F)ccc2C(F)(F)F)c(C(F)(F)F)c1 ; *c1cnc(-c2cc(C(F)(F)F)cc(C(F)(F)F)c2)nc1 ; *c1ccc(-c2cc(F)nc(F)c2)c(F)c1 ; *c1ccc(-c2c(F)c(F)c(C(F)(F)F)c(F)c2F)cc1C(F)(F)F] 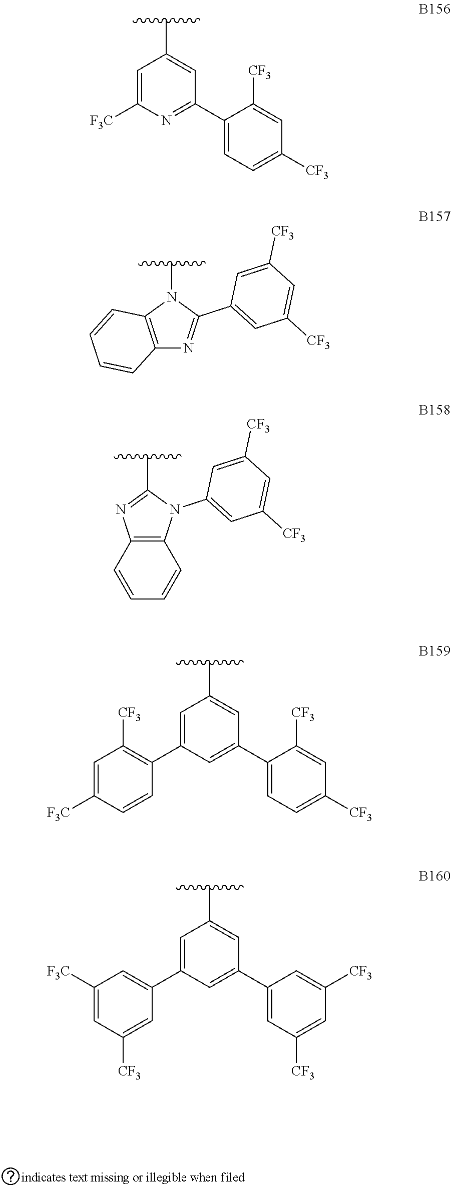 [SMILES: *c1cc(-c2ccc(C(F)(F)F)cc2C(F)(F)F)nc(C(F)(F)F)c1 ; *n1c(-c2cc(C(F)(F)F)cc(C(F)(F)F)c2)nc2ccccc21 ; *c1nc2ccccc2n1-c1cc(C(F)(F)F)cc(C(F)(F)F)c1 ; *c1cc(-c2ccc(C(F)(F)F)cc2C(F)(F)F)cc(-c2ccc(C(F)(F)F)cc2C(F)(F)F)c1 ; *c1cc(-c2cc(C(F)(F)F)cc(C(F)(F)F)c2)cc(-c2cc(C(F)(F)F)cc(C(F)(F)F)c2)c1]
hydrogens in the compounds with the above structures can be partially or completely replaced by deuterium; and
“ [symbol] ” represents the position of connection of the above structures to other structures.

13. The compound according to claim 12, characterized in that the organic compound is selected from the group consisting of compounds P1-1 to P1-1028, compounds P2-1 to P2-1028, compounds P3-1 to P3-1028, and compounds P4-1 to P4-1028;
the compounds P1-1 to P1-1028 have structures represented by formula I-1-1; wherein R¹ and R² are the same, and W, Ar¹, R¹, R², X, and L correspond to atoms or groups selected from the following table, respectively:

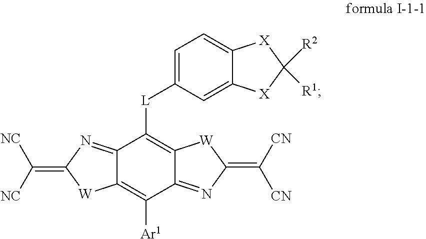

| Compound No. | W | Ar¹ | R¹ and R² | X | L |
|---|---|---|---|---|---|
| P1-1 | O | A2 | A1 | O | Single bond |
| P1-2 | O | A2 | A1 | O | O |
| P1-3 | O | A2 | A1 | O | L1 |
| P1-4 | O | A2 | A1 | O | L5 |
| P1-5 | O | A2 | A1 | O | L9 |
| P1-6 | O | A2 | A1 | O | L18 |
| P1-7 | O | A2 | A1 | O | L22 |
| P1-8 | O | A2 | A1 | O | L27 |
| P1-9 | O | A2 | A1 | O | L29 |
| P1-10 | O | A2 | A1 | O | L36 |
| P1-11 | O | A2 | A1 | O | L40 |
| P1-12 | O | A2 | A1 | O | L53 |
| P1-13 | O | A2 | A1 | O | L59 |
| P1-14 | O | A2 | A1 | O | L65 |
| P1-15 | O | A2 | A1 | O | L68 |
| P1-16 | O | A2 | A1 | O | L119 |
| P1-17 | O | A2 | A1 | O | L128 |
| P1-18 | O | A2 | A1 | O | L148 |
| P1-19 | O | B4 | A1 | O | Single bond |
| P1-20 | O | B4 | A1 | O | O |
| P1-21 | O | B4 | A1 | O | L1 |
| P1-22 | O | B4 | A1 | O | L5 |
| P1-23 | O | B4 | A1 | O | L9 |
| P1-24 | O | B4 | A1 | O | L18 |
| P1-25 | O | B4 | A1 | O | L22 |
| P1-26 | O | B4 | A1 | O | L27 |
| P1-27 | O | B4 | A1 | O | L29 |
| P1-28 | O | B4 | A1 | O | L36 |
| P1-29 | O | B4 | A1 | O | L40 |
| P1-30 | O | B4 | A1 | O | L53 |
| P1-31 | O | B4 | A1 | O | L59 |
| P1-32 | O | B4 | A1 | O | L65 |
| P1-33 | O | B4 | A1 | O | L68 |
| P1-34 | O | B4 | A1 | O | L119 |
| P1-35 | O | B4 | A1 | O | L128 |
| P1-36 | O | B4 | A1 | O | L148 |
| P1-37 | O | B23 | A1 | O | Single bond |
| P1-38 | O | B23 | A1 | O | O |
| P1-39 | O | B23 | A1 | O | L1 |
| P1-40 | O | B23 | A1 | O | L5 |
| P1-41 | O | B23 | A1 | O | L9 |
| P1-42 | O | B23 | A1 | O | L18 |
| P1-43 | O | B23 | A1 | O | L22 |
| P1-44 | O | B23 | A1 | O | L27 |
| P1-45 | O | B23 | A1 | O | L29 |
| P1-46 | O | B23 | A1 | O | L36 |
| P1-47 | O | B23 | A1 | O | L40 |
| P1-48 | O | B23 | A1 | O | L53 |
| P1-49 | O | B23 | A1 | O | L59 |
| P1-50 | O | B23 | A1 | O | L65 |
| P1-51 | O | B23 | A1 | O | L68 |
| P1-52 | O | B23 | A1 | O | L119 |
| P1-53 | O | B23 | A1 | O | L128 |
| P1-54 | O | B23 | A1 | O | L148 |
| P1-55 | O | B36 | A1 | O | Single bond |
| P1-56 | O | B36 | A1 | O | O |
| P1-57 | O | B36 | A1 | O | L1 |
| P1-58 | O | B36 | A1 | O | L5 |
| P1-59 | O | B36 | A1 | O | L9 |
| P1-60 | O | B36 | A1 | O | L18 |
| P1-61 | O | B36 | A1 | O | L22 |
| P1-62 | O | B36 | A1 | O | L27 |
| P1-63 | O | B36 | A1 | O | L29 |
| P1-64 | O | B36 | A1 | O | L36 |
| P1-65 | O | B36 | A1 | O | L40 |
| P1-66 | O | B36 | A1 | O | L53 |
| P1-67 | O | B36 | A1 | O | L59 |
| P1-68 | O | B36 | A1 | O | L65 |
| P1-69 | O | B36 | A1 | O | L68 |
| P1-70 | O | B36 | A1 | O | L119 |
| P1-71 | O | B36 | A1 | O | L128 |
| P1-72 | O | B36 | A1 | O | L148 |
| P1-73 | O | B44 | A1 | O | Single bond |
| P1-74 | O | B44 | A1 | O | O |
| P1-75 | O | B44 | A1 | O | L1 |
| P1-76 | O | B44 | A1 | O | L5 |
| P1-77 | O | B44 | A1 | O | L9 |
| P1-78 | O | B44 | A1 | O | L18 |
| P1-79 | O | B44 | A1 | O | L22 |
| P1-80 | O | B44 | A1 | O | L27 |
| P1-81 | O | B44 | A1 | O | L29 |
| P1-82 | O | B44 | A1 | O | L36 |
| P1-83 | O | B44 | A1 | O | L40 |
| P1-84 | O | B44 | A1 | O | L53 |
| P1-85 | O | B44 | A1 | O | L59 |
| P1-86 | O | B44 | A1 | O | L65 |
| P1-87 | O | B44 | A1 | O | L68 |
| P1-88 | O | B44 | A1 | O | L119 |
| P1-89 | O | B44 | A1 | O | L128 |
| P1-90 | O | B44 | A1 | O | L148 |
| P1-91 | O | B92 | A1 | O | Single bond |
| P1-92 | O | B92 | A1 | O | O |
| P1-93 | O | B92 | A1 | O | L1 |
| P1-94 | O | B92 | A1 | O | L5 |
| P1-95 | O | B92 | A1 | O | L9 |
| P1-96 | O | B92 | A1 | O | L18 |
| P1-97 | O | B92 | A1 | O | L22 |
| P1-98 | O | B92 | A1 | O | L27 |
| P1-99 | O | B92 | A1 | O | L29 |
| P1-100 | O | B92 | A1 | O | L36 |
| P1-101 | O | B92 | A1 | O | L40 |
| P1-102 | O | B92 | A1 | O | L53 |
| P1-103 | O | B92 | A1 | O | L59 |
| P1-104 | O | B92 | A1 | O | L65 |
| P1-105 | O | B92 | A1 | O | L68 |
| P1-106 | O | B92 | A1 | O | L119 |
| P1-107 | O | B92 | A1 | O | L128 |
| P1-108 | O | B92 | A1 | O | L148 |
| P1-109 | O | A2 | A2 | O | Single bond |
| P1-110 | O | A2 | A2 | O | O |
| P1-111 | O | A2 | A2 | O | L1 |
| P1-112 | O | A2 | A2 | O | L2 |
| P1-113 | O | A2 | A2 | O | L3 |
| P1-114 | O | A2 | A2 | O | L4 |
| P1-115 | O | A2 | A2 | O | L5 |
| P1-116 | O | A2 | A2 | O | L6 |
| P1-117 | O | A2 | A2 | O | L7 |
| P1-118 | O | A2 | A2 | O | L9 |
| P1-119 | O | A2 | A2 | O | L11 |
| P1-120 | O | A2 | A2 | O | L14 |
| P1-121 | O | A2 | A2 | O | L18 |
| P1-122 | O | A2 | A2 | O | L22 |
| P1-123 | O | A2 | A2 | O | L27 |
| P1-124 | O | A2 | A2 | O | L28 |
| P1-125 | O | A2 | A2 | O | L29 |
| P1-126 | O | A2 | A2 | O | L30 |
| P1-127 | O | A2 | A2 | O | L32 |
| P1-128 | O | A2 | A2 | O | L36 |
| P1-129 | O | A2 | A2 | O | L40 |
| P1-130 | O | A2 | A2 | O | L48 |
| P1-131 | O | A2 | A2 | O | L53 |
| P1-132 | O | A2 | A2 | O | L59 |
| P1-133 | O | A2 | A2 | O | L65 |
| P1-134 | O | A2 | A2 | O | L68 |
| P1-135 | O | A2 | A2 | O | L79 |
| P1-136 | O | A2 | A2 | O | L89 |
| P1-137 | O | A2 | A2 | O | L102 |
| P1-138 | O | A2 | A2 | O | L111 |
| P1-139 | O | A2 | A2 | O | L119 |
| P1-140 | O | A2 | A2 | O | L128 |
| P1-141 | O | A2 | A2 | O | L148 |
| P1-142 | O | A2 | A2 | O | L156 |
| P1-143 | O | A2 | A2 | O | L167 |
| P1-144 | O | A2 | A2 | O | L169 |
| P1-145 | O | A2 | A2 | O | L172 |
| P1-146 | O | B4 | A2 | O | Single bond |
| P1-147 | O | B4 | A2 | O | O |
| P1-148 | O | B4 | A2 | O | L1 |
| P1-149 | O | B4 | A2 | O | L2 |
| P1-150 | O | B4 | A2 | O | L3 |
| P1-151 | O | B4 | A2 | O | L4 |
| P1-152 | O | B4 | A2 | O | L5 |
| P1-153 | O | B4 | A2 | O | L6 |
| P1-154 | O | B4 | A2 | O | L7 |
| P1-155 | O | B4 | A2 | O | L9 |
| P1-156 | O | B4 | A2 | O | L11 |
| P1-157 | O | B4 | A2 | O | L14 |
| P1-158 | O | B4 | A2 | O | L18 |
| P1-159 | O | B4 | A2 | O | L22 |
| P1-160 | O | B4 | A2 | O | L27 |
| P1-161 | O | B4 | A2 | O | L28 |
| P1-162 | O | B4 | A2 | O | L29 |
| P1-163 | O | B4 | A2 | O | L30 |
| P1-164 | O | B4 | A2 | O | L32 |
| P1-165 | O | B4 | A2 | O | L36 |
| P1-166 | O | B4 | A2 | O | L40 |
| P1-167 | O | B4 | A2 | O | L48 |
| P1-168 | O | B4 | A2 | O | L53 |
| P1-169 | O | B4 | A2 | O | L59 |
| P1-170 | O | B4 | A2 | O | L65 |
| P1-171 | O | B4 | A2 | O | L68 |
| P1-172 | O | B4 | A2 | O | L79 |
| P1-173 | O | B4 | A2 | O | L89 |
| P1-174 | O | B4 | A2 | O | L102 |
| P1-175 | O | B4 | A2 | O | L111 |
| P1-176 | O | B4 | A2 | O | L119 |
| P1-177 | O | B4 | A2 | O | L128 |
| P1-178 | O | B4 | A2 | O | L148 |
| P1-179 | O | B4 | A2 | O | L156 |
| P1-180 | O | B4 | A2 | O | L167 |
| P1-181 | O | B4 | A2 | O | L169 |
| P1-182 | O | B4 | A2 | O | L172 |
| P1-183 | O | B23 | A2 | O | Single bond |
| P1-184 | O | B23 | A2 | O | O |
| P1-185 | O | B23 | A2 | O | L1 |
| P1-186 | O | B23 | A2 | O | L2 |
| P1-187 | O | B23 | A2 | O | L3 |
| P1-188 | O | B23 | A2 | O | L4 |
| P1-189 | O | B23 | A2 | O | L5 |
| P1-190 | O | B23 | A2 | O | L6 |
| P1-191 | O | B23 | A2 | O | L7 |
| P1-192 | O | B23 | A2 | O | L9 |
| P1-193 | O | B23 | A2 | O | L11 |
| P1-194 | O | B23 | A2 | O | L14 |
| P1-195 | O | B23 | A2 | O | L18 |
| P1-196 | O | B23 | A2 | O | L22 |
| P1-197 | O | B23 | A2 | O | L27 |
| P1-198 | O | B23 | A2 | O | L28 |
| P1-199 | O | B23 | A2 | O | L29 |
| P1-200 | O | B23 | A2 | O | L30 |
| P1-201 | O | B23 | A2 | O | L32 |
| P1-202 | O | B23 | A2 | O | L36 |
| P1-203 | O | B23 | A2 | O | L40 |
| P1-204 | O | B23 | A2 | O | L48 |
| P1-205 | O | B23 | A2 | O | L53 |
| P1-206 | O | B23 | A2 | O | L59 |
| P1-207 | O | B23 | A2 | O | L65 |
| P1-208 | O | B23 | A2 | O | L68 |
| P1-209 | O | B23 | A2 | O | L79 |
| P1-210 | O | B23 | A2 | O | L89 |
| P1-211 | O | B23 | A2 | O | L102 |
| P1-212 | O | B23 | A2 | O | L111 |
| P1-213 | O | B23 | A2 | O | L119 |
| P1-214 | O | B23 | A2 | O | L128 |
| P1-215 | O | B23 | A2 | O | L148 |
| P1-216 | O | B23 | A2 | O | L156 |
| P1-217 | O | B23 | A2 | O | L167 |
| P1-218 | O | B23 | A2 | O | L169 |
| P1-219 | O | B23 | A2 | O | L172 |
| P1-220 | O | B36 | A2 | O | Single bond |
| P1-221 | O | B36 | A2 | O | O |
| P1-222 | O | B36 | A2 | O | L1 |
| P1-223 | O | B36 | A2 | O | L2 |
| P1-224 | O | B36 | A2 | O | L3 |
| P1-225 | O | B36 | A2 | O | L4 |
| P1-226 | O | B36 | A2 | O | L5 |
| P1-227 | O | B36 | A2 | O | L6 |
| P1-228 | O | B36 | A2 | O | L7 |
| P1-229 | O | B36 | A2 | O | L9 |
| P1-230 | O | B36 | A2 | O | L11 |
| P1-231 | O | B36 | A2 | O | L14 |
| P1-232 | O | B36 | A2 | O | L18 |
| P1-233 | O | B36 | A2 | O | L22 |
| P1-234 | O | B36 | A2 | O | L27 |
| P1-235 | O | B36 | A2 | O | L28 |
| P1-236 | O | B36 | A2 | O | L29 |
| P1-237 | O | B36 | A2 | O | L30 |
| P1-238 | O | B36 | A2 | O | L32 |
| P1-239 | O | B36 | A2 | O | L36 |
| P1-240 | O | B36 | A2 | O | L40 |
| P1-241 | O | B36 | A2 | O | L48 |
| P1-242 | O | B36 | A2 | O | L53 |
| P1-243 | O | B36 | A2 | O | L59 |
| P1-244 | O | B36 | A2 | O | L65 |
| P1-245 | O | B36 | A2 | O | L68 |
| P1-246 | O | B36 | A2 | O | L79 |
| P1-247 | O | B36 | A2 | O | L89 |
| P1-248 | O | B36 | A2 | O | L102 |
| P1-249 | O | B36 | A2 | O | L111 |
| P1-250 | O | B36 | A2 | O | L119 |
| P1-251 | O | B36 | A2 | O | L128 |
| P1-252 | O | B36 | A2 | O | L148 |
| P1-253 | O | B36 | A2 | O | L156 |
| P1-254 | O | B36 | A2 | O | L167 |
| P1-255 | O | B36 | A2 | O | L169 |
| P1-256 | O | B36 | A2 | O | L172 |
| P1-257 | O | B44 | A2 | O | Single bond |
| P1-258 | O | B44 | A2 | O | O |
| P1-259 | O | B44 | A2 | O | L1 |
| P1-260 | O | B44 | A2 | O | L2 |
| P1-261 | O | B44 | A2 | O | L3 |
| P1-262 | O | B44 | A2 | O | L4 |
| P1-263 | O | B44 | A2 | O | L5 |
| P1-264 | O | B44 | A2 | O | L6 |
| P1-265 | O | B44 | A2 | O | L7 |
| P1-266 | O | B44 | A2 | O | L9 |
| P1-267 | O | B44 | A2 | O | L11 |
| P1-268 | O | B44 | A2 | O | L14 |
| P1-269 | O | B44 | A2 | O | L18 |
| P1-270 | O | B44 | A2 | O | L22 |
| P1-271 | O | B44 | A2 | O | L27 |
| P1-272 | O | B44 | A2 | O | L28 |
| P1-273 | O | B44 | A2 | O | L29 |
| P1-274 | O | B44 | A2 | O | L30 |
| P1-275 | O | B44 | A2 | O | L32 |
| P1-276 | O | B44 | A2 | O | L36 |
| P1-277 | O | B44 | A2 | O | L40 |
| P1-278 | O | B44 | A2 | O | L48 |
| P1-279 | O | B44 | A2 | O | L53 |
| P1-280 | O | B44 | A2 | O | L59 |
| P1-281 | O | B44 | A2 | O | L65 |
| P1-282 | O | B44 | A2 | O | L68 |
| P1-283 | O | B44 | A2 | O | L79 |
| P1-284 | O | B44 | A2 | O | L89 |
| P1-285 | O | B44 | A2 | O | L102 |
| P1-286 | O | B44 | A2 | O | L111 |
| P1-287 | O | B44 | A2 | O | L119 |
| P1-288 | O | B44 | A2 | O | L128 |
| P1-289 | O | B44 | A2 | O | L148 |
| P1-290 | O | B44 | A2 | O | L156 |
| P1-291 | O | B44 | A2 | O | L167 |
| P1-292 | O | B44 | A2 | O | L169 |
| P1-293 | O | B44 | A2 | O | L172 |
| P1-294 | O | B92 | A2 | O | Single bond |
| P1-295 | O | B92 | A2 | O | O |
| P1-296 | O | B92 | A2 | O | L1 |
| P1-297 | O | B92 | A2 | O | L2 |
| P1-298 | O | B92 | A2 | O | L3 |
| P1-299 | O | B92 | A2 | O | L4 |
| P1-300 | O | B92 | A2 | O | L5 |
| P1-301 | O | B92 | A2 | O | L6 |
| P1-302 | O | B92 | A2 | O | L7 |
| P1-303 | O | B92 | A2 | O | L9 |
| P1-304 | O | B92 | A2 | O | L11 |
| P1-305 | O | B92 | A2 | O | L14 |
| P1-306 | O | B92 | A2 | O | L18 |
| P1-307 | O | B92 | A2 | O | L22 |
| P1-308 | O | B92 | A2 | O | L27 |
| P1-309 | O | B92 | A2 | O | L28 |
| P1-310 | O | B92 | A2 | O | L29 |
| P1-311 | O | B92 | A2 | O | L30 |
| P1-312 | O | B92 | A2 | O | L32 |
| P1-313 | O | B92 | A2 | O | L36 |
| P1-314 | O | B92 | A2 | O | L40 |
| P1-315 | O | B92 | A2 | O | L48 |
| P1-316 | O | B92 | A2 | O | L53 |
| P1-317 | O | B92 | A2 | O | L59 |
| P1-318 | O | B92 | A2 | O | L65 |
| P1-319 | O | B92 | A2 | O | L68 |
| P1-320 | O | B92 | A2 | O | L79 |
| P1-321 | O | B92 | A2 | O | L89 |
| P1-322 | O | B92 | A2 | O | L102 |
| P1-323 | O | B92 | A2 | O | L111 |
| P1-324 | O | B92 | A2 | O | L119 |
| P1-325 | O | B92 | A2 | O | L128 |
| P1-326 | O | B92 | A2 | O | L148 |
| P1-327 | O | B92 | A2 | O | L156 |
| P1-328 | O | B92 | A2 | O | L167 |
| P1-329 | O | B92 | A2 | O | L169 |
| P1-330 | O | B92 | A2 | O | L172 |
| P1-331 | O | A2 | A3 | O | Single bond |
| P1-332 | O | A2 | A3 | O | O |
| P1-333 | O | A2 | A3 | O | L1 |
| P1-334 | O | A2 | A3 | O | L2 |
| P1-335 | O | A2 | A3 | O | L3 |
| P1-336 | O | A2 | A3 | O | L4 |
| P1-337 | O | A2 | A3 | O | L5 |
| P1-338 | O | A2 | A3 | O | L6 |
| P1-339 | O | A2 | A3 | O | L7 |
| P1-340 | O | A2 | A3 | O | L9 |
| P1-341 | O | A2 | A3 | O | L11 |
| P1-342 | O | A2 | A3 | O | L14 |
| P1-343 | O | A2 | A3 | O | L18 |
| P1-344 | O | A2 | A3 | O | L22 |
| P1-345 | O | A2 | A3 | O | L27 |
| P1-346 | O | A2 | A3 | O | L28 |
| P1-347 | O | A2 | A3 | O | L29 |
| P1-348 | O | A2 | A3 | O | L30 |
| P1-349 | O | A2 | A3 | O | L32 |
| P1-350 | O | A2 | A3 | O | L36 |
| P1-351 | O | A2 | A3 | O | L40 |
| P1-352 | O | A2 | A3 | O | L48 |
| P1-353 | O | A2 | A3 | O | L53 |
| P1-354 | O | A2 | A3 | O | L59 |
| P1-355 | O | A2 | A3 | O | L65 |
| P1-356 | O | A2 | A3 | O | L68 |
| P1-357 | O | A2 | A3 | O | L79 |
| P1-358 | O | A2 | A3 | O | L89 |
| P1-359 | O | A2 | A3 | O | L102 |
| P1-360 | O | A2 | A3 | O | L111 |
| P1-361 | O | A2 | A3 | O | L119 |
| P1-362 | O | A2 | A3 | O | L128 |
| P1-363 | O | A2 | A3 | O | L148 |
| P1-364 | O | A2 | A3 | O | L156 |
| P1-365 | O | A2 | A3 | O | L167 |
| P1-366 | O | A2 | A3 | O | L169 |
| P1-367 | O | A2 | A3 | O | L172 |
| P1-368 | O | B4 | A3 | O | Single bond |
| P1-369 | O | B4 | A3 | O | O |
| P1-370 | O | B4 | A3 | O | L1 |
| P1-371 | O | B4 | A3 | O | L2 |
| P1-372 | O | B4 | A3 | O | L3 |
| P1-373 | O | B4 | A3 | O | L4 |
| P1-374 | O | B4 | A3 | O | L5 |
| P1-375 | O | B4 | A3 | O | L6 |
| P1-376 | O | B4 | A3 | O | L7 |
| P1-377 | O | B4 | A3 | O | L9 |
| P1-378 | O | B4 | A3 | O | L11 |
| P1-379 | O | B4 | A3 | O | L14 |
| P1-380 | O | B4 | A3 | O | L18 |
| P1-381 | O | B4 | A3 | O | L22 |
| P1-382 | O | B4 | A3 | O | L27 |
| P1-383 | O | B4 | A3 | O | L28 |
| P1-384 | O | B4 | A3 | O | L29 |
| P1-385 | O | B4 | A3 | O | L30 |
| P1-386 | O | B4 | A3 | O | L32 |
| P1-387 | O | B4 | A3 | O | L36 |
| P1-388 | O | B4 | A3 | O | L40 |
| P1-389 | O | B4 | A3 | O | L48 |
| P1-390 | O | B4 | A3 | O | L53 |
| P1-391 | O | B4 | A3 | O | L59 |
| P1-392 | O | B4 | A3 | O | L65 |
| P1-393 | O | B4 | A3 | O | L68 |
| P1-394 | O | B4 | A3 | O | L79 |
| P1-395 | O | B4 | A3 | O | L89 |
| P1-396 | O | B4 | A3 | O | L102 |
| P1-397 | O | B4 | A3 | O | L111 |
| P1-398 | O | B4 | A3 | O | L119 |
| P1-399 | O | B4 | A3 | O | L128 |
| P1-400 | O | B4 | A3 | O | L148 |
| P1-401 | O | B4 | A3 | O | L156 |
| P1-402 | O | B4 | A3 | O | L167 |
| P1-403 | O | B4 | A3 | O | L169 |
| P1-404 | O | B4 | A3 | O | L172 |
| P1-405 | O | B23 | A3 | O | Single bond |
| P1-406 | O | B23 | A3 | O | O |
| P1-407 | O | B23 | A3 | O | L1 |
| P1-408 | O | B23 | A3 | O | L2 |
| P1-409 | O | B23 | A3 | O | L3 |
| P1-410 | O | B23 | A3 | O | L4 |
| P1-411 | O | B23 | A3 | O | L5 |
| P1-412 | O | B23 | A3 | O | L6 |
| P1-413 | O | B23 | A3 | O | L7 |
| P1-414 | O | B23 | A3 | O | L9 |
| P1-415 | O | B23 | A3 | O | L11 |
| P1-416 | O | B23 | A3 | O | L14 |
| P1-417 | O | B23 | A3 | O | L18 |
| P1-418 | O | B23 | A3 | O | L22 |
| P1-419 | O | B23 | A3 | O | L27 |
| P1-420 | O | B23 | A3 | O | L28 |
| P1-421 | O | B23 | A3 | O | L29 |
| P1-422 | O | B23 | A3 | O | L30 |
| P1-423 | O | B23 | A3 | O | L32 |
| P1-424 | O | B23 | A3 | O | L36 |
| P1-425 | O | B23 | A3 | O | L40 |
| P1-426 | O | B23 | A3 | O | L48 |
| P1-427 | O | B23 | A3 | O | L53 |
| P1-428 | O | B23 | A3 | O | L59 |
| P1-429 | O | B23 | A3 | O | L65 |
| P1-430 | O | B23 | A3 | O | L68 |
| P1-431 | O | B23 | A3 | O | L79 |
| P1-432 | O | B23 | A3 | O | L89 |
| P1-433 | O | B23 | A3 | O | L102 |
| P1-434 | O | B23 | A3 | O | L111 |
| P1-435 | O | B23 | A3 | O | L119 |
| P1-436 | O | B23 | A3 | O | L128 |
| P1-437 | O | B23 | A3 | O | L148 |
| P1-438 | O | B23 | A3 | O | L156 |
| P1-439 | O | B23 | A3 | O | L167 |
| P1-440 | O | B23 | A3 | O | L169 |
| P1-441 | O | B23 | A3 | O | L172 |
| P1-442 | O | B36 | A3 | O | Single bond |
| P1-443 | O | B36 | A3 | O | O |
| P1-444 | O | B36 | A3 | O | L1 |
| P1-445 | O | B36 | A3 | O | L2 |
| P1-446 | O | B36 | A3 | O | L3 |
| P1-447 | O | B36 | A3 | O | L4 |
| P1-448 | O | B36 | A3 | O | L5 |
| P1-449 | O | B36 | A3 | O | L6 |
| P1-450 | O | B36 | A3 | O | L7 |
| P1-451 | O | B36 | A3 | O | L9 |
| P1-452 | O | B36 | A3 | O | L11 |
| P1-453 | O | B36 | A3 | O | L14 |
| P1-454 | O | B36 | A3 | O | L18 |
| P1-455 | O | B36 | A3 | O | L22 |
| P1-456 | O | B36 | A3 | O | L27 |
| P1-457 | O | B36 | A3 | O | L28 |
| P1-458 | O | B36 | A3 | O | L29 |
| P1-459 | O | B36 | A3 | O | L30 |
| P1-460 | O | B36 | A3 | O | L32 |
| P1-461 | O | B36 | A3 | O | L36 |
| P1-462 | O | B36 | A3 | O | L40 |
| P1-463 | O | B36 | A3 | O | L48 |
| P1-464 | O | B36 | A3 | O | L53 |
| P1-465 | O | B36 | A3 | O | L59 |
| P1-466 | O | B36 | A3 | O | L65 |
| P1-467 | O | B36 | A3 | O | L68 |
| P1-468 | O | B36 | A3 | O | L79 |
| P1-469 | O | B36 | A3 | O | L89 |
| P1-470 | O | B36 | A3 | O | L102 |
| P1-471 | O | B36 | A3 | O | L111 |
| P1-472 | O | B36 | A3 | O | L119 |
| P1-473 | O | B36 | A3 | O | L128 |
| P1-474 | O | B36 | A3 | O | L148 |
| P1-475 | O | B36 | A3 | O | L156 |
| P1-476 | O | B36 | A3 | O | L167 |
| P1-477 | O | B36 | A3 | O | L169 |
| P1-478 | O | B36 | A3 | O | L172 |
| P1-479 | O | B44 | A3 | O | Single bond |
| P1-480 | O | B44 | A3 | O | O |
| P1-481 | O | B44 | A3 | O | L1 |
| P1-482 | O | B44 | A3 | O | L2 |
| P1-483 | O | B44 | A3 | O | L3 |
| P1-484 | O | B44 | A3 | O | L4 |
| P1-485 | O | B44 | A3 | O | L5 |
| P1-486 | O | B44 | A3 | O | L6 |
| P1-487 | O | B44 | A3 | O | L7 |
| P1-488 | O | B44 | A3 | O | L9 |
| P1-489 | O | B44 | A3 | O | L11 |
| P1-490 | O | B44 | A3 | O | L14 |
| P1-491 | O | B44 | A3 | O | L18 |
| P1-492 | O | B44 | A3 | O | L22 |
| P1-493 | O | B44 | A3 | O | L27 |
| P1-494 | O | B44 | A3 | O | L28 |
| P1-495 | O | B44 | A3 | O | L29 |
| P1-496 | O | B44 | A3 | O | L30 |
| P1-497 | O | B44 | A3 | O | L32 |
| P1-498 | O | B44 | A3 | O | L36 |
| P1-499 | O | B44 | A3 | O | L40 |
| P1-500 | O | B44 | A3 | O | L48 |
| P1-501 | O | B44 | A3 | O | L53 |
| P1-502 | O | B44 | A3 | O | L59 |
| P1-503 | O | B44 | A3 | O | L65 |
| P1-504 | O | B44 | A3 | O | L68 |
| P1-505 | O | B44 | A3 | O | L79 |
| P1-506 | O | B44 | A3 | O | L89 |
| P1-507 | O | B44 | A3 | O | L102 |
| P1-508 | O | B44 | A3 | O | L111 |
| P1-509 | O | B44 | A3 | O | L119 |
| P1-510 | O | B44 | A3 | O | L128 |
| P1-511 | O | B44 | A3 | O | L148 |
| P1-512 | O | B44 | A3 | O | L156 |
| P1-513 | O | B44 | A3 | O | L167 |
| P1-514 | O | B44 | A3 | O | L169 |
| P1-515 | O | B44 | A3 | O | L172 |
| P1-516 | O | B92 | A3 | O | Single bond |
| P1-517 | O | B92 | A3 | O | O |
| P1-518 | O | B92 | A3 | O | L1 |
| P1-519 | O | B92 | A3 | O | L2 |
| P1-520 | O | B92 | A3 | O | L3 |
| P1-521 | O | B92 | A3 | O | L4 |
| P1-522 | O | B92 | A3 | O | L5 |
| P1-523 | O | B92 | A3 | O | L6 |
| P1-524 | O | B92 | A3 | O | L7 |
| P1-525 | O | B92 | A3 | O | L9 |
| P1-526 | O | B92 | A3 | O | L11 |
| P1-527 | O | B92 | A3 | O | L14 |
| P1-528 | O | B92 | A3 | O | L18 |
| P1-529 | O | B92 | A3 | O | L22 |
| P1-530 | O | B92 | A3 | O | L27 |
| P1-531 | O | B92 | A3 | O | L28 |
| P1-532 | O | B92 | A3 | O | L29 |
| P1-533 | O | B92 | A3 | O | L30 |
| P1-534 | O | B92 | A3 | O | L32 |
| P1-535 | O | B92 | A3 | O | L36 |
| P1-536 | O | B92 | A3 | O | L40 |
| P1-537 | O | B92 | A3 | O | L48 |
| P1-538 | O | B92 | A3 | O | L53 |
| P1-539 | O | B92 | A3 | O | L59 |
| P1-540 | O | B92 | A3 | O | L65 |
| P1-541 | O | B92 | A3 | O | L68 |
| P1-542 | O | B92 | A3 | O | L79 |
| P1-543 | O | B92 | A3 | O | L89 |
| P1-544 | O | B92 | A3 | O | L102 |
| P1-545 | O | B92 | A3 | O | L111 |
| P1-546 | O | B92 | A3 | O | L119 |
| P1-547 | O | B92 | A3 | O | L128 |
| P1-548 | O | B92 | A3 | O | L148 |
| P1-549 | O | B92 | A3 | O | L156 |
| P1-550 | O | B92 | A3 | O | L167 |
| P1-551 | O | B92 | A3 | O | L169 |
| P1-552 | O | B92 | A3 | O | L172 |
| P1-553 | O | A2 | A12 | O | Single bond |
| P1-554 | O | A2 | A12 | O | O |
| P1-555 | O | A2 | A12 | O | L1 |
| P1-556 | O | A2 | A12 | O | L5 |
| P1-557 | O | A2 | A12 | O | L9 |
| P1-558 | O | A2 | A12 | O | L18 |
| P1-559 | O | A2 | A12 | O | L22 |
| P1-560 | O | A2 | A12 | O | L27 |
| P1-561 | O | A2 | A12 | O | L29 |
| P1-562 | O | A2 | A12 | O | L36 |
| P1-563 | O | A2 | A12 | O | L40 |
| P1-564 | O | A2 | A12 | O | L53 |
| P1-565 | O | A2 | A12 | O | L59 |
| P1-566 | O | A2 | A12 | O | L65 |
| P1-567 | O | A2 | A12 | O | L68 |
| P1-568 | O | A2 | A12 | O | L119 |
| P1-569 | O | A2 | A12 | O | L128 |
| P1-570 | O | A2 | A12 | O | L148 |
| P1-571 | O | B4 | A12 | O | Single bond |
| P1-572 | O | B4 | A12 | O | O |
| P1-573 | O | B4 | A12 | O | L1 |
| P1-574 | O | B4 | A12 | O | L5 |
| P1-575 | O | B4 | A12 | O | L9 |
| P1-576 | O | B4 | A12 | O | L18 |
| P1-577 | O | B4 | A12 | O | L22 |
| P1-578 | O | B4 | A12 | O | L27 |
| P1-579 | O | B4 | A12 | O | L29 |
| P1-580 | O | B4 | A12 | O | L36 |
| P1-581 | O | B4 | A12 | O | L40 |
| P1-582 | O | B4 | A12 | O | L53 |
| P1-583 | O | B4 | A12 | O | L59 |
| P1-584 | O | B4 | A12 | O | L65 |
| P1-585 | O | B4 | A12 | O | L68 |
| P1-586 | O | B4 | A12 | O | L119 |
| P1-587 | O | B4 | A12 | O | L128 |
| P1-588 | O | B4 | A12 | O | L148 |
| P1-589 | O | B23 | A12 | O | Single bond |
| P1-590 | O | B23 | A12 | O | O |
| P1-591 | O | B23 | A12 | O | L1 |
| P1-592 | O | B23 | A12 | O | L5 |
| P1-593 | O | B23 | A12 | O | L9 |
| P1-594 | O | B23 | A12 | O | L18 |
| P1-595 | O | B23 | A12 | O | L22 |
| P1-596 | O | B23 | A12 | O | L27 |
| P1-597 | O | B23 | A12 | O | L29 |
| P1-598 | O | B23 | A12 | O | L36 |
| P1-599 | O | B23 | A12 | O | L40 |
| P1-600 | O | B23 | A12 | O | L53 |
| P1-601 | O | B23 | A12 | O | L59 |
| P1-602 | O | B23 | A12 | O | L65 |
| P1-603 | O | B23 | A12 | O | L68 |
| P1-604 | O | B23 | A12 | O | L119 |
| P1-605 | O | B23 | A12 | O | L128 |
| P1-606 | O | B23 | A12 | O | L148 |
| P1-607 | O | B36 | A12 | O | Single bond |
| P1-608 | O | B36 | A12 | O | O |
| P1-609 | O | B36 | A12 | O | L1 |
| P1-610 | O | B36 | A12 | O | L5 |
| P1-611 | O | B36 | A12 | O | L9 |
| P1-612 | O | B36 | A12 | O | L18 |
| P1-613 | O | B36 | A12 | O | L22 |
| P1-614 | O | B36 | A12 | O | L27 |
| P1-615 | O | B36 | A12 | O | L29 |
| P1-616 | O | B36 | A12 | O | L36 |
| P1-617 | O | B36 | A12 | O | L40 |
| P1-618 | O | B36 | A12 | O | L53 |
| P1-619 | O | B36 | A12 | O | L59 |
| P1-620 | O | B36 | A12 | O | L65 |
| P1-621 | O | B36 | A12 | O | L68 |
| P1-622 | O | B36 | A12 | O | L119 |
| P1-623 | O | B36 | A12 | O | L128 |
| P1-624 | O | B36 | A12 | O | L148 |
| P1-625 | O | B44 | A12 | O | Single bond |
| P1-626 | O | B44 | A12 | O | O |
| P1-627 | O | B44 | A12 | O | L1 |
| P1-628 | O | B44 | A12 | O | L5 |
| P1-629 | O | B44 | A12 | O | L9 |
| P1-630 | O | B44 | A12 | O | L18 |
| P1-631 | O | B44 | A12 | O | L22 |
| P1-632 | O | B44 | A12 | O | L27 |
| P1-633 | O | B44 | A12 | O | L29 |
| P1-634 | O | B44 | A12 | O | L36 |
| P1-635 | O | B44 | A12 | O | L40 |
| P1-636 | O | B44 | A12 | O | L53 |
| P1-637 | O | B44 | A12 | O | L59 |
| P1-638 | O | B44 | A12 | O | L65 |
| P1-639 | O | B44 | A12 | O | L68 |
| P1-640 | O | B44 | A12 | O | L119 |
| P1-641 | O | B44 | A12 | O | L128 |
| P1-642 | O | B44 | A12 | O | L148 |
| P1-643 | O | B92 | A12 | O | Single bond |
| P1-644 | O | B92 | A12 | O | O |
| P1-645 | O | B92 | A12 | O | L1 |
| P1-646 | O | B92 | A12 | O | L5 |
| P1-647 | O | B92 | A12 | O | L9 |
| P1-648 | O | B92 | A12 | O | L18 |
| P1-649 | O | B92 | A12 | O | L22 |
| P1-650 | O | B92 | A12 | O | L27 |
| P1-651 | O | B92 | A12 | O | L29 |
| P1-652 | O | B92 | A12 | O | L36 |
| P1-653 | O | B92 | A12 | O | L40 |
| P1-654 | O | B92 | A12 | O | L53 |
| P1-655 | O | B92 | A12 | O | L59 |
| P1-656 | O | B92 | A12 | O | L65 |
| P1-657 | O | B92 | A12 | O | L68 |
| P1-658 | O | B92 | A12 | O | L119 |
| P1-659 | O | B92 | A12 | O | L128 |
| P1-660 | O | B92 | A12 | O | L148 |
| P1-661 | O | A2 | B41 | O | Single bond |
| P1-662 | O | A2 | B41 | O | O |
| P1-663 | O | A2 | B41 | O | L1 |
| P1-664 | O | A2 | B41 | O | L5 |
| P1-665 | O | A2 | B41 | O | L9 |
| P1-666 | O | A2 | B41 | O | L18 |
| P1-667 | O | A2 | B41 | O | L22 |
| P1-668 | O | A2 | B41 | O | L27 |
| P1-669 | O | A2 | B41 | O | L29 |
| P1-670 | O | A2 | B41 | O | L36 |
| P1-671 | O | A2 | B41 | O | L40 |
| P1-672 | O | A2 | B41 | O | L53 |
| P1-673 | O | A2 | B41 | O | L59 |
| P1-674 | O | A2 | B41 | O | L65 |
| P1-675 | O | A2 | B41 | O | L68 |
| P1-676 | O | A2 | B41 | O | L119 |
| P1-677 | O | A2 | B41 | O | L128 |
| P1-678 | O | A2 | B41 | O | L148 |
| P1-679 | O | B4 | B41 | O | Single bond |
| P1-680 | O | B4 | B41 | O | O |
| P1-681 | O | B4 | B41 | O | L1 |
| P1-682 | O | B4 | B41 | O | L5 |
| P1-683 | O | B4 | B41 | O | L9 |
| P1-684 | O | B4 | B41 | O | L18 |
| P1-685 | O | B4 | B41 | O | L22 |
| P1-686 | O | B4 | B41 | O | L27 |
| P1-687 | O | B4 | B41 | O | L29 |
| P1-688 | O | B4 | B41 | O | L36 |
| P1-689 | O | B4 | B41 | O | L40 |
| P1-690 | O | B4 | B41 | O | L53 |
| P1-691 | O | B4 | B41 | O | L59 |
| P1-692 | O | B4 | B41 | O | L65 |
| P1-693 | O | B4 | B41 | O | L68 |
| P1-694 | O | B4 | B41 | O | L119 |
| P1-695 | O | B4 | B41 | O | L128 |
| P1-696 | O | B4 | B41 | O | L148 |
| P1-697 | O | B23 | B41 | O | Single bond |
| P1-698 | O | B23 | B41 | O | O |
| P1-699 | O | B23 | B41 | O | L1 |
| P1-700 | O | B23 | B41 | O | L5 |
| P1-701 | O | B23 | B41 | O | L9 |
| P1-702 | O | B23 | B41 | O | L18 |
| P1-703 | O | B23 | B41 | O | L22 |
| P1-704 | O | B23 | B41 | O | L27 |
| P1-705 | O | B23 | B41 | O | L29 |
| P1-706 | O | B23 | B41 | O | L36 |
| P1-707 | O | B23 | B41 | O | L40 |
| P1-708 | O | B23 | B41 | O | L53 |
| P1-709 | O | B23 | B41 | O | L59 |
| P1-710 | O | B23 | B41 | O | L65 |
| P1-711 | O | B23 | B41 | O | L68 |
| P1-712 | O | B23 | B41 | O | L119 |
| P1-713 | O | B23 | B41 | O | L128 |
| P1-714 | O | B23 | B41 | O | L148 |
| P1-715 | O | B36 | B41 | O | Single bond |
| P1-716 | O | B36 | B41 | O | O |
| P1-717 | O | B36 | B41 | O | L1 |
| P1-718 | O | B36 | B41 | O | L5 |
| P1-719 | O | B36 | B41 | O | L9 |
| P1-720 | O | B36 | B41 | O | L18 |
| P1-721 | O | B36 | B41 | O | L22 |
| P1-722 | O | B36 | B41 | O | L27 |
| P1-723 | O | B36 | B41 | O | L29 |
| P1-724 | O | B36 | B41 | O | L36 |
| P1-725 | O | B36 | B41 | O | L40 |
| P1-726 | O | B36 | B41 | O | L53 |
| P1-727 | O | B36 | B41 | O | L59 |
| P1-728 | O | B36 | B41 | O | L65 |
| P1-729 | O | B36 | B41 | O | L68 |
| P1-730 | O | B36 | B41 | O | L119 |
| P1-731 | O | B36 | B41 | O | L128 |
| P1-732 | O | B36 | B41 | O | L148 |
| P1-733 | O | B44 | B41 | O | Single bond |
| P1-734 | O | B44 | B41 | O | O |
| P1-735 | O | B44 | B41 | O | L1 |
| P1-736 | O | B44 | B41 | O | L5 |
| P1-737 | O | B44 | B41 | O | L9 |
| P1-738 | O | B44 | B41 | O | L18 |
| P1-739 | O | B44 | B41 | O | L22 |
| P1-740 | O | B44 | B41 | O | L27 |
| P1-741 | O | B44 | B41 | O | L29 |
| P1-742 | O | B44 | B41 | O | L36 |
| P1-743 | O | B44 | B41 | O | L40 |
| P1-744 | O | B44 | B41 | O | L53 |
| P1-745 | O | B44 | B41 | O | L59 |
| P1-746 | O | B44 | B41 | O | L65 |
| P1-747 | O | B44 | B41 | O | L68 |
| P1-748 | O | B44 | B41 | O | L119 |
| P1-749 | O | B44 | B41 | O | L128 |
| P1-750 | O | B44 | B41 | O | L148 |
| P1-751 | O | B92 | B41 | O | Single bond |
| P1-752 | O | B92 | B41 | O | O |
| P1-753 | O | B92 | B41 | O | L1 |
| P1-754 | O | B92 | B41 | O | L5 |
| P1-755 | O | B92 | B41 | O | L9 |
| P1-756 | O | B92 | B41 | O | L18 |
| P1-757 | O | B92 | B41 | O | L22 |
| P1-758 | O | B92 | B41 | O | L27 |
| P1-759 | O | B92 | B41 | O | L29 |
| P1-760 | O | B92 | B41 | O | L36 |
| P1-761 | O | B92 | B41 | O | L40 |
| P1-762 | O | B92 | B41 | O | L53 |
| P1-763 | O | B92 | B41 | O | L59 |
| P1-764 | O | B92 | B41 | O | L65 |
| P1-765 | O | B92 | B41 | O | L68 |
| P1-766 | O | B92 | B41 | O | L119 |
| P1-767 | O | B92 | B41 | O | L128 |
| P1-768 | O | B92 | B41 | O | L148 |
| P1-769 | O | A2 | B56 | O | Single bond |
| P1-770 | O | A2 | B56 | O | O |
| P1-771 | O | A2 | B56 | O | L1 |
| P1-772 | O | A2 | B56 | O | L5 |
| P1-773 | O | A2 | B56 | O | L9 |
| P1-774 | O | A2 | B56 | O | L18 |
| P1-775 | O | A2 | B56 | O | L22 |
| P1-776 | O | A2 | B56 | O | L27 |
| P1-777 | O | A2 | B56 | O | L29 |
| P1-778 | O | A2 | B56 | O | L36 |
| P1-779 | O | A2 | B56 | O | L40 |
| P1-780 | O | A2 | B56 | O | L53 |
| P1-781 | O | A2 | B56 | O | L59 |
| P1-782 | O | A2 | B56 | O | L65 |
| P1-783 | O | A2 | B56 | O | L68 |
| P1-784 | O | A2 | B56 | O | L119 |
| P1-785 | O | A2 | B56 | O | L128 |
| P1-786 | O | A2 | B56 | O | L148 |
| P1-787 | O | B4 | B56 | O | Single bond |
| P1-788 | O | B4 | B56 | O | O |
| P1-789 | O | B4 | B56 | O | L1 |
| P1-790 | O | B4 | B56 | O | L5 |
| P1-791 | O | B4 | B56 | O | L9 |
| P1-792 | O | B4 | B56 | O | L18 |
| P1-793 | O | B4 | B56 | O | L22 |
| P1-794 | O | B4 | B56 | O | L27 |
| P1-795 | O | B4 | B56 | O | L29 |
| P1-796 | O | B4 | B56 | O | L36 |
| P1-797 | O | B4 | B56 | O | L40 |
| P1-798 | O | B4 | B56 | O | L53 |
| P1-799 | O | B4 | B56 | O | L59 |
| P1-800 | O | B4 | B56 | O | L65 |
| P1-801 | O | B4 | B56 | O | L68 |
| P1-802 | O | B4 | B56 | O | L119 |
| P1-803 | O | B4 | B56 | O | L128 |
| P1-804 | O | B4 | B56 | O | L148 |
| P1-805 | O | B23 | B56 | O | Single bond |
| P1-806 | O | B23 | B56 | O | O |
| P1-807 | O | B23 | B56 | O | L1 |
| P1-808 | O | B23 | B56 | O | L5 |
| P1-809 | O | B23 | B56 | O | L9 |
| P1-810 | O | B23 | B56 | O | L18 |
| P1-811 | O | B23 | B56 | O | L22 |
| P1-812 | O | B23 | B56 | O | L27 |
| P1-813 | O | B23 | B56 | O | L29 |
| P1-814 | O | B23 | B56 | O | L36 |
| P1-815 | O | B23 | B56 | O | L40 |
| P1-816 | O | B23 | B56 | O | L53 |
| P1-817 | O | B23 | B56 | O | L59 |
| P1-818 | O | B23 | B56 | O | L65 |
| P1-819 | O | B23 | B56 | O | L68 |
| P1-820 | O | B23 | B56 | O | L119 |
| P1-821 | O | B23 | B56 | O | L128 |
| P1-822 | O | B23 | B56 | O | L148 |
| P1-823 | O | B36 | B56 | O | Single bond |
| P1-824 | O | B36 | B56 | O | O |
| P1-825 | O | B36 | B56 | O | L1 |
| P1-826 | O | B36 | B56 | O | L5 |
| P1-827 | O | B36 | B56 | O | L9 |
| P1-828 | O | B36 | B56 | O | L18 |
| P1-829 | O | B36 | B56 | O | L22 |
| P1-830 | O | B36 | B56 | O | L27 |
| P1-831 | O | B36 | B56 | O | L29 |
| P1-832 | O | B36 | B56 | O | L36 |
| P1-833 | O | B36 | B56 | O | L40 |
| P1-834 | O | B36 | B56 | O | L53 |
| P1-835 | O | B36 | B56 | O | L59 |
| P1-836 | O | B36 | B56 | O | L65 |
| P1-837 | O | B36 | B56 | O | L68 |
| P1-838 | O | B36 | B56 | O | L119 |
| P1-839 | O | B36 | B56 | O | L128 |
| P1-840 | O | B36 | B56 | O | L148 |
| P1-841 | O | B44 | B56 | O | Single bond |
| P1-842 | O | B44 | B56 | O | O |
| P1-843 | O | B44 | B56 | O | L1 |
| P1-844 | O | B44 | B56 | O | L5 |
| P1-845 | O | B44 | B56 | O | L9 |
| P1-846 | O | B44 | B56 | O | L18 |
| P1-847 | O | B44 | B56 | O | L22 |
| P1-848 | O | B44 | B56 | O | L27 |
| P1-849 | O | B44 | B56 | O | L29 |
| P1-850 | O | B44 | B56 | O | L36 |
| P1-851 | O | B44 | B56 | O | L40 |
| P1-852 | O | B44 | B56 | O | L53 |
| P1-853 | O | B44 | B56 | O | L59 |
| P1-854 | O | B44 | B56 | O | L65 |
| P1-855 | O | B44 | B56 | O | L68 |
| P1-856 | O | B44 | B56 | O | L119 |
| P1-857 | O | B44 | B56 | O | L128 |
| P1-858 | O | B44 | B56 | O | L148 |
| P1-859 | O | B92 | B56 | O | Single bond |
| P1-860 | O | B92 | B56 | O | O |
| P1-861 | O | B92 | B56 | O | L1 |
| P1-862 | O | B92 | B56 | O | L5 |
| P1-863 | O | B92 | B56 | O | L9 |
| P1-864 | O | B92 | B56 | O | L18 |
| P1-865 | O | B92 | B56 | O | L22 |
| P1-866 | O | B92 | B56 | O | L27 |
| P1-867 | O | B92 | B56 | O | L29 |
| P1-868 | O | B92 | B56 | O | L36 |
| P1-869 | O | B92 | B56 | O | L40 |
| P1-870 | O | B92 | B56 | O | L53 |
| P1-871 | O | B92 | B56 | O | L59 |
| P1-872 | O | B92 | B56 | O | L65 |
| P1-873 | O | B92 | B56 | O | L68 |
| P1-874 | O | B92 | B56 | O | L119 |
| P1-875 | O | B92 | B56 | O | L128 |
| P1-876 | O | B92 | B56 | O | L148 |
| P1-877 | O | B44 | A2 | S | Single bond |
| P1-878 | O | B44 | A2 | S | L5 |
| P1-879 | O | B44 | A2 | S | L6 |
| P1-880 | O | B44 | A2 | S | L27 |
| P1-881 | O | B44 | A2 | S | L29 |
| P1-882 | O | B44 | A2 | S | L36 |
| P1-883 | O | B92 | A2 | S | Single bond |
| P1-884 | O | B92 | A2 | S | L5 |
| P1-885 | O | B92 | A2 | S | L6 |
| P1-886 | O | B92 | A2 | S | L27 |
| P1-887 | O | B92 | A2 | S | L29 |
| P1-888 | O | B92 | A2 | S | L36 |
| P1-889 | O | B44 | A3 | S | Single bond |
| P1-890 | O | B44 | A3 | S | L5 |
| P1-891 | O | B44 | A3 | S | L6 |
| P1-892 | O | B44 | A3 | S | L27 |
| P1-893 | O | B44 | A3 | S | L29 |
| P1-894 | O | B44 | A3 | S | L36 |
| P1-895 | O | B92 | A3 | S | Single bond |
| P1-896 | O | B92 | A3 | S | L5 |
| P1-897 | O | B92 | A3 | S | L6 |
| P1-898 | O | B92 | A3 | S | L27 |
| P1-899 | O | B92 | A3 | S | L29 |
| P1-900 | O | B92 | A3 | S | L36 |
| P1-901 | O | B44 | A2 | Se | L6 |
| P1-902 | O | B44 | A2 | Se | L36 |
| P1-903 | O | B92 | A2 | Se | L6 |
| P1-904 | O | B92 | A2 | Se | L36 |
| P1-905 | O | B44 | A3 | Se | L6 |
| P1-906 | O | B44 | A3 | Se | L36 |
| P1-907 | O | B92 | A3 | Se | L6 |
| P1-908 | O | B92 | A3 | Se | L36 |
| P1-909 | O | B44 | A2 | S═O | L6 |
| P1-910 | O | B44 | A2 | S═O | L36 |
| P1-911 | O | B92 | A2 | S═O | L6 |
| P1-912 | O | B92 | A2 | S═O | L36 |
| P1-913 | O | B44 | A3 | S═O | L6 |
| P1-914 | O | B44 | A3 | S═O | L36 |
| P1-915 | O | B92 | A3 | S═O | L6 |
| P1-916 | O | B92 | A3 | S═O | L36 |
| P1-917 | O | B44 | A2 | O═S═O | Single bond |
| P1-918 | O | B44 | A2 | O═S═O | L5 |
| P1-919 | O | B44 | A2 | O═S═O | L6 |
| P1-920 | O | B44 | A2 | O═S═O | L27 |
| P1-921 | O | B44 | A2 | O═S═O | L29 |
| P1-922 | O | B44 | A2 | O═S═O | L36 |
| P1-923 | O | B92 | A2 | O═S═O | Single bond |
| P1-924 | O | B92 | A2 | O═S═O | L5 |
| P1-925 | O | B92 | A2 | O═S═O | L6 |
| P1-926 | O | B92 | A2 | O═S═O | L27 |
| P1-927 | O | B92 | A2 | O═S═O | L29 |
| P1-928 | O | B92 | A2 | O═S═O | L36 |
| P1-929 | O | B44 | A3 | O═S═O | Single bond |
| P1-930 | O | B44 | A3 | O═S═O | L5 |
| P1-931 | O | B44 | A3 | O═S═O | L6 |
| P1-932 | O | B44 | A3 | O═S═O | L27 |
| P1-933 | O | B44 | A3 | O═S═O | L29 |
| P1-934 | O | B44 | A3 | O═S═O | L36 |
| P1-935 | O | B92 | A3 | O═S═O | Single bond |
| P1-936 | O | B92 | A3 | O═S═O | L5 |
| P1-937 | O | B92 | A3 | O═S═O | L6 |
| P1-938 | O | B92 | A3 | O═S═O | L27 |
| P1-939 | O | B92 | A3 | O═S═O | L29 |
| P1-940 | O | B92 | A3 | O═S═O | L36 |
| P1-941 | S | B44 | A2 | O | Single bond |
| P1-942 | S | B44 | A2 | O | L5 |
| P1-943 | S | B44 | A2 | O | L6 |
| P1-944 | S | B44 | A2 | O | L27 |
| P1-945 | S | B44 | A2 | O | L29 |
| P1-946 | S | B44 | A2 | O | L36 |
| P1-947 | S | B92 | A2 | O | Single bond |
| P1-948 | S | B92 | A2 | O | L5 |
| P1-949 | S | B92 | A2 | O | L6 |
| P1-950 | S | B92 | A2 | O | L27 |
| P1-951 | S | B92 | A2 | O | L29 |
| P1-952 | S | B92 | A2 | O | L36 |
| P1-953 | S | B44 | A3 | O | Single bond |
| P1-954 | S | B44 | A3 | O | L5 |
| P1-955 | S | B44 | A3 | O | L6 |
| P1-956 | S | B44 | A3 | O | L27 |
| P1-957 | S | B44 | A3 | O | L29 |
| P1-958 | S | B44 | A3 | O | L36 |
| P1-959 | S | B92 | A3 | O | Single bond |
| P1-960 | S | B92 | A3 | O | L5 |
| P1-961 | S | B92 | A3 | O | L6 |
| P1-962 | S | B92 | A3 | O | L27 |
| P1-963 | S | B92 | A3 | O | L29 |
| P1-964 | S | B92 | A3 | O | L36 |
| P1-965 | Se | B44 | A2 | O | L5 |
| P1-966 | Se | B44 | A2 | O | L6 |
| P1-967 | Se | B44 | A2 | O | L27 |
| P1-968 | Se | B44 | A2 | O | L29 |
| P1-969 | Se | B44 | A2 | O | L36 |
| P1-970 | Se | B92 | A2 | O | L5 |
| P1-971 | Se | B92 | A2 | O | L6 |
| P1-972 | Se | B92 | A2 | O | L27 |
| P1-973 | Se | B92 | A2 | O | L29 |
| P1-974 | Se | B92 | A2 | O | L36 |
| P1-975 | Se | B44 | A3 | O | L5 |
| P1-976 | Se | B44 | A3 | O | L6 |
| P1-977 | Se | B44 | A3 | O | L27 |
| P1-978 | Se | B44 | A3 | O | L29 |
| P1-979 | Se | B44 | A3 | O | L36 |
| P1-980 | Se | B92 | A3 | O | L5 |
| P1-981 | Se | B92 | A3 | O | L6 |
| P1-982 | Se | B92 | A3 | O | L27 |
| P1-983 | Se | B92 | A3 | O | L29 |
| P1-984 | Se | B92 | A3 | O | L36 |
| P1-985 | S | B44 | A2 | O═S═O | Single bond |
| P1-986 | S | B44 | A2 | O═S═O | L5 |
| P1-987 | S | B44 | A2 | O═S═O | L6 |
| P1-988 | S | B44 | A2 | O═S═O | L27 |
| P1-989 | S | B44 | A2 | O═S═O | L29 |
| P1-990 | S | B44 | A2 | O═S═O | L36 |
| P1-991 | S | B92 | A2 | O═S═O | Single bond |
| P1-992 | S | B92 | A2 | O═S═O | L5 |
| P1-993 | S | B92 | A2 | O═S═O | L6 |
| P1-994 | S | B92 | A2 | O═S═O | L27 |
| P1-995 | S | B92 | A2 | O═S═O | L29 |
| P1-996 | S | B92 | A2 | O═S═O | L36 |
| P1-997 | S | B44 | A3 | O═S═O | Single bond |
| P1-998 | S | B44 | A3 | O═S═O | L5 |
| P1-999 | S | B44 | A3 | O═S═O | L6 |
| P1-1000 | S | B44 | A3 | O═S═O | L27 |
| P1-1001 | S | B44 | A3 | O═S═O | L29 |
| P1-1002 | S | B44 | A3 | O═S═O | L36 |
| P1-1003 | S | B92 | A3 | O═S═O | Single bond |
| P1-1004 | S | B92 | A3 | O═S═O | L5 |
| P1-1005 | S | B92 | A3 | O═S═O | L6 |
| P1-1006 | S | B92 | A3 | O═S═O | L27 |
| P1-1007 | S | B92 | A3 | O═S═O | L29 |
| P1-1008 | S | B92 | A3 | O═S═O | L36 |
| P1-1009 | Se | B44 | A2 | O═S═O | L5 |
| P1-1010 | Se | B44 | A2 | O═S═O | L6 |
| P1-1011 | Se | B44 | A2 | O═S═O | L27 |
| P1-1012 | Se | B44 | A2 | O═S═O | L29 |
| P1-1013 | Se | B44 | A2 | O═S═O | L36 |
| P1-1014 | Se | B92 | A2 | O═S═O | L5 |
| P1-1015 | Se | B92 | A2 | O═S═O | L6 |
| P1-1016 | Se | B92 | A2 | O═S═O | L27 |
| P1-1017 | Se | B92 | A2 | O═S═O | L29 |
| P1-1018 | Se | B92 | A2 | O═S═O | L36 |
| P1-1019 | Se | B44 | A3 | O═S═O | L5 |
| P1-1020 | Se | B44 | A3 | O═S═O | L6 |
| P1-1021 | Se | B44 | A3 | O═S═O | L27 |
| P1-1022 | Se | B44 | A3 | O═S═O | L29 |
| P1-1023 | Se | B44 | A3 | O═S═O | L36 |
| P1-1024 | Se | B92 | A3 | O═S═O | L5 |
| P1-1025 | Se | B92 | A3 | O═S═O | L6 |
| P1-1026 | Se | B92 | A3 | O═S═O | L27 |
| P1-1027 | Se | B92 | A3 | O═S═O | L29 |
| P1-1028 | Se | B92 | A3 | O═S═O | L36 |

the compounds P2-1 to P2-1028 have structures represented by formula I-1-2; wherein R¹ and R² are the same, and W, Ar¹, n¹, R², X, and L correspond to atoms or groups selected from the following table, respectively:

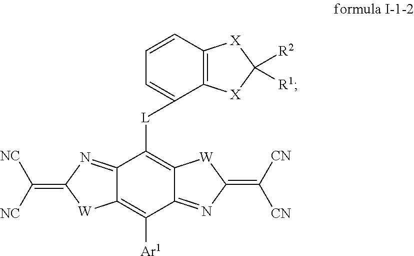

| Compound No. | W | Ar¹ | R¹ and R² | X | L |
|---|---|---|---|---|---|
| P2-1 | O | A2 | A1 | O | Single bond |
| P2-2 | O | A2 | A1 | O | O |
| P2-3 | O | A2 | A1 | O | L1 |
| P2-4 | O | A2 | A1 | O | L5 |
| P2-5 | O | A2 | A1 | O | L9 |
| P2-6 | O | A2 | A1 | O | L18 |
| P2-7 | O | A2 | A1 | O | L22 |
| P2-8 | O | A2 | A1 | O | L27 |
| P2-9 | O | A2 | A1 | O | L29 |
| P2-10 | O | A2 | A1 | O | L36 |
| P2-11 | O | A2 | A1 | O | L40 |
| P2-12 | O | A2 | A1 | O | L53 |
| P2-13 | O | A2 | A1 | O | L59 |
| P2-14 | O | A2 | A1 | O | L65 |
| P2-15 | O | A2 | A1 | O | L68 |
| P2-16 | O | A2 | A1 | O | L119 |
| P2-17 | O | A2 | A1 | O | L128 |
| P2-18 | O | A2 | A1 | O | L148 |
| P2-19 | O | B4 | A1 | O | Single bond |
| P2-20 | O | B4 | A1 | O | O |
| P2-21 | O | B4 | A1 | O | L1 |
| P2-22 | O | B4 | A1 | O | L5 |
| P2-23 | O | B4 | A1 | O | L9 |
| P2-24 | O | B4 | A1 | O | L18 |
| P2-25 | O | B4 | A1 | O | L22 |
| P2-26 | O | B4 | A1 | O | L27 |
| P2-27 | O | B4 | A1 | O | L29 |
| P2-28 | O | B4 | A1 | O | L36 |
| P2-29 | O | B4 | A1 | O | L40 |
| P2-30 | O | B4 | A1 | O | L53 |
| P2-31 | O | B4 | A1 | O | L59 |
| P2-32 | O | B4 | A1 | O | L65 |
| P2-33 | O | B4 | A1 | O | L68 |
| P2-34 | O | B4 | A1 | O | L119 |
| P2-35 | O | B4 | A1 | O | L128 |
| P2-36 | O | B4 | A1 | O | L148 |
| P2-37 | O | B23 | A1 | O | Single bond |
| P2-38 | O | B23 | A1 | O | O |
| P2-39 | O | B23 | A1 | O | L1 |
| P2-40 | O | B23 | A1 | O | L5 |
| P2-41 | O | B23 | A1 | O | L9 |
| P2-42 | O | B23 | A1 | O | L18 |
| P2-43 | O | B23 | A1 | O | L22 |
| P2-44 | O | B23 | A1 | O | L27 |
| P2-45 | O | B23 | A1 | O | L29 |
| P2-46 | O | B23 | A1 | O | L36 |
| P2-47 | O | B23 | A1 | O | L40 |
| P2-48 | O | B23 | A1 | O | L53 |
| P2-49 | O | B23 | A1 | O | L59 |
| P2-50 | O | B23 | A1 | O | L65 |
| P2-51 | O | B23 | A1 | O | L68 |
| P2-52 | O | B23 | A1 | O | L119 |
| P2-53 | O | B23 | A1 | O | L128 |
| P2-54 | O | B23 | A1 | O | L148 |
| P2-55 | O | B36 | A1 | O | Single bond |
| P2-56 | O | B36 | A1 | O | O |
| P2-57 | O | B36 | A1 | O | L1 |
| P2-58 | O | B36 | A1 | O | L5 |
| P2-59 | O | B36 | A1 | O | L9 |
| P2-60 | O | B36 | A1 | O | L18 |
| P2-61 | O | B36 | A1 | O | L22 |
| P2-62 | O | B36 | A1 | O | L27 |
| P2-63 | O | B36 | A1 | O | L29 |
| P2-64 | O | B36 | A1 | O | L36 |
| P2-65 | O | B36 | A1 | O | L40 |
| P2-66 | O | B36 | A1 | O | L53 |
| P2-67 | O | B36 | A1 | O | L59 |
| P2-68 | O | B36 | A1 | O | L65 |
| P2-69 | O | B36 | A1 | O | L68 |
| P2-70 | O | B36 | A1 | O | L119 |
| P2-71 | O | B36 | A1 | O | L128 |
| P2-72 | O | B36 | A1 | O | L148 |
| P2-73 | O | B44 | A1 | O | Single bond |
| P2-74 | O | B44 | A1 | O | O |
| P2-75 | O | B44 | A1 | O | L1 |
| P2-76 | O | B44 | A1 | O | L5 |
| P2-77 | O | B44 | A1 | O | L9 |
| P2-78 | O | B44 | A1 | O | L18 |
| P2-79 | O | B44 | A1 | O | L22 |
| P2-80 | O | B44 | A1 | O | L27 |
| P2-81 | O | B44 | A1 | O | L29 |
| P2-82 | O | B44 | A1 | O | L36 |
| P2-83 | O | B44 | A1 | O | L40 |
| P2-84 | O | B44 | A1 | O | L53 |
| P2-85 | O | B44 | A1 | O | L59 |
| P2-86 | O | B44 | A1 | O | L65 |
| P2-87 | O | B44 | A1 | O | L68 |
| P2-88 | O | B44 | A1 | O | L119 |
| P2-89 | O | B44 | A1 | O | L128 |
| P2-90 | O | B44 | A1 | O | L148 |
| P2-91 | O | B92 | A1 | O | Single bond |
| P2-92 | O | B92 | A1 | O | O |
| P2-93 | O | B92 | A1 | O | L1 |
| P2-94 | O | B92 | A1 | O | L5 |
| P2-95 | O | B92 | A1 | O | L9 |
| P2-96 | O | B92 | A1 | O | L18 |
| P2-97 | O | B92 | A1 | O | L22 |
| P2-98 | O | B92 | A1 | O | L27 |
| P2-99 | O | B92 | A1 | O | L29 |
| P2-100 | O | B92 | A1 | O | L36 |
| P2-101 | O | B92 | A1 | O | L40 |
| P2-102 | O | B92 | A1 | O | L53 |
| P2-103 | O | B92 | A1 | O | L59 |
| P2-104 | O | B92 | A1 | O | L65 |
| P2-105 | O | B92 | A1 | O | L68 |
| P2-106 | O | B92 | A1 | O | L119 |
| P2-107 | O | B92 | A1 | O | L128 |
| P2-108 | O | B92 | A1 | O | L148 |
| P2-109 | O | A2 | A2 | O | Single bond |
| P2-110 | O | A2 | A2 | O | O |
| P2-111 | O | A2 | A2 | O | L1 |
| P2-112 | O | A2 | A2 | O | L2 |
| P2-113 | O | A2 | A2 | O | L3 |
| P2-114 | O | A2 | A2 | O | L4 |
| P2-115 | O | A2 | A2 | O | L5 |
| P2-116 | O | A2 | A2 | O | L6 |
| P2-117 | O | A2 | A2 | O | L7 |
| P2-118 | O | A2 | A2 | O | L9 |
| P2-119 | O | A2 | A2 | O | L11 |
| P2-120 | O | A2 | A2 | O | L14 |
| P2-121 | O | A2 | A2 | O | L18 |
| P2-122 | O | A2 | A2 | O | L22 |
| P2-123 | O | A2 | A2 | O | L27 |
| P2-124 | O | A2 | A2 | O | L28 |
| P2-125 | O | A2 | A2 | O | L29 |
| P2-126 | O | A2 | A2 | O | L30 |
| P2-127 | O | A2 | A2 | O | L32 |
| P2-128 | O | A2 | A2 | O | L36 |
| P2-129 | O | A2 | A2 | O | L40 |
| P2-130 | O | A2 | A2 | O | L48 |
| P2-131 | O | A2 | A2 | O | L53 |
| P2-132 | O | A2 | A2 | O | L59 |
| P2-133 | O | A2 | A2 | O | L65 |
| P2-134 | O | A2 | A2 | O | L68 |
| P2-135 | O | A2 | A2 | O | L79 |
| P2-136 | O | A2 | A2 | O | L89 |
| P2-137 | O | A2 | A2 | O | L102 |
| P2-138 | O | A2 | A2 | O | L111 |
| P2-139 | O | A2 | A2 | O | L119 |
| P2-140 | O | A2 | A2 | O | L128 |
| P2-141 | O | A2 | A2 | O | L148 |
| P2-142 | O | A2 | A2 | O | L156 |
| P2-143 | O | A2 | A2 | O | L167 |
| P2-144 | O | A2 | A2 | O | L169 |
| P2-145 | O | A2 | A2 | O | L172 |
| P2-146 | O | B4 | A2 | O | Single bond |
| P2-147 | O | B4 | A2 | O | O |
| P2-148 | O | B4 | A2 | O | L1 |
| P2-149 | O | B4 | A2 | O | L2 |
| P2-150 | O | B4 | A2 | O | L3 |
| P2-151 | O | B4 | A2 | O | L4 |
| P2-152 | O | B4 | A2 | O | L5 |
| P2-153 | O | B4 | A2 | O | L6 |
| P2-154 | O | B4 | A2 | O | L7 |
| P2-155 | O | B4 | A2 | O | L9 |
| P2-156 | O | B4 | A2 | O | L11 |
| P2-157 | O | B4 | A2 | O | L14 |
| P2-158 | O | B4 | A2 | O | L18 |
| P2-159 | O | B4 | A2 | O | L22 |
| P2-160 | O | B4 | A2 | O | L27 |
| P2-161 | O | B4 | A2 | O | L28 |
| P2-162 | O | B4 | A2 | O | L29 |
| P2-163 | O | B4 | A2 | O | L30 |
| P2-164 | O | B4 | A2 | O | L32 |
| P2-165 | O | B4 | A2 | O | L36 |
| P2-166 | O | B4 | A2 | O | L40 |
| P2-167 | O | B4 | A2 | O | L48 |
| P2-168 | O | B4 | A2 | O | L53 |
| P2-169 | O | B4 | A2 | O | L59 |
| P2-170 | O | B4 | A2 | O | L65 |
| P2-171 | O | B4 | A2 | O | L68 |
| P2-172 | O | B4 | A2 | O | L79 |
| P2-173 | O | B4 | A2 | O | L89 |
| P2-174 | O | B4 | A2 | O | L102 |
| P2-175 | O | B4 | A2 | O | L111 |
| P2-176 | O | B4 | A2 | O | L119 |
| P2-177 | O | B4 | A2 | O | L128 |
| P2-178 | O | B4 | A2 | O | L148 |
| P2-179 | O | B4 | A2 | O | L156 |
| P2-180 | O | B4 | A2 | O | L167 |
| P2-181 | O | B4 | A2 | O | L169 |
| P2-182 | O | B4 | A2 | O | L172 |
| P2-183 | O | B23 | A2 | O | Single bond |
| P2-184 | O | B23 | A2 | O | O |
| P2-185 | O | B23 | A2 | O | L1 |
| P2-186 | O | B23 | A2 | O | L2 |
| P2-187 | O | B23 | A2 | O | L3 |
| P2-188 | O | B23 | A2 | O | L4 |
| P2-189 | O | B23 | A2 | O | L5 |
| P2-190 | O | B23 | A2 | O | L6 |
| P2-191 | O | B23 | A2 | O | L7 |
| P2-192 | O | B23 | A2 | O | L9 |
| P2-193 | O | B23 | A2 | O | L11 |
| P2-194 | O | B23 | A2 | O | L14 |
| P2-195 | O | B23 | A2 | O | L18 |
| P2-196 | O | B23 | A2 | O | L22 |
| P2-197 | O | B23 | A2 | O | L27 |
| P2-198 | O | B23 | A2 | O | L28 |
| P2-199 | O | B23 | A2 | O | L29 |
| P2-200 | O | B23 | A2 | O | L30 |
| P2-201 | O | B23 | A2 | O | L32 |
| P2-202 | O | B23 | A2 | O | L36 |
| P2-203 | O | B23 | A2 | O | L40 |
| P2-204 | O | B23 | A2 | O | L48 |
| P2-205 | O | B23 | A2 | O | L53 |
| P2-206 | O | B23 | A2 | O | L59 |
| P2-207 | O | B23 | A2 | O | L65 |
| P2-208 | O | B23 | A2 | O | L68 |
| P2-209 | O | B23 | A2 | O | L79 |
| P2-210 | O | B23 | A2 | O | L89 |
| P2-211 | O | B23 | A2 | O | L102 |
| P2-212 | O | B23 | A2 | O | L111 |
| P2-213 | O | B23 | A2 | O | L119 |
| P2-214 | O | B23 | A2 | O | L128 |
| P2-215 | O | B23 | A2 | O | L148 |
| P2-216 | O | B23 | A2 | O | L156 |
| P2-217 | O | B23 | A2 | O | L167 |
| P2-218 | O | B23 | A2 | O | L169 |
| P2-219 | O | B23 | A2 | O | L172 |
| P2-220 | O | B36 | A2 | O | Single bond |
| P2-221 | O | B36 | A2 | O | O |
| P2-222 | O | B36 | A2 | O | L1 |
| P2-223 | O | B36 | A2 | O | L2 |
| P2-224 | O | B36 | A2 | O | L3 |
| P2-225 | O | B36 | A2 | O | L4 |
| P2-226 | O | B36 | A2 | O | L5 |
| P2-227 | O | B36 | A2 | O | L6 |
| P2-228 | O | B36 | A2 | O | L7 |
| P2-229 | O | B36 | A2 | O | L9 |
| P2-230 | O | B36 | A2 | O | L11 |
| P2-231 | O | B36 | A2 | O | L14 |
| P2-232 | O | B36 | A2 | O | L18 |
| P2-233 | O | B36 | A2 | O | L22 |
| P2-234 | O | B36 | A2 | O | L27 |
| P2-235 | O | B36 | A2 | O | L28 |
| P2-236 | O | B36 | A2 | O | L29 |
| P2-237 | O | B36 | A2 | O | L30 |
| P2-238 | O | B36 | A2 | O | L32 |
| P2-239 | O | B36 | A2 | O | L36 |
| P2-240 | O | B36 | A2 | O | L40 |
| P2-241 | O | B36 | A2 | O | L48 |
| P2-242 | O | B36 | A2 | O | L53 |
| P2-243 | O | B36 | A2 | O | L59 |
| P2-244 | O | B36 | A2 | O | L65 |
| P2-245 | O | B36 | A2 | O | L68 |
| P2-246 | O | B36 | A2 | O | L79 |
| P2-247 | O | B36 | A2 | O | L89 |
| P2-248 | O | B36 | A2 | O | L102 |
| P2-249 | O | B36 | A2 | O | L111 |
| P2-250 | O | B36 | A2 | O | L119 |
| P2-251 | O | B36 | A2 | O | L128 |
| P2-252 | O | B36 | A2 | O | L148 |
| P2-253 | O | B36 | A2 | O | L156 |
| P2-254 | O | B36 | A2 | O | L167 |
| P2-255 | O | B36 | A2 | O | L169 |
| P2-256 | O | B36 | A2 | O | L172 |
| P2-257 | O | B44 | A2 | O | Single bond |
| P2-258 | O | B44 | A2 | O | O |
| P2-259 | O | B44 | A2 | O | L1 |
| P2-260 | O | B44 | A2 | O | L2 |
| P2-261 | O | B44 | A2 | O | L3 |
| P2-262 | O | B44 | A2 | O | L4 |
| P2-263 | O | B44 | A2 | O | L5 |
| P2-264 | O | B44 | A2 | O | L6 |
| P2-265 | O | B44 | A2 | O | L7 |
| P2-266 | O | B44 | A2 | O | L9 |
| P2-267 | O | B44 | A2 | O | L11 |
| P2-268 | O | B44 | A2 | O | L14 |
| P2-269 | O | B44 | A2 | O | L18 |
| P2-270 | O | B44 | A2 | O | L22 |
| P2-271 | O | B56 | A2 | O | L27 |
| P2-272 | O | B44 | A2 | O | L28 |
| P2-273 | O | B44 | A2 | O | L29 |
| P2-274 | O | B44 | A2 | O | L30 |
| P2-275 | O | B44 | A2 | O | L32 |
| P2-276 | O | B44 | A2 | O | L36 |
| P2-277 | O | B44 | A2 | O | L40 |
| P2-278 | O | B44 | A2 | O | L48 |
| P2-279 | O | B44 | A2 | O | L53 |
| P2-280 | O | B44 | A2 | O | L59 |
| P2-281 | O | B44 | A2 | O | L65 |
| P2-282 | O | B44 | A2 | O | L68 |
| P2-283 | O | B44 | A2 | O | L79 |
| P2-284 | O | B44 | A2 | O | L89 |
| P2-285 | O | B44 | A2 | O | L102 |
| P2-286 | O | B44 | A2 | O | L111 |
| P2-287 | O | B44 | A2 | O | L119 |
| P2-288 | O | B44 | A2 | O | L128 |
| P2-289 | O | B44 | A2 | O | L148 |
| P2-290 | O | B44 | A2 | O | L156 |
| P2-291 | O | B44 | A2 | O | L167 |
| P2-292 | O | B44 | A2 | O | L169 |
| P2-293 | O | B44 | A2 | O | L172 |
| P2-294 | O | B126 | A2 | O | Single bond |
| P2-295 | O | B92 | A2 | O | O |
| P2-296 | O | B92 | A2 | O | L1 |
| P2-297 | O | B92 | A2 | O | L2 |
| P2-298 | O | B92 | A2 | O | L3 |
| P2-299 | O | B92 | A2 | O | L4 |
| P2-300 | O | B92 | A2 | O | L5 |
| P2-301 | O | B92 | A2 | O | L6 |
| P2-302 | O | B92 | A2 | O | L7 |
| P2-303 | O | B92 | A2 | O | L9 |
| P2-304 | O | B92 | A2 | O | L11 |
| P2-305 | O | B92 | A2 | O | L14 |
| P2-306 | O | B92 | A2 | O | L18 |
| P2-307 | O | B92 | A2 | O | L22 |
| P2-308 | O | B92 | A2 | O | L27 |
| P2-309 | O | B92 | A2 | O | L28 |
| P2-310 | O | B92 | A2 | O | L29 |
| P2-311 | O | B92 | A2 | O | L30 |
| P2-312 | O | B92 | A2 | O | L32 |
| P2-313 | O | B92 | A2 | O | L36 |
| P2-314 | O | B92 | A2 | O | L40 |
| P2-315 | O | B92 | A2 | O | L48 |
| P2-316 | O | B92 | A2 | O | L53 |
| P2-317 | O | B92 | A2 | O | L59 |
| P2-318 | O | B92 | A2 | O | L65 |
| P2-319 | O | B92 | A2 | O | L68 |
| P2-320 | O | B92 | A2 | O | L79 |
| P2-321 | O | B92 | A2 | O | L89 |
| P2-322 | O | B92 | A2 | O | L102 |
| P2-323 | O | B92 | A2 | O | L111 |
| P2-324 | O | B92 | A2 | O | L119 |
| P2-325 | O | B92 | A2 | O | L128 |
| P2-326 | O | B92 | A2 | O | L148 |
| P2-327 | O | B92 | A2 | O | L156 |
| P2-328 | O | B92 | A2 | O | L167 |
| P2-329 | O | B92 | A2 | O | L169 |
| P2-330 | O | B92 | A2 | O | L172 |
| P2-331 | O | A2 | A3 | O | Single bond |
| P2-332 | O | A2 | A3 | O | O |
| P2-333 | O | A2 | A3 | O | L1 |
| P2-334 | O | A2 | A3 | O | L2 |
| P2-335 | O | A2 | A3 | O | L3 |
| P2-336 | O | A2 | A3 | O | L4 |
| P2-337 | O | A2 | A3 | O | L5 |
| P2-338 | O | A2 | A3 | O | L6 |
| P2-339 | O | A2 | A3 | O | L7 |
| P2-340 | O | A2 | A3 | O | L9 |
| P2-341 | O | A2 | A3 | O | L11 |
| P2-342 | O | A2 | A3 | O | L14 |
| P2-343 | O | A2 | A3 | O | L18 |
| P2-344 | O | A2 | A3 | O | L22 |
| P2-345 | O | A2 | A3 | O | L27 |
| P2-346 | O | A2 | A3 | O | L28 |
| P2-347 | O | A2 | A3 | O | L29 |
| P2-348 | O | A2 | A3 | O | L30 |
| P2-349 | O | A2 | A3 | O | L32 |
| P2-350 | O | A2 | A3 | O | L36 |
| P2-351 | O | A2 | A3 | O | L40 |
| P2-352 | O | A2 | A3 | O | L48 |
| P2-353 | O | A2 | A3 | O | L53 |
| P2-354 | O | A2 | A3 | O | L59 |
| P2-355 | O | A2 | A3 | O | L65 |
| P2-356 | O | A2 | A3 | O | L68 |
| P2-357 | O | A2 | A3 | O | L79 |
| P2-358 | O | A2 | A3 | O | L89 |
| P2-359 | O | A2 | A3 | O | L102 |
| P2-360 | O | A2 | A3 | O | L111 |
| P2-361 | O | A2 | A3 | O | L119 |
| P2-362 | O | A2 | A3 | O | L128 |
| P2-363 | O | A2 | A3 | O | L148 |
| P2-364 | O | A2 | A3 | O | L156 |
| P2-365 | O | A2 | A3 | O | L167 |
| P2-366 | O | A2 | A3 | O | L169 |
| P2-367 | O | A2 | A3 | O | L172 |
| P2-368 | O | B4 | A3 | O | Single bond |
| P2-369 | O | B4 | A3 | O | O |
| P2-370 | O | B4 | A3 | O | L1 |
| P2-371 | O | B4 | A3 | O | L2 |
| P2-372 | O | B4 | A3 | O | L3 |
| P2-373 | O | B4 | A3 | O | L4 |
| P2-374 | O | B4 | A3 | O | L5 |
| P2-375 | O | B4 | A3 | O | L6 |
| P2-376 | O | B4 | A3 | O | L7 |
| P2-377 | O | B4 | A3 | O | L9 |
| P2-378 | O | B4 | A3 | O | L11 |
| P2-379 | O | B4 | A3 | O | L14 |
| P2-380 | O | B4 | A3 | O | L18 |
| P2-381 | O | B4 | A3 | O | L22 |
| P2-382 | O | B4 | A3 | O | L27 |
| P2-383 | O | B4 | A3 | O | L28 |
| P2-384 | O | B4 | A3 | O | L29 |
| P2-385 | O | B4 | A3 | O | L30 |
| P2-386 | O | B4 | A3 | O | L32 |
| P2-387 | O | B4 | A3 | O | L36 |
| P2-388 | O | B4 | A3 | O | L40 |
| P2-389 | O | B4 | A3 | O | L48 |
| P2-390 | O | B4 | A3 | O | L53 |
| P2-391 | O | B4 | A3 | O | L59 |
| P2-392 | O | B4 | A3 | O | L65 |
| P2-393 | O | B4 | A3 | O | L68 |
| P2-394 | O | B4 | A3 | O | L79 |
| P2-395 | O | B4 | A3 | O | L89 |
| P2-396 | O | B4 | A3 | O | L102 |
| P2-397 | O | B4 | A3 | O | L111 |
| P2-398 | O | B4 | A3 | O | L119 |
| P2-399 | O | B4 | A3 | O | L128 |
| P2-400 | O | B4 | A3 | O | L148 |
| P2-401 | O | B4 | A3 | O | L156 |
| P2-402 | O | B4 | A3 | O | L167 |
| P2-403 | O | B4 | A3 | O | L169 |
| P2-404 | O | B4 | A3 | O | L172 |
| P2-405 | O | B23 | A3 | O | Single bond |
| P2-406 | O | B23 | A3 | O | O |
| P2-407 | O | B23 | A3 | O | L1 |
| P2-408 | O | B23 | A3 | O | L2 |
| P2-409 | O | B23 | A3 | O | L3 |
| P2-410 | O | B23 | A3 | O | L4 |
| P2-411 | O | B23 | A3 | O | L5 |
| P2-412 | O | B23 | A3 | O | L6 |
| P2-413 | O | B23 | A3 | O | L7 |
| P2-414 | O | B23 | A3 | O | L9 |
| P2-415 | O | B23 | A3 | O | L11 |
| P2-416 | O | B23 | A3 | O | L14 |
| P2-417 | O | B23 | A3 | O | L18 |
| P2-418 | O | B23 | A3 | O | L22 |
| P2-419 | O | B23 | A3 | O | L27 |
| P2-420 | O | B23 | A3 | O | L28 |
| P2-421 | O | B23 | A3 | O | L29 |
| P2-422 | O | B23 | A3 | O | L30 |
| P2-423 | O | B23 | A3 | O | L32 |
| P2-424 | O | B23 | A3 | O | L36 |
| P2-425 | O | B23 | A3 | O | L40 |
| P2-426 | O | B23 | A3 | O | L48 |
| P2-427 | O | B23 | A3 | O | L53 |
| P2-428 | O | B23 | A3 | O | L59 |
| P2-429 | O | B23 | A3 | O | L65 |
| P2-430 | O | B23 | A3 | O | L68 |
| P2-431 | O | B23 | A3 | O | L79 |
| P2-432 | O | B23 | A3 | O | L89 |
| P2-433 | O | B23 | A3 | O | L102 |
| P2-434 | O | B23 | A3 | O | L111 |
| P2-435 | O | B23 | A3 | O | L119 |
| P2-436 | O | B23 | A3 | O | L128 |
| P2-437 | O | B23 | A3 | O | L148 |
| P2-438 | O | B23 | A3 | O | L156 |
| P2-439 | O | B23 | A3 | O | L167 |
| P2-440 | O | B23 | A3 | O | L169 |
| P2-441 | O | B23 | A3 | O | L172 |
| P2-442 | O | B36 | A3 | O | Single bond |
| P2-443 | O | B36 | A3 | O | O |
| P2-444 | O | B36 | A3 | O | L1 |
| P2-445 | O | B36 | A3 | O | L2 |
| P2-446 | O | B36 | A3 | O | L3 |
| P2-447 | O | B36 | A3 | O | L4 |
| P2-448 | O | B36 | A3 | O | L5 |
| P2-449 | O | B36 | A3 | O | L6 |
| P2-450 | O | B36 | A3 | O | L7 |
| P2-451 | O | B36 | A3 | O | L9 |
| P2-452 | O | B36 | A3 | O | L11 |
| P2-453 | O | B36 | A3 | O | L14 |
| P2-454 | O | B36 | A3 | O | L18 |
| P2-455 | O | B36 | A3 | O | L22 |
| P2-456 | O | B36 | A3 | O | L27 |
| P2-457 | O | B36 | A3 | O | L28 |
| P2-458 | O | B36 | A3 | O | L29 |
| P2-459 | O | B36 | A3 | O | L30 |
| P2-460 | O | B36 | A3 | O | L32 |
| P2-461 | O | B36 | A3 | O | L36 |
| P2-462 | O | B36 | A3 | O | L40 |
| P2-463 | O | B36 | A3 | O | L48 |
| P2-464 | O | B36 | A3 | O | L53 |
| P2-465 | O | B36 | A3 | O | L59 |
| P2-466 | O | B36 | A3 | O | L65 |
| P2-467 | O | B36 | A3 | O | L68 |
| P2-468 | O | B36 | A3 | O | L79 |
| P2-469 | O | B36 | A3 | O | L89 |
| P2-470 | O | B36 | A3 | O | L102 |
| P2-471 | O | B36 | A3 | O | L111 |
| P2-472 | O | B36 | A3 | O | L119 |
| P2-473 | O | B36 | A3 | O | L128 |
| P2-474 | O | B36 | A3 | O | L148 |
| P2-475 | O | B36 | A3 | O | L156 |
| P2-476 | O | B36 | A3 | O | L167 |
| P2-477 | O | B36 | A3 | O | L169 |
| P2-478 | O | B36 | A3 | O | L172 |
| P2-479 | O | B44 | A3 | O | Single bond |
| P2-480 | O | B44 | A3 | O | O |
| P2-481 | O | B44 | A3 | O | L1 |
| P2-482 | O | B44 | A3 | O | L2 |
| P2-483 | O | B44 | A3 | O | L3 |
| P2-484 | O | B44 | A3 | O | L4 |
| P2-485 | O | B44 | A3 | O | L5 |
| P2-486 | O | B44 | A3 | O | L6 |
| P2-487 | O | B44 | A3 | O | L7 |
| P2-488 | O | B44 | A3 | O | L9 |
| P2-489 | O | B44 | A3 | O | L11 |
| P2-490 | O | B44 | A3 | O | L14 |
| P2-491 | O | B44 | A3 | O | L18 |
| P2-492 | O | B44 | A3 | O | L22 |
| P2-493 | O | B44 | A3 | O | L27 |
| P2-494 | O | B44 | A3 | O | L28 |
| P2-495 | O | B44 | A3 | O | L29 |
| P2-496 | O | B44 | A3 | O | L30 |
| P2-497 | O | B44 | A3 | O | L32 |
| P2-498 | O | B44 | A3 | O | L36 |
| P2-499 | O | B44 | A3 | O | L40 |
| P2-500 | O | B44 | A3 | O | L48 |
| P2-501 | O | B44 | A3 | O | L53 |
| P2-502 | O | B44 | A3 | O | L59 |
| P2-503 | O | B44 | A3 | O | L65 |
| P2-504 | O | B44 | A3 | O | L68 |
| P2-505 | O | B44 | A3 | O | L79 |
| P2-506 | O | B44 | A3 | O | L89 |
| P2-507 | O | B44 | A3 | O | L102 |
| P2-508 | O | B44 | A3 | O | L111 |
| P2-509 | O | B44 | A3 | O | L119 |
| P2-510 | O | B44 | A3 | O | L128 |
| P2-511 | O | B44 | A3 | O | L148 |
| P2-512 | O | B44 | A3 | O | L156 |
| P2-513 | O | B44 | A3 | O | L167 |
| P2-514 | O | B44 | A3 | O | L169 |
| P2-515 | O | B44 | A3 | O | L172 |
| P2-516 | O | B92 | A3 | O | Single bond |
| P2-517 | O | B92 | A3 | O | O |
| P2-518 | O | B92 | A3 | O | L1 |
| P2-519 | O | B92 | A3 | O | L2 |
| P2-520 | O | B92 | A3 | O | L3 |
| P2-521 | O | B92 | A3 | O | L4 |
| P2-522 | O | B92 | A3 | O | L5 |
| P2-523 | O | B92 | A3 | O | L6 |
| P2-524 | O | B92 | A3 | O | L7 |
| P2-525 | O | B92 | A3 | O | L9 |
| P2-526 | O | B92 | A3 | O | L11 |
| P2-527 | O | B92 | A3 | O | L14 |
| P2-528 | O | B92 | A3 | O | L18 |
| P2-529 | O | B92 | A3 | O | L22 |
| P2-530 | O | B92 | A3 | O | L27 |
| P2-531 | O | B92 | A3 | O | L28 |
| P2-532 | O | B92 | A3 | O | L29 |
| P2-533 | O | B92 | A3 | O | L30 |
| P2-534 | O | B92 | A3 | O | L32 |
| P2-535 | O | B92 | A3 | O | L36 |
| P2-536 | O | B92 | A3 | O | L40 |
| P2-537 | O | B92 | A3 | O | L48 |
| P2-538 | O | B92 | A3 | O | L53 |
| P2-539 | O | B92 | A3 | O | L59 |
| P2-540 | O | B92 | A3 | O | L65 |
| P2-541 | O | B92 | A3 | O | L68 |
| P2-542 | O | B92 | A3 | O | L79 |
| P2-543 | O | B92 | A3 | O | L89 |
| P2-544 | O | B92 | A3 | O | L102 |
| P2-545 | O | B92 | A3 | O | L111 |
| P2-546 | O | B92 | A3 | O | L119 |
| P2-547 | O | B92 | A3 | O | L128 |
| P2-548 | O | B92 | A3 | O | L148 |
| P2-549 | O | B92 | A3 | O | L156 |
| P2-550 | O | B92 | A3 | O | L167 |
| P2-551 | O | B92 | A3 | O | L169 |
| P2-552 | O | B92 | A3 | O | L172 |
| P2-553 | O | A2 | A12 | O | Single bond |
| P2-554 | O | A2 | A12 | O | O |
| P2-555 | O | A2 | A12 | O | L1 |
| P2-556 | O | A2 | A12 | O | L5 |
| P2-557 | O | A2 | A12 | O | L9 |
| P2-558 | O | A2 | A12 | O | L18 |
| P2-559 | O | A2 | A12 | O | L22 |
| P2-560 | O | A2 | A12 | O | L27 |
| P2-561 | O | A2 | A12 | O | L29 |
| P2-562 | O | A2 | A12 | O | L36 |
| P2-563 | O | A2 | A12 | O | L40 |
| P2-564 | O | A2 | A12 | O | L53 |
| P2-565 | O | A2 | A12 | O | L59 |
| P2-566 | O | A2 | A12 | O | L65 |
| P2-567 | O | A2 | A12 | O | L68 |
| P2-568 | O | A2 | A12 | O | L119 |
| P2-569 | O | A2 | A12 | O | L128 |
| P2-570 | O | A2 | A12 | O | L148 |
| P2-571 | O | B4 | A12 | O | Single bond |
| P2-572 | O | B4 | A12 | O | O |
| P2-573 | O | B4 | A12 | O | L1 |
| P2-574 | O | B4 | A12 | O | L5 |
| P2-575 | O | B4 | A12 | O | L9 |
| P2-576 | O | B4 | A12 | O | L18 |
| P2-577 | O | B4 | A12 | O | L22 |
| P2-578 | O | B4 | A12 | O | L27 |
| P2-579 | O | B4 | A12 | O | L29 |
| P2-580 | O | B4 | A12 | O | L36 |
| P2-581 | O | B4 | A12 | O | L40 |
| P2-582 | O | B4 | A12 | O | L53 |
| P2-583 | O | B4 | A12 | O | L59 |
| P2-584 | O | B4 | A12 | O | L65 |
| P2-585 | O | B4 | A12 | O | L68 |
| P2-586 | O | B4 | A12 | O | L119 |
| P2-587 | O | B4 | A12 | O | L128 |
| P2-588 | O | B4 | A12 | O | L148 |
| P2-589 | O | B23 | A12 | O | Single bond |
| P2-590 | O | B23 | A12 | O | O |
| P2-591 | O | B23 | A12 | O | L1 |
| P2-592 | O | B23 | A12 | O | L5 |
| P2-593 | O | B23 | A12 | O | L9 |
| P2-594 | O | B23 | A12 | O | L18 |
| P2-595 | O | B23 | A12 | O | L22 |
| P2-596 | O | B23 | A12 | O | L27 |
| P2-597 | O | B23 | A12 | O | L29 |
| P2-598 | O | B23 | A12 | O | L36 |
| P2-599 | O | B23 | A12 | O | L40 |
| P2-600 | O | B23 | A12 | O | L53 |
| P2-601 | O | B23 | A12 | O | L59 |
| P2-602 | O | B23 | A12 | O | L65 |
| P2-603 | O | B23 | A12 | O | L68 |
| P2-604 | O | B23 | A12 | O | L119 |
| P2-605 | O | B23 | A12 | O | L128 |
| P2-606 | O | B23 | A12 | O | L148 |
| P2-607 | O | B36 | A12 | O | Single bond |
| P2-608 | O | B36 | A12 | O | O |
| P2-609 | O | B36 | A12 | O | L1 |
| P2-610 | O | B36 | A12 | O | L5 |
| P2-611 | O | B36 | A12 | O | L9 |
| P2-612 | O | B36 | A12 | O | L18 |
| P2-613 | O | B36 | A12 | O | L22 |
| P2-614 | O | B36 | A12 | O | L27 |
| P2-615 | O | B36 | A12 | O | L29 |
| P2-616 | O | B36 | A12 | O | L36 |
| P2-617 | O | B36 | A12 | O | L40 |
| P2-618 | O | B36 | A12 | O | L53 |
| P2-619 | O | B36 | A12 | O | L59 |
| P2-620 | O | B36 | A12 | O | L65 |
| P2-621 | O | B36 | A12 | O | L68 |
| P2-622 | O | B36 | A12 | O | L119 |
| P2-623 | O | B36 | A12 | O | L128 |
| P2-624 | O | B36 | A12 | O | L148 |
| P2-625 | O | B44 | A12 | O | Single bond |
| P2-626 | O | B44 | A12 | O | O |
| P2-627 | O | B44 | A12 | O | L1 |
| P2-628 | O | B44 | A12 | O | L5 |
| P2-629 | O | B44 | A12 | O | L9 |
| P2-630 | O | B44 | A12 | O | L18 |
| P2-631 | O | B44 | A12 | O | L22 |
| P2-632 | O | B44 | A12 | O | L27 |
| P2-633 | O | B44 | A12 | O | L29 |
| P2-634 | O | B44 | A12 | O | L36 |
| P2-635 | O | B44 | A12 | O | L40 |
| P2-636 | O | B44 | A12 | O | L53 |
| P2-637 | O | B44 | A12 | O | L59 |
| P2-638 | O | B44 | A12 | O | L65 |
| P2-639 | O | B44 | A12 | O | L68 |
| P2-640 | O | B44 | A12 | O | L119 |
| P2-641 | O | B44 | A12 | O | L128 |
| P2-642 | O | B44 | A12 | O | L148 |
| P2-643 | O | B92 | A12 | O | Single bond |
| P2-644 | O | B92 | A12 | O | O |
| P2-645 | O | B92 | A12 | O | L1 |
| P2-646 | O | B92 | A12 | O | L5 |
| P2-647 | O | B92 | A12 | O | L9 |
| P2-648 | O | B92 | A12 | O | L18 |
| P2-649 | O | B92 | A12 | O | L22 |
| P2-650 | O | B92 | A12 | O | L27 |
| P2-651 | O | B92 | A12 | O | L29 |
| P2-652 | O | B92 | A12 | O | L36 |
| P2-653 | O | B92 | A12 | O | L40 |
| P2-654 | O | B92 | A12 | O | L53 |
| P2-655 | O | B92 | A12 | O | L59 |
| P2-656 | O | B92 | A12 | O | L65 |
| P2-657 | O | B92 | A12 | O | L68 |
| P2-658 | O | B92 | A12 | O | L119 |
| P2-659 | O | B92 | A12 | O | L128 |
| P2-660 | O | B92 | A12 | O | L148 |
| P2-661 | O | A2 | B41 | O | Single bond |
| P2-662 | O | A2 | B41 | O | O |
| P2-663 | O | A2 | B41 | O | L1 |
| P2-664 | O | A2 | B41 | O | L5 |
| P2-665 | O | A2 | B41 | O | L9 |
| P2-666 | O | A2 | B41 | O | L18 |
| P2-667 | O | A2 | B41 | O | L22 |
| P2-668 | O | A2 | B41 | O | L27 |
| P2-669 | O | A2 | B41 | O | L29 |
| P2-670 | O | A2 | B41 | O | L36 |
| P2-671 | O | A2 | B41 | O | L40 |
| P2-672 | O | A2 | B41 | O | L53 |
| P2-673 | O | A2 | B41 | O | L59 |
| P2-674 | O | A2 | B41 | O | L65 |
| P2-675 | O | A2 | B41 | O | L68 |
| P2-676 | O | A2 | B41 | O | L119 |
| P2-677 | O | A2 | B41 | O | L128 |
| P2-678 | O | A2 | B41 | O | L148 |
| P2-679 | O | B4 | B41 | O | Single bond |
| P2-680 | O | B4 | B41 | O | O |
| P2-681 | O | B4 | B41 | O | L1 |
| P2-682 | O | B4 | B41 | O | L5 |
| P2-683 | O | B4 | B41 | O | L9 |
| P2-684 | O | B4 | B41 | O | L18 |
| P2-685 | O | B4 | B41 | O | L22 |
| P2-686 | O | B4 | B41 | O | L27 |
| P2-687 | O | B4 | B41 | O | L29 |
| P2-688 | O | B4 | B41 | O | L36 |
| P2-689 | O | B4 | B41 | O | L40 |
| P2-690 | O | B4 | B41 | O | L53 |
| P2-691 | O | B4 | B41 | O | L59 |
| P2-692 | O | B4 | B41 | O | L65 |
| P2-693 | O | B4 | B41 | O | L68 |
| P2-694 | O | B4 | B41 | O | L119 |
| P2-695 | O | B4 | B41 | O | L128 |
| P2-696 | O | B4 | B41 | O | L148 |
| P2-697 | O | B23 | B41 | O | Single bond |
| P2-698 | O | B23 | B41 | O | O |
| P2-699 | O | B23 | B41 | O | L1 |
| P2-700 | O | B23 | B41 | O | L5 |
| P2-701 | O | B23 | B41 | O | L9 |
| P2-702 | O | B23 | B41 | O | L18 |
| P2-703 | O | B23 | B41 | O | L22 |
| P2-704 | O | B23 | B41 | O | L27 |
| P2-705 | O | B23 | B41 | O | L29 |
| P2-706 | O | B23 | B41 | O | L36 |
| P2-707 | O | B23 | B41 | O | L40 |
| P2-708 | O | B23 | B41 | O | L53 |
| P2-709 | O | B23 | B41 | O | L59 |
| P2-710 | O | B23 | B41 | O | L65 |
| P2-711 | O | B23 | B41 | O | L68 |
| P2-712 | O | B23 | B41 | O | L119 |
| P2-713 | O | B23 | B41 | O | L128 |
| P2-714 | O | B23 | B41 | O | L148 |
| P2-715 | O | B36 | B41 | O | Single bond |
| P2-716 | O | B36 | B41 | O | O |
| P2-717 | O | B36 | B41 | O | L1 |
| P2-718 | O | B36 | B41 | O | L5 |
| P2-719 | O | B36 | B41 | O | L9 |
| P2-720 | O | B36 | B41 | O | L18 |
| P2-721 | O | B36 | B41 | O | L22 |
| P2-722 | O | B36 | B41 | O | L27 |
| P2-723 | O | B36 | B41 | O | L29 |
| P2-724 | O | B36 | B41 | O | L36 |
| P2-725 | O | B36 | B41 | O | L40 |
| P2-726 | O | B36 | B41 | O | L53 |
| P2-727 | O | B36 | B41 | O | L59 |
| P2-728 | O | B36 | B41 | O | L65 |
| P2-729 | O | B36 | B41 | O | L68 |
| P2-730 | O | B36 | B41 | O | L119 |
| P2-731 | O | B36 | B41 | O | L128 |
| P2-732 | O | B36 | B41 | O | L148 |
| P2-733 | O | B44 | B41 | O | Single bond |
| P2-734 | O | B44 | B41 | O | O |
| P2-735 | O | B44 | B41 | O | L1 |
| P2-736 | O | B44 | B41 | O | L5 |
| P2-737 | O | B44 | B41 | O | L9 |
| P2-738 | O | B44 | B41 | O | L18 |
| P2-739 | O | B44 | B41 | O | L22 |
| P2-740 | O | B44 | B41 | O | L27 |
| P2-741 | O | B44 | B41 | O | L29 |
| P2-742 | O | B44 | B41 | O | L36 |
| P2-743 | O | B44 | B41 | O | L40 |
| P2-744 | O | B44 | B41 | O | L53 |
| P2-745 | O | B44 | B41 | O | L59 |
| P2-746 | O | B44 | B41 | O | L65 |
| P2-747 | O | B44 | B41 | O | L68 |
| P2-748 | O | B44 | B41 | O | L119 |
| P2-749 | O | B44 | B41 | O | L128 |
| P2-750 | O | B44 | B41 | O | L148 |
| P2-751 | O | B92 | B41 | O | Single bond |
| P2-752 | O | B92 | B41 | O | O |
| P2-753 | O | B92 | B41 | O | L1 |
| P2-754 | O | B92 | B41 | O | L5 |
| P2-755 | O | B92 | B41 | O | L9 |
| P2-756 | O | B92 | B41 | O | L18 |
| P2-757 | O | B92 | B41 | O | L22 |
| P2-758 | O | B92 | B41 | O | L27 |
| P2-759 | O | B92 | B41 | O | L29 |
| P2-760 | O | B92 | B41 | O | L36 |
| P2-761 | O | B92 | B41 | O | L40 |
| P2-762 | O | B92 | B41 | O | L53 |
| P2-763 | O | B92 | B41 | O | L59 |
| P2-764 | O | B92 | B41 | O | L65 |
| P2-765 | O | B92 | B41 | O | L68 |
| P2-766 | O | B92 | B41 | O | L119 |
| P2-767 | O | B92 | B41 | O | L128 |
| P2-768 | O | B92 | B41 | O | L148 |
| P2-769 | O | A2 | B56 | O | Single bond |
| P2-770 | O | A2 | B56 | O | O |
| P2-771 | O | A2 | B56 | O | L1 |
| P2-772 | O | A2 | B56 | O | L5 |
| P2-773 | O | A2 | B56 | O | L9 |
| P2-774 | O | A2 | B56 | O | L18 |
| P2-775 | O | A2 | B56 | O | L22 |
| P2-776 | O | A2 | B56 | O | L27 |
| P2-777 | O | A2 | B56 | O | L29 |
| P2-778 | O | A2 | B56 | O | L36 |
| P2-779 | O | A2 | B56 | O | L40 |
| P2-780 | O | A2 | B56 | O | L53 |
| P2-781 | O | A2 | B56 | O | L59 |
| P2-782 | O | A2 | B56 | O | L65 |
| P2-783 | O | A2 | B56 | O | L68 |
| P2-784 | O | A2 | B56 | O | L119 |
| P2-785 | O | A2 | B56 | O | L128 |
| P2-786 | O | A2 | B56 | O | L148 |
| P2-787 | O | B4 | B56 | O | Single bond |
| P2-788 | O | B4 | B56 | O | O |
| P2-789 | O | B4 | B56 | O | L1 |
| P2-790 | O | B4 | B56 | O | L5 |
| P2-791 | O | B4 | B56 | O | L9 |
| P2-792 | O | B4 | B56 | O | L18 |
| P2-793 | O | B4 | B56 | O | L22 |
| P2-794 | O | B4 | B56 | O | L27 |
| P2-795 | O | B4 | B56 | O | L29 |
| P2-796 | O | B4 | B56 | O | L36 |
| P2-797 | O | B4 | B56 | O | L40 |
| P2-798 | O | B4 | B56 | O | L53 |
| P2-799 | O | B4 | B56 | O | L59 |
| P2-800 | O | B4 | B56 | O | L65 |
| P2-801 | O | B4 | B56 | O | L68 |
| P2-802 | O | B4 | B56 | O | L119 |
| P2-803 | O | B4 | B56 | O | L128 |
| P2-804 | O | B4 | B56 | O | L148 |
| P2-805 | O | B23 | B56 | O | Single bond |
| P2-806 | O | B23 | B56 | O | O |
| P2-807 | O | B23 | B56 | O | L1 |
| P2-808 | O | B23 | B56 | O | L5 |
| P2-809 | O | B23 | B56 | O | L9 |
| P2-810 | O | B23 | B56 | O | L18 |
| P2-811 | O | B23 | B56 | O | L22 |
| P2-812 | O | B23 | B56 | O | L27 |
| P2-813 | O | B23 | B56 | O | L29 |
| P2-814 | O | B23 | B56 | O | L36 |
| P2-815 | O | B23 | B56 | O | L40 |
| P2-816 | O | B23 | B56 | O | L53 |
| P2-817 | O | B23 | B56 | O | L59 |
| P2-818 | O | B23 | B56 | O | L65 |
| P2-819 | O | B23 | B56 | O | L68 |
| P2-820 | O | B23 | B56 | O | L119 |
| P2-821 | O | B23 | B56 | O | L128 |
| P2-822 | O | B23 | B56 | O | L148 |
| P2-823 | O | B36 | B56 | O | Single bond |
| P2-824 | O | B36 | B56 | O | O |
| P2-825 | O | B36 | B56 | O | L1 |
| P2-826 | O | B36 | B56 | O | L5 |
| P2-827 | O | B36 | B56 | O | L9 |
| P2-828 | O | B36 | B56 | O | L18 |
| P2-829 | O | B36 | B56 | O | L22 |
| P2-830 | O | B36 | B56 | O | L27 |
| P2-831 | O | B36 | B56 | O | L29 |
| P2-832 | O | B36 | B56 | O | L36 |
| P2-833 | O | B36 | B56 | O | L40 |
| P2-834 | O | B36 | B56 | O | L53 |
| P2-835 | O | B36 | B56 | O | L59 |
| P2-836 | O | B36 | B56 | O | L65 |
| P2-837 | O | B36 | B56 | O | L68 |
| P2-838 | O | B36 | B56 | O | L119 |
| P2-839 | O | B36 | B56 | O | L128 |
| P2-840 | O | B36 | B56 | O | L148 |
| P2-841 | O | B44 | B56 | O | Single bond |
| P2-842 | O | B44 | B56 | O | O |
| P2-843 | O | B44 | B56 | O | L1 |
| P2-844 | O | B44 | B56 | O | L5 |
| P2-845 | O | B44 | B56 | O | L9 |
| P2-846 | O | B44 | B56 | O | L18 |
| P2-847 | O | B44 | B56 | O | L22 |
| P2-848 | O | B44 | B56 | O | L27 |
| P2-849 | O | B44 | B56 | O | L29 |
| P2-850 | O | B44 | B56 | O | L36 |
| P2-851 | O | B44 | B56 | O | L40 |
| P2-852 | O | B44 | B56 | O | L53 |
| P2-853 | O | B44 | B56 | O | L59 |
| P2-854 | O | B44 | B56 | O | L65 |
| P2-855 | O | B44 | B56 | O | L68 |
| P2-856 | O | B44 | B56 | O | L119 |
| P2-857 | O | B44 | B56 | O | L128 |
| P2-858 | O | B44 | B56 | O | L148 |
| P2-859 | O | B92 | B56 | O | Single bond |
| P2-860 | O | B92 | B56 | O | O |
| P2-861 | O | B92 | B56 | O | L1 |
| P2-862 | O | B92 | B56 | O | L5 |
| P2-863 | O | B92 | B56 | O | L9 |
| P2-864 | O | B92 | B56 | O | L18 |
| P2-865 | O | B92 | B56 | O | L22 |
| P2-866 | O | B92 | B56 | O | L27 |
| P2-867 | O | B92 | B56 | O | L29 |
| P2-868 | O | B92 | B56 | O | L36 |
| P2-869 | O | B92 | B56 | O | L40 |
| P2-870 | O | B92 | B56 | O | L53 |
| P2-871 | O | B92 | B56 | O | L59 |
| P2-872 | O | B92 | B56 | O | L65 |
| P2-873 | O | B92 | B56 | O | L68 |
| P2-874 | O | B92 | B56 | O | L119 |
| P2-875 | O | B92 | B56 | O | L128 |
| P2-876 | O | B92 | B56 | O | L148 |
| P2-877 | O | B44 | A2 | S | Single bond |
| P2-878 | O | B44 | A2 | S | L5 |
| P2-879 | O | B44 | A2 | S | L6 |
| P2-880 | O | B44 | A2 | S | L27 |
| P2-881 | O | B44 | A2 | S | L29 |
| P2-882 | O | B44 | A2 | S | L36 |
| P2-883 | O | B92 | A2 | S | Single bond |
| P2-884 | O | B92 | A2 | S | L5 |
| P2-885 | O | B92 | A2 | S | L6 |
| P2-886 | O | B92 | A2 | S | L27 |
| P2-887 | O | B92 | A2 | S | L29 |
| P2-888 | O | B92 | A2 | S | L36 |
| P2-889 | O | B44 | A3 | S | Single bond |
| P2-890 | O | B44 | A3 | S | L5 |
| P2-891 | O | B44 | A3 | S | L6 |
| P2-892 | O | B44 | A3 | S | L27 |
| P2-893 | O | B44 | A3 | S | L29 |
| P2-894 | O | B44 | A3 | S | L36 |
| P2-895 | O | B92 | A3 | S | Single bond |
| P2-896 | O | B92 | A3 | S | L5 |
| P2-897 | O | B92 | A3 | S | L6 |
| P2-898 | O | B92 | A3 | S | L27 |
| P2-899 | O | B92 | A3 | S | L29 |
| P2-900 | O | B92 | A3 | S | L36 |
| P2-901 | O | B44 | A2 | Se | L6 |
| P2-902 | O | B44 | A2 | Se | L36 |
| P2-903 | O | B92 | A2 | Se | L6 |
| P2-904 | O | B92 | A2 | Se | L36 |
| P2-905 | O | B44 | A3 | Se | L6 |
| P2-906 | O | B44 | A3 | Se | L36 |
| P2-907 | O | B92 | A3 | Se | L6 |
| P2-908 | O | B92 | A3 | Se | L36 |
| P2-909 | O | B44 | A2 | S═O | L6 |
| P2-910 | O | B44 | A2 | S═O | L36 |
| P2-911 | O | B92 | A2 | S═O | L6 |
| P2-912 | O | B92 | A2 | S═O | L36 |
| P2-913 | O | B44 | A3 | S═O | L6 |
| P2-914 | O | B44 | A3 | S═O | L36 |
| P2-915 | O | B92 | A3 | S═O | L6 |
| P2-916 | O | B92 | A3 | S═O | L36 |
| P2-917 | O | B44 | A2 | O═S═O | Single bond |
| P2-918 | O | B44 | A2 | O═S═O | L5 |
| P2-919 | O | B44 | A2 | O═S═O | L6 |
| P2-920 | O | B44 | A2 | O═S═O | L27 |
| P2-921 | O | B44 | A2 | O═S═O | L29 |
| P2-922 | O | B44 | A2 | O═S═O | L36 |
| P2-923 | O | B92 | A2 | O═S═O | Single bond |
| P2-924 | O | B92 | A2 | O═S═O | L5 |
| P2-925 | O | B92 | A2 | O═S═O | L6 |
| P2-926 | O | B92 | A2 | O═S═O | L27 |
| P2-927 | O | B92 | A2 | O═S═O | L29 |
| P2-928 | O | B92 | A2 | O═S═O | L36 |
| P2-929 | O | B44 | A3 | O═S═O | Single bond |
| P2-930 | O | B44 | A3 | O═S═O | L5 |
| P2-931 | O | B44 | A3 | O═S═O | L6 |
| P2-932 | O | B44 | A3 | O═S═O | L27 |
| P2-933 | O | B44 | A3 | O═S═O | L29 |
| P2-934 | O | B44 | A3 | O═S═O | L36 |
| P2-935 | O | B92 | A3 | O═S═O | Single bond |
| P2-936 | O | B92 | A3 | O═S═O | L5 |
| P2-937 | O | B92 | A3 | O═S═O | L6 |
| P2-938 | O | B92 | A3 | O═S═O | L27 |
| P2-939 | O | B92 | A3 | O═S═O | L29 |
| P2-940 | O | B92 | A3 | O═S═O | L36 |
| P2-941 | S | B44 | A2 | O | Single bond |
| P2-942 | S | B44 | A2 | O | L5 |
| P2-943 | S | B44 | A2 | O | L6 |
| P2-944 | S | B44 | A2 | O | L27 |
| P2-945 | S | B44 | A2 | O | L29 |
| P2-946 | S | B44 | A2 | O | L36 |
| P2-947 | S | B92 | A2 | O | Single bond |
| P2-948 | S | B92 | A2 | O | L5 |
| P2-949 | S | B92 | A2 | O | L6 |
| P2-950 | S | B92 | A2 | O | L27 |
| P2-951 | S | B92 | A2 | O | L29 |
| P2-952 | S | B92 | A2 | O | L36 |
| P2-953 | S | B44 | A3 | O | Single bond |
| P2-954 | S | B44 | A3 | O | L5 |
| P2-955 | S | B44 | A3 | O | L6 |
| P2-956 | S | B44 | A3 | O | L27 |
| P2-957 | S | B44 | A3 | O | L29 |
| P2-958 | S | B44 | A3 | O | L36 |
| P2-959 | S | B92 | A3 | O | Single bond |
| P2-960 | S | B92 | A3 | O | L5 |
| P2-961 | S | B92 | A3 | O | L6 |
| P2-962 | S | B92 | A3 | O | L27 |
| P2-963 | S | B92 | A3 | O | L29 |
| P2-964 | S | B92 | A3 | O | L36 |
| P2-965 | Se | B44 | A2 | O | L5 |
| P2-966 | Se | B44 | A2 | O | L6 |
| P2-967 | Se | B44 | A2 | O | L27 |
| P2-968 | Se | B44 | A2 | O | L29 |
| P2-969 | Se | B44 | A2 | O | L36 |
| P2-970 | Se | B92 | A2 | O | L5 |
| P2-971 | Se | B92 | A2 | O | L6 |
| P2-972 | Se | B92 | A2 | O | L27 |
| P2-973 | Se | B92 | A2 | O | L29 |
| P2-974 | Se | B92 | A2 | O | L36 |
| P2-975 | Se | B44 | A3 | O | L5 |
| P2-976 | Se | B44 | A3 | O | L6 |
| P2-977 | Se | B44 | A3 | O | L27 |
| P2-978 | Se | B44 | A3 | O | L29 |
| P2-979 | Se | B44 | A3 | O | L36 |
| P2-980 | Se | B92 | A3 | O | L5 |
| P2-981 | Se | B92 | A3 | O | L6 |
| P2-982 | Se | B92 | A3 | O | L27 |
| P2-983 | Se | B92 | A3 | O | L29 |
| P2-984 | Se | B92 | A3 | O | L36 |
| P2-985 | S | B44 | A2 | O═S═O | Single bond |
| P2-986 | S | B44 | A2 | O═S═O | L5 |
| P2-987 | S | B44 | A2 | O═S═O | L6 |
| P2-988 | S | B44 | A2 | O═S═O | L27 |
| P2-989 | S | B44 | A2 | O═S═O | L29 |
| P2-990 | S | B44 | A2 | O═S═O | L36 |
| P2-991 | S | B92 | A2 | O═S═O | Single bond |
| P2-992 | S | B92 | A2 | O═S═O | L5 |
| P2-993 | S | B92 | A2 | O═S═O | L6 |
| P2-994 | S | B92 | A2 | O═S═O | L27 |
| P2-995 | S | B92 | A2 | O═S═O | L29 |
| P2-996 | S | B92 | A2 | O═S═O | L36 |
| P2-997 | S | B44 | A3 | O═S═O | Single bond |
| P2-998 | S | B44 | A3 | O═S═O | L5 |
| P2-999 | S | B44 | A3 | O═S═O | L6 |
| P2-1000 | S | B44 | A3 | O═S═O | L27 |
| P2-1001 | S | B44 | A3 | O═S═O | L29 |
| P2-1002 | S | B44 | A3 | O═S═O | L36 |
| P2-1003 | S | B92 | A3 | O═S═O | Single bond |
| P2-1004 | S | B92 | A3 | O═S═O | L5 |
| P2-1005 | S | B92 | A3 | O═S═O | L6 |
| P2-1006 | S | B92 | A3 | O═S═O | L27 |
| P2-1007 | S | B92 | A3 | O═S═O | L29 |
| P2-1008 | S | B92 | A3 | O═S═O | L36 |
| P2-1009 | Se | B44 | A2 | O═S═O | L5 |
| P2-1010 | Se | B44 | A2 | O═S═O | L6 |
| P2-1011 | Se | B44 | A2 | O═S═O | L27 |
| P2-1012 | Se | B44 | A2 | O═S═O | L29 |
| P2-1013 | Se | B44 | A2 | O═S═O | L36 |
| P2-1014 | Se | B92 | A2 | O═S═O | L5 |
| P2-1015 | Se | B92 | A2 | O═S═O | L6 |
| P2-1016 | Se | B92 | A2 | O═S═O | L27 |
| P2-1017 | Se | B92 | A2 | O═S═O | L29 |
| P2-1018 | Se | B92 | A2 | O═S═O | L36 |
| P2-1019 | Se | B44 | A3 | O═S═O | L5 |
| P2-1020 | Se | B44 | A3 | O═S═O | L6 |
| P2-1021 | Se | B44 | A3 | O═S═O | L27 |
| P2-1022 | Se | B44 | A3 | O═S═O | L29 |
| P2-1023 | Se | B44 | A3 | O═S═O | L36 |
| P2-1024 | Se | B92 | A3 | O═S═O | L5 |
| P2-1025 | Se | B92 | A3 | O═S═O | L6 |
| P2-1026 | Se | B92 | A3 | O═S═O | L27 |
| P2-1027 | Se | B92 | A3 | O═S═O | L29 |
| P2-1028 | Se | B92 | A3 | O═S═O | L36 |

the compounds P3-1 to P3-1028 have structures represented by formula I-2-1; wherein R¹ and R² are the same, and W, Z, R¹, R², X, and L correspond to atoms or groups selected from the following table, respectively:

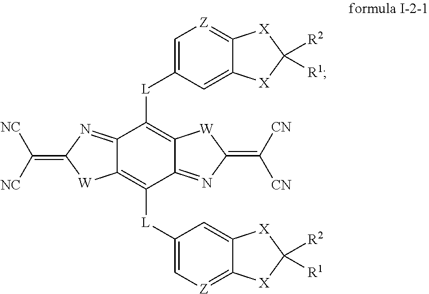

| | | | R¹ | | |
|---|---|---|---|---|---|
| | | | and | | |
| Compound No. | W | Z | R² | X | L |
| P3-1 | O | C-A1 | A1 | O | Single |
| | | | | | bond |
| P3-2 | O | C-A1 | A1 | O | O |
| P3-3 | O | C-A1 | A1 | O | L1 |
| P3-4 | O | C-A1 | A1 | O | L5 |
| P3-5 | O | C-A1 | A1 | O | L9 |
| P3-6 | O | C-A1 | A1 | O | L18 |
| P3-7 | O | C-A1 | A1 | O | L22 |
| P3-8 | O | C-A1 | A1 | O | L27 |
| P3-9 | O | C-A1 | A1 | O | L29 |
| P3-10 | O | C-A1 | A1 | O | L36 |
| P3-11 | O | C-A1 | A1 | O | L40 |
| P3-12 | O | C-A1 | A1 | O | L53 |
| P3-13 | O | C-A1 | A1 | O | L59 |
| P3-14 | O | C-A1 | A1 | O | L65 |
| P3-15 | O | C-A1 | A1 | O | L68 |
| P3-16 | O | C-A1 | A1 | O | L119 |
| P3-17 | O | C-A1 | A1 | O | L128 |
| P3-18 | O | C-A1 | A1 | O | L148 |
| P3-19 | O | C-A2 | A1 | O | Single |
| | | | | | bond |
| P3-20 | O | C-A2 | A1 | O | O |
| P3-21 | O | C-A2 | A1 | O | L1 |
| P3-22 | O | C-A2 | A1 | O | L5 |
| P3-23 | O | C-A2 | A1 | O | L9 |
| P3-24 | O | C-A2 | A1 | O | L18 |
| P3-25 | O | C-A2 | A1 | O | L22 |
| P3-26 | O | C-A2 | A1 | O | L27 |
| P3-27 | O | C-A2 | A1 | O | L29 |
| P3-28 | O | C-A2 | A1 | O | L36 |
| P3-29 | O | C-A2 | A1 | O | L40 |
| P3-30 | O | C-A2 | A1 | O | L53 |
| P3-31 | O | C-A2 | A1 | O | L59 |
| P3-32 | O | C-A2 | A1 | O | L65 |
| P3-33 | O | C-A2 | A1 | O | L68 |
| P3-34 | O | C-A2 | A1 | O | L119 |
| P3-35 | O | C-A2 | A1 | O | L128 |
| P3-36 | O | C-A2 | A1 | O | L148 |
| P3-37 | O | C-A3 | A1 | O | Single |
| | | | | | bond |
| P3-38 | O | C-A3 | A1 | O | O |
| P3-39 | O | C-A3 | A1 | O | L1 |
| P3-40 | O | C-A3 | A1 | O | L5 |
| P3-41 | O | C-A3 | A1 | O | L9 |
| P3-42 | O | C-A3 | A1 | O | L18 |
| P3-43 | O | C-A3 | A1 | O | L22 |
| P3-44 | O | C-A3 | A1 | O | L27 |
| P3-45 | O | C-A3 | A1 | O | L29 |
| P3-46 | O | C-A3 | A1 | O | L36 |
| P3-47 | O | C-A3 | A1 | O | L40 |
| P3-48 | O | C-A3 | A1 | O | L53 |
| P3-49 | O | C-A3 | A1 | O | L59 |
| P3-50 | O | C-A3 | A1 | O | L65 |
| P3-51 | O | C-A3 | A1 | O | L68 |
| P3-52 | O | C-A3 | A1 | O | L119 |
| P3-53 | O | C-A3 | A1 | O | L128 |
| P3-54 | O | C-A3 | A1 | O | L148 |
| P3-55 | O | C-B41 | A1 | O | Single |
| | | | | | bond |
| P3-56 | O | C-B41 | A1 | O | O |
| P3-57 | O | C-B41 | A1 | O | L1 |
| P3-58 | O | C-B41 | A1 | O | L5 |
| P3-59 | O | C-B41 | A1 | O | L9 |
| P3-60 | O | C-B41 | A1 | O | L18 |
| P3-61 | O | C-B41 | A1 | O | L22 |
| P3-62 | O | C-B41 | A1 | O | L27 |
| P3-63 | O | C-B41 | A1 | O | L29 |
| P3-64 | O | C-B41 | A1 | O | L36 |
| P3-65 | O | C-B41 | A1 | O | L40 |
| P3-66 | O | C-B41 | A1 | O | L53 |
| P3-67 | O | C-B41 | A1 | O | L59 |
| P3-68 | O | C-B41 | A1 | O | L65 |
| P3-69 | O | C-B41 | A1 | O | L68 |
| P3-70 | O | C-B41 | A1 | O | L119 |
| P3-71 | O | C-B41 | A1 | O | L128 |
| P3-72 | O | C-B41 | A1 | O | L148 |
| P3-73 | O | C-B56 | A1 | O | Single |
| | | | | | bond |
| P3-74 | O | C-B56 | A1 | O | O |
| P3-75 | O | C-B56 | A1 | O | L1 |
| P3-76 | O | C-B56 | A1 | O | L5 |
| P3-77 | O | C-B56 | A1 | O | L9 |
| P3-78 | O | C-B56 | A1 | O | L18 |
| P3-79 | O | C-B56 | A1 | O | L22 |
| P3-80 | O | C-B56 | A1 | O | L27 |
| P3-81 | O | C-B56 | A1 | O | L29 |
| P3-82 | O | C-B56 | A1 | O | L36 |
| P3-83 | O | C-B56 | A1 | O | L40 |
| P3-84 | O | C-B56 | A1 | O | L53 |
| P3-85 | O | C-B56 | A1 | O | L59 |
| P3-86 | O | C-B56 | A1 | O | L65 |
| P3-87 | O | C-B56 | A1 | O | L68 |
| P3-88 | O | C-B56 | A1 | O | L119 |
| P3-89 | O | C-B56 | A1 | O | L128 |
| P3-90 | O | C-B56 | A1 | O | L148 |
| P3-91 | O | N | A1 | O | Single |
| | | | | | bond |
| P3-92 | O | N | A1 | O | O |
| P3-93 | O | N | A1 | O | L1 |
| P3-94 | O | N | A1 | O | L5 |
| P3-95 | O | N | A1 | O | L9 |
| P3-96 | O | N | A1 | O | L18 |
| P3-97 | O | N | A1 | O | L22 |
| P3-98 | O | N | A1 | O | L27 |
| P3-99 | O | N | A1 | O | L29 |
| P3-100 | O | N | A1 | O | L36 |
| P3-101 | O | N | A1 | O | L40 |
| P3-102 | O | N | A1 | O | L53 |
| P3-103 | O | N | A1 | O | L59 |
| P3-104 | O | N | A1 | O | L65 |
| P3-105 | O | N | A1 | O | L68 |
| P3-106 | O | N | A1 | O | L119 |
| P3-107 | O | N | A1 | O | L128 |
| P3-108 | O | N | A1 | O | L148 |
| P3-109 | O | C-A1 | A2 | O | Single |
| | | | | | bond |
| P3-110 | O | C-A1 | A2 | O | O |
| P3-111 | O | C-A1 | A2 | O | L1 |
| P3-112 | O | C-A1 | A2 | O | L2 |
| P3-113 | O | C-A1 | A2 | O | L3 |
| P3-114 | O | C-A1 | A2 | O | L4 |
| P3-115 | O | C-A1 | A2 | O | L5 |
| P3-116 | O | C-A1 | A2 | O | L6 |
| P3-117 | O | C-A1 | A2 | O | L7 |
| P3-118 | O | C-A1 | A2 | O | L9 |
| P3-119 | O | C-A1 | A2 | O | L11 |
| P3-120 | O | C-A1 | A2 | O | L14 |
| P3-121 | O | C-A1 | A2 | O | L18 |
| P3-122 | O | C-A1 | A2 | O | L22 |
| P3-123 | O | C-A1 | A2 | O | L27 |
| P3-124 | O | C-A1 | A2 | O | L28 |
| P3-125 | O | C-A1 | A2 | O | L29 |
| P3-126 | O | C-A1 | A2 | O | L30 |
| P3-127 | O | C-A1 | A2 | O | L32 |
| P3-128 | O | C-A1 | A2 | O | L36 |
| P3-129 | O | C-A1 | A2 | O | L40 |
| P3-130 | O | C-A1 | A2 | O | L48 |
| P3-131 | O | C-A1 | A2 | O | L53 |
| P3-132 | O | C-A1 | A2 | O | L59 |
| P3-133 | O | C-A1 | A2 | O | L65 |
| P3-134 | O | C-A1 | A2 | O | L68 |
| P3-135 | O | C-A1 | A2 | O | L79 |
| P3-136 | O | C-A1 | A2 | O | L89 |
| P3-137 | O | C-A1 | A2 | O | L102 |
| P3-138 | O | C-A1 | A2 | O | L111 |
| P3-139 | O | C-A1 | A2 | O | L119 |
| P3-140 | O | C-A1 | A2 | O | L128 |
| P3-141 | O | C-A1 | A2 | O | L148 |
| P3-142 | O | C-A1 | A2 | O | L156 |
| P3-143 | O | C-A1 | A2 | O | L167 |
| P3-144 | O | C-A1 | A2 | O | L169 |
| P3-145 | O | C-A1 | A2 | O | L172 |
| P3-146 | O | C-A2 | A2 | O | Single |
| | | | | | bond |
| P3-147 | O | C-A2 | A2 | O | O |
| P3-148 | O | C-A2 | A2 | O | L1 |
| P3-149 | O | C-A2 | A2 | O | L2 |
| P3-150 | O | C-A2 | A2 | O | L3 |
| P3-151 | O | C-A2 | A2 | O | L4 |
| P3-152 | O | C-A2 | A2 | O | L5 |
| P3-153 | O | C-A2 | A2 | O | L6 |
| P3-154 | O | C-A2 | A2 | O | L7 |
| P3-155 | O | C-A2 | A2 | O | L9 |
| P3-156 | O | C-A2 | A2 | O | L11 |
| P3-157 | O | C-A2 | A2 | O | L14 |
| P3-158 | O | C-A2 | A2 | O | L18 |
| P3-159 | O | C-A2 | A2 | O | L22 |
| P3-160 | O | C-A2 | A2 | O | L27 |
| P3-161 | O | C-A2 | A2 | O | L28 |
| P3-162 | O | C-A2 | A2 | O | L29 |
| P3-163 | O | C-A2 | A2 | O | L30 |
| P3-164 | O | C-A2 | A2 | O | L32 |
| P3-165 | O | C-A2 | A2 | O | L36 |
| P3-166 | O | C-A2 | A2 | O | L40 |
| P3-167 | O | C-A2 | A2 | O | L48 |
| P3-168 | O | C-A2 | A2 | O | L53 |
| P3-169 | O | C-A2 | A2 | O | L59 |
| P3-170 | O | C-A2 | A2 | O | L65 |
| P3-171 | O | C-A2 | A2 | O | L68 |
| P3-172 | O | C-A2 | A2 | O | L79 |
| P3-173 | O | C-A2 | A2 | O | L89 |
| P3-174 | O | C-A2 | A2 | O | L102 |
| P3-175 | O | C-A2 | A2 | O | L111 |
| P3-176 | O | C-A2 | A2 | O | L119 |
| P3-177 | O | C-A2 | A2 | O | L128 |
| P3-178 | O | C-A2 | A2 | O | L148 |
| P3-179 | O | C-A2 | A2 | O | L156 |
| P3-180 | O | C-A2 | A2 | O | L167 |
| P3-181 | O | C-A2 | A2 | O | L169 |
| P3-182 | O | C-A2 | A2 | O | L172 |
| P3-183 | O | C-A3 | A2 | O | Single |
| | | | | | bond |
| P3-184 | O | C-A3 | A2 | O | O |
| P3-185 | O | C-A3 | A2 | O | L1 |
| P3-186 | O | C-A3 | A2 | O | L2 |
| P3-187 | O | C-A3 | A2 | O | L3 |
| P3-188 | O | C-A3 | A2 | O | L4 |
| P3-189 | O | C-A3 | A2 | O | L5 |
| P3-190 | O | C-A3 | A2 | O | L6 |
| P3-191 | O | C-A3 | A2 | O | L7 |
| P3-192 | O | C-A3 | A2 | O | L9 |
| P3-193 | O | C-A3 | A2 | O | L11 |
| P3-194 | O | C-A3 | A2 | O | L14 |
| P3-195 | O | C-A3 | A2 | O | L18 |
| P3-196 | O | C-A3 | A2 | O | L22 |
| P3-197 | O | C-A3 | A2 | O | L27 |
| P3-198 | O | C-A3 | A2 | O | L28 |
| P3-199 | O | C-A3 | A2 | O | L29 |
| P3-200 | O | C-A3 | A2 | O | L30 |
| P3-201 | O | C-A3 | A2 | O | L32 |
| P3-202 | O | C-A3 | A2 | O | L36 |
| P3-203 | O | C-A3 | A2 | O | L40 |
| P3-204 | O | C-A3 | A2 | O | L48 |
| P3-205 | O | C-A3 | A2 | O | L53 |
| P3-206 | O | C-A3 | A2 | O | L59 |
| P3-207 | O | C-A3 | A2 | O | L65 |
| P3-208 | O | C-A3 | A2 | O | L68 |
| P3-209 | O | C-A3 | A2 | O | L79 |
| P3-210 | O | C-A3 | A2 | O | L89 |
| P3-211 | O | C-A3 | A2 | O | L102 |
| P3-212 | O | C-A3 | A2 | O | L111 |
| P3-213 | O | C-A3 | A2 | O | L119 |
| P3-214 | O | C-A3 | A2 | O | L128 |
| P3-215 | O | C-A3 | A2 | O | L148 |
| P3-216 | O | C-A3 | A2 | O | L156 |
| P3-217 | O | C-A3 | A2 | O | L167 |
| P3-218 | O | C-A3 | A2 | O | L169 |
| P3-219 | O | C-A3 | A2 | O | L172 |
| P3-220 | O | C-B41 | A2 | O | Single |
| | | | | | bond |
| P3-221 | O | C-B41 | A2 | O | O |
| P3-222 | O | C-B41 | A2 | O | L1 |
| P3-223 | O | C-B41 | A2 | O | L2 |
| P3-224 | O | C-B41 | A2 | O | L3 |
| P3-225 | O | C-B41 | A2 | O | L4 |
| P3-226 | O | C-B41 | A2 | O | L5 |
| P3-227 | O | C-B41 | A2 | O | L6 |
| P3-228 | O | C-B41 | A2 | O | L7 |
| P3-229 | O | C-B41 | A2 | O | L9 |
| P3-230 | O | C-B41 | A2 | O | L11 |
| P3-231 | O | C-B41 | A2 | O | L14 |
| P3-232 | O | C-B41 | A2 | O | L18 |
| P3-233 | O | C-B41 | A2 | O | L22 |
| P3-234 | O | C-B41 | A2 | O | L27 |
| P3-235 | O | C-B41 | A2 | O | L28 |
| P3-236 | O | C-B41 | A2 | O | L29 |
| P3-237 | O | C-B41 | A2 | O | L30 |
| P3-238 | O | C-B41 | A2 | O | L32 |
| P3-239 | O | C-B41 | A2 | O | L36 |
| P3-240 | O | C-B41 | A2 | O | L40 |
| P3-241 | O | C-B41 | A2 | O | L48 |
| P3-242 | O | C-B41 | A2 | O | L53 |
| P3-243 | O | C-B41 | A2 | O | L59 |
| P3-244 | O | C-B41 | A2 | O | L65 |
| P3-245 | O | C-B41 | A2 | O | L68 |
| P3-246 | O | C-B41 | A2 | O | L79 |
| P3-247 | O | C-B41 | A2 | O | L89 |
| P3-248 | O | C-B41 | A2 | O | L102 |
| P3-249 | O | C-B41 | A2 | O | L111 |
| P3-250 | O | C-B41 | A2 | O | L119 |
| P3-251 | O | C-B41 | A2 | O | L128 |
| P3-252 | O | C-B41 | A2 | O | L148 |
| P3-253 | O | C-B41 | A2 | O | L156 |
| P3-254 | O | C-B41 | A2 | O | L167 |
| P3-255 | O | C-B41 | A2 | O | L169 |
| P3-256 | O | C-B41 | A2 | O | L172 |
| P3-257 | O | C-B56 | A2 | O | Single |
| | | | | | bond |
| P3-258 | O | C-B56 | A2 | O | O |
| P3-259 | O | C-B56 | A2 | O | L1 |
| P3-260 | O | C-B56 | A2 | O | L2 |
| P3-261 | O | C-B56 | A2 | O | L3 |
| P3-262 | O | C-B56 | A2 | O | L4 |
| P3-263 | O | C-B56 | A2 | O | L5 |
| P3-264 | O | C-B56 | A2 | O | L6 |
| P3-265 | O | C-B56 | A2 | O | L7 |
| P3-266 | O | C-B56 | A2 | O | L9 |
| P3-267 | O | C-B56 | A2 | O | L11 |
| P3-268 | O | C-B56 | A2 | O | L14 |
| P3-269 | O | C-B56 | A2 | O | L18 |
| P3-270 | O | C-B56 | A2 | O | L22 |
| P3-271 | O | C-B56 | A2 | O | L27 |
| P3-272 | O | C-B56 | A2 | O | L28 |
| P3-273 | O | C-B56 | A2 | O | L29 |
| P3-274 | O | C-B56 | A2 | O | L30 |
| P3-275 | O | C-B56 | A2 | O | L32 |
| P3-276 | O | C-B56 | A2 | O | L36 |
| P3-277 | O | C-B56 | A2 | O | L40 |
| P3-278 | O | C-B56 | A2 | O | L48 |
| P3-279 | O | C-B56 | A2 | O | L53 |
| P3-280 | O | C-B56 | A2 | O | L59 |
| P3-281 | O | C-B56 | A2 | O | L65 |
| P3-282 | O | C-B56 | A2 | O | L68 |
| P3-283 | O | C-B56 | A2 | O | L79 |
| P3-284 | O | C-B56 | A2 | O | L89 |
| P3-285 | O | C-B56 | A2 | O | L102 |
| P3-286 | O | C-B56 | A2 | O | L111 |
| P3-287 | O | C-B56 | A2 | O | L119 |
| P3-288 | O | C-B56 | A2 | O | L128 |
| P3-289 | O | C-B56 | A2 | O | L148 |
| P3-290 | O | C-B56 | A2 | O | L156 |
| P3-291 | O | C-B56 | A2 | O | L167 |
| P3-292 | O | C-B56 | A2 | O | L169 |
| P3-293 | O | C-B56 | A2 | O | L172 |
| P3-294 | O | N | A2 | O | Single |
| | | | | | bond |
| P3-295 | O | N | A2 | O | O |
| P3-296 | O | N | A2 | O | L1 |
| P3-297 | O | N | A2 | O | L2 |
| P3-298 | O | N | A2 | O | L3 |
| P3-299 | O | N | A2 | O | L4 |
| P3-300 | O | N | A2 | O | L5 |
| P3-301 | O | N | A2 | O | L6 |
| P3-302 | O | N | A2 | O | L7 |
| P3-303 | O | N | A2 | O | L9 |
| P3-304 | O | N | A2 | O | L11 |
| P3-305 | O | N | A2 | O | L14 |
| P3-306 | O | N | A2 | O | L18 |
| P3-307 | O | N | A2 | O | L22 |
| P3-308 | O | N | A2 | O | L27 |
| P3-309 | O | N | A2 | O | L28 |
| P3-310 | O | N | A2 | O | L29 |
| P3-311 | O | N | A2 | O | L30 |
| P3-312 | O | N | A2 | O | L32 |
| P3-313 | O | N | A2 | O | L36 |
| P3-314 | O | N | A2 | O | L40 |
| P3-315 | O | N | A2 | O | L48 |
| P3-316 | O | N | A2 | O | L53 |
| P3-317 | O | N | A2 | O | L59 |
| P3-318 | O | N | A2 | O | L65 |
| P3-319 | O | N | A2 | O | L68 |
| P3-320 | O | N | A2 | O | L79 |
| P3-321 | O | N | A2 | O | L89 |
| P3-322 | O | N | A2 | O | L102 |
| P3-323 | O | N | A2 | O | L111 |
| P3-324 | O | N | A2 | O | L119 |
| P3-325 | O | N | A2 | O | L128 |
| P3-326 | O | N | A2 | O | L148 |
| P3-327 | O | N | A2 | O | L156 |
| P3-328 | O | N | A2 | O | L167 |
| P3-329 | O | N | A2 | O | L169 |
| P3-330 | O | N | A2 | O | L172 |
| P3-331 | O | C-A1 | A3 | O | Single |
| | | | | | bond |
| P3-332 | O | C-A1 | A3 | O | O |
| P3-333 | O | C-A1 | A3 | O | L1 |
| P3-334 | O | C-A1 | A3 | O | L2 |
| P3-335 | O | C-A1 | A3 | O | L3 |
| P3-336 | O | C-A1 | A3 | O | L4 |
| P3-337 | O | C-A1 | A3 | O | L5 |
| P3-338 | O | C-A1 | A3 | O | L6 |
| P3-339 | O | C-A1 | A3 | O | L7 |
| P3-340 | O | C-A1 | A3 | O | L9 |
| P3-341 | O | C-A1 | A3 | O | L11 |
| P3-342 | O | C-A1 | A3 | O | L14 |
| P3-343 | O | C-A1 | A3 | O | L18 |
| P3-344 | O | C-A1 | A3 | O | L22 |
| P3-345 | O | C-A1 | A3 | O | L27 |
| P3-346 | O | C-A1 | A3 | O | L28 |
| P3-347 | O | C-A1 | A3 | O | L29 |
| P3-348 | O | C-A1 | A3 | O | L30 |
| P3-349 | O | C-A1 | A3 | O | L32 |
| P3-350 | O | C-A1 | A3 | O | L36 |
| P3-351 | O | C-A1 | A3 | O | L40 |
| P3-352 | O | C-A1 | A3 | O | L48 |
| P3-353 | O | C-A1 | A3 | O | L53 |
| P3-354 | O | C-A1 | A3 | O | L59 |
| P3-355 | O | C-A1 | A3 | O | L65 |
| P3-356 | O | C-A1 | A3 | O | L68 |
| P3-357 | O | C-A1 | A3 | O | L79 |
| P3-358 | O | C-A1 | A3 | O | L89 |
| P3-359 | O | C-A1 | A3 | O | L102 |
| P3-360 | O | C-A1 | A3 | O | L111 |
| P3-361 | O | C-A1 | A3 | O | L119 |
| P3-362 | O | C-A1 | A3 | O | L128 |
| P3-363 | O | C-A1 | A3 | O | L148 |
| P3-364 | O | C-A1 | A3 | O | L156 |
| P3-365 | O | C-A1 | A3 | O | L167 |
| P3-366 | O | C-A1 | A3 | O | L169 |
| P3-367 | O | C-A1 | A3 | O | L172 |
| P3-368 | O | C-A2 | A3 | O | Single |
| | | | | | bond |
| P3-369 | O | C-A2 | A3 | O | O |
| P3-370 | O | C-A2 | A3 | O | L1 |
| P3-371 | O | C-A2 | A3 | O | L2 |
| P3-372 | O | C-A2 | A3 | O | L3 |
| P3-373 | O | C-A2 | A3 | O | L4 |
| P3-374 | O | C-A2 | A3 | O | L5 |
| P3-375 | O | C-A2 | A3 | O | L6 |
| P3-376 | O | C-A2 | A3 | O | L7 |
| P3-377 | O | C-A2 | A3 | O | L9 |
| P3-378 | O | C-A2 | A3 | O | L11 |
| P3-379 | O | C-A2 | A3 | O | L14 |
| P3-380 | O | C-A2 | A3 | O | L18 |
| P3-381 | O | C-A2 | A3 | O | L22 |
| P3-382 | O | C-A2 | A3 | O | L27 |
| P3-383 | O | C-A2 | A3 | O | L28 |
| P3-384 | O | C-A2 | A3 | O | L29 |
| P3-385 | O | C-A2 | A3 | O | L30 |
| P3-386 | O | C-A2 | A3 | O | L32 |
| P3-387 | O | C-A2 | A3 | O | L36 |
| P3-388 | O | C-A2 | A3 | O | L40 |
| P3-389 | O | C-A2 | A3 | O | L48 |
| P3-390 | O | C-A2 | A3 | O | L53 |
| P3-391 | O | C-A2 | A3 | O | L59 |
| P3-392 | O | C-A2 | A3 | O | L65 |
| P3-393 | O | C-A2 | A3 | O | L68 |
| P3-394 | O | C-A2 | A3 | O | L79 |
| P3-395 | O | C-A2 | A3 | O | L89 |
| P3-396 | O | C-A2 | A3 | O | L102 |
| P3-397 | O | C-A2 | A3 | O | L111 |
| P3-398 | O | C-A2 | A3 | O | L119 |
| P3-399 | O | C-A2 | A3 | O | L128 |
| P3-400 | O | C-A2 | A3 | O | L148 |
| P3-401 | O | C-A2 | A3 | O | L156 |
| P3-402 | O | C-A2 | A3 | O | L167 |
| P3-403 | O | C-A2 | A3 | O | L169 |
| P3-404 | O | C-A2 | A3 | O | L172 |
| P3-405 | O | C-A3 | A3 | O | Single |
| | | | | | bond |
| P3-406 | O | C-A3 | A3 | O | O |
| P3-407 | O | C-A3 | A3 | O | L1 |
| P3-408 | O | C-A3 | A3 | O | L2 |
| P3-409 | O | C-A3 | A3 | O | L3 |
| P3-410 | O | C-A3 | A3 | O | L4 |
| P3-411 | O | C-A3 | A3 | O | L5 |
| P3-412 | O | C-A3 | A3 | O | L6 |
| P3-413 | O | C-A3 | A3 | O | L7 |
| P3-414 | O | C-A3 | A3 | O | L9 |
| P3-415 | O | C-A3 | A3 | O | L11 |
| P3-416 | O | C-A3 | A3 | O | L14 |
| P3-417 | O | C-A3 | A3 | O | L18 |
| P3-418 | O | C-A3 | A3 | O | L22 |
| P3-419 | O | C-A3 | A3 | O | L27 |
| P3-420 | O | C-A3 | A3 | O | L28 |
| P3-421 | O | C-A3 | A3 | O | L29 |
| P3-422 | O | C-A3 | A3 | O | L30 |
| P3-423 | O | C-A3 | A3 | O | L32 |
| P3-424 | O | C-A3 | A3 | O | L36 |
| P3-425 | O | C-A3 | A3 | O | L40 |
| P3-426 | O | C-A3 | A3 | O | L48 |
| P3-427 | O | C-A3 | A3 | O | L53 |
| P3-428 | O | C-A3 | A3 | O | L59 |
| P3-429 | O | C-A3 | A3 | O | L65 |
| P3-430 | O | C-A3 | A3 | O | L68 |
| P3-431 | O | C-A3 | A3 | O | L79 |
| P3-432 | O | C-A3 | A3 | O | L89 |
| P3-433 | O | C-A3 | A3 | O | L102 |
| P3-434 | O | C-A3 | A3 | O | L111 |
| P3-435 | O | C-A3 | A3 | O | L119 |
| P3-436 | O | C-A3 | A3 | O | L128 |
| P3-437 | O | C-A3 | A3 | O | L148 |
| P3-438 | O | C-A3 | A3 | O | L156 |
| P3-439 | O | C-A3 | A3 | O | L167 |
| P3-440 | O | C-A3 | A3 | O | L169 |
| P3-441 | O | C-A3 | A3 | O | L172 |
| P3-442 | O | C-B41 | A3 | O | Single |
| | | | | | bond |
| P3-443 | O | C-B41 | A3 | O | O |
| P3-444 | O | C-B41 | A3 | O | L1 |
| P3-445 | O | C-B41 | A3 | O | L2 |
| P3-446 | O | C-B41 | A3 | O | L3 |
| P3-447 | O | C-B41 | A3 | O | L4 |
| P3-448 | O | C-B41 | A3 | O | L5 |
| P3-449 | O | C-B41 | A3 | O | L6 |
| P3-450 | O | C-B41 | A3 | O | L7 |
| P3-451 | O | C-B41 | A3 | O | L9 |
| P3-452 | O | C-B41 | A3 | O | L11 |
| P3-453 | O | C-B41 | A3 | O | L14 |
| P3-454 | O | C-B41 | A3 | O | L18 |
| P3-455 | O | C-B41 | A3 | O | L22 |
| P3-456 | O | C-B41 | A3 | O | L27 |
| P3-457 | O | C-B41 | A3 | O | L28 |
| P3-458 | O | C-B41 | A3 | O | L29 |
| P3-459 | O | C-B41 | A3 | O | L30 |
| P3-460 | O | C-B41 | A3 | O | L32 |
| P3-461 | O | C-B41 | A3 | O | L36 |
| P3-462 | O | C-B41 | A3 | O | L40 |
| P3-463 | O | C-B41 | A3 | O | L48 |
| P3-464 | O | C-B41 | A3 | O | L53 |
| P3-465 | O | C-B41 | A3 | O | L59 |
| P3-466 | O | C-B41 | A3 | O | L65 |
| P3-467 | O | C-B41 | A3 | O | L68 |
| P3-468 | O | C-B41 | A3 | O | L79 |
| P3-469 | O | C-B41 | A3 | O | L89 |
| P3-470 | O | C-B41 | A3 | O | L102 |
| P3-471 | O | C-B41 | A3 | O | L111 |
| P3-472 | O | C-B41 | A3 | O | L119 |
| P3-473 | O | C-B41 | A3 | O | L128 |
| P3-474 | O | C-B41 | A3 | O | L148 |
| P3-475 | O | C-B41 | A3 | O | L156 |
| P3-476 | O | C-B41 | A3 | O | L167 |
| P3-477 | O | C-B41 | A3 | O | L169 |
| P3-478 | O | C-B41 | A3 | O | L172 |
| P3-479 | O | C-B56 | A3 | O | Single |
| | | | | | bond |
| P3-480 | O | C-B56 | A3 | O | O |
| P3-481 | O | C-B56 | A3 | O | L1 |
| P3-482 | O | C-B56 | A3 | O | L2 |
| P3-483 | O | C-B56 | A3 | O | L3 |
| P3-484 | O | C-B56 | A3 | O | L4 |
| P3-485 | O | C-B56 | A3 | O | L5 |
| P3-486 | O | C-B56 | A3 | O | L6 |
| P3-487 | O | C-B56 | A3 | O | L7 |
| P3-488 | O | C-B56 | A3 | O | L9 |
| P3-489 | O | C-B56 | A3 | O | L11 |
| P3-490 | O | C-B56 | A3 | O | L14 |
| P3-491 | O | C-B56 | A3 | O | L18 |
| P3-492 | O | C-B56 | A3 | O | L22 |
| P3-493 | O | C-B56 | A3 | O | L27 |
| P3-494 | O | C-B56 | A3 | O | L28 |
| P3-495 | O | C-B56 | A3 | O | L29 |
| P3-496 | O | C-B56 | A3 | O | L30 |
| P3-497 | O | C-B56 | A3 | O | L32 |
| P3-498 | O | C-B56 | A3 | O | L36 |
| P3-499 | O | C-B56 | A3 | O | L40 |
| P3-500 | O | C-B56 | A3 | O | L48 |
| P3-501 | O | C-B56 | A3 | O | L53 |
| P3-502 | O | C-B56 | A3 | O | L59 |
| P3-503 | O | C-B56 | A3 | O | L65 |
| P3-504 | O | C-B56 | A3 | O | L68 |
| P3-505 | O | C-B56 | A3 | O | L79 |
| P3-506 | O | C-B56 | A3 | O | L89 |
| P3-507 | O | C-B56 | A3 | O | L102 |
| P3-508 | O | C-B56 | A3 | O | L111 |
| P3-509 | O | C-B56 | A3 | O | L119 |
| P3-510 | O | C-B56 | A3 | O | L128 |
| P3-511 | O | C-B56 | A3 | O | L148 |
| P3-512 | O | C-B56 | A3 | O | L156 |
| P3-513 | O | C-B56 | A3 | O | L167 |
| P3-514 | O | C-B56 | A3 | O | L169 |
| P3-515 | O | C-B56 | A3 | O | L172 |
| P3-516 | O | N | A3 | O | Single |
| | | | | | bond |
| P3-517 | O | N | A3 | O | O |
| P3-518 | O | N | A3 | O | L1 |
| P3-519 | O | N | A3 | O | L2 |
| P3-520 | O | N | A3 | O | L3 |
| P3-521 | O | N | A3 | O | L4 |
| P3-522 | O | N | A3 | O | L5 |
| P3-523 | O | N | A3 | O | L6 |
| P3-524 | O | N | A3 | O | L7 |
| P3-525 | O | N | A3 | O | L9 |
| P3-526 | O | N | A3 | O | L11 |
| P3-527 | O | N | A3 | O | L14 |
| P3-528 | O | N | A3 | O | L18 |
| P3-529 | O | N | A3 | O | L22 |
| P3-530 | O | N | A3 | O | L27 |
| P3-531 | O | N | A3 | O | L28 |
| P3-532 | O | N | A3 | O | L29 |
| P3-533 | O | N | A3 | O | L30 |
| P3-534 | O | N | A3 | O | L32 |
| P3-535 | O | N | A3 | O | L36 |
| P3-536 | O | N | A3 | O | L40 |
| P3-537 | O | N | A3 | O | L48 |
| P3-538 | O | N | A3 | O | L53 |
| P3-539 | O | N | A3 | O | L59 |
| P3-540 | O | N | A3 | O | L65 |
| P3-541 | O | N | A3 | O | L68 |
| P3-542 | O | N | A3 | O | L79 |
| P3-543 | O | N | A3 | O | L89 |
| P3-544 | O | N | A3 | O | L102 |
| P3-545 | O | N | A3 | O | L111 |
| P3-546 | O | N | A3 | O | L119 |
| P3-547 | O | N | A3 | O | L128 |
| P3-548 | O | N | A3 | O | L148 |
| P3-549 | O | N | A3 | O | L156 |
| P3-550 | O | N | A3 | O | L167 |
| P3-551 | O | N | A3 | O | L169 |
| P3-552 | O | N | A3 | O | L172 |
| P3-553 | O | C-A1 | A12 | O | Single |
| | | | | | bond |
| P3-554 | O | C-A1 | A12 | O | O |
| P3-555 | O | C-A1 | A12 | O | L1 |
| P3-556 | O | C-A1 | A12 | O | L5 |
| P3-557 | O | C-A1 | A12 | O | L9 |
| P3-558 | O | C-A1 | A12 | O | L18 |
| P3-559 | O | C-A1 | A12 | O | L22 |
| P3-560 | O | C-A1 | A12 | O | L27 |
| P3-561 | O | C-A1 | A12 | O | L29 |
| P3-562 | O | C-A1 | A12 | O | L36 |
| P3-563 | O | C-A1 | A12 | O | L40 |
| P3-564 | O | C-A1 | A12 | O | L53 |
| P3-565 | O | C-A1 | A12 | O | L59 |
| P3-566 | O | C-A1 | A12 | O | L65 |
| P3-567 | O | C-A1 | A12 | O | L68 |
| P3-568 | O | C-A1 | A12 | O | L119 |
| P3-569 | O | C-A1 | A12 | O | L128 |
| P3-570 | O | C-A1 | A12 | O | L148 |
| P3-571 | O | C-A2 | A12 | O | Single |
| | | | | | bond |
| P3-572 | O | C-A2 | A12 | O | O |
| P3-573 | O | C-A2 | A12 | O | L1 |
| P3-574 | O | C-A2 | A12 | O | L5 |
| P3-575 | O | C-A2 | A12 | O | L9 |
| P3-576 | O | C-A2 | A12 | O | L18 |
| P3-577 | O | C-A2 | A12 | O | L22 |
| P3-578 | O | C-A2 | A12 | O | L27 |
| P3-579 | O | C-A2 | A12 | O | L29 |
| P3-580 | O | C-A2 | A12 | O | L36 |
| P3-581 | O | C-A2 | A12 | O | L40 |
| P3-582 | O | C-A2 | A12 | O | L53 |
| P3-583 | O | C-A2 | A12 | O | L59 |
| P3-584 | O | C-A2 | A12 | O | L65 |
| P3-585 | O | C-A2 | A12 | O | L68 |
| P3-586 | O | C-A2 | A12 | O | L119 |
| P3-587 | O | C-A2 | A12 | O | L128 |
| P3-588 | O | C-A2 | A12 | O | L148 |
| P3-589 | O | C-A3 | A12 | O | Single |
| | | | | | bond |
| P3-590 | O | C-A3 | A12 | O | O |
| P3-591 | O | C-A3 | A12 | O | L1 |
| P3-592 | O | C-A3 | A12 | O | L5 |
| P3-593 | O | C-A3 | A12 | O | L9 |
| P3-594 | O | C-A3 | A12 | O | L18 |
| P3-595 | O | C-A3 | A12 | O | L22 |
| P3-596 | O | C-A3 | A12 | O | L27 |
| P3-597 | O | C-A3 | A12 | O | L29 |
| P3-598 | O | C-A3 | A12 | O | L36 |
| P3-599 | O | C-A3 | A12 | O | L40 |
| P3-600 | O | C-A3 | A12 | O | L53 |
| P3-601 | O | C-A3 | A12 | O | L59 |
| P3-602 | O | C-A3 | A12 | O | L65 |
| P3-603 | O | C-A3 | A12 | O | L68 |
| P3-604 | O | C-A3 | A12 | O | L119 |
| P3-605 | O | C-A3 | A12 | O | L128 |
| P3-606 | O | C-A3 | A12 | O | L148 |
| P3-607 | O | C-B41 | A12 | O | Single |
| | | | | | bond |
| P3-608 | O | C-B41 | A12 | O | O |
| P3-609 | O | C-B41 | A12 | O | L1 |
| P3-610 | O | C-B41 | A12 | O | L5 |
| P3-611 | O | C-B41 | A12 | O | L9 |
| P3-612 | O | C-B41 | A12 | O | L18 |
| P3-613 | O | C-B41 | A12 | O | L22 |
| P3-614 | O | C-B41 | A12 | O | L27 |
| P3-615 | O | C-B41 | A12 | O | L29 |
| P3-616 | O | C-B41 | A12 | O | L36 |
| P3-617 | O | C-B41 | A12 | O | L40 |
| P3-618 | O | C-B41 | A12 | O | L53 |
| P3-619 | O | C-B41 | A12 | O | L59 |
| P3-620 | O | C-B41 | A12 | O | L65 |
| P3-621 | O | C-B41 | A12 | O | L68 |
| P3-622 | O | C-B41 | A12 | O | L119 |
| P3-623 | O | C-B41 | A12 | O | L128 |
| P3-624 | O | C-B41 | A12 | O | L148 |
| P3-625 | O | C-B56 | A12 | O | Single |
| | | | | | bond |
| P3-626 | O | C-B56 | A12 | O | 0 |
| P3-627 | O | C-B56 | A12 | O | L1 |
| P3-628 | O | C-B56 | A12 | O | L5 |
| P3-629 | O | C-B56 | A12 | O | L9 |
| P3-630 | O | C-B56 | A12 | O | L18 |
| P3-631 | O | C-B56 | A12 | O | L22 |
| P3-632 | O | C-B56 | A12 | O | L27 |
| P3-633 | O | C-B56 | A12 | O | L29 |
| P3-634 | O | C-B56 | A12 | O | L36 |
| P3-635 | O | C-B56 | A12 | O | L40 |
| P3-636 | O | C-B56 | A12 | O | L53 |
| P3-637 | O | C-B56 | A12 | O | L59 |
| P3-638 | O | C-B56 | A12 | O | L65 |
| P3-639 | O | C-B56 | A12 | O | L68 |
| P3-640 | O | C-B56 | A12 | O | L119 |
| P3-641 | O | C-B56 | A12 | O | L128 |
| P3-642 | O | C-B56 | A12 | O | L148 |
| P3-643 | O | N | A12 | O | Single |
| | | | | | bond |
| P3-644 | O | N | A12 | O | O |
| P3-645 | O | N | A12 | O | L1 |
| P3-646 | O | N | A12 | O | L5 |
| P3-647 | O | N | A12 | O | L9 |
| P3-648 | O | N | A12 | O | L18 |
| P3-649 | O | N | A12 | O | L22 |
| P3-650 | O | N | A12 | O | L27 |
| P3-651 | O | N | A12 | O | L29 |
| P3-652 | O | N | A12 | O | L36 |
| P3-653 | O | N | A12 | O | L40 |
| P3-654 | O | N | A12 | O | L53 |
| P3-655 | O | N | A12 | O | L59 |
| P3-656 | O | N | A12 | O | L65 |
| P3-657 | O | N | A12 | O | L68 |
| P3-658 | O | N | A12 | O | L119 |
| P3-659 | O | N | A12 | O | L128 |
| P3-660 | O | N | A12 | O | L148 |
| P3-661 | O | C-A1 | B41 | O | Single |
| | | | | | bond |
| P3-662 | O | C-A1 | B41 | O | O |
| P3-663 | O | C-A1 | B41 | O | L1 |
| P3-664 | O | C-A1 | B41 | O | L5 |
| P3-665 | O | C-A1 | B41 | O | L9 |
| P3-666 | O | C-A1 | B41 | O | L18 |
| P3-667 | O | C-A1 | B41 | O | L22 |
| P3-668 | O | C-A1 | B41 | O | L27 |
| P3-669 | O | C-A1 | B41 | O | L29 |
| P3-670 | O | C-A1 | B41 | O | L36 |
| P3-671 | O | C-A1 | B41 | O | L40 |
| P3-672 | O | C-A1 | B41 | O | L53 |
| P3-673 | O | C-A1 | B41 | O | L59 |
| P3-674 | O | C-A1 | B41 | O | L65 |
| P3-675 | O | C-A1 | B41 | O | L68 |
| P3-676 | O | C-A1 | B41 | O | L119 |
| P3-677 | O | C-A1 | B41 | O | L128 |
| P3-678 | O | C-A1 | B41 | O | L148 |
| P3-679 | O | C-A2 | B41 | O | Single |
| | | | | | bond |
| P3-680 | O | C-A2 | B41 | O | O |
| P3-681 | O | C-A2 | B41 | O | L1 |
| P3-682 | O | C-A2 | B41 | O | L5 |
| P3-683 | O | C-A2 | B41 | O | L9 |
| P3-684 | O | C-A2 | B41 | O | L18 |
| P3-685 | O | C-A2 | B41 | O | L22 |
| P3-686 | O | C-A2 | B41 | O | L27 |
| P3-687 | O | C-A2 | B41 | O | L29 |
| P3-688 | O | C-A2 | B41 | O | L36 |
| P3-689 | O | C-A2 | B41 | O | L40 |
| P3-690 | O | C-A2 | B41 | O | L53 |
| P3-691 | O | C-A2 | B41 | O | L59 |
| P3-692 | O | C-A2 | B41 | O | L65 |
| P3-693 | O | C-A2 | B41 | O | L68 |
| P3-694 | O | C-A2 | B41 | O | L119 |
| P3-695 | O | C-A2 | B41 | O | L128 |
| P3-696 | O | C-A2 | B41 | O | L148 |
| P3-697 | O | C-A3 | B41 | O | Single |
| | | | | | bond |
| P3-698 | O | C-A3 | B41 | O | O |
| P3-699 | O | C-A3 | B41 | O | L1 |
| P3-700 | O | C-A3 | B41 | O | L5 |
| P3-701 | O | C-A3 | B41 | O | L9 |
| P3-702 | O | C-A3 | B41 | O | L18 |
| P3-703 | O | C-A3 | B41 | O | L22 |
| P3-704 | O | C-A3 | B41 | O | L27 |
| P3-705 | O | C-A3 | B41 | O | L29 |
| P3-706 | O | C-A3 | B41 | O | L36 |
| P3-707 | O | C-A3 | B41 | O | L40 |
| P3-708 | O | C-A3 | B41 | O | L53 |
| P3-709 | O | C-A3 | B41 | O | L59 |
| P3-710 | O | C-A3 | B41 | O | L65 |
| P3-711 | O | C-A3 | B41 | O | L68 |
| P3-712 | O | C-A3 | B41 | O | L119 |
| P3-713 | O | C-A3 | B41 | O | L128 |
| P3-714 | O | C-A3 | B41 | O | L148 |
| P3-715 | O | C-B41 | B41 | O | Single |
| | | | | | bond |
| P3-716 | O | C-B41 | B41 | O | O |
| P3-717 | O | C-B41 | B41 | O | L1 |
| P3-718 | O | C-B41 | B41 | O | L5 |
| P3-719 | O | C-B41 | B41 | O | L9 |
| P3-720 | O | C-B41 | B41 | O | L18 |
| P3-721 | O | C-B41 | B41 | O | L22 |
| P3-722 | O | C-B41 | B41 | O | L27 |
| P3-723 | O | C-B41 | B41 | O | L29 |
| P3-724 | O | C-B41 | B41 | O | L36 |
| P3-725 | O | C-B41 | B41 | O | L40 |
| P3-726 | O | C-B41 | B41 | O | L53 |
| P3-727 | O | C-B41 | B41 | O | L59 |
| P3-728 | O | C-B41 | B41 | O | L65 |
| P3-729 | O | C-B41 | B41 | O | L68 |
| P3-730 | O | C-B41 | B41 | O | L119 |
| P3-731 | O | C-B41 | B41 | O | L128 |
| P3-732 | O | C-B41 | B41 | O | L148 |
| P3-733 | O | C-B56 | B41 | O | Single |
| | | | | | bond |
| P3-734 | O | C-B56 | B41 | O | O |
| P3-735 | O | C-B56 | B41 | O | L1 |
| P3-736 | O | C-B56 | B41 | O | L5 |
| P3-737 | O | C-B56 | B41 | O | L9 |
| P3-738 | O | C-B56 | B41 | O | L18 |
| P3-739 | O | C-B56 | B41 | O | L22 |
| P3-740 | O | C-B56 | B41 | O | L27 |
| P3-741 | O | C-B56 | B41 | O | L29 |
| P3-742 | O | C-B56 | B41 | O | L36 |
| P3-743 | O | C-B56 | B41 | O | L40 |
| P3-744 | O | C-B56 | B41 | O | L53 |
| P3-745 | O | C-B56 | B41 | O | L59 |
| P3-746 | O | C-B56 | B41 | O | L65 |
| P3-747 | O | C-B56 | B41 | O | L68 |
| P3-748 | O | C-B56 | B41 | O | L119 |
| P3-749 | O | C-B56 | B41 | O | L128 |
| P3-750 | O | C-B56 | B41 | O | L148 |
| P3-751 | O | N | B41 | O | Single |
| | | | | | bond |
| P3-752 | O | N | B41 | O | O |
| P3-753 | O | N | B41 | O | L1 |
| P3-754 | O | N | B41 | O | L5 |
| P3-755 | O | N | B41 | O | L9 |
| P3-756 | O | N | B41 | O | L18 |
| P3-757 | O | N | B41 | O | L22 |
| P3-758 | O | N | B41 | O | L27 |
| P3-759 | O | N | B41 | O | L29 |
| P3-760 | O | N | B41 | O | L36 |
| P3-761 | O | N | B41 | O | L40 |
| P3-762 | O | N | B41 | O | L53 |
| P3-763 | O | N | B41 | O | L59 |
| P3-764 | O | N | B41 | O | L65 |
| P3-765 | O | N | B41 | O | L68 |
| P3-766 | O | N | B41 | O | L119 |
| P3-767 | O | N | B41 | O | L128 |
| P3-768 | O | N | B41 | O | L148 |
| P3-769 | O | C-A1 | B56 | O | Single |
| | | | | | bond |
| P3-770 | O | C-A1 | B56 | O | O |
| P3-771 | O | C-A1 | B56 | O | L1 |
| P3-772 | O | C-A1 | B56 | O | L5 |
| P3-773 | O | C-A1 | B56 | O | L9 |
| P3-774 | O | C-A1 | B56 | O | L18 |
| P3-775 | O | C-A1 | B56 | O | L22 |
| P3-776 | O | C-A1 | B56 | O | L27 |
| P3-777 | O | C-A1 | B56 | O | L29 |
| P3-778 | O | C-A1 | B56 | O | L36 |
| P3-779 | O | C-A1 | B56 | O | L40 |
| P3-780 | O | C-A1 | B56 | O | L53 |
| P3-781 | O | C-A1 | B56 | O | L59 |
| P3-782 | O | C-A1 | B56 | O | L65 |
| P3-783 | O | C-A1 | B56 | O | L68 |
| P3-784 | O | C-A1 | B56 | O | L119 |
| P3-785 | O | C-A1 | B56 | O | L128 |
| P3-786 | O | C-A1 | B56 | O | L148 |
| P3-787 | O | C-A2 | B56 | O | Single |
| | | | | | bond |
| P3-788 | O | C-A2 | B56 | O | O |
| P3-789 | O | C-A2 | B56 | O | L1 |
| P3-790 | O | C-A2 | B56 | O | L5 |
| P3-791 | O | C-A2 | B56 | O | L9 |
| P3-792 | O | C-A2 | B56 | O | L18 |
| P3-793 | O | C-A2 | B56 | O | L22 |
| P3-794 | O | C-A2 | B56 | O | L27 |
| P3-795 | O | C-A2 | B56 | O | L29 |
| P3-796 | O | C-A2 | B56 | O | L36 |
| P3-797 | O | C-A2 | B56 | O | L40 |
| P3-798 | O | C-A2 | B56 | O | L53 |
| P3-799 | O | C-A2 | B56 | O | L59 |
| P3-800 | O | C-A2 | B56 | O | L65 |
| P3-801 | O | C-A2 | B56 | O | L68 |
| P3-802 | O | C-A2 | B56 | O | L119 |
| P3-803 | O | C-A2 | B56 | O | L128 |
| P3-804 | O | C-A2 | B56 | O | L148 |
| P3-805 | O | C-A3 | B56 | O | Single |
| | | | | | bond |
| P3-806 | O | C-A3 | B56 | O | O |
| P3-807 | O | C-A3 | B56 | O | L1 |
| P3-808 | O | C-A3 | B56 | O | L5 |
| P3-809 | O | C-A3 | B56 | O | L9 |
| P3-810 | O | C-A3 | B56 | O | L18 |
| P3-811 | O | C-A3 | B56 | O | L22 |
| P3-812 | O | C-A3 | B56 | O | L27 |
| P3-813 | O | C-A3 | B56 | O | L29 |
| P3-814 | O | C-A3 | B56 | O | L36 |
| P3-815 | O | C-A3 | B56 | O | L40 |
| P3-816 | O | C-A3 | B56 | O | L53 |
| P3-817 | O | C-A3 | B56 | O | L59 |
| P3-818 | O | C-A3 | B56 | O | L65 |
| P3-819 | O | C-A3 | B56 | O | L68 |
| P3-820 | O | C-A3 | B56 | O | L119 |
| P3-821 | O | C-A3 | B56 | O | L128 |
| P3-822 | O | C-A3 | B56 | O | L148 |
| P3-823 | O | C-B41 | B56 | O | Single |
| | | | | | bond |
| P3-824 | O | C-B41 | B56 | O | O |
| P3-825 | O | C-B41 | B56 | O | L1 |
| P3-826 | O | C-B41 | B56 | O | L5 |
| P3-827 | O | C-B41 | B56 | O | L9 |
| P3-828 | O | C-B41 | B56 | O | L18 |
| P3-829 | O | C-B41 | B56 | O | L22 |
| P3-830 | O | C-B41 | B56 | O | L27 |
| P3-831 | O | C-B41 | B56 | O | L29 |
| P3-832 | O | C-B41 | B56 | O | L36 |
| P3-833 | O | C-B41 | B56 | O | L40 |
| P3-834 | O | C-B41 | B56 | O | L53 |
| P3-835 | O | C-B41 | B56 | O | L59 |
| P3-836 | O | C-B41 | B56 | O | L65 |
| P3-837 | O | C-B41 | B56 | O | L68 |
| P3-838 | O | C-B41 | B56 | O | L119 |
| P3-839 | O | C-B41 | B56 | O | L128 |
| P3-840 | O | C-B41 | B56 | O | L148 |
| P3-841 | O | C-B56 | B56 | O | Single |
| | | | | | bond |
| P3-842 | O | C-B56 | B56 | O | O |
| P3-843 | O | C-B56 | B56 | O | L1 |
| P3-844 | O | C-B56 | B56 | O | L5 |
| P3-845 | O | C-B56 | B56 | O | L9 |
| P3-846 | O | C-B56 | B56 | O | L18 |
| P3-847 | O | C-B56 | B56 | O | L22 |
| P3-848 | O | C-B56 | B56 | O | L27 |
| P3-849 | O | C-B56 | B56 | O | L29 |
| P3-850 | O | C-B56 | B56 | O | L36 |
| P3-851 | O | C-B56 | B56 | O | L40 |
| P3-852 | O | C-B56 | B56 | O | L53 |
| P3-853 | O | C-B56 | B56 | O | L59 |
| P3-854 | O | C-B56 | B56 | O | L65 |
| P3-855 | O | C-B56 | B56 | O | L68 |
| P3-856 | O | C-B56 | B56 | O | L119 |
| P3-857 | O | C-B56 | B56 | O | L128 |
| P3-858 | O | C-B56 | B56 | O | L148 |
| P3-859 | O | N | B56 | O | Single |
| | | | | | bond |
| P3-860 | O | N | B56 | O | O |
| P3-861 | O | N | B56 | O | L1 |
| P3-862 | O | N | B56 | O | L5 |
| P3-863 | O | N | B56 | O | L9 |
| P3-864 | O | N | B56 | O | L18 |
| P3-865 | O | N | B56 | O | L22 |
| P3-866 | O | N | B56 | O | L27 |
| P3-867 | O | N | B56 | O | L29 |
| P3-868 | O | N | B56 | O | L36 |
| P3-869 | O | N | B56 | O | L40 |
| P3-870 | O | N | B56 | O | L53 |
| P3-871 | O | N | B56 | O | L59 |
| P3-872 | O | N | B56 | O | L65 |
| P3-873 | O | N | B56 | O | L68 |
| P3-874 | O | N | B56 | O | L119 |
| P3-875 | O | N | B56 | O | L128 |
| P3-876 | O | N | B56 | O | L148 |
| P3-877 | O | C-A1 | A2 | S | Single |
| | | | | | bond |
| P3-878 | O | C-A1 | A2 | S | L5 |
| P3-879 | O | C-A1 | A2 | S | L6 |
| P3-880 | O | C-A1 | A2 | S | L27 |
| P3-881 | O | C-A1 | A2 | S | L29 |
| P3-882 | O | C-A1 | A2 | S | L36 |
| P3-883 | O | C-B41 | A2 | S | Single |
| | | | | | bond |
| P3-884 | O | C-B41 | A2 | S | L5 |
| P3-885 | O | C-B41 | A2 | S | L6 |
| P3-886 | O | C-B41 | A2 | S | L27 |
| P3-887 | O | C-B41 | A2 | S | L29 |
| P3-888 | O | C-B41 | A2 | S | L36 |
| P3-889 | O | C-A1 | A3 | S | Single |
| | | | | | bond |
| P3-890 | O | C-A1 | A3 | S | L5 |
| P3-891 | O | C-A1 | A3 | S | L6 |
| P3-892 | O | C-A1 | A3 | S | L27 |
| P3-893 | O | C-A1 | A3 | S | L29 |
| P3-894 | O | C-A1 | A3 | S | L36 |
| P3-895 | O | C-B41 | A3 | S | Single |
| | | | | | bond |
| P3-896 | O | C-B41 | A3 | S | L5 |
| P3-897 | O | C-B41 | A3 | S | L6 |
| P3-898 | O | C-B41 | A3 | S | L27 |
| P3-899 | O | C-B41 | A3 | S | L29 |
| P3-900 | O | C-B41 | A3 | S | L36 |
| P3-901 | O | C-A1 | A2 | Se | L6 |
| P3-902 | O | C-A1 | A2 | Se | L36 |
| P3-903 | O | C-B41 | A2 | Se | L6 |
| P3-904 | O | C-B41 | A2 | Se | L36 |
| P3-905 | O | C-A1 | A3 | Se | L6 |
| P3-906 | O | C-A1 | A3 | Se | L36 |
| P3-907 | O | C-B41 | A3 | Se | L6 |
| P3-908 | O | C-B41 | A3 | Se | L36 |
| P3-909 | O | C-A1 | A2 | S═O | L6 |
| P3-910 | O | C-A1 | A2 | S═O | L36 |
| P3-911 | O | C-B41 | A2 | S═O | L6 |
| P3-912 | O | C-B41 | A2 | S═O | L36 |
| P3-913 | O | C-A1 | A3 | S═O | L6 |
| P3-914 | O | C-A1 | A3 | S═O | L36 |
| P3-915 | O | C-B41 | A3 | S═O | L6 |
| P3-916 | O | C-B41 | A3 | S═O | L36 |
| P3-917 | O | C-A1 | A2 | O═S═O | Single |
| | | | | | bond |
| P3-918 | O | C-A1 | A2 | O═S═O | L5 |
| P3-919 | O | C-A1 | A2 | O═S═O | L6 |
| P3-920 | O | C-A1 | A2 | O═S═O | L27 |
| P3-921 | O | C-A1 | A2 | O═S═O | L29 |
| P3-922 | O | C-A1 | A2 | O═S═O | L36 |
| P3-923 | O | C-B41 | A2 | O═S═O | Single |
| | | | | | bond |
| P3-924 | O | C-B41 | A2 | O═S═O | L5 |
| P3-925 | O | C-B41 | A2 | O═S═O | L6 |
| P3-926 | O | C-B41 | A2 | O═S═O | L27 |
| P3-927 | O | C-B41 | A2 | O═S═O | L29 |
| P3-928 | O | C-B41 | A2 | O═S═O | L36 |
| P3-929 | O | C-A1 | A3 | O═S═O | Single |
| | | | | | bond |
| P3-930 | O | C-A1 | A3 | O═S═O | L5 |
| P3-931 | O | C-A1 | A3 | O═S═O | L6 |
| P3-932 | O | C-A1 | A3 | O═S═O | L27 |
| P3-933 | O | C-A1 | A3 | O═S═O | L29 |
| P3-934 | O | C-A1 | A3 | O═S═O | L36 |
| P3-935 | O | C-B41 | A3 | O═S═O | Single |
| | | | | | bond |
| P3-936 | O | C-B41 | A3 | O═S═O | L5 |
| P3-937 | O | C-B41 | A3 | O═S═O | L6 |
| P3-938 | O | C-B41 | A3 | O═S═O | L27 |
| P3-939 | O | C-B41 | A3 | O═S═O | L29 |
| P3-940 | O | C-B41 | A3 | O═S═O | L36 |
| P3-941 | S | C-A1 | A2 | O | Single |
| | | | | | bond |
| P3-942 | S | C-A1 | A2 | O | L5 |
| P3-943 | S | C-A1 | A2 | O | L6 |
| P3-944 | S | C-A1 | A2 | O | L27 |
| P3-945 | S | C-A1 | A2 | O | L29 |
| P3-946 | S | C-A1 | A2 | O | L36 |
| P3-947 | S | C-B41 | A2 | O | Single |
| | | | | | bond |
| P3-948 | S | C-B41 | A2 | O | L5 |
| P3-949 | S | C-B41 | A2 | O | L6 |
| P3-950 | S | C-B41 | A2 | O | L27 |
| P3-951 | S | C-B41 | A2 | O | L29 |
| P3-952 | S | C-B41 | A2 | O | L36 |
| bond | | | | | |
| P3-953 | S | C-A1 | A3 | O | Single |
| | | | | | bond |
| P3-954 | S | C-A1 | A3 | O | L5 |
| P3-955 | S | C-A1 | A3 | O | L6 |
| P3-956 | S | C-A1 | A3 | O | L27 |
| P3-957 | S | C-A1 | A3 | O | L29 |
| P3-958 | S | C-A1 | A3 | O | L36 |
| P3-959 | S | C-B41 | A3 | O | Single |
| | | | | | bond |
| P3-960 | S | C-B41 | A3 | O | L5 |
| P3-961 | S | C-B41 | A3 | O | L6 |
| P3-962 | S | C-B41 | A3 | O | L27 |
| P3-963 | S | C-B41 | A3 | O | L29 |
| P3-964 | S | C-B41 | A3 | O | L36 |
| P3-965 | Se | C-A1 | A2 | O | L5 |
| P3-966 | Se | C-A1 | A2 | O | L6 |
| P3-967 | Se | C-A1 | A2 | O | L27 |
| P3-968 | Se | C-A1 | A2 | O | L29 |
| P3-969 | Se | C-A1 | A2 | O | L36 |
| P3-970 | Se | C-B41 | A2 | O | L5 |
| P3-971 | Se | C-B41 | A2 | O | L6 |
| P3-972 | Se | C-B41 | A2 | O | L27 |
| P3-973 | Se | C-B41 | A2 | O | L29 |
| P3-974 | Se | C-B41 | A2 | O | L36 |
| P3-975 | Se | C-A1 | A3 | O | L5 |
| P3-976 | Se | C-A1 | A3 | O | L6 |
| P3-977 | Se | C-A1 | A3 | O | L27 |
| P3-978 | Se | C-A1 | A3 | O | L29 |
| P3-979 | Se | C-A1 | A3 | O | L36 |
| P3-980 | Se | C-B41 | A3 | O | L5 |
| P3-981 | Se | C-B41 | A3 | O | L6 |
| P3-982 | Se | C-B41 | A3 | O | L27 |
| P3-983 | Se | C-B41 | A3 | O | L29 |
| P3-984 | Se | C-B41 | A3 | O | L36 |
| P3-985 | S | C-A1 | A2 | O═S═O | Single |
| | | | | | bond |
| P3-986 | S | C-A1 | A2 | O═S═O | L5 |
| P3-987 | S | C-A1 | A2 | O═S═O | L6 |
| P3-988 | S | C-A1 | A2 | O═S═O | L27 |
| P3-989 | S | C-A1 | A2 | O═S═O | L29 |
| P3-990 | S | C-A1 | A2 | O═S═O | L36 |
| P3-991 | S | C-B41 | A2 | O═S═O | Single |
| | | | | | bond |
| P3-992 | S | C-B41 | A2 | O═S═O | L5 |
| P3-993 | S | C-B41 | A2 | O═S═O | L6 |
| P3-994 | S | C-B41 | A2 | O═S═O | L27 |
| P3-995 | S | C-B41 | A2 | O═S═O | L29 |
| P3-996 | S | C-B41 | A2 | O═S═O | L36 |
| P3-997 | S | C-A1 | A3 | O═S═O | Single |
| | | | | | bond |
| P3-998 | S | C-A1 | A3 | O═S═O | L5 |
| P3-999 | S | C-A1 | A3 | O═S═O | L6 |
| P3-1000 | S | C-A1 | A3 | O═S═O | L27 |
| P3-1001 | S | C-A1 | A3 | O═S═O | L29 |
| P3-1002 | S | C-A1 | A3 | O═S═O | L36 |
| P3-1003 | S | C-B41 | A3 | O═S═O | Single |
| | | | | | bond |
| P3-1004 | S | C-B41 | A3 | O═S═O | L5 |
| P3-1005 | S | C-B41 | A3 | O═S═O | L6 |
| P3-1006 | S | C-B41 | A3 | O═S═O | L27 |
| P3-1007 | S | C-B41 | A3 | O═S═O | L29 |
| P3-1008 | S | C-B41 | A3 | O═S═O | L36 |
| P3-1009 | Se | C-A1 | A2 | O═S═O | L5 |
| P3-1010 | Se | C-A1 | A2 | O═S═O | L6 |
| P3-1011 | Se | C-A1 | A2 | O═S═O | L27 |
| P3-1012 | Se | C-A1 | A2 | O═S═O | L29 |
| P3-1013 | Se | C-A1 | A2 | O═S═O | L36 |
| P3-1014 | Se | C-B41 | A2 | O═S═O | L5 |
| P3-1015 | Se | C-B41 | A2 | O═S═O | L6 |
| P3-1016 | Se | C-B41 | A2 | O═S═O | L27 |
| P3-1017 | Se | C-B41 | A2 | O═S═O | L29 |
| P3-1018 | Se | C-B41 | A2 | O═S═O | L36 |
| P3-1019 | Se | C-A1 | A3 | O═S═O | L5 |
| P3-1020 | Se | C-A1 | A3 | O═S═O | L6 |
| P3-1021 | Se | C-A1 | A3 | O═S═O | L27 |
| P3-1022 | Se | C-A1 | A3 | O═S═O | L29 |
| P3-1023 | Se | C-A1 | A3 | O═S═O | L36 |
| P3-1024 | Se | C-B41 | A3 | O═S═O | L5 |
| P3-1025 | Se | C-B41 | A3 | O═S═O | L6 |
| P3-1026 | Se | C-B41 | A3 | O═S═O | L27 |
| P3-1027 | Se | C-B41 | A3 | O═S═O | L29 |
| P3-1028 | Se | C-B41 | A3 | O═S═O | L36 |

the compounds P4-1 to P4-1028 have structures represented by formula I-2-2; wherein R¹ and R² are the same, and W, Z, R¹, R², X, and L correspond to atoms or groups selected from the following table, respectively:

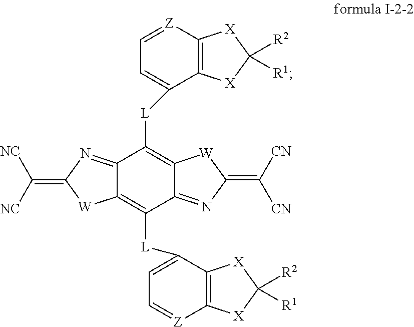

| | | | R¹ | | |
|---|---|---|---|---|---|
| | | | and | | |
| Compound No. | W | Z | R² | X | L |
| P4-1 | O | C-A1 | A1 | O | Single |
| | | | | | bond |
| P4-2 | O | C-A1 | A1 | O | O |
| P4-3 | O | C-A1 | A1 | O | L1 |
| P4-4 | O | C-A1 | A1 | O | L5 |
| P4-5 | O | C-A1 | A1 | O | L9 |
| P4-6 | O | C-A1 | A1 | O | L18 |
| P4-7 | O | C-A1 | A1 | O | L22 |
| P4-8 | O | C-A1 | A1 | O | L27 |
| P4-9 | O | C-A1 | A1 | O | L29 |
| P4-10 | O | C-A1 | A1 | O | L36 |
| P4-11 | O | C-A1 | A1 | O | L40 |
| P4-12 | O | C-A1 | A1 | O | L53 |
| P4-13 | O | C-A1 | A1 | O | L59 |
| P4-14 | O | C-A1 | A1 | O | L65 |
| P4-15 | O | C-A1 | A1 | O | L68 |
| P4-16 | O | C-A1 | A1 | O | L119 |
| P4-17 | O | C-A1 | A1 | O | L128 |
| P4-18 | O | C-A1 | A1 | O | L148 |
| P4-19 | O | C-A2 | A1 | O | Single |
| | | | | | bond |
| P4-20 | O | C-A2 | A1 | O | O |
| P4-21 | O | C-A2 | A1 | O | L1 |
| P4-22 | O | C-A2 | A1 | O | L5 |
| P4-23 | O | C-A2 | A1 | O | L9 |
| P4-24 | O | C-A2 | A1 | O | L18 |
| P4-25 | O | C-A2 | A1 | O | L22 |
| P4-26 | O | C-A2 | A1 | O | L27 |
| P4-27 | O | C-A2 | A1 | O | L29 |
| P4-28 | O | C-A2 | A1 | O | L36 |
| P4-29 | O | C-A2 | A1 | O | L40 |
| P4-30 | O | C-A2 | A1 | O | L53 |
| P4-31 | O | C-A2 | A1 | O | L59 |
| P4-32 | O | C-A2 | A1 | O | L65 |
| P4-33 | O | C-A2 | A1 | O | L68 |
| P4-34 | O | C-A2 | A1 | O | L119 |
| P4-35 | O | C-A2 | A1 | O | L128 |
| P4-36 | O | C-A2 | A1 | O | L148 |
| P4-37 | O | C-A3 | A1 | O | Single |
| | | | | | bond |
| P4-38 | O | C-A3 | A1 | O | O |
| P4-39 | O | C-A3 | A1 | O | L1 |
| P4-40 | O | C-A3 | A1 | O | L5 |
| P4-41 | O | C-A3 | A1 | O | L9 |
| P4-42 | O | C-A3 | A1 | O | L18 |
| P4-43 | O | C-A3 | A1 | O | L22 |
| P4-44 | O | C-A3 | A1 | O | L27 |
| P4-45 | O | C-A3 | A1 | O | L29 |
| P4-46 | O | C-A3 | A1 | O | L36 |
| P4-47 | O | C-A3 | A1 | O | L40 |
| P4-48 | O | C-A3 | A1 | O | L53 |
| P4-49 | O | C-A3 | A1 | O | L59 |
| P4-50 | O | C-A3 | A1 | O | L65 |
| P4-51 | O | C-A3 | A1 | O | L68 |
| P4-52 | O | C-A3 | A1 | O | L119 |
| P4-53 | O | C-A3 | A1 | O | L128 |
| P4-54 | O | C-A3 | A1 | O | L148 |
| P4-55 | O | C-B41 | A1 | O | Single |
| | | | | | bond |
| P4-56 | O | C-B41 | A1 | O | O |
| P4-57 | O | C-B41 | A1 | O | L1 |
| P4-58 | O | C-B41 | A1 | O | L5 |
| P4-59 | O | C-B41 | A1 | O | L9 |
| P4-60 | O | C-B41 | A1 | O | L18 |
| P4-61 | O | C-B41 | A1 | O | L22 |
| P4-62 | O | C-B41 | A1 | O | L27 |
| P4-63 | O | C-B41 | A1 | O | L29 |
| P4-64 | O | C-B41 | A1 | O | L36 |
| P4-65 | O | C-B41 | A1 | O | L40 |
| P4-66 | O | C-B41 | A1 | O | L53 |
| P4-67 | O | C-B41 | A1 | O | L59 |
| P4-68 | O | C-B41 | A1 | O | L65 |
| P4-69 | O | C-B41 | A1 | O | L68 |
| P4-70 | O | C-B41 | A1 | O | L119 |
| P4-71 | O | C-B41 | A1 | O | L128 |
| P4-72 | O | C-B41 | A1 | O | L148 |
| P4-73 | O | C-B56 | A1 | O | Single |
| | | | | | bond |
| P4-74 | O | C-B56 | A1 | O | C |
| P4-75 | O | C-B56 | A1 | O | L1 |
| P4-76 | O | C-B56 | A1 | O | L5 |
| P4-77 | O | C-B56 | A1 | O | L9 |
| P4-78 | O | C-B56 | A1 | O | L18 |
| P4-79 | O | C-B56 | A1 | O | L22 |
| P4-80 | O | C-B56 | A1 | O | L27 |
| P4-81 | O | C-B56 | A1 | O | L29 |
| P4-82 | O | C-B56 | A1 | O | L36 |
| P4-83 | O | C-B56 | A1 | O | L40 |
| P4-84 | O | C-B56 | A1 | O | L53 |
| P4-85 | O | C-B56 | A1 | O | L59 |
| P4-86 | O | C-B56 | A1 | O | L65 |
| P4-87 | O | C-B56 | A1 | O | L68 |
| P4-88 | O | C-B56 | A1 | O | L119 |
| P4-89 | O | C-B56 | A1 | O | L128 |
| P4-90 | O | C-B56 | A1 | O | L148 |
| P4-91 | O | N | A1 | O | Single |
| | | | | | bond |
| P4-92 | O | N | A1 | O | O |
| P4-93 | O | N | A1 | O | L1 |
| P4-94 | O | N | A1 | O | L5 |
| P4-95 | O | N | A1 | O | L9 |
| P4-96 | O | N | A1 | O | L18 |
| P4-97 | O | N | A1 | O | L22 |
| P4-98 | O | N | A1 | O | L27 |
| P4-99 | O | N | A1 | O | L29 |
| P4-100 | O | N | A1 | O | L36 |
| P4-101 | O | N | A1 | O | L40 |
| P4-102 | O | N | A1 | O | L53 |
| P4-103 | O | N | A1 | O | L59 |
| P4-104 | O | N | A1 | O | L65 |
| P4-105 | O | N | A1 | O | L68 |
| P4-106 | O | N | A1 | O | L119 |
| P4-107 | O | N | A1 | O | L128 |
| P4-108 | O | N | A1 | O | L148 |
| P4-109 | O | C-A1 | A2 | O | Single |
| | | | | | bond |
| P4-110 | O | C-A1 | A2 | O | O |
| P4-111 | O | C-A1 | A2 | O | L1 |
| P4-112 | O | C-A1 | A2 | O | L2 |
| P4-113 | O | C-A1 | A2 | O | L3 |
| P4-114 | O | C-A1 | A2 | O | L4 |
| P4-115 | O | C-A1 | A2 | O | L5 |
| P4-116 | O | C-A1 | A2 | O | L6 |
| P4-117 | O | C-A1 | A2 | O | L7 |
| P4-118 | O | C-A1 | A2 | O | L9 |
| P4-119 | O | C-A1 | A2 | O | L11 |
| P4-120 | O | C-A1 | A2 | O | L14 |
| P4-121 | O | C-A1 | A2 | O | L18 |
| P4-122 | O | C-A1 | A2 | O | L22 |
| P4-123 | O | C-A1 | A2 | O | L27 |
| P4-124 | O | C-A1 | A2 | O | L28 |
| P4-125 | O | C-A1 | A2 | O | L29 |
| P4-126 | O | C-A1 | A2 | O | L30 |
| P4-127 | O | C-A1 | A2 | O | L32 |
| P4-128 | O | C-A1 | A2 | O | L36 |
| P4-129 | O | C-A1 | A2 | O | L40 |
| P4-130 | O | C-A1 | A2 | O | L48 |
| P4-131 | O | C-A1 | A2 | O | L53 |
| P4-132 | O | C-A1 | A2 | O | L59 |
| P4-133 | O | C-A1 | A2 | O | L65 |
| P4-134 | O | C-A1 | A2 | O | L68 |
| P4-135 | O | C-A1 | A2 | O | L79 |
| P4-136 | O | C-A1 | A2 | O | L88 |
| P4-137 | O | C-A1 | A2 | O | L102 |
| P4-138 | O | C-A1 | A2 | O | L111 |
| P4-139 | O | C-A1 | A2 | O | L119 |
| P4-140 | O | C-A1 | A2 | O | L128 |
| P4-141 | O | C-A1 | A2 | O | L148 |
| P4-142 | O | C-A1 | A2 | O | L156 |
| P4-143 | O | C-A1 | A2 | O | L167 |
| P4-144 | O | C-A1 | A2 | O | L169 |
| P4-145 | O | C-A1 | A2 | O | L172 |
| P4-146 | O | C-A2 | A2 | O | Single |
| | | | | | bond |
| P4-147 | O | C-A2 | A2 | O | O |
| P4-148 | O | C-A2 | A2 | O | L1 |
| P4-149 | O | C-A2 | A2 | O | L2 |
| P4-150 | O | C-A2 | A2 | O | L3 |
| P4-151 | O | C-A2 | A2 | O | L4 |
| P4-152 | O | C-A2 | A2 | O | L5 |
| P4-153 | O | C-A2 | A2 | O | L6 |
| P4-154 | O | C-A2 | A2 | O | L7 |
| P4-155 | O | C-A2 | A2 | O | L9 |
| P4-156 | O | C-A2 | A2 | O | L11 |
| P4-157 | O | C-A2 | A2 | O | L14 |
| P4-158 | O | C-A2 | A2 | O | L18 |
| P4-159 | O | C-A2 | A2 | O | L22 |
| P4-160 | O | C-A2 | A2 | O | L27 |
| P4-161 | O | C-A2 | A2 | O | L28 |
| P4-162 | O | C-A2 | A2 | O | L29 |
| P4-163 | O | C-A2 | A2 | O | L30 |
| P4-164 | O | C-A2 | A2 | O | L32 |
| P4-165 | O | C-A2 | A2 | O | L36 |
| P4-166 | O | C-A2 | A2 | O | L40 |
| P4-167 | O | C-A2 | A2 | O | L48 |
| P4-168 | O | C-A2 | A2 | O | L53 |
| P4-169 | O | C-A2 | A2 | O | L59 |
| P4-170 | O | C-A2 | A2 | O | L65 |
| P4-171 | O | C-A2 | A2 | O | L68 |
| P4-172 | O | C-A2 | A2 | O | L79 |
| P4-173 | O | C-A2 | A2 | O | L89 |
| P4-174 | O | C-A2 | A2 | O | L102 |
| P4-175 | O | C-A2 | A2 | O | L111 |
| P4-176 | O | C-A2 | A2 | O | L119 |
| P4-177 | O | C-A2 | A2 | O | L128 |
| P4-178 | O | C-A2 | A2 | O | L148 |
| P4-179 | O | C-A2 | A2 | O | L156 |
| P4-180 | O | C-A2 | A2 | O | L167 |
| P4-181 | O | C-A2 | A2 | O | L169 |
| P4-182 | O | C-A2 | A2 | O | L172 |
| P4-183 | O | C-A3 | A2 | O | Single |
| | | | | | bond |
| P4-184 | O | C-A3 | A2 | O | O |
| P4-185 | O | C-A3 | A2 | O | L1 |
| P4-186 | O | C-A3 | A2 | O | L2 |
| P4-187 | O | C-A3 | A2 | O | L3 |
| P4-188 | O | C-A3 | A2 | O | L4 |
| P4-189 | O | C-A3 | A2 | O | L5 |
| P4-190 | O | C-A3 | A2 | O | L6 |
| P4-191 | O | C-A3 | A2 | O | L7 |
| P4-192 | O | C-A3 | A2 | O | L9 |
| P4-193 | O | C-A3 | A2 | O | L11 |
| P4-194 | O | C-A3 | A2 | O | L14 |
| P4-195 | O | C-A3 | A2 | O | L18 |
| P4-196 | O | C-A3 | A2 | O | L22 |
| P4-197 | O | C-A3 | A2 | O | L27 |
| P4-198 | O | C-A3 | A2 | O | L28 |
| P4-199 | O | C-A3 | A2 | O | L29 |
| P4-200 | O | C-A3 | A2 | O | L30 |
| P4-201 | O | C-A3 | A2 | O | L32 |
| P4-202 | O | C-A3 | A2 | O | L36 |
| P4-203 | O | C-A3 | A2 | O | L40 |
| P4-204 | O | C-A3 | A2 | O | L48 |
| P4-205 | O | C-A3 | A2 | O | L53 |
| P4-206 | O | C-A3 | A2 | O | L59 |
| P4-207 | O | C-A3 | A2 | O | L65 |
| P4-208 | O | C-A3 | A2 | O | L68 |
| P4-209 | O | C-A3 | A2 | O | L79 |
| P4-210 | O | C-A3 | A2 | O | L89 |
| P4-211 | O | C-A3 | A2 | O | L102 |
| P4-212 | O | C-A3 | A2 | O | L111 |
| P4-213 | O | C-A3 | A2 | O | L119 |
| P4-214 | O | C-A3 | A2 | O | L128 |
| P4-215 | O | C-A3 | A2 | O | L148 |
| P4-216 | O | C-A3 | A2 | O | L156 |
| P4-217 | O | C-A3 | A2 | O | L167 |
| P4-218 | O | C-A3 | A2 | O | L169 |
| P4-219 | O | C-A3 | A2 | O | L172 |
| P4-220 | O | C-B41 | A2 | O | Single |
| | | | | | bond |
| P4-221 | O | C-B41 | A2 | O | O |
| P4-222 | O | C-B41 | A2 | O | L1 |
| P4-223 | O | C-B41 | A2 | O | L2 |
| P4-224 | O | C-B41 | A2 | O | L3 |
| P4-225 | O | C-B41 | A2 | O | L4 |
| P4-226 | O | C-B41 | A2 | O | L5 |
| P4-227 | O | C-B41 | A2 | O | L6 |
| P4-228 | O | C-B41 | A2 | O | L7 |
| P4-229 | O | C-B41 | A2 | O | L9 |
| P4-230 | O | C-B41 | A2 | O | L11 |
| P4-231 | O | C-B41 | A2 | O | L14 |
| P4-232 | O | C-B41 | A2 | O | L18 |
| P4-233 | O | C-B41 | A2 | O | L22 |
| P4-234 | O | C-B41 | A2 | O | L27 |
| P4-235 | O | C-B41 | A2 | O | L28 |
| P4-236 | O | C-B41 | A2 | O | L29 |
| P4-237 | O | C-B41 | A2 | O | L30 |
| P4-238 | O | C-B41 | A2 | O | L32 |
| P4-239 | O | C-B41 | A2 | O | L36 |
| P4-240 | O | C-B41 | A2 | O | L40 |
| P4-241 | O | C-B41 | A2 | O | L48 |
| P4-242 | O | C-B41 | A2 | O | L53 |
| P4-243 | O | C-B41 | A2 | O | L59 |
| P4-244 | O | C-B41 | A2 | O | L65 |
| P4-245 | O | C-B41 | A2 | O | L68 |
| P4-246 | O | C-B41 | A2 | O | L79 |
| P4-247 | O | C-B41 | A2 | O | L89 |
| P4-248 | O | C-B41 | A2 | O | L102 |
| P4-249 | O | C-B41 | A2 | O | L111 |
| P4-250 | O | C-B41 | A2 | O | L119 |
| P4-251 | O | C-B41 | A2 | O | L128 |
| P4-252 | O | C-B41 | A2 | O | L148 |
| P4-253 | O | C-B41 | A2 | O | L156 |
| P4-254 | O | C-B41 | A2 | O | L167 |
| P4-255 | O | C-B41 | A2 | O | L169 |
| P4-256 | O | C-B41 | A2 | O | L172 |
| P4-257 | O | C-B56 | A2 | O | Single |
| | | | | | bond |
| P4-258 | O | C-B56 | A2 | O | O |
| P4-259 | O | C-B56 | A2 | O | L1 |
| P4-260 | O | C-B56 | A2 | O | L2 |
| P4-261 | O | C-B56 | A2 | O | L3 |
| P4-262 | O | C-B56 | A2 | O | L4 |
| P4-263 | O | C-B56 | A2 | O | L5 |
| P4-264 | O | C-B56 | A2 | O | L6 |
| P4-265 | O | C-B56 | A2 | O | L7 |
| P4-266 | O | C-B56 | A2 | O | L9 |
| P4-267 | O | C-B56 | A2 | O | L11 |
| P4-268 | O | C-B56 | A2 | O | L14 |
| P4-269 | O | C-B56 | A2 | O | L18 |
| P4-270 | O | C-B56 | A2 | O | L22 |
| P4-271 | O | C-B56 | A2 | O | L27 |
| P4-272 | O | C-B56 | A2 | O | L28 |
| P4-273 | O | C-B56 | A2 | O | L29 |
| P4-274 | O | C-B56 | A2 | O | L30 |
| P4-275 | O | C-B56 | A2 | O | L32 |
| P4-276 | O | C-B56 | A2 | O | L36 |
| P4-277 | O | C-B56 | A2 | O | L40 |
| P4-278 | O | C-B56 | A2 | O | L48 |
| P4-279 | O | C-B56 | A2 | O | L53 |
| P4-280 | O | C-B56 | A2 | O | L59 |
| P4-281 | O | C-B56 | A2 | O | L65 |
| P4-282 | O | C-B56 | A2 | O | L68 |
| P4-283 | O | C-B56 | A2 | O | L79 |
| P4-284 | O | C-B56 | A2 | O | L89 |
| P4-285 | O | C-B56 | A2 | O | L102 |
| P4-286 | O | C-B56 | A2 | O | L111 |
| P4-287 | O | C-B56 | A2 | O | L119 |
| P4-288 | O | C-B56 | A2 | O | L128 |
| P4-289 | O | C-B56 | A2 | O | L148 |
| P4-290 | O | C-B56 | A2 | O | L156 |
| P4-291 | O | C-B56 | A2 | O | L167 |
| P4-292 | O | C-B56 | A2 | O | L169 |
| P4-293 | O | C-B56 | A2 | O | L172 |
| P4-294 | O | N | A2 | O | Single |
| | | | | | bond |
| P4-295 | O | N | A2 | O | O |
| P4-296 | O | N | A2 | O | L1 |
| P4-297 | O | N | A2 | O | L2 |
| P4-298 | O | N | A2 | O | L3 |
| P4-299 | O | N | A2 | O | L4 |
| P4-300 | O | N | A2 | O | L5 |
| P4-301 | O | N | A2 | O | L6 |
| P4-302 | O | N | A2 | O | L7 |
| P4-303 | O | N | A2 | O | L9 |
| P4-304 | O | N | A2 | O | L11 |
| P4-305 | O | N | A2 | O | L14 |
| P4-306 | O | N | A2 | O | L18 |
| P4-307 | O | N | A2 | O | L22 |
| P4-308 | O | N | A2 | O | L27 |
| P4-309 | O | N | A2 | O | L28 |
| P4-310 | O | N | A2 | O | L29 |
| P4-311 | O | N | A2 | O | L30 |
| P4-312 | O | N | A2 | O | L32 |
| P4-313 | O | N | A2 | O | L36 |
| P4-314 | O | N | A2 | O | L40 |
| P4-315 | O | N | A2 | O | L48 |
| P4-316 | O | N | A2 | O | L53 |
| P4-317 | O | N | A2 | O | L59 |
| P4-318 | O | N | A2 | O | L65 |
| P4-319 | O | N | A2 | O | L68 |
| P4-320 | O | N | A2 | O | L79 |
| P4-321 | O | N | A2 | O | L89 |
| P4-322 | O | N | A2 | O | L102 |
| P4-323 | O | N | A2 | O | L111 |
| P4-324 | O | N | A2 | O | L119 |
| P4-325 | O | N | A2 | O | L128 |
| P4-326 | O | N | A2 | O | L148 |
| P4-327 | O | N | A2 | O | L156 |
| P4-328 | O | N | A2 | O | L167 |
| P4-329 | O | N | A2 | O | L169 |
| P4-330 | O | N | A2 | O | L172 |
| P4-331 | O | C-A1 | A3 | O | Single |
| | | | | | bond |
| P4-332 | O | C-A1 | A3 | O | O |
| P4-333 | O | C-A1 | A3 | O | L1 |
| P4-334 | O | C-A1 | A3 | O | L2 |
| P4-335 | O | C-A1 | A3 | O | L3 |
| P4-336 | O | C-A1 | A3 | O | L4 |
| P4-337 | O | C-A1 | A3 | O | L5 |
| P4-338 | O | C-A1 | A3 | O | L6 |
| P4-339 | O | C-A1 | A3 | O | L7 |
| P4-340 | O | C-A1 | A3 | O | L9 |
| P4-341 | O | C-A1 | A3 | O | L11 |
| P4-342 | O | C-A1 | A3 | O | L14 |
| P4-343 | O | C-A1 | A3 | O | L18 |
| P4-344 | O | C-A1 | A3 | O | L22 |
| P4-345 | O | C-A1 | A3 | O | L27 |
| P4-346 | O | C-A1 | A3 | O | L28 |
| P4-347 | O | C-A1 | A3 | O | L29 |
| P4-348 | O | C-A1 | A3 | O | L30 |
| P4-349 | O | C-A1 | A3 | O | L32 |
| P4-350 | O | C-A1 | A3 | O | L36 |
| P4-351 | O | C-A1 | A3 | O | L40 |
| P4-352 | O | C-A1 | A3 | O | L48 |
| P4-353 | O | C-A1 | A3 | O | L53 |
| P4-354 | O | C-A1 | A3 | O | L59 |
| P4-355 | O | C-A1 | A3 | O | L65 |
| P4-356 | O | C-A1 | A3 | O | L68 |
| P4-357 | O | C-A1 | A3 | O | L79 |
| P4-358 | O | C-A1 | A3 | O | L89 |
| P4-359 | O | C-A1 | A3 | O | L102 |
| P4-360 | O | C-A1 | A3 | O | L111 |
| P4-361 | O | C-A1 | A3 | O | L119 |
| P4-362 | O | C-A1 | A3 | O | L128 |
| P4-363 | O | C-A1 | A3 | O | L148 |
| P4-364 | O | C-A1 | A3 | O | L156 |
| P4-365 | O | C-A1 | A3 | O | L167 |
| P4-366 | O | C-A1 | A3 | O | L169 |
| P4-367 | O | C-A1 | A3 | O | L172 |
| P4-368 | O | C-A2 | A3 | O | Single |
| | | | | | bond |
| P4-369 | O | C-A2 | A3 | O | O |
| P4-370 | O | C-A2 | A3 | O | L1 |
| P4-371 | O | C-A2 | A3 | O | L2 |
| P4-372 | O | C-A2 | A3 | O | L3 |
| P4-373 | O | C-A2 | A3 | O | L4 |
| P4-374 | O | C-A2 | A3 | O | L5 |
| P4-375 | O | C-A2 | A3 | O | L6 |
| P4-376 | O | C-A2 | A3 | O | L7 |
| P4-377 | O | C-A2 | A3 | O | L9 |
| P4-378 | O | C-A2 | A3 | O | L11 |
| P4-379 | O | C-A2 | A3 | O | L14 |
| P4-380 | O | C-A2 | A3 | O | L18 |
| P4-381 | O | C-A2 | A3 | O | L22 |
| P4-382 | O | C-A2 | A3 | O | L27 |
| P4-383 | O | C-A2 | A3 | O | L28 |
| P4-384 | O | C-A2 | A3 | O | L29 |
| P4-385 | O | C-A2 | A3 | O | L30 |
| P4-386 | O | C-A2 | A3 | O | L32 |
| P4-387 | O | C-A2 | A3 | O | L36 |
| P4-388 | O | C-A2 | A3 | O | L40 |
| P4-389 | O | C-A2 | A3 | O | L48 |
| P4-390 | O | C-A2 | A3 | O | L53 |
| P4-391 | O | C-A2 | A3 | O | L59 |
| P4-392 | O | C-A2 | A3 | O | L65 |
| P4-393 | O | C-A2 | A3 | O | L68 |
| P4-394 | O | C-A2 | A3 | O | L79 |
| P4-395 | O | C-A2 | A3 | O | L89 |
| P4-396 | O | C-A2 | A3 | O | L102 |
| P4-397 | O | C-A2 | A3 | O | L111 |
| P4-398 | O | C-A2 | A3 | O | L119 |
| P4-399 | O | C-A2 | A3 | O | L128 |
| P4-400 | O | C-A2 | A3 | O | L148 |
| P4-401 | O | C-A2 | A3 | O | L156 |
| P4-402 | O | C-A2 | A3 | O | L167 |
| P4-403 | O | C-A2 | A3 | O | L169 |
| P4-404 | O | C-A2 | A3 | O | L172 |
| P4-405 | O | C-A3 | A3 | O | Single |
| | | | | | bond |
| P4-406 | O | C-A3 | A3 | O | O |
| P4-407 | O | C-A3 | A3 | O | L1 |
| P4-408 | O | C-A3 | A3 | O | L2 |
| P4-409 | O | C-A3 | A3 | O | L3 |
| P4-410 | O | C-A3 | A3 | O | L4 |
| P4-411 | O | C-A3 | A3 | O | L5 |
| P4-412 | O | C-A3 | A3 | O | L6 |
| P4-413 | O | C-A3 | A3 | O | L7 |
| P4-414 | O | C-A3 | A3 | O | L9 |
| P4-415 | O | C-A3 | A3 | O | L11 |
| P4-416 | O | C-A3 | A3 | O | L14 |
| P4-417 | O | C-A3 | A3 | O | L18 |
| P4-418 | O | C-A3 | A3 | O | L22 |
| P4-419 | O | C-A3 | A3 | O | L27 |
| P4-420 | O | C-A3 | A3 | O | L28 |
| P4-421 | O | C-A3 | A3 | O | L29 |
| P4-422 | O | C-A3 | A3 | O | L30 |
| P4-423 | O | C-A3 | A3 | O | L32 |
| P4-424 | O | C-A3 | A3 | O | L36 |
| P4-425 | O | C-A3 | A3 | O | L40 |
| P4-426 | O | C-A3 | A3 | O | L48 |
| P4-427 | O | C-A3 | A3 | O | L53 |
| P4-428 | O | C-A3 | A3 | O | L59 |
| P4-429 | O | C-A3 | A3 | O | L65 |
| P4-430 | O | C-A3 | A3 | O | L68 |
| P4-431 | O | C-A3 | A3 | O | L79 |
| P4-432 | O | C-A3 | A3 | O | L89 |
| P4-433 | O | C-A3 | A3 | O | L102 |
| P4-434 | O | C-A3 | A3 | O | L111 |
| P4-435 | O | C-A3 | A3 | O | L119 |
| P4-436 | O | C-A3 | A3 | O | L128 |
| P4-437 | O | C-A3 | A3 | O | L148 |
| P4-438 | O | C-A3 | A3 | O | L156 |
| P4-439 | O | C-A3 | A3 | O | L167 |
| P4-440 | O | C-A3 | A3 | O | L169 |
| P4-441 | O | C-A3 | A3 | O | L172 |
| P4-442 | O | C-B41 | A3 | O | Single |
| | | | | | bond |
| P4-443 | O | C-B41 | A3 | O | O |
| P4-444 | O | C-B41 | A3 | O | L1 |
| P4-445 | O | C-B41 | A3 | O | L2 |
| P4-446 | O | C-B41 | A3 | O | L3 |
| P4-447 | O | C-B41 | A3 | O | L4 |
| P4-448 | O | C-B41 | A3 | O | L5 |
| P4-449 | O | C-B41 | A3 | O | L6 |
| P4-450 | O | C-B41 | A3 | O | L7 |
| P4-451 | O | C-B41 | A3 | O | L9 |
| P4-452 | O | C-B41 | A3 | O | L11 |
| P4-453 | O | C-B41 | A3 | O | L14 |
| P4-454 | O | C-B41 | A3 | O | L18 |
| P4-455 | O | C-B41 | A3 | O | L22 |
| P4-456 | O | C-B41 | A3 | O | L27 |
| P4-457 | O | C-B41 | A3 | O | L28 |
| P4-458 | O | C-B41 | A3 | O | L29 |
| P4-459 | O | C-B41 | A3 | O | L30 |
| P4-460 | O | C-B41 | A3 | O | L32 |
| P4-461 | O | C-B41 | A3 | O | L36 |
| P4-462 | O | C-B41 | A3 | O | L40 |
| P4-463 | O | C-B41 | A3 | O | L48 |
| P4-464 | O | C-B41 | A3 | O | L53 |
| P4-465 | O | C-B41 | A3 | O | L59 |
| P4-466 | O | C-B41 | A3 | O | L65 |
| P4-467 | O | C-B41 | A3 | O | L68 |
| P4-468 | O | C-B41 | A3 | O | L79 |
| P4-469 | O | C-B41 | A3 | O | L89 |
| P4-470 | O | C-B41 | A3 | O | L102 |
| P4-471 | O | C-B41 | A3 | O | L111 |
| P4-472 | O | C-B41 | A3 | O | L119 |
| P4-473 | O | C-B41 | A3 | O | L128 |
| P4-474 | O | C-B41 | A3 | O | L148 |
| P4-475 | O | C-B41 | A3 | O | L156 |
| P4-476 | O | C-B41 | A3 | O | L167 |
| P4-477 | O | C-B41 | A3 | O | L169 |
| P4-478 | O | C-B41 | A3 | O | L172 |
| P4-479 | O | C-B56 | A3 | O | Single |
| | | | | | bond |
| P4-480 | O | C-B56 | A3 | O | O |
| P4-481 | O | C-B56 | A3 | O | L1 |
| P4-482 | O | C-B56 | A3 | O | L2 |
| P4-483 | O | C-B56 | A3 | O | L3 |
| P4-484 | O | C-B56 | A3 | O | L4 |
| P4-485 | O | C-B56 | A3 | O | L5 |
| P4-486 | O | C-B56 | A3 | O | L6 |
| P4-487 | O | C-B56 | A3 | O | L7 |
| P4-488 | O | C-B56 | A3 | O | L9 |
| P4-489 | O | C-B56 | A3 | O | L11 |
| P4-490 | O | C-B56 | A3 | O | L14 |
| P4-491 | O | C-B56 | A3 | O | L18 |
| P4-492 | O | C-B56 | A3 | O | L22 |
| P4-493 | O | C-B56 | A3 | O | L27 |
| P4-494 | O | C-B56 | A3 | O | L28 |
| P4-495 | O | C-B56 | A3 | O | L29 |
| P4-496 | O | C-B56 | A3 | O | L30 |
| P4-497 | O | C-B56 | A3 | O | L32 |
| P4-498 | O | C-B56 | A3 | O | L36 |
| P4-499 | O | C-B56 | A3 | O | L40 |
| P4-500 | O | C-B56 | A3 | O | L48 |
| P4-501 | O | C-B56 | A3 | O | L53 |
| P4-502 | O | C-B56 | A3 | O | L59 |
| P4-503 | O | C-B56 | A3 | O | L65 |
| P4-504 | O | C-B56 | A3 | O | L68 |
| P4-505 | O | C-B56 | A3 | O | L79 |
| P4-506 | O | C-B56 | A3 | O | L89 |
| P4-507 | O | C-B56 | A3 | O | L102 |
| P4-508 | O | C-B56 | A3 | O | L111 |
| P4-509 | O | C-B56 | A3 | O | L119 |
| P4-510 | O | C-B56 | A3 | O | L128 |
| P4-511 | O | C-B56 | A3 | O | L148 |
| P4-512 | O | C-B56 | A3 | O | L156 |
| P4-513 | O | C-B56 | A3 | O | L167 |
| P4-514 | O | C-B56 | A3 | O | L169 |
| P4-515 | O | C-B56 | A3 | O | L172 |
| P4-516 | O | N | A3 | O | Single |
| | | | | | bond |
| P4-517 | O | N | A3 | O | O |
| P4-518 | O | N | A3 | O | L1 |
| P4-519 | O | N | A3 | O | L2 |
| P4-520 | O | N | A3 | O | L3 |
| P4-521 | O | N | A3 | O | L4 |
| P4-522 | O | N | A3 | O | L5 |
| P4-523 | O | N | A3 | O | L6 |
| P4-524 | O | N | A3 | O | L7 |
| P4-525 | O | N | A3 | O | L9 |
| P4-526 | O | N | A3 | O | L11 |
| P4-527 | O | N | A3 | O | L14 |
| P4-528 | O | N | A3 | O | L18 |
| P4-529 | O | N | A3 | O | L22 |
| P4-530 | O | N | A3 | O | L27 |
| P4-531 | O | N | A3 | O | L28 |
| P4-532 | O | N | A3 | O | L29 |
| P4-533 | O | N | A3 | O | L30 |
| P4-534 | O | N | A3 | O | L32 |
| P4-535 | O | N | A3 | O | L36 |
| P4-536 | O | N | A3 | O | L40 |
| P4-537 | O | N | A3 | O | L48 |
| P4-538 | O | N | A3 | O | L53 |
| P4-539 | O | N | A3 | O | L59 |
| P4-540 | O | N | A3 | O | L65 |
| P4-541 | O | N | A3 | O | L68 |
| P4-542 | O | N | A3 | O | L79 |
| P4-543 | O | N | A3 | O | L89 |
| P4-544 | O | N | A3 | O | L102 |
| P4-545 | O | N | A3 | O | L111 |
| P4-546 | O | N | A3 | O | L119 |
| P4-547 | O | N | A3 | O | L128 |
| P4-548 | O | N | A3 | O | L148 |
| P4-549 | O | N | A3 | O | L156 |
| P4-550 | O | N | A3 | O | L167 |
| P4-551 | O | N | A3 | O | L169 |
| P4-552 | O | N | A3 | O | L172 |
| P4-553 | O | C-A1 | A12 | O | Single |
| | | | | | bond |
| P4-554 | O | C-A1 | A12 | O | O |
| P4-555 | O | C-A1 | A12 | O | L1 |
| P4-556 | O | C-A1 | A12 | O | L5 |
| P4-557 | O | C-A1 | A12 | O | L9 |
| P4-558 | O | C-A1 | A12 | O | L18 |
| P4-559 | O | C-A1 | A12 | O | L22 |
| P4-560 | O | C-A1 | A12 | O | L27 |
| P4-561 | O | C-A1 | A12 | O | L29 |
| P4-562 | O | C-A1 | A12 | O | L36 |
| P4-563 | O | C-A1 | A12 | O | L40 |
| P4-564 | O | C-A1 | A12 | O | L53 |
| P4-565 | O | C-A1 | A12 | O | L59 |
| P4-566 | O | C-A1 | A12 | O | L65 |
| P4-567 | O | C-A1 | A12 | O | L68 |
| P4-568 | O | C-A1 | A12 | O | L119 |
| P4-569 | O | C-A1 | A12 | O | L128 |
| P4-570 | O | C-A1 | A12 | O | L148 |
| P4-571 | O | C-A2 | A12 | O | Single |
| | | | | | bond |
| P4-572 | O | C-A2 | A12 | O | O |
| P4-573 | O | C-A2 | A12 | O | L1 |
| P4-574 | O | C-A2 | A12 | O | L5 |
| P4-575 | O | C-A2 | A12 | O | L9 |
| P4-576 | O | C-A2 | A12 | O | L18 |
| P4-577 | O | C-A2 | A12 | O | L22 |
| P4-578 | O | C-A2 | A12 | O | L27 |
| P4-579 | O | C-A2 | A12 | O | L29 |
| P4-580 | O | C-A2 | A12 | O | L36 |
| P4-581 | O | C-A2 | A12 | O | L40 |
| P4-582 | O | C-A2 | A12 | O | L53 |
| P4-583 | O | C-A2 | A12 | O | L59 |
| P4-584 | O | C-A2 | A12 | O | L65 |
| P4-585 | O | C-A2 | A12 | O | L68 |
| P4-586 | O | C-A2 | A12 | O | L119 |
| P4-587 | O | C-A2 | A12 | O | L128 |
| P4-588 | O | C-A2 | A12 | O | L148 |
| P4-589 | O | C-A3 | A12 | O | Single |
| | | | | | bond |
| P4-590 | O | C-A3 | A12 | O | O |
| P4-591 | O | C-A3 | A12 | O | L1 |
| P4-592 | O | C-A3 | A12 | O | L5 |
| P4-593 | O | C-A3 | A12 | O | L9 |
| P4-594 | O | C-A3 | A12 | O | L18 |
| P4-595 | O | C-A3 | A12 | O | L22 |
| P4-596 | O | C-A3 | A12 | O | L27 |
| P4-597 | O | C-A3 | A12 | O | L29 |
| P4-598 | O | C-A3 | A12 | O | L36 |
| P4-599 | O | C-A3 | A12 | O | L40 |
| P4-600 | O | C-A3 | A12 | O | L53 |
| P4-601 | O | C-A3 | A12 | O | L59 |
| P4-602 | O | C-A3 | A12 | O | L65 |
| P4-603 | O | C-A3 | A12 | O | L68 |
| P4-604 | O | C-A3 | A12 | O | L119 |
| P4-605 | O | C-A3 | A12 | O | L128 |
| P4-606 | O | C-A3 | A12 | O | L148 |
| P4-607 | O | C-B41 | A12 | O | Single |
| | | | | | bond |
| P4-608 | O | C-B41 | A12 | O | O |
| P4-609 | O | C-B41 | A12 | O | L1 |
| P4-610 | O | C-B41 | A12 | O | L5 |
| P4-611 | O | C-B41 | A12 | O | L9 |
| P4-612 | O | C-B41 | A12 | O | L18 |
| P4-613 | O | C-B41 | A12 | O | L22 |
| P4-614 | O | C-B41 | A12 | O | L27 |
| P4-615 | O | C-B41 | A12 | O | L29 |
| P4-616 | O | C-B41 | A12 | O | L36 |
| P4-617 | O | C-B41 | A12 | O | L40 |
| P4-618 | O | C-B41 | A12 | O | L53 |
| P4-619 | O | C-B41 | A12 | O | L59 |
| P4-620 | O | C-B41 | A12 | O | L65 |
| P4-621 | O | C-B41 | A12 | O | L68 |
| P4-622 | O | C-B41 | A12 | O | L119 |
| P4-623 | O | C-B41 | A12 | O | L128 |
| P4-624 | O | C-B41 | A12 | O | L148 |
| P4-625 | O | C-B56 | A12 | O | Single |
| | | | | | bond |
| P4-626 | O | C-B56 | A12 | O | O |
| P4-627 | O | C-B56 | A12 | O | L1 |
| P4-628 | O | C-B56 | A12 | O | L5 |
| P4-629 | O | C-B56 | A12 | O | L9 |
| P4-630 | O | C-B56 | A12 | O | L18 |
| P4-631 | O | C-B56 | A12 | O | L22 |
| P4-632 | O | C-B56 | A12 | O | L27 |
| P4-633 | O | C-B56 | A12 | O | L29 |
| P4-634 | O | C-B56 | A12 | O | L36 |
| P4-635 | O | C-B56 | A12 | O | L40 |
| P4-636 | O | C-B56 | A12 | O | L53 |
| P4-637 | O | C-B56 | A12 | O | L59 |
| P4-638 | O | C-B56 | A12 | O | L65 |
| P4-639 | O | C-B56 | A12 | O | L68 |
| P4-640 | O | C-B56 | A12 | O | L119 |
| P4-641 | O | C-B56 | A12 | O | L128 |
| P4-642 | O | C-B56 | A12 | O | L148 |
| P4-643 | O | N | A12 | O | Single |
| | | | | | bond |
| P4-644 | O | N | A12 | O | O |
| P4-645 | O | N | A12 | O | L1 |
| P4-646 | O | N | A12 | O | L5 |
| P4-647 | O | N | A12 | O | L9 |
| P4-648 | O | N | A12 | O | L18 |
| P4-649 | O | N | A12 | O | L22 |
| P4-650 | O | N | A12 | O | L27 |
| P4-651 | O | N | A12 | O | L29 |
| P4-652 | O | N | A12 | O | L36 |
| P4-653 | O | N | A12 | O | L40 |
| P4-654 | O | N | A12 | O | L53 |
| P4-655 | O | N | A12 | O | L59 |
| P4-656 | O | N | A12 | O | L65 |
| P4-657 | O | N | A12 | O | L68 |
| P4-658 | O | N | A12 | O | L119 |
| P4-659 | O | N | A12 | O | L128 |
| P4-660 | O | N | A12 | O | L148 |
| P4-661 | O | C-A1 | B41 | O | Single |
| | | | | | bond |
| P4-662 | O | C-A1 | B41 | O | O |
| P4-663 | O | C-A1 | B41 | O | L1 |
| P4-664 | O | C-A1 | B41 | O | L5 |
| P4-665 | O | C-A1 | B41 | O | L9 |
| P4-666 | O | C-A1 | B41 | O | L18 |
| P4-667 | O | C-A1 | B41 | O | L22 |
| P4-668 | O | C-A1 | B41 | O | L27 |
| P4-669 | O | C-A1 | B41 | O | L29 |
| P4-670 | O | C-A1 | B41 | O | L36 |
| P4-671 | O | C-A1 | B41 | O | L40 |
| P4-672 | O | C-A1 | B41 | O | L53 |
| P4-673 | O | C-A1 | B41 | O | L59 |
| P4-674 | O | C-A1 | B41 | O | L65 |
| P4-675 | O | C-A1 | B41 | O | L68 |
| P4-676 | O | C-A1 | B41 | O | L119 |
| P4-677 | O | C-A1 | B41 | O | L128 |
| P4-678 | O | C-A1 | B41 | O | L148 |
| P4-679 | O | C-A2 | B41 | O | Single |
| | | | | | bond |
| P4-680 | O | C-A2 | B41 | O | O |
| P4-681 | O | C-A2 | B41 | O | L1 |
| P4-682 | O | C-A2 | B41 | O | L5 |
| P4-683 | O | C-A2 | B41 | O | L9 |
| P4-684 | O | C-A2 | B41 | O | L18 |
| P4-685 | O | C-A2 | B41 | O | L22 |
| P4-686 | O | C-A2 | B41 | O | L27 |
| P4-687 | O | C-A2 | B41 | O | L29 |
| P4-688 | O | C-A2 | B41 | O | L36 |
| P4-689 | O | C-A2 | B41 | O | L40 |
| P4-690 | O | C-A2 | B41 | O | L53 |
| P4-691 | O | C-A2 | B41 | O | L59 |
| P4-692 | O | C-A2 | B41 | O | L65 |
| P4-693 | O | C-A2 | B41 | O | L68 |
| P4-694 | O | C-A2 | B41 | O | L119 |
| P4-695 | O | C-A2 | B41 | O | L128 |
| P4-696 | O | C-A2 | B41 | O | L148 |
| P4-697 | O | C-A3 | B41 | O | Single |
| | | | | | bond |
| P4-698 | O | C-A3 | B41 | O | O |
| P4-699 | O | C-A3 | B41 | O | L1 |
| P4-700 | O | C-A3 | B41 | O | L5 |
| P4-701 | O | C-A3 | B41 | O | L9 |
| P4-702 | O | C-A3 | B41 | O | L18 |
| P4-703 | O | C-A3 | B41 | O | L22 |
| P4-704 | O | C-A3 | B41 | O | L27 |
| P4-705 | O | C-A3 | B41 | O | L29 |
| P4-706 | O | C-A3 | B41 | O | L36 |
| P4-707 | O | C-A3 | B41 | O | L40 |
| P4-708 | O | C-A3 | B41 | O | L53 |
| P4-709 | O | C-A3 | B41 | O | L59 |
| P4-710 | O | C-A3 | B41 | O | L65 |
| P4-711 | O | C-A3 | B41 | O | L68 |
| P4-712 | O | C-A3 | B41 | O | L119 |
| P4-713 | O | C-A3 | B41 | O | L128 |
| P4-714 | O | C-A3 | B41 | O | L148 |
| P4-715 | O | C-B41 | B41 | O | Single |
| | | | | | bond |
| P4-716 | O | C-B41 | B41 | O | O |
| P4-717 | O | C-B41 | B41 | O | L1 |
| P4-718 | O | C-B41 | B41 | O | L5 |
| P4-719 | O | C-B41 | B41 | O | L9 |
| P4-720 | O | C-B41 | B41 | O | L18 |
| P4-721 | O | C-B41 | B41 | O | L22 |
| P4-722 | O | C-B41 | B41 | O | L27 |
| P4-723 | O | C-B41 | B41 | O | L29 |
| P4-724 | O | C-B41 | B41 | O | L36 |
| P4-725 | O | C-B41 | B41 | O | L40 |
| P4-726 | O | C-B41 | B41 | O | L53 |
| P4-727 | O | C-B41 | B41 | O | L59 |
| P4-728 | O | C-B41 | B41 | O | L65 |
| P4-729 | O | C-B41 | B41 | O | L68 |
| P4-730 | O | C-B41 | B41 | O | L119 |
| P4-731 | O | C-B41 | B41 | O | L128 |
| P4-732 | O | C-B41 | B41 | O | L148 |
| P4-733 | O | C-B56 | B41 | O | Single |
| | | | | | bond |
| P4-734 | O | C-B56 | B41 | O | O |
| P4-735 | O | C-B56 | B41 | O | L1 |
| P4-736 | O | C-B56 | B41 | O | L5 |
| P4-737 | O | C-B56 | B41 | O | L9 |
| P4-738 | O | C-B56 | B41 | O | L18 |
| P4-739 | O | C-B56 | B41 | O | L22 |
| P4-740 | O | C-B56 | B41 | O | L27 |
| P4-741 | O | C-B56 | B41 | O | L29 |
| P4-742 | O | C-B56 | B41 | O | L36 |
| P4-743 | O | C-B56 | B41 | O | L40 |
| P4-744 | O | C-B56 | B41 | O | L53 |
| P4-745 | O | C-B56 | B41 | O | L59 |
| P4-746 | O | C-B56 | B41 | O | L65 |
| P4-747 | O | C-B56 | B41 | O | L68 |
| P4-748 | O | C-B56 | B41 | O | L119 |
| P4-749 | O | C-B56 | B41 | O | L128 |
| P4-750 | O | C-B56 | B41 | O | L148 |
| P4-751 | O | N | B41 | O | Single |
| | | | | | bond |
| P4-752 | O | N | B41 | O | O |
| P4-753 | O | N | B41 | O | L1 |
| P4-754 | O | N | B41 | O | L5 |
| P4-755 | O | N | B41 | O | L9 |
| P4-756 | O | N | B41 | O | L18 |
| P4-757 | O | N | B41 | O | L22 |
| P4-758 | O | N | B41 | O | L27 |
| P4-759 | O | N | B41 | O | L29 |
| P4-760 | O | N | B41 | O | L36 |
| P4-761 | O | N | B41 | O | L40 |
| P4-762 | O | N | B41 | O | L53 |
| P4-763 | O | N | B41 | O | L59 |
| P4-764 | O | N | B41 | O | L65 |
| P4-765 | O | N | B41 | O | L68 |
| P4-766 | O | N | B41 | O | L119 |
| P4-767 | O | N | B41 | O | L128 |
| P4-768 | O | N | B41 | O | L148 |
| P4-769 | O | C-A1 | B56 | O | Single |
| | | | | | bond |
| P4-770 | O | C-A1 | B56 | O | O |
| P4-771 | O | C-A1 | B56 | O | L1 |
| P4-772 | O | C-A1 | B56 | O | L5 |
| P4-773 | O | C-A1 | B56 | O | L9 |
| P4-774 | O | C-A1 | B56 | O | L18 |
| P4-775 | O | C-A1 | B56 | O | L22 |
| P4-776 | O | C-A1 | B56 | O | L27 |
| P4-777 | O | C-A1 | B56 | O | L29 |
| P4-778 | O | C-A1 | B56 | O | L36 |
| P4-779 | O | C-A1 | B56 | O | L40 |
| P4-780 | O | C-A1 | B56 | O | L53 |
| P4-781 | O | C-A1 | B56 | O | L59 |
| P4-782 | O | C-A1 | B56 | O | L65 |
| P4-783 | O | C-A1 | B56 | O | L68 |
| P4-784 | O | C-A1 | B56 | O | L119 |
| P4-785 | O | C-A1 | B56 | O | L128 |
| P4-786 | O | C-A1 | B56 | O | L148 |
| P4-787 | O | C-A2 | B56 | O | Single |
| | | | | | bond |
| P4-788 | O | C-A2 | B56 | O | O |
| P4-789 | O | C-A2 | B56 | O | L1 |
| P4-790 | O | C-A2 | B56 | O | L5 |
| P4-791 | O | C-A2 | B56 | O | L9 |
| P4-792 | O | C-A2 | B56 | O | L18 |
| P4-793 | O | C-A2 | B56 | O | L22 |
| P4-794 | O | C-A2 | B56 | O | L27 |
| P4-795 | O | C-A2 | B56 | O | L29 |
| P4-796 | O | C-A2 | B56 | O | L36 |
| P4-797 | O | C-A2 | B56 | O | L40 |
| P4-798 | O | C-A2 | B56 | O | L53 |
| P4-799 | O | C-A2 | B56 | O | L59 |
| P4-800 | O | C-A2 | B56 | O | L65 |
| P4-801 | O | C-A2 | B56 | O | L68 |
| P4-802 | O | C-A2 | B56 | O | L119 |
| P4-803 | O | C-A2 | B56 | O | L128 |
| P4-804 | O | C-A2 | B56 | O | L148 |
| P4-805 | O | C-A3 | B56 | O | Single |
| | | | | | bond |
| P4-806 | O | C-A3 | B56 | O | O |
| P4-807 | O | C-A3 | B56 | O | L1 |
| P4-808 | O | C-A3 | B56 | O | L5 |
| P4-809 | O | C-A3 | B56 | O | L9 |
| P4-810 | O | C-A3 | B56 | O | L18 |
| P4-811 | O | C-A3 | B56 | O | L22 |
| P4-812 | O | C-A3 | B56 | O | L27 |
| P4-813 | O | C-A3 | B56 | O | L29 |
| P4-814 | O | C-A3 | B56 | O | L36 |
| P4-815 | O | C-A3 | B56 | O | L40 |
| P4-816 | O | C-A3 | B56 | O | L53 |
| P4-817 | O | C-A3 | B56 | O | L59 |
| P4-818 | O | C-A3 | B56 | O | L65 |
| P4-819 | O | C-A3 | B56 | O | L68 |
| P4-820 | O | C-A3 | B56 | O | L119 |
| P4-821 | O | C-A3 | B56 | O | L128 |
| P4-822 | O | C-A3 | B56 | O | L148 |
| P4-823 | O | C-B41 | B56 | O | Single |
| | | | | | bond |
| P4-824 | O | C-B41 | B56 | O | O |
| P4-825 | O | C-B41 | B56 | O | L1 |
| P4-826 | O | C-B41 | B56 | O | L5 |
| P4-827 | O | C-B41 | B56 | O | L9 |
| P4-828 | O | C-B41 | B56 | O | L18 |
| P4-829 | O | C-B41 | B56 | O | L22 |
| P4-830 | O | C-B41 | B56 | O | L27 |
| P4-831 | O | C-B41 | B56 | O | L29 |
| P4-832 | O | C-B41 | B56 | O | L36 |
| P4-833 | O | C-B41 | B56 | O | L40 |
| P4-834 | O | C-B41 | B56 | O | L53 |
| P4-835 | O | C-B41 | B56 | O | L59 |
| P4-836 | O | C-B41 | B56 | O | L65 |
| P4-837 | O | C-B41 | B56 | O | L68 |
| P4-838 | O | C-B41 | B56 | O | L119 |
| P4-839 | O | C-B41 | B56 | O | L128 |
| P4-840 | O | C-B41 | B56 | O | L148 |
| P4-841 | O | C-B56 | B56 | O | Single |
| | | | | | bond |
| P4-842 | O | C-B56 | B56 | O | O |
| P4-843 | O | C-B56 | B56 | O | L1 |
| P4-844 | O | C-B56 | B56 | O | L5 |
| P4-845 | O | C-B56 | B56 | O | L9 |
| P4-846 | O | C-B56 | B56 | O | L18 |
| P4-847 | O | C-B56 | B56 | O | L22 |
| P4-848 | O | C-B56 | B56 | O | L27 |
| P4-849 | O | C-B56 | B56 | O | L29 |
| P4-850 | O | C-B56 | B56 | O | L36 |
| P4-851 | O | C-B56 | B56 | O | L40 |
| P4-852 | O | C-B56 | B56 | O | L53 |
| P4-853 | O | C-B56 | B56 | O | L59 |
| P4-854 | O | C-B56 | B56 | O | L65 |
| P4-855 | O | C-B56 | B56 | O | L68 |
| P4-856 | O | C-B56 | B56 | O | L119 |
| P4-857 | O | C-B56 | B56 | O | L128 |
| P4-858 | O | C-B56 | B56 | O | L148 |
| P4-859 | O | N | B56 | O | Single |
| | | | | | bond |
| P4-860 | O | N | B56 | O | O |
| P4-861 | O | N | B56 | O | L1 |
| P4-862 | O | N | B56 | O | L5 |
| P4-863 | O | N | B56 | O | L9 |
| P4-864 | O | N | B56 | O | L18 |
| P4-865 | O | N | B56 | O | L22 |
| P4-866 | O | N | B56 | O | L27 |
| P4-867 | O | N | B56 | O | L29 |
| P4-868 | O | N | B56 | O | L36 |
| P4-869 | O | N | B56 | O | L40 |
| P4-870 | O | N | B56 | O | L53 |
| P4-871 | O | N | B56 | O | L59 |
| P4-872 | O | N | B56 | O | L65 |
| P4-873 | O | N | B56 | O | L68 |
| P4-874 | O | N | B56 | O | L119 |
| P4-875 | O | N | B56 | O | L128 |
| P4-876 | O | N | B56 | O | L148 |
| P4-877 | O | C-A1 | A2 | S | Single |
| | | | | | bond |
| P4-878 | O | C-A1 | A2 | S | L5 |
| P4-879 | O | C-A1 | A2 | S | L6 |
| P4-880 | O | C-A1 | A2 | S | L27 |
| P4-881 | O | C-A1 | A2 | S | L29 |
| P4-882 | O | C-A1 | A2 | S | L36 |
| P4-883 | O | C-B41 | A2 | S | Single |
| | | | | | bond |
| P4-884 | O | C-B41 | A2 | S | L5 |
| P4-885 | O | C-B41 | A2 | S | L6 |
| P4-886 | O | C-B41 | A2 | S | L27 |
| P4-887 | O | C-B41 | A2 | S | L29 |
| P4-888 | O | C-B41 | A2 | S | L36 |
| P4-889 | O | C-A1 | A3 | S | Single |
| | | | | | bond |
| P4-890 | O | C-A1 | A3 | S | L5 |
| P4-891 | O | C-A1 | A3 | S | L6 |
| P4-892 | O | C-A1 | A3 | S | L27 |
| P4-893 | O | C-A1 | A3 | S | L29 |
| P4-894 | O | C-A1 | A3 | S | L36 |
| P4-895 | O | C-B41 | A3 | S | Single |
| | | | | | bond |
| P4-896 | O | C-B41 | A3 | S | L5 |
| P4-897 | O | C-B41 | A3 | S | L6 |
| P4-898 | O | C-B41 | A3 | S | L27 |
| P4-899 | O | C-B41 | A3 | S | L29 |
| P4-900 | O | C-B41 | A3 | S | L36 |
| P4-901 | O | C-A1 | A2 | Se | L6 |
| P4-902 | O | C-A1 | A2 | Se | L36 |
| P4-903 | O | C-B41 | A2 | Se | L6 |
| P4-904 | O | C-B41 | A2 | Se | L36 |
| P4-905 | O | C-A1 | A3 | Se | L6 |
| P4-906 | O | C-A1 | A3 | Se | L36 |
| P4-907 | O | C-B41 | A3 | Se | L6 |
| P4-908 | O | C-B41 | A3 | Se | L36 |
| P4-909 | O | C-A1 | A2 | S═O | L6 |
| P4-910 | O | C-A1 | A2 | S═O | L36 |
| P4-911 | O | C-B41 | A2 | S═O | L6 |
| P4-912 | O | C-B41 | A2 | S═O | L36 |
| P4-913 | O | C-A1 | A3 | S═O | L6 |
| P4-914 | O | C-A1 | A3 | S═O | L36 |
| P4-915 | O | C-B41 | A3 | S═O | L6 |
| P4-916 | O | C-B41 | A3 | S═O | L36 |
| P4-917 | O | C-A1 | A2 | O═S═O | Single |
| | | | | | bond |
| P4-918 | O | C-A1 | A2 | O═S═O | L5 |
| P4-919 | O | C-A1 | A2 | O═S═O | L6 |
| P4-920 | O | C-A1 | A2 | O═S═O | L27 |
| P4-921 | O | C-A1 | A2 | O═S═O | L29 |
| P4-922 | O | C-A1 | A2 | O═S═O | L36 |
| P4-923 | O | C-B41 | A2 | O═S═O | Single |
| | | | | | bond |
| P4-924 | O | C-B41 | A2 | O═S═O | L5 |
| P4-925 | O | C-B41 | A2 | O═S═O | L6 |
| P4-926 | O | C-B41 | A2 | O═S═O | L27 |
| P4-927 | O | C-B41 | A2 | O═S═O | L29 |
| P4-928 | O | C-B41 | A2 | O═S═O | L36 |
| P4-929 | O | C-A1 | A3 | O═S═O | Single |
| | | | | | bond |
| P4-930 | O | C-A1 | A3 | O═S═O | L5 |
| P4-931 | O | C-A1 | A3 | O═S═O | L6 |
| P4-932 | O | C-A1 | A3 | O═S═O | L27 |
| P4-933 | O | C-A1 | A3 | O═S═O | L29 |
| P4-934 | O | C-A1 | A3 | O═S═O | L36 |
| P4-935 | O | C-B41 | A3 | O═S═O | Single |
| | | | | | bond |
| P4-936 | O | C-B41 | A3 | O═S═O | L5 |
| P4-937 | O | C-B41 | A3 | O═S═O | L6 |
| P4-938 | O | C-B41 | A3 | O═S═O | L27 |
| P4-939 | O | C-B41 | A3 | O═S═O | L29 |
| P4-940 | O | C-B41 | A3 | O═S═O | L36 |
| P4-941 | S | C-A1 | A2 | O | Single |
| | | | | | bond |
| P4-942 | S | C-A1 | A2 | O | L5 |
| P4-943 | S | C-A1 | A2 | O | L6 |
| P4-944 | S | C-A1 | A2 | O | L27 |
| P4-945 | S | C-A1 | A2 | O | L29 |
| P4-946 | S | C-A1 | A2 | O | L36 |
| P4-947 | S | C-B41 | A2 | O | Single |
| | | | | | bond |
| P4-948 | S | C-B41 | A2 | O | L5 |
| P4-949 | S | C-B41 | A2 | O | L6 |
| P4-950 | S | C-B41 | A2 | O | L27 |
| P4-951 | S | C-B41 | A2 | O | L29 |
| P4-952 | S | C-B41 | A2 | O | L36 |
| P4-953 | S | C-A1 | A3 | O | Single |
| | | | | | bond |
| P4-954 | S | C-A1 | A3 | O | L5 |
| P4-955 | S | C-A1 | A3 | O | L6 |
| P4-956 | S | C-A1 | A3 | O | L27 |
| P4-957 | S | C-A1 | A3 | O | L29 |
| P4-958 | S | C-A1 | A3 | O | L36 |
| P4-959 | S | C-B41 | A3 | O | Single |
| | | | | | bond |
| P4-960 | S | C-B41 | A3 | O | L5 |
| P4-961 | S | C-B41 | A3 | O | L6 |
| P4-962 | S | C-B41 | A3 | O | L27 |
| P4-963 | S | C-B41 | A3 | O | L29 |
| P4-964 | S | C-B41 | A3 | O | L36 |
| P4-965 | Se | C-A1 | A2 | O | L5 |
| P4-966 | Se | C-A1 | A2 | O | L6 |
| P4-967 | Se | C-A1 | A2 | O | L27 |
| P4-968 | Se | C-A1 | A2 | O | L29 |
| P4-969 | Se | C-A1 | A2 | O | L36 |
| P4-970 | Se | C-B41 | A2 | O | L5 |
| P4-971 | Se | C-B41 | A2 | O | L6 |
| P4-972 | Se | C-B41 | A2 | O | L27 |
| P4-973 | Se | C-B41 | A2 | O | L29 |
| P4-974 | Se | C-B41 | A2 | O | L36 |
| P4-975 | Se | C-A1 | A3 | O | L5 |
| P4-976 | Se | C-A1 | A3 | O | L6 |
| P4-977 | Se | C-A1 | A3 | O | L27 |
| P4-978 | Se | C-A1 | A3 | O | L29 |
| P4-979 | Se | C-A1 | A3 | O | L36 |
| P4-980 | Se | C-B41 | A3 | O | L5 |
| P4-981 | Se | C-B41 | A3 | O | L6 |
| P4-982 | Se | C-B41 | A3 | O | L27 |
| P4-983 | Se | C-B41 | A3 | O | L29 |
| P4-984 | Se | C-B41 | A3 | O | L36 |
| P4-985 | S | C-A1 | A2 | O═S═O | Single |
| | | | | | bond |
| P4-986 | S | C-A1 | A2 | O═S═O | L5 |
| P4-987 | S | C-A1 | A2 | O═S═O | L6 |
| P4-988 | S | C-A1 | A2 | O═S═O | L27 |
| P4-989 | S | C-A1 | A2 | O═S═O | L29 |
| P4-990 | S | C-A1 | A2 | O═S═O | L36 |
| P4-991 | S | C-B41 | A2 | O═S═O | Single |
| | | | | | bond |
| P4-992 | S | C-B41 | A2 | O═S═O | L5 |
| P4-993 | S | C-B41 | A2 | O═S═O | L6 |
| P4-994 | S | C-B41 | A2 | O═S═O | L27 |
| P4-995 | S | C-B41 | A2 | O═S═O | L29 |
| P4-996 | S | C-B41 | A2 | O═S═O | L36 |
| P4-997 | S | C-A1 | A3 | O═S═O | Single |
| | | | | | bond |
| P4-998 | S | C-A1 | A3 | O═S═O | L5 |
| P4-999 | S | C-A1 | A3 | O═S═O | L6 |
| P4-1000 | S | C-A1 | A3 | O═S═O | L27 |
| P4-1001 | S | C-A1 | A3 | O═S═O | L29 |
| P4-1002 | S | C-A1 | A3 | O═S═O | L36 |
| P4-1003 | S | C-B41 | A3 | O═S═O | Single |
| | | | | | bond |
| P4-1004 | S | C-B41 | A3 | O═S═O | L5 |
| P4-1005 | S | C-B41 | A3 | O═S═O | L6 |
| P4-1006 | S | C-B41 | A3 | O═S═O | L27 |
| P4-1007 | S | C-B41 | A3 | O═S═O | L29 |
| P4-1008 | S | C-B41 | A3 | O═S═O | L36 |
| P4-1009 | Se | C-A1 | A2 | O═S═O | L5 |
| P4-1010 | Se | C-A1 | A2 | O═S═O | L6 |
| P4-1011 | Se | C-A1 | A2 | O═S═O | L27 |
| P4-1012 | Se | C-A1 | A2 | O═S═O | L29 |
| P4-1013 | Se | C-A1 | A2 | O═S═O | L36 |
| P4-1014 | Se | C-B41 | A2 | O═S═O | L5 |
| P4-1015 | Se | C-B41 | A2 | O═S═O | L6 |
| P4-1016 | Se | C-B41 | A2 | O═S═O | L27 |
| P4-1017 | Se | C-B41 | A2 | O═S═O | L29 |
| P4-1018 | Se | C-B41 | A2 | O═S═O | L36 |
| P4-1019 | Se | C-A1 | A3 | O═S═O | L5 |
| P4-1020 | Se | C-A1 | A3 | O═S═O | L6 |
| P4-1021 | Se | C-A1 | A3 | O═S═O | L27 |
| P4-1022 | Se | C-A1 | A3 | O═S═O | L29 |
| P4-1023 | Se | C-A1 | A3 | O═S═O | L36 |
| P4-1024 | Se | C-B41 | A3 | O═S═O | L5 |
| P4-1025 | Se | C-B41 | A3 | O═S═O | L6 |
| P4-1026 | Se | C-B41 | A3 | O═S═O | L27 |
| P4-1027 | Se | C-B41 | A3 | O═S═O | L29 |
| P4-1028 | Se | C-B41 | A3 | O═S═O | L36 |

hydrogens in the compounds with the above structures can be partially or completely replaced by deuterium.

14. An organic electroluminescent device, characterized by comprising:
an anode,
a cathode,
and an organic layer arranged between the anode and the cathode, wherein the organic layer comprises the compound according to claim 1.

15. The organic electroluminescent device according to claim 14, characterized in that the organic layer is a hole injection layer or a hole transport layer, and the hole injection layer or the hole transport layer is formed from the compound according to claim 1 alone.

16. The electroluminescent device according to claim 14, characterized in that the organic layer is a hole injection layer or a hole transport layer, and the hole injection layer or the hole transport layer further comprises at least one hole transport material, the mass doping ratio of the organic compound to the at least one hole transport material being 10:1 to 1:100.

17. The organic electroluminescent device of claim 14, characterized in that the organic electroluminescent device comprises at least two light-emitting units, and the organic layer is a charge generation layer arranged between the at least two light-emitting units, wherein the charge generation layer comprises a p-type charge generation layer and an n-type charge generation layer;
the p-type charge generation layer comprises the compound according to claim 1; and
the p-type charge generation layer further comprises at least one hole transport material, the mass doping ratio of the organic compound to the at least one hole transport material being 10:1 to 1:100.

18. The organic electroluminescent device according to claim 17, characterized in that the charge generation layer further comprises a buffer layer arranged between the p-type charge generation layer and the n-type charge generation layer, and the buffer layer comprises the compound according to claim 1.

19. A display assembly, characterized by comprising the organic electroluminescent device according to claim 1.
